(19) United States
(12) Patent Application Publication
(10) Pub. No.: US 2009/0209022 A1
(43) Pub. Date: Aug. 20, 2009

(54) Crystal Structure of Ump Kinase and Uses Thereof

(75) Inventors: Jason Breed, Cheshire (GB); Peter Doig, Waltham, MA (US); Charles J. Eyermann, Waltham, MA (US); Richard Pauptit, Cheshire (GB); Julie Tucker, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/573,139

(22) PCT Filed: Aug. 15, 2005
(86) PCT No.: PCT/GB2005/003187

§ 371 Date: Feb. 2, 2007

Related U.S. Application Data
(60) Provisional application No. 60/601,765, filed on Aug. 16, 2004.

(51) Int. Cl. C12N 9/12; G06G 7/48

(52) U.S. Cl. 435/194; 703/12

(57) ABSTRACT

The present invention relates crystals of UMP kinase and computer-assisted methods for screening, identifying, and designing inhibitors and allosteric modulators of UMP kinase.

### FIELD OF THE INVENTION

The present invention relates to crystals of UMP kinase and computer-assisted methods for screening, identifying, and designing inhibitors and allosteric modulators of UMP kinase.

### BACKGROUND

UMP kinases catalyse the reversible transfer of a phosphate group from ATP to UMP to produce UDP, an essential step in the pyrimidine nucleotide synthesis pathway leading to the production of UTP. The primary structure of bacterial UMP kinases diverges sufficiently from that of other bacterial and eukaryotic nucleotide monophosphate kinases (Serina et al., 1995, Biochemistry, 34:5066-5074; Barzu et al., 1999, Paths to Pyrimidines, 7:86-95; Labesse et al., 2002, Biochem. Biophys. Res. Commun., 294:173-179; Gagyi et al., 2003, Eur. J. Biochem., 270:3196-3204) that they are of interest as targets for designing or identifying antibacterial agents. Eukaryotic UMP-CMP kinases are monomers in solution. The structures of several UMP-CMP kinases from eukaryotes have been determined (Mueller-Dieckmann et al., 1994, J. Biol., 236:362-367; Scheffzek et al., 1996, Biochemistry, 35:9716-9727; Schlichting et al., 1999, Nature Struct. Biol., 6:721-723). UMP kinases from bacterial species (products of the pyrH gene) are homohexamers in solution, whose activity is controlled allosterically by GTP (activator) and UTP (inhibitor). Sequence alignments show that bacterial UMP kinases display homology over limited regions of sequence with N-acetylglutamate kinases, carbamate kinases, aspartokinases, glutamate 5-kinase and pyrroline-5-carboxylate synthase (Labesse et al., 2002, supra; Gagyi et al., 2003, supra).

The crystal structure of P. furiosus carbamate kinase-like carbamoyl phosphate synthetase has been described (CK-like CPS, protein data bank (pdb) entry 1e19; Ramón-Maiques et al., 2000, J. Mol. Biol., 299:463-476). The crystal structure of E. coli N-acetyl-glutamate kinase has been described (NAGK, pdb entries lgs5, 1gsj, 1oh9, 1oha and 1ohb; Ramón-Maiques et al., 2002, Structure, 10:329-342; Gil-Ortiz et al., 2003, J. Mol. Biol., 331:231-244). Labesse et al. (2002, supra) proposed a homology model for the E. coli UMP kinase (pyrH). Gagyi et al. (2003, supra) proposed a homology model for the B. subtilis UMP kinase (pyrH). WO 03/025004 describes polypeptide targets for pathogenic bacteria.

### SUMMARY

Disclosed herein are the three-dimensional structure of UMP kinase (pyrH) from bacteria in complex with GTP and phosphate; binding sites of pyrH; methods for identifying and/or designing compounds or agents that bind PyrH, including ligands, drugs, or inhibitors that partially or totally inhibit pyrH activity, proteins and small organic molecules that bind pyrH; methods for crystallizing pyrH; computer-assisted methods for identifying, screening, and/or designing agents that bind pyrH; and NMR spectroscopy assisted methods for confirming agents proposed to bind pyrH.

### BRIEF DESCRIPTION OF THE DRAWINGS

FIG. 1 depicts the three dimensional structure of one molecule of the pyrH homohexameric assembly, depicting structural elements and the topology of the pyrH fold. Alpha-helices are labelled A through H, and beta-strands are labelled 1 through 11.

FIGS. 2 and 3 depict a cartoon depiction of two orthogonal views of the pyrH hexamer showing the active sites (outer surface of the hexamer, labelled X) and the allosteric binding sites (central cavity of the hexamer, labelled A). The bound phosphate ions at the predicted active centre, and the GTP molecules (modelled as GDP) at the allosteric binding sites, are shown in ball-and-stick representation throughout.

FIG. 4 depicts the conformational flexibility observed amongst the eight molecules present in the pyrH crystal asymmetric unit.

FIG. 5 shows the Nuclear Magnetic Resonance (NMR) TROSY-HSQC spectrum of 0.4 mM ²H/¹⁵N/¹³C-labelled H. influenzae in the presence of 1 mM UTP. They and x axes represent chemical shift in the nitrogen and proton dimensions, respectively, in ppm units.

FIG. 6 depicts an alignment of the amino acid sequences of pyrH from Streptococcus pneumoniae (SPN; SEQ ID NO:1); Streptococcus pyogenes (SPY; SEQ ID NO:2); Staphylococcus aureus (SAU; SEQ ID NO:3); Staphylococcus epidermidis (SEP; SEQ ID NO:4); Bacillus subtilis (BSU; SEQ ID NO:5); Neisseria meningitides (NME; SEQ ID NO: 6); Escherichia coli (ECO; SEQ ID NO:7); Haemophilus influenzae (HIN; SEQ ID NO:8); Chlamydia pneumoniae (CPN; SEQ ID NO:9); and Mycoplasma pneumoniae (MPN; SEQ ID NO:10).

FIG. 7 is a listing of the three-dimensional atomic coordinates of the crystal structure of pyrH from H. influenzae complexed with GTP and phosphate.

### DETAILED DESCRIPTION

The present invention is based upon the crystallization of H. influenzae pyrh, and the determination of the crystal structure (three-dimensional structure) of a complex of H. influenzae pyrH with GTP and phosphate.

### PyrH Polypeptides, Crystals and Space Groups

The present invention provides information relating to an isolated polypeptide of pyrH, or a portion of a polypeptide of pyrH, which functions as a binding site when folded in the proper 3-D orientation. As used herein, the term “isolated” in reference to polypeptides, means a polypeptide or a portion thereof which, by virtue of its origin or manipulation, has been removed from its natural state, or is otherwise not in its natural state. By “isolated” it is further meant a protein that is: (i) synthesized chemically; (ii) expressed in a host cell and purified away from associated and contaminating proteins; or (iii) purified away from associated and contaminating proteins. The term generally means a polypeptide that has been separated from other proteins and nucleic acids with which it naturally occurs. In some embodiments of the present invention, the polypeptide is also separated from substances such as antibodies or gel matrices (for example, polyacrylamide) that are used to purify it.

Each of the isolated polypeptide sequences can be a native sequence of pyrH, or a sequence that is at least 40%, 45%, 55%, 60%, 65%, 70%, 75%, 80%, 85%, 90%, 95%, 98% or 99% homologous to the amino acid sequence represented by SEQ ID NO:14.

The isolated pyrH can be a variant of pyrH. In one example, the variant may have an amino acid sequence that is different by one or more amino acid substitutions from the sequence disclosed in SEQ ID NO:14. Embodiments which comprise amino acid deletions and/or additions are also contemplated. The variant may have conservative changes (amino acid similarity), wherein a substituted amino acid has structural or chemical properties similar to those of the amino acid residue it replaces (e.g., the replacement of leucine with isoleucine). Guidance in determining which and how many amino acid residues may be substituted, inserted, or deleted without abolishing biological or pharmacological activity may be reasonably inferred in view of this disclosure and may further be found using computer programs well known in the art, for example, DNAStar® software.

Amino acid substitutions may be made, for instance, on the basis of similarity in polarity, charge, solubility, hydrophobicity, hydrophilicity, and/or the amphipathic nature of the residues as long as a biological and/or pharmacological activity of the native molecule is retained.

Example substitutions are set forth in Table 1 as follows:

TABLE 1

| Original residue | Example conservative substitutions |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

In the present invention, “amino acid homology” is a measure of the identity of primary amino acid sequences. In order to characterize the homology, subject sequences are aligned so that the highest percentage homology (match) is obtained, after introducing gaps, if necessary, to achieve maximum percent homology. N- or C-terminal extensions shall not be construed as affecting homology. “Identity” per se has an art-recognized meaning and can be calculated using published techniques. Computer program methods to determine identity between two sequences include, for example, DNAStar® software (DNAStar Inc., Madison, WI); the GCG® program package (Devereux et al., 1984, Nucl. Acids Res., 12:387); BLASTP, BLASTN, FASTA (Altschul et al., 1990, J. Mol. Biol., 215:403). Homology (identity or similarity) as defined herein is determined using the computer program, BLAST 2 Sequences (Tatusova and Madden, 1999, FEMS Microbiol. Lett. 174:247-250; available from the NCBI), employing default settings for all parameters, such that percentage identity and/or similarity are calculated over the full length of the aligned sequences, and that gaps in homology of up to about 90% of the total number of nucleotides or amino acids in the reference sequence are allowed.

The invention also includes a crystal of pyrH. In one embodiment, the crystal is pyrH complexed with phosphate and guanosine triphosphate (GTP). The pyrH can be from any bacteria including H. influenzae.

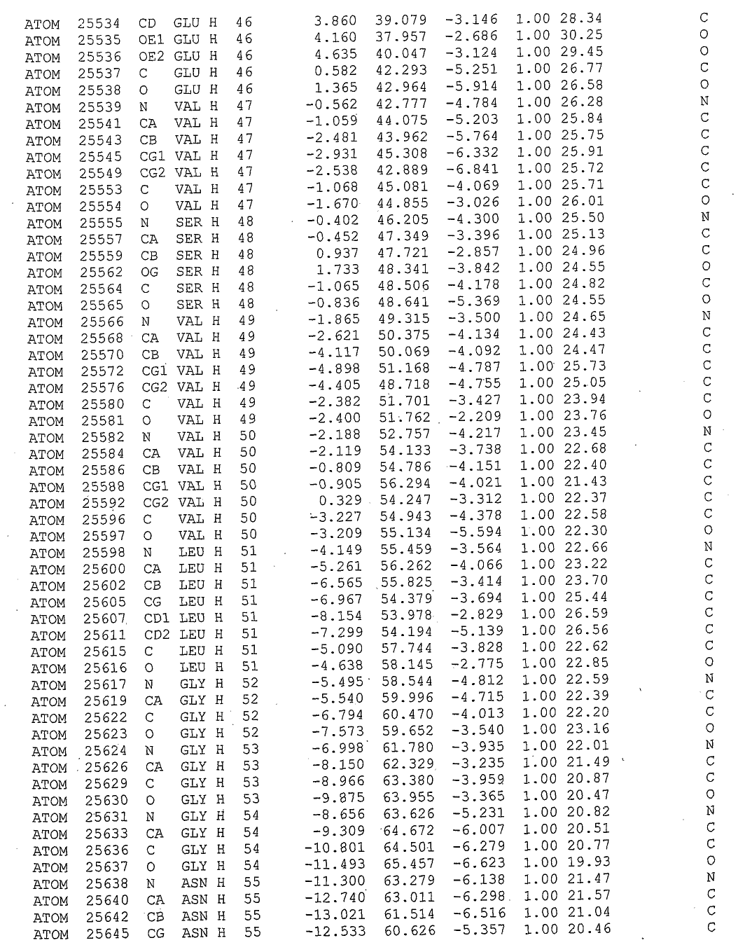

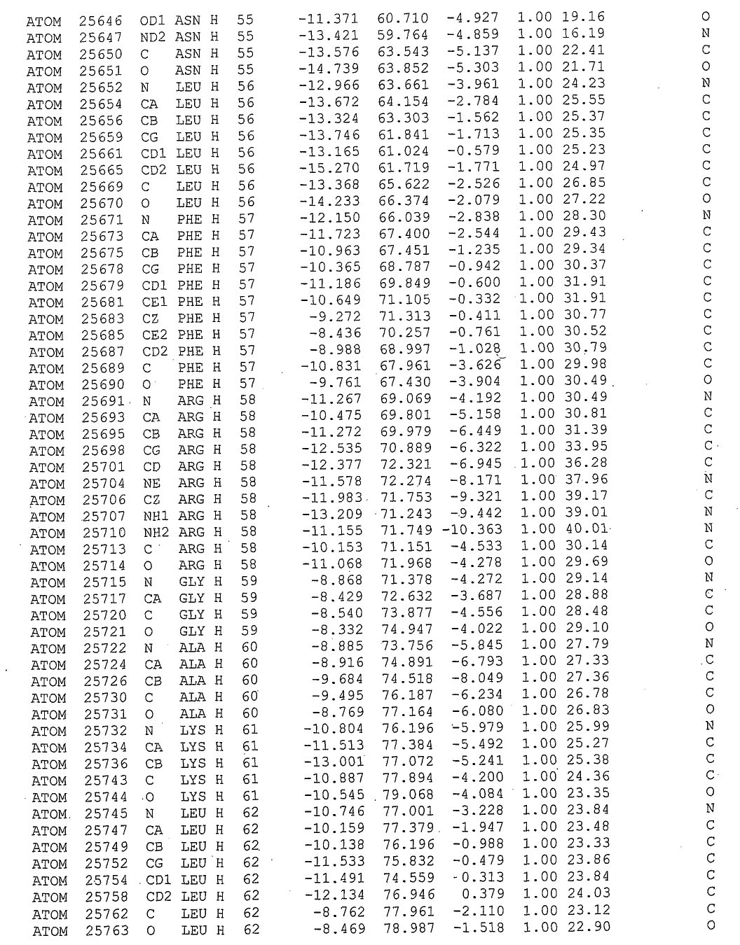

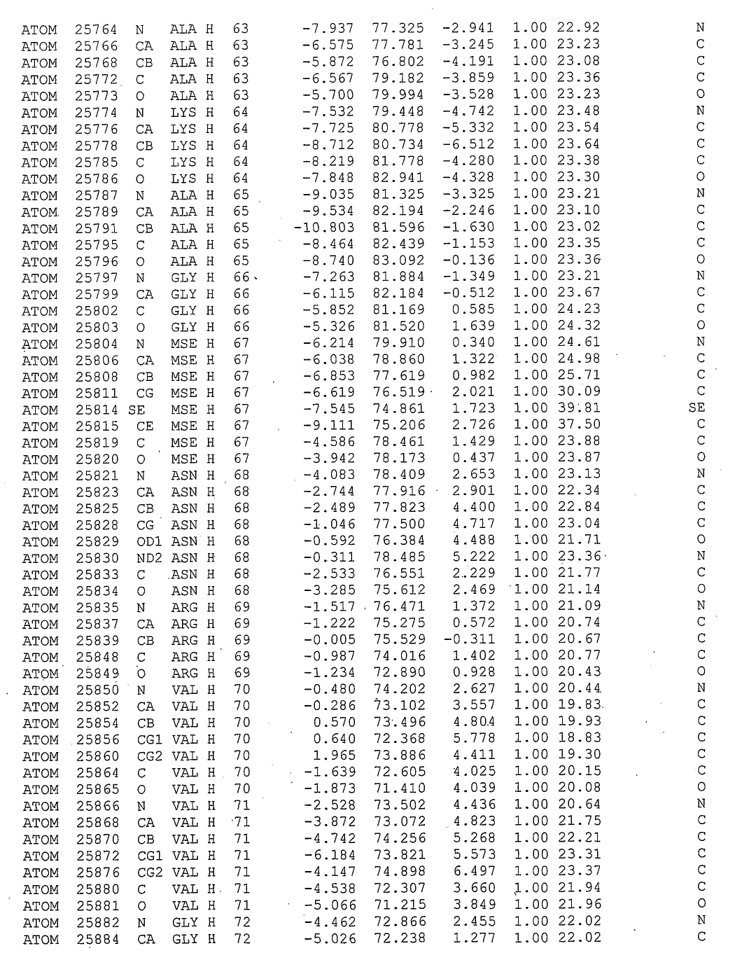

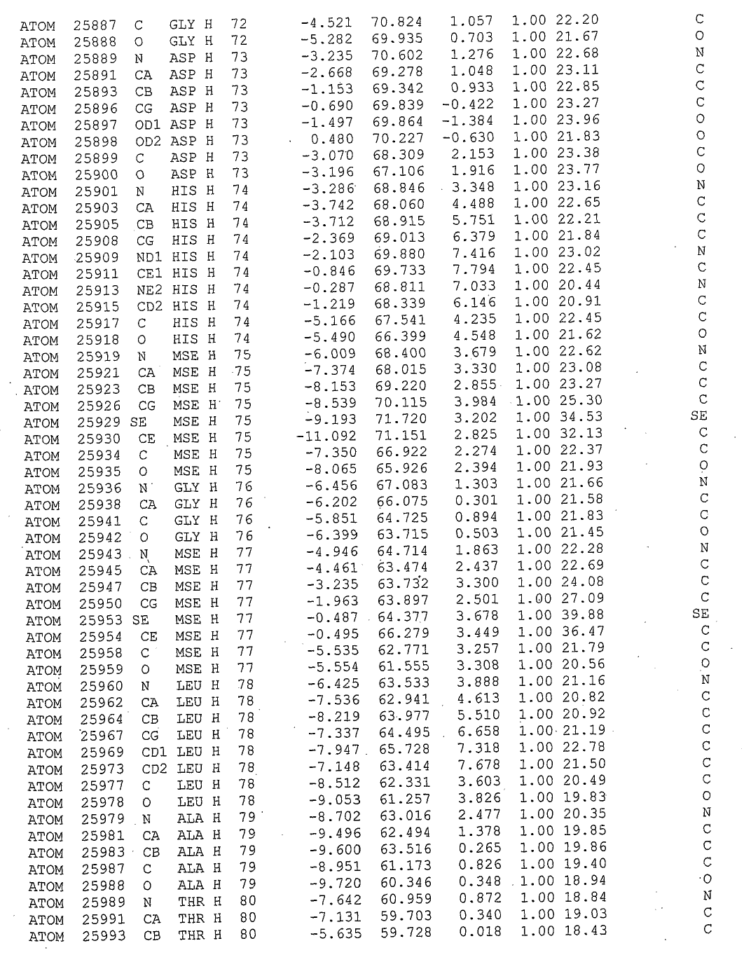

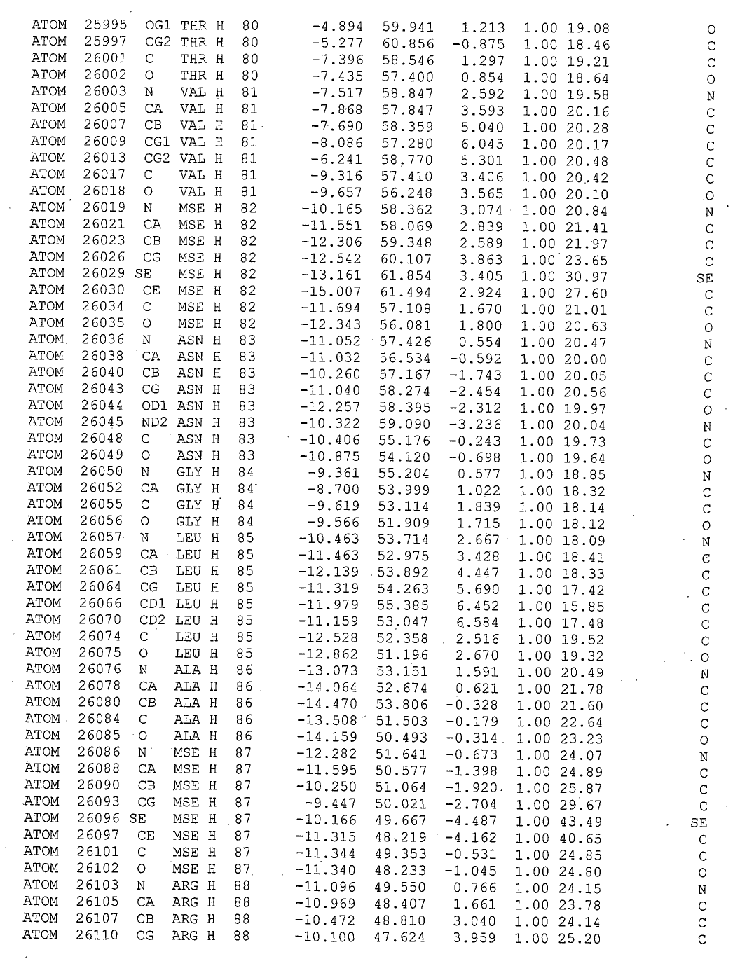

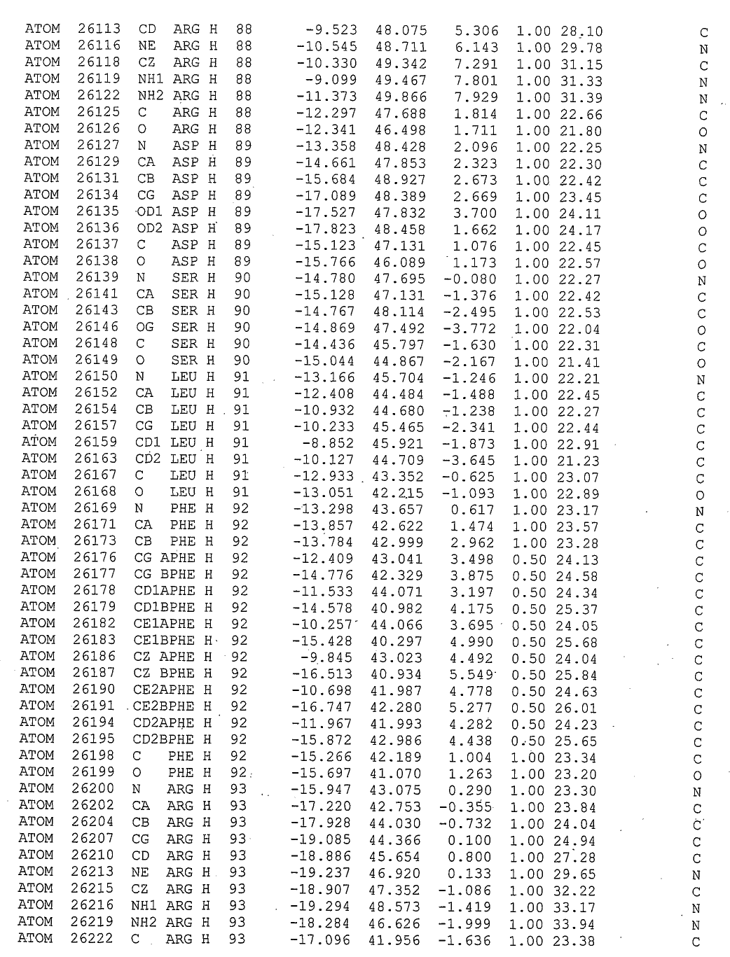

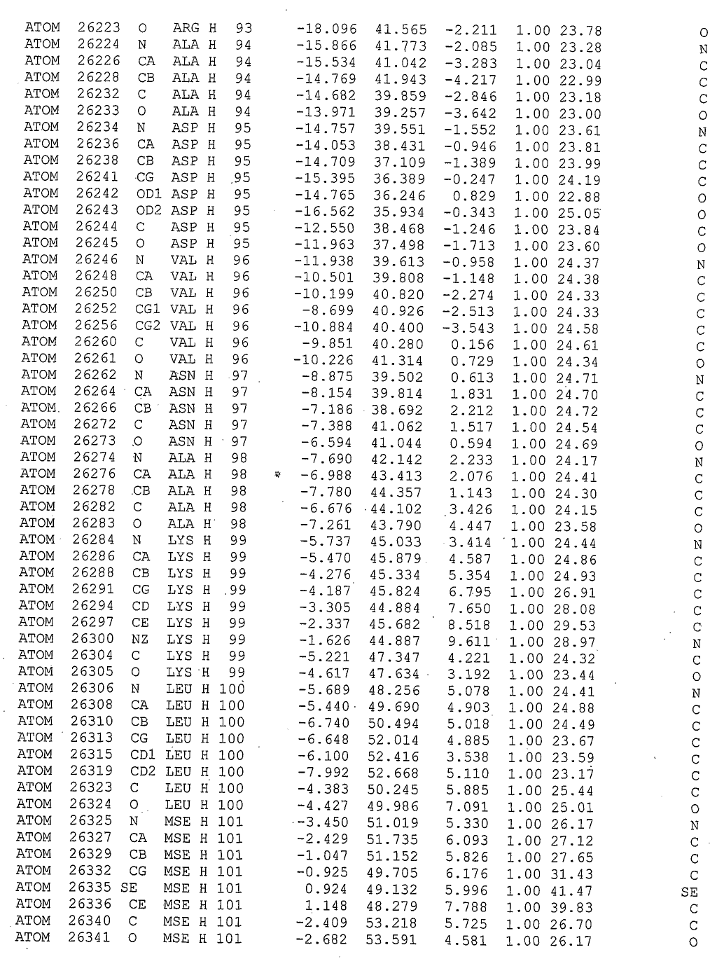

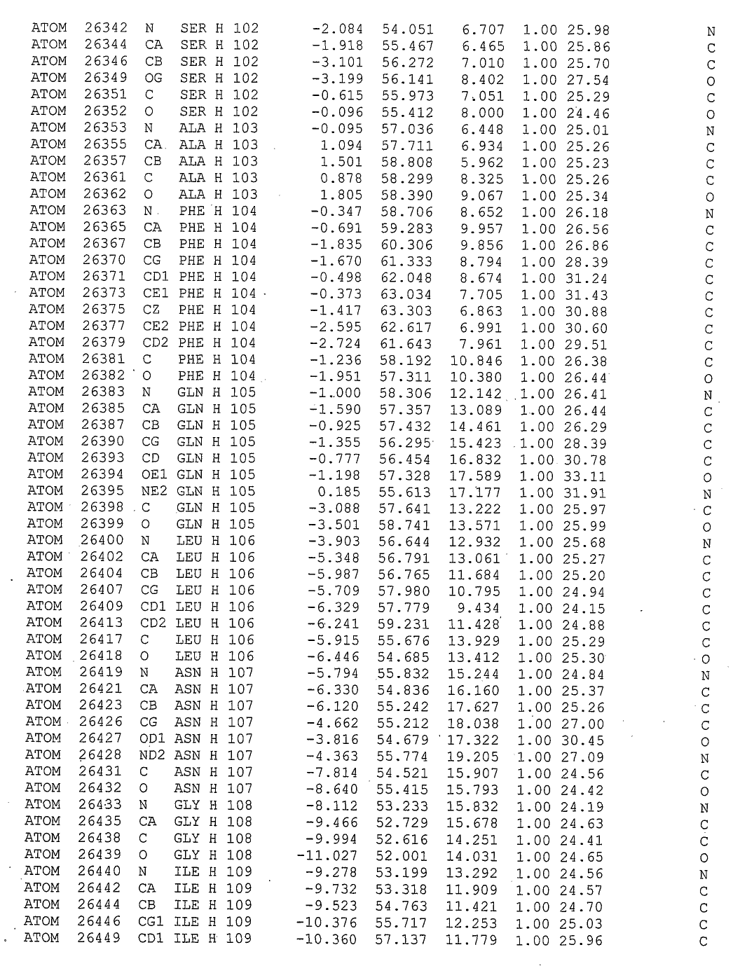

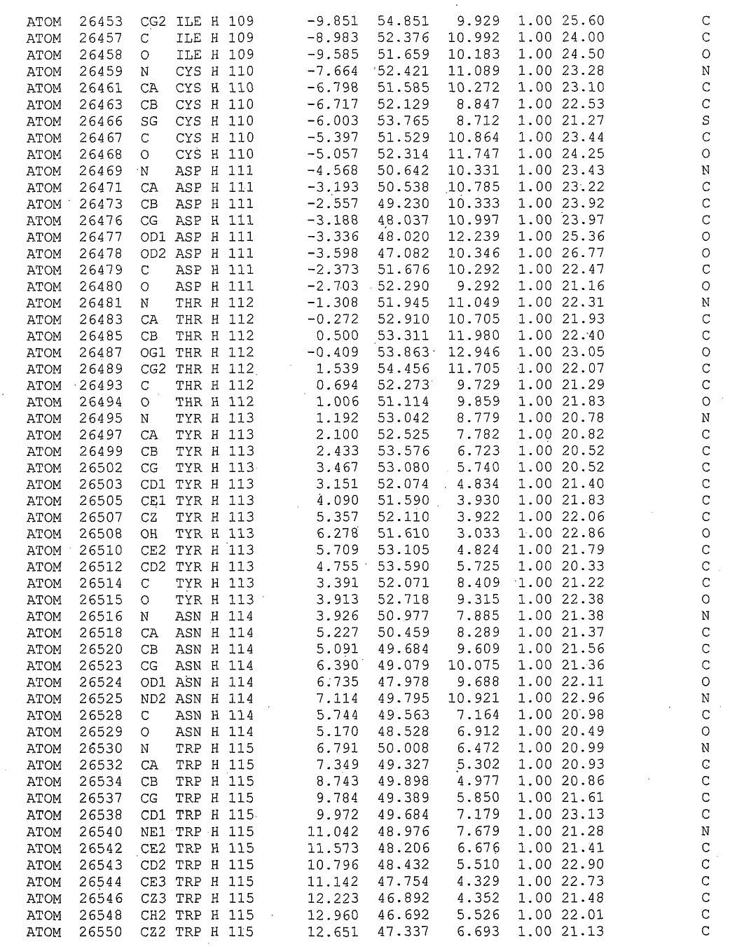

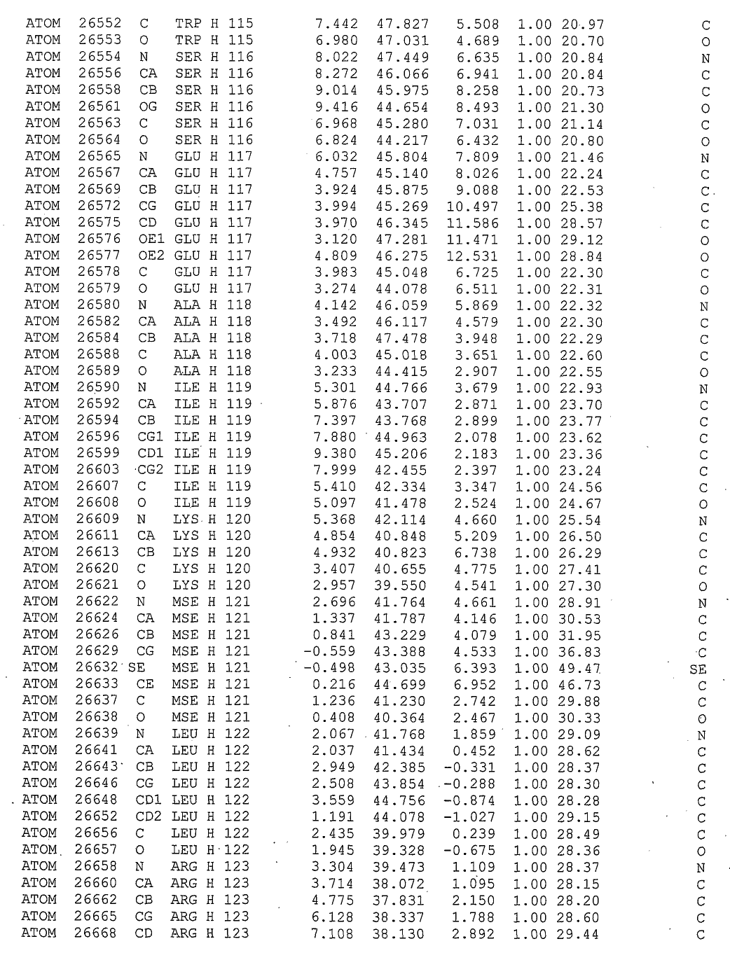

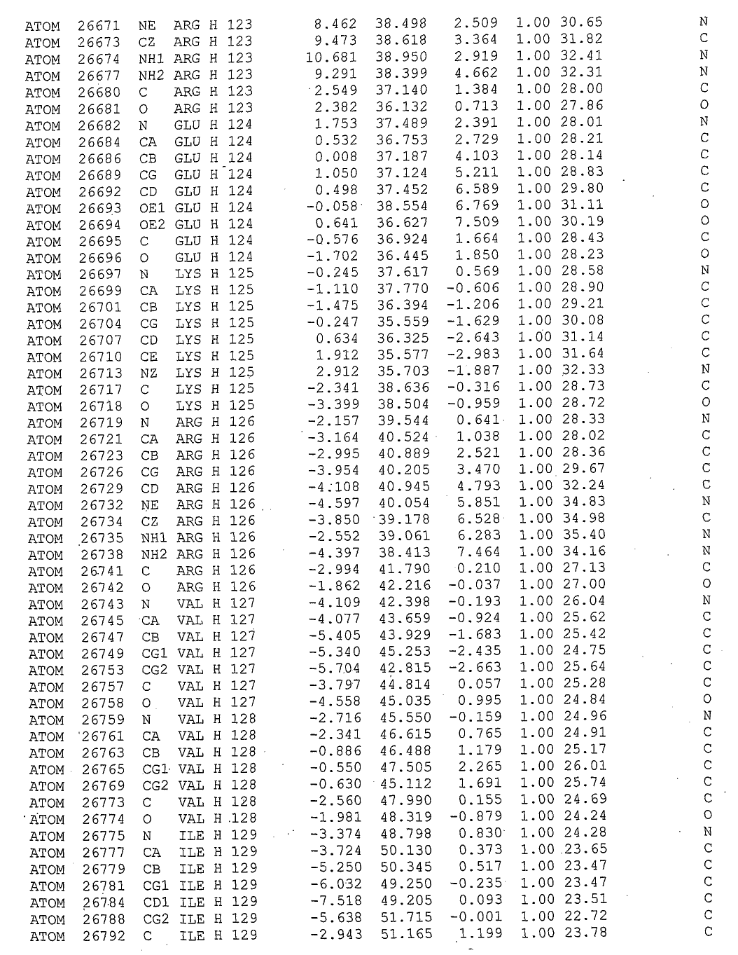

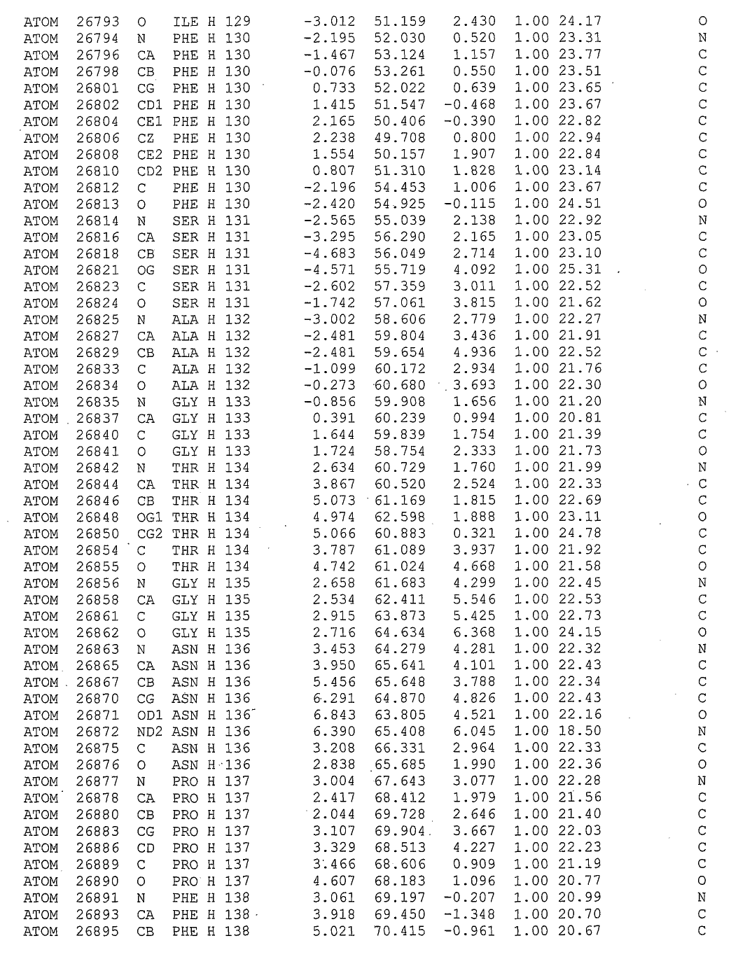

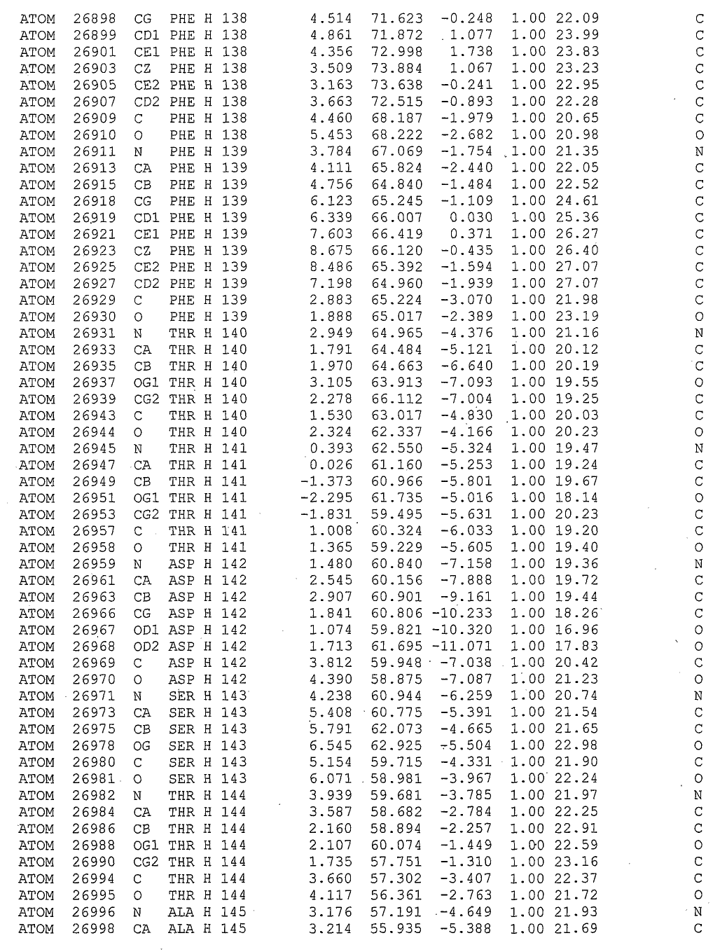

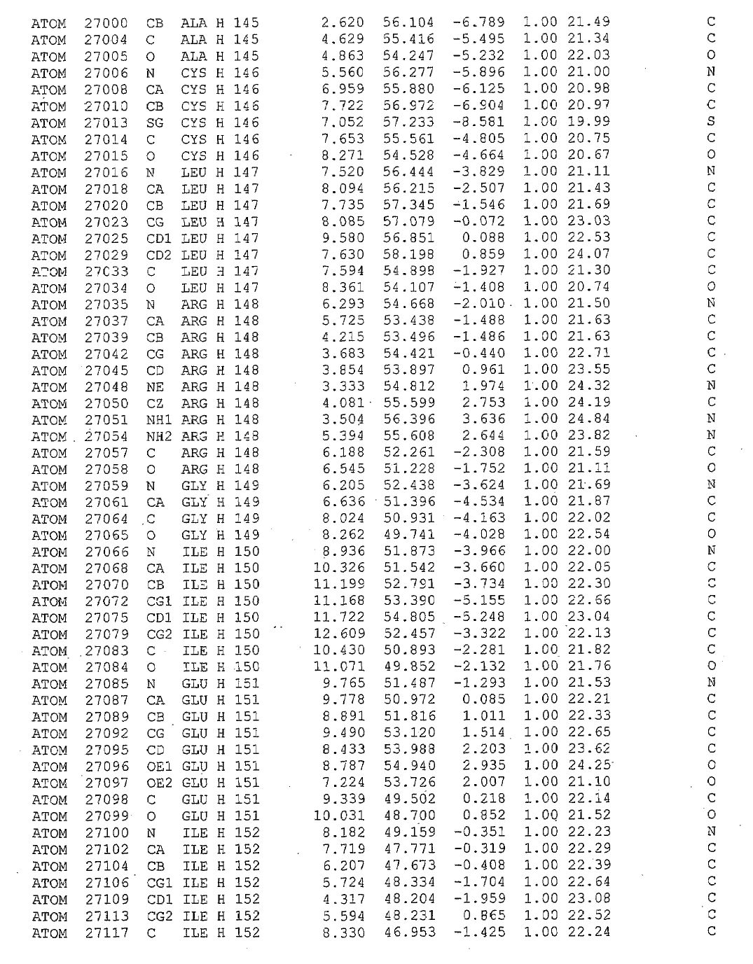

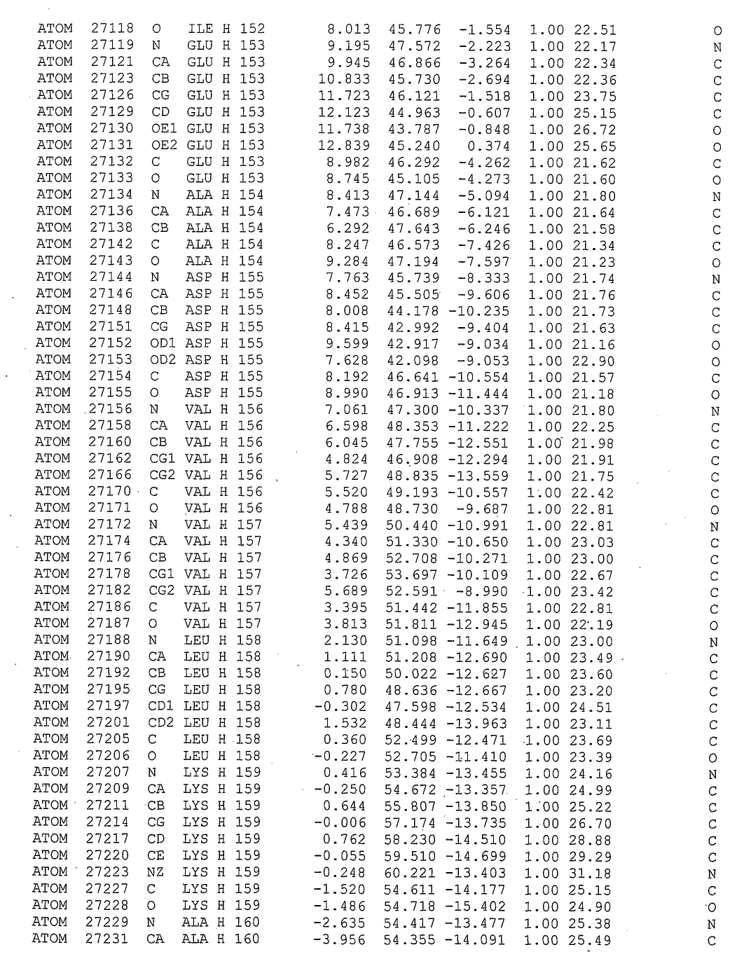

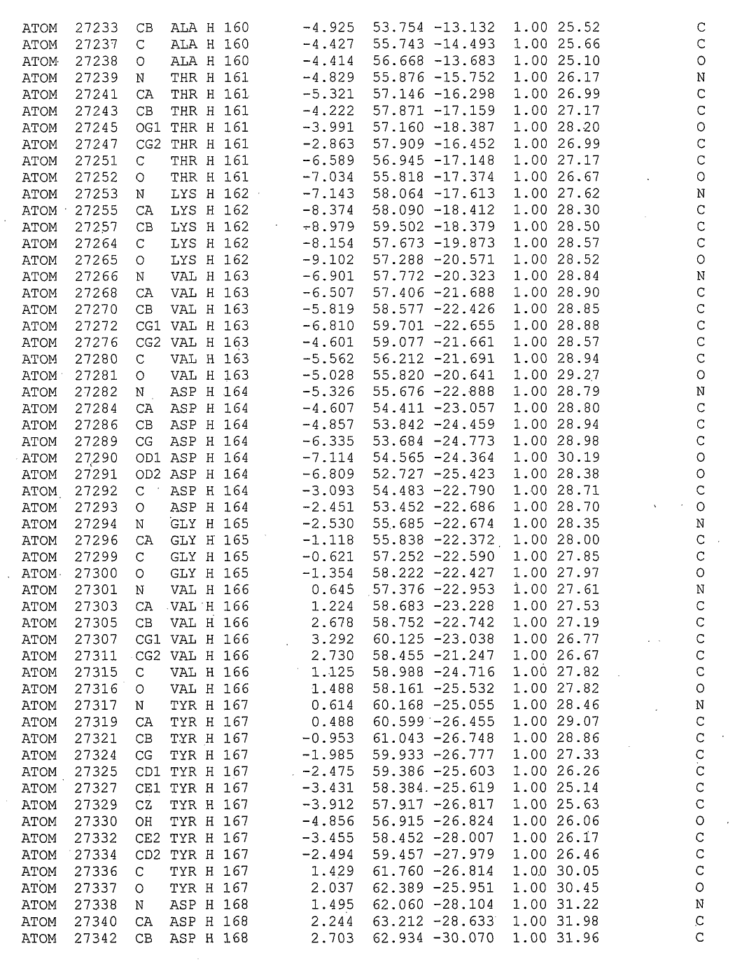

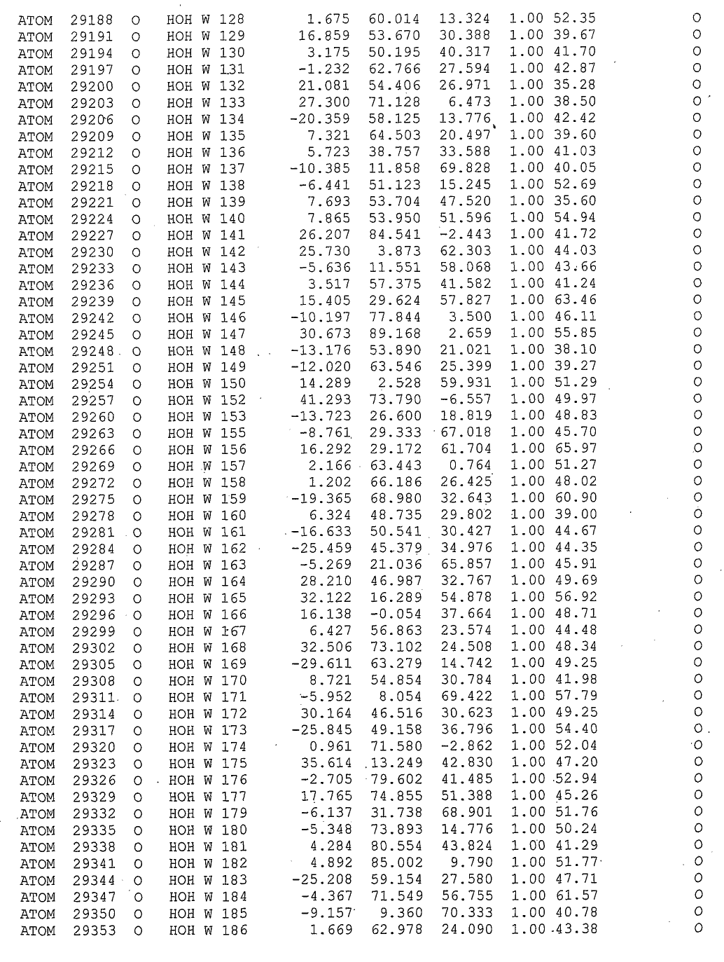

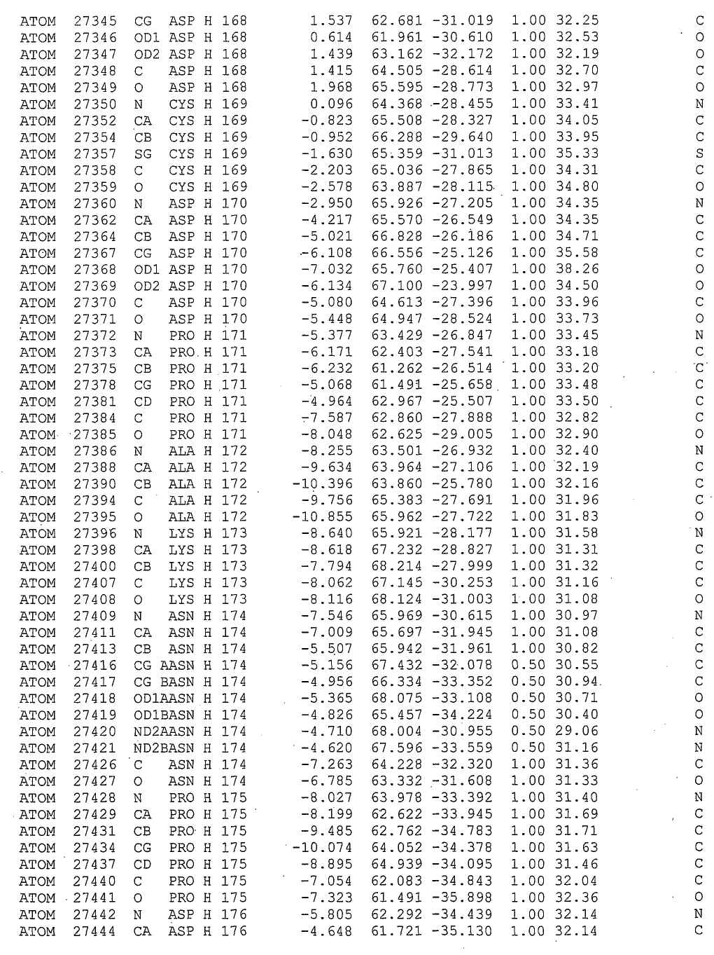

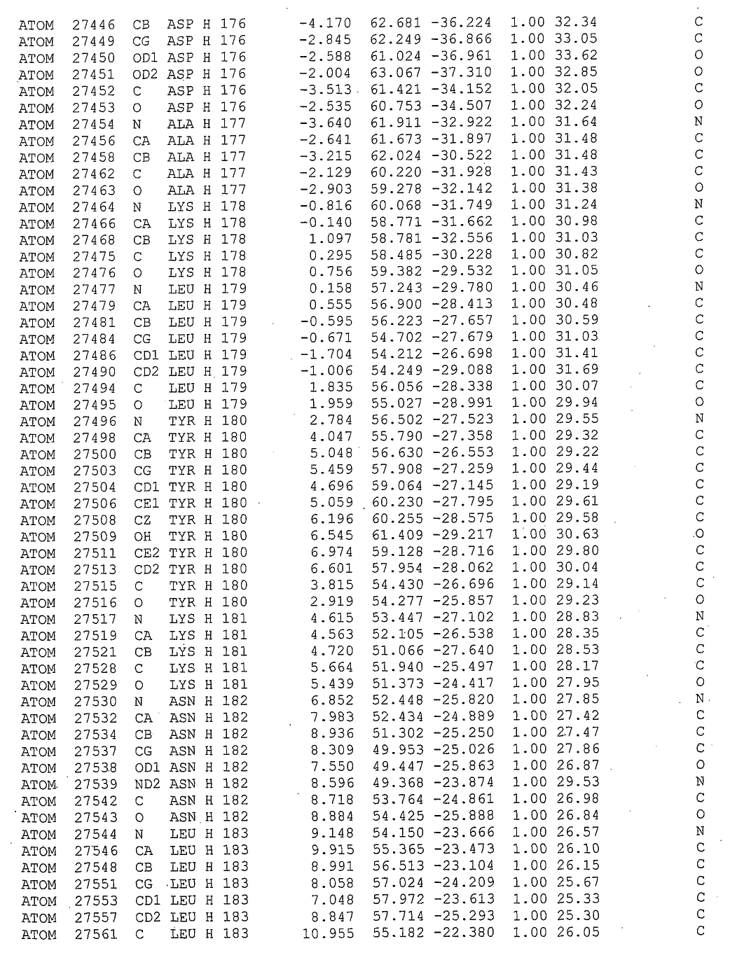

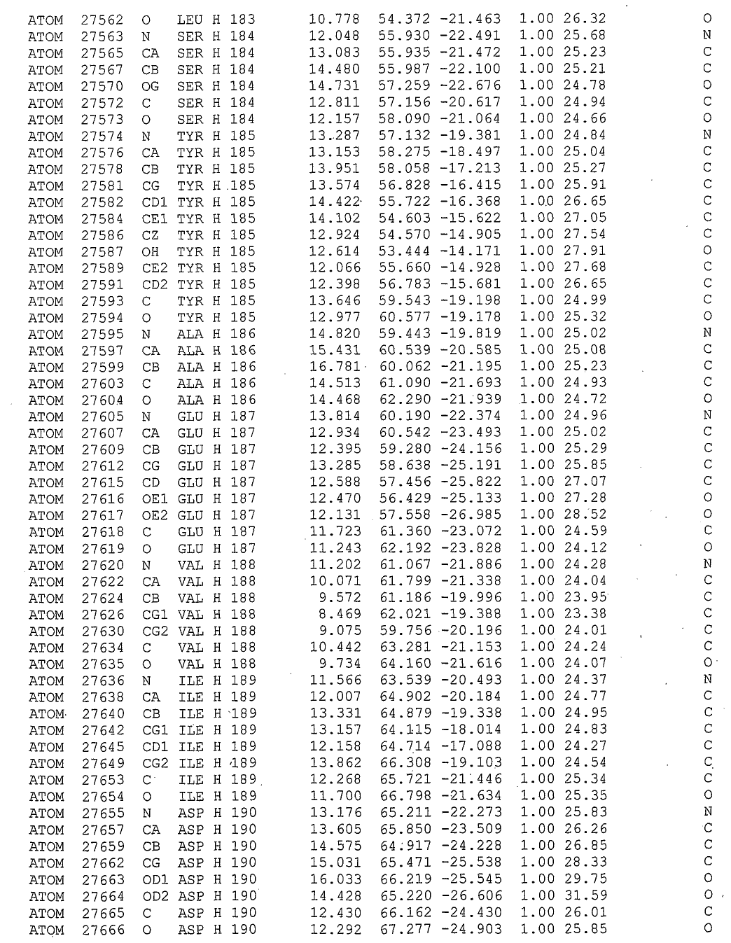

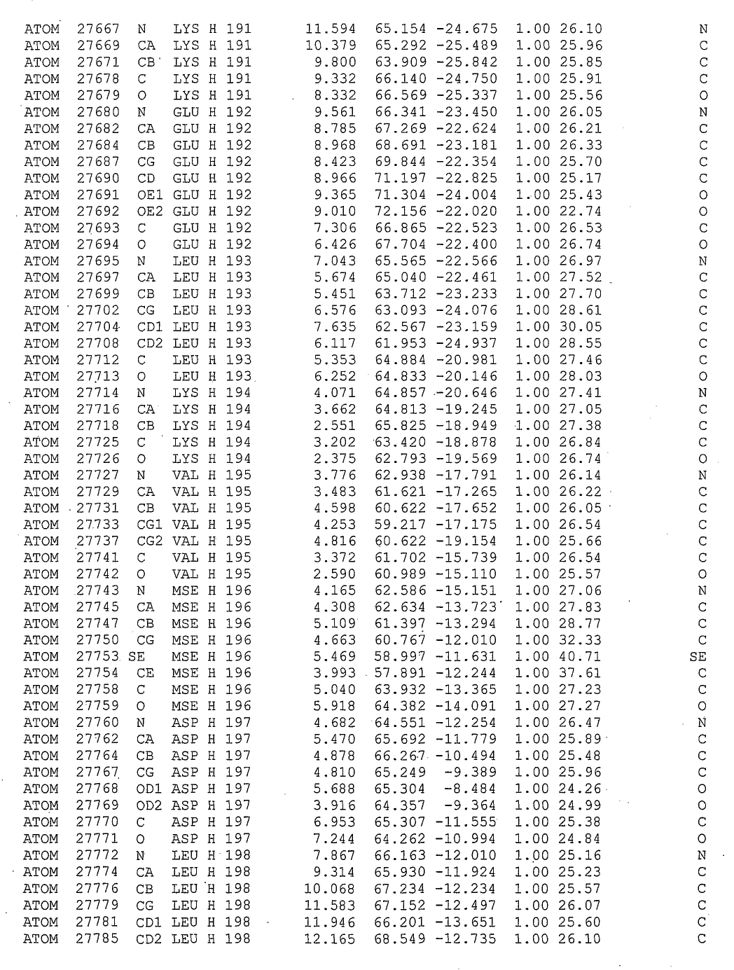

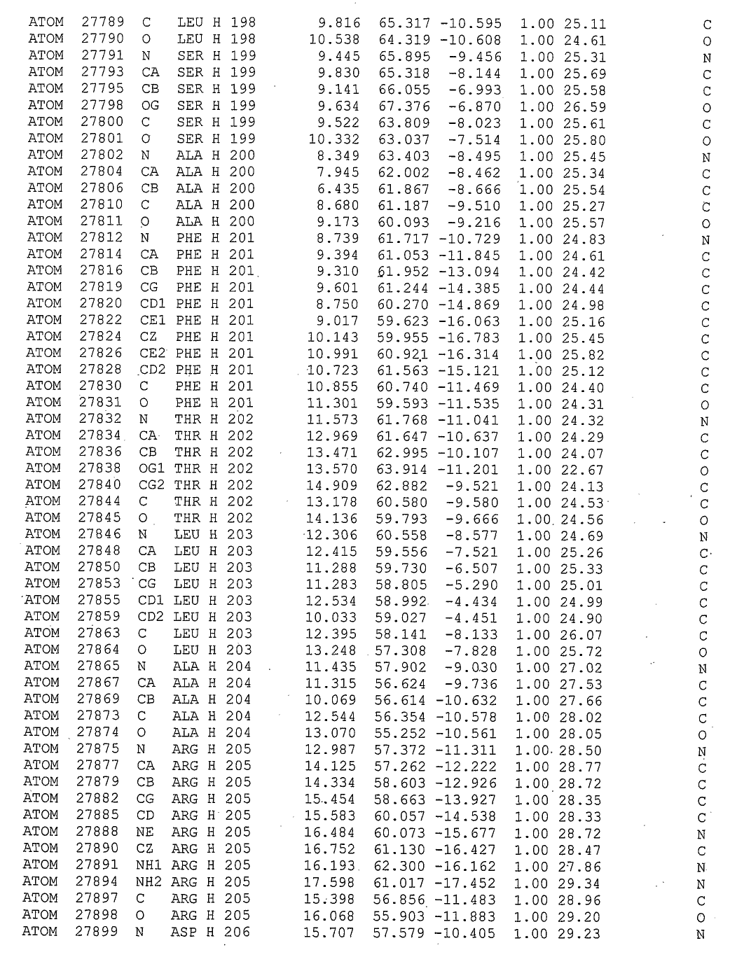

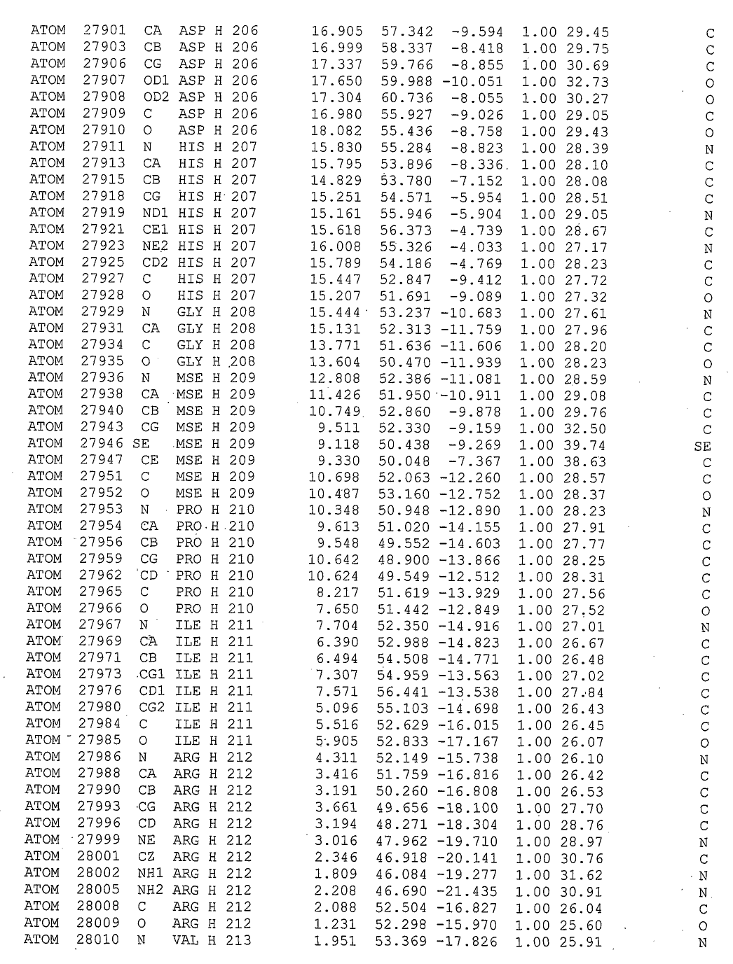

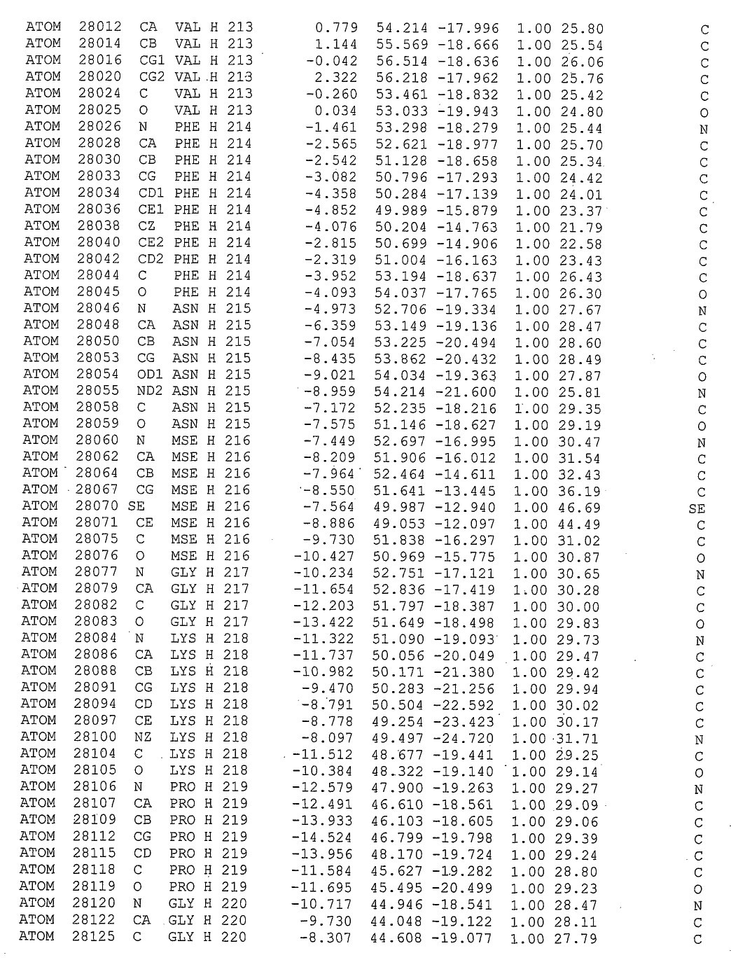

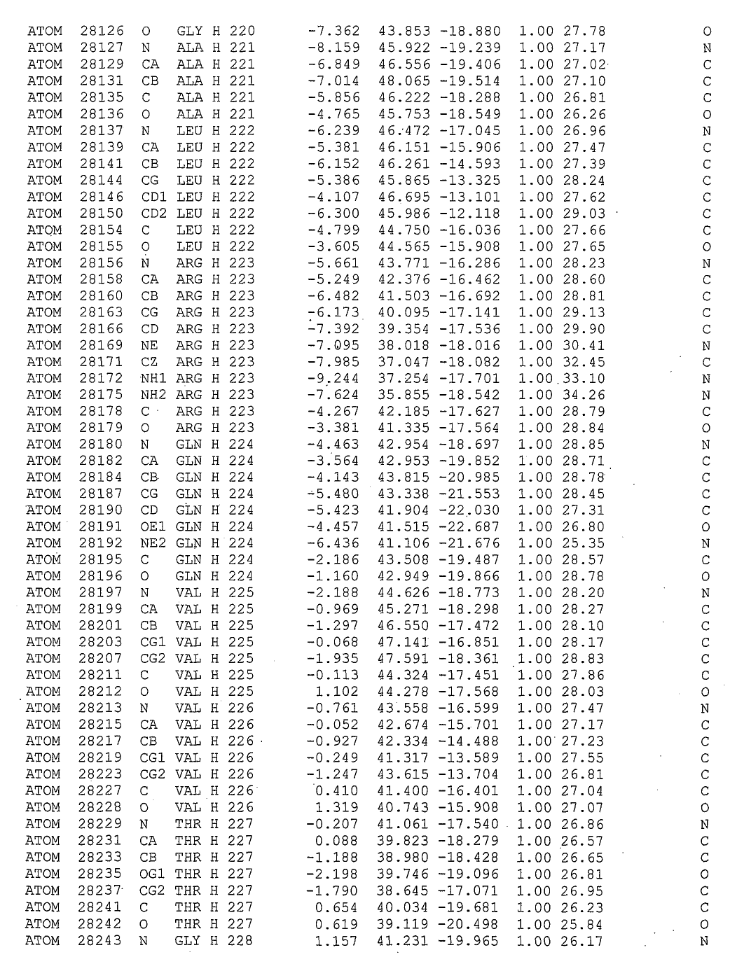

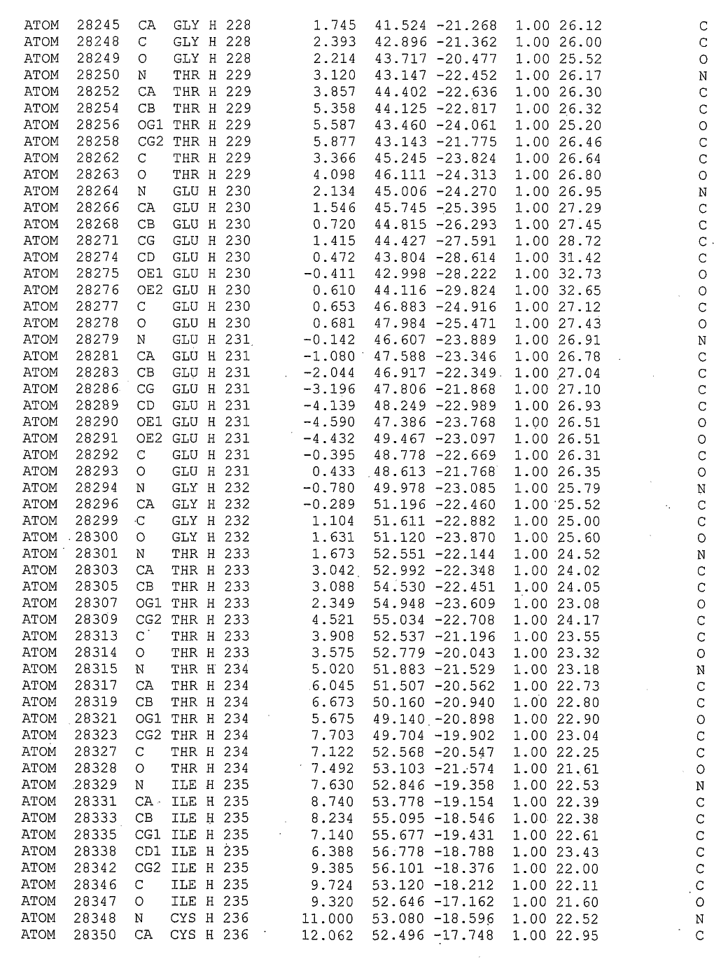

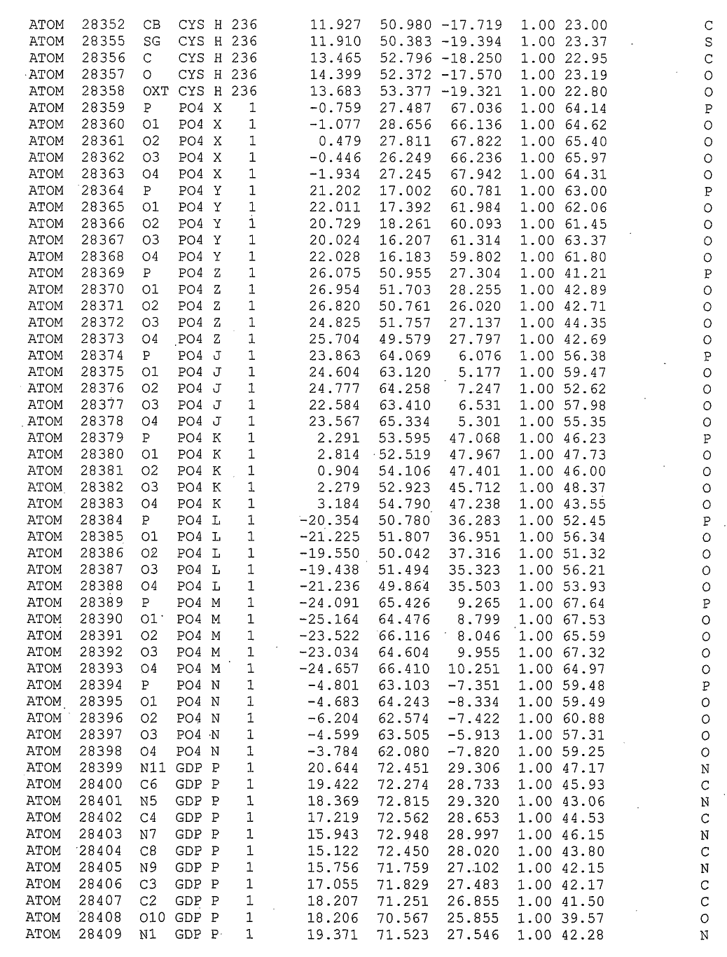

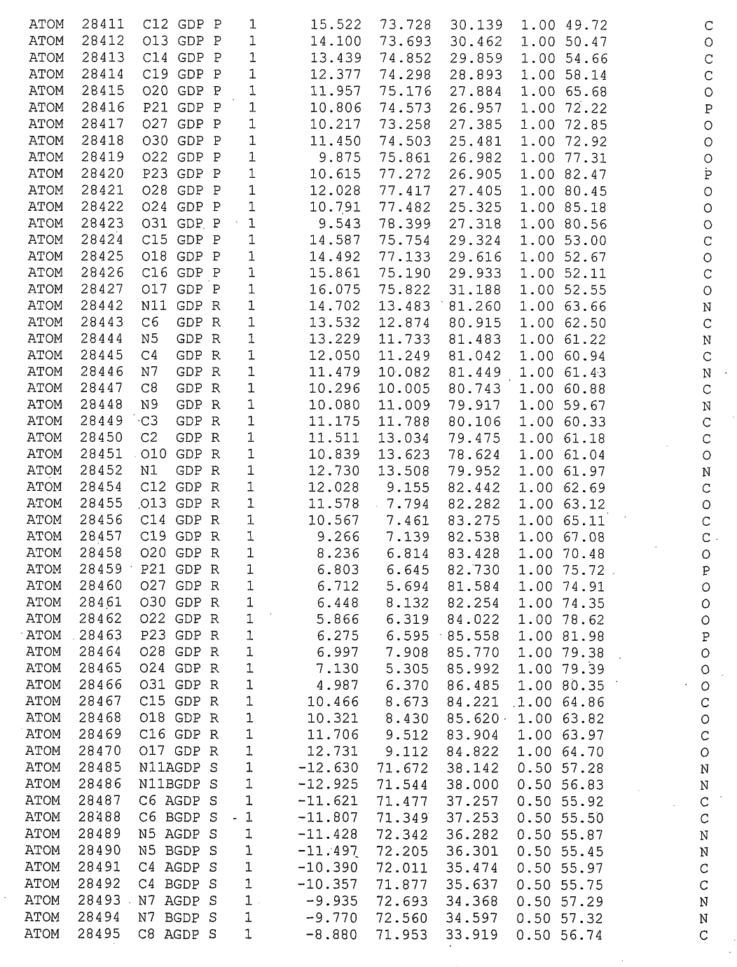

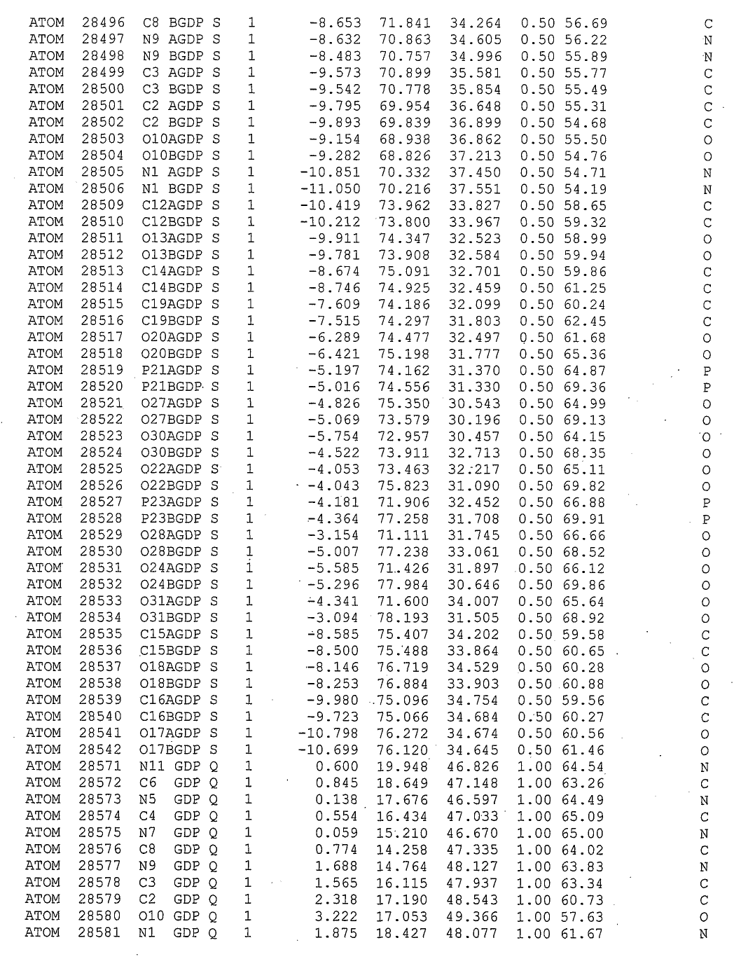

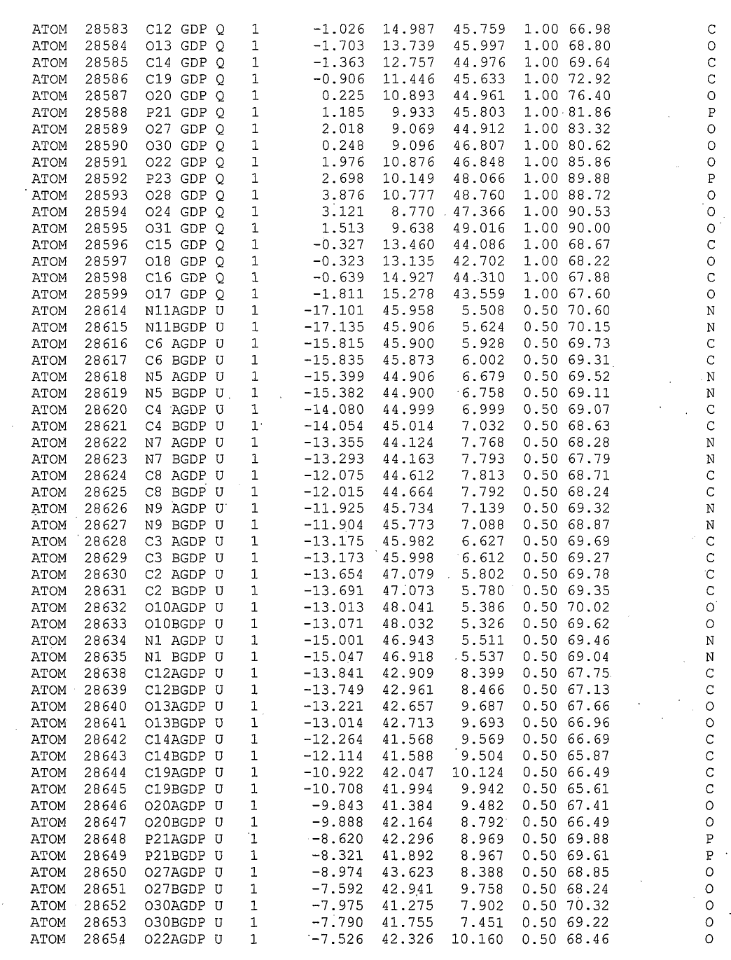

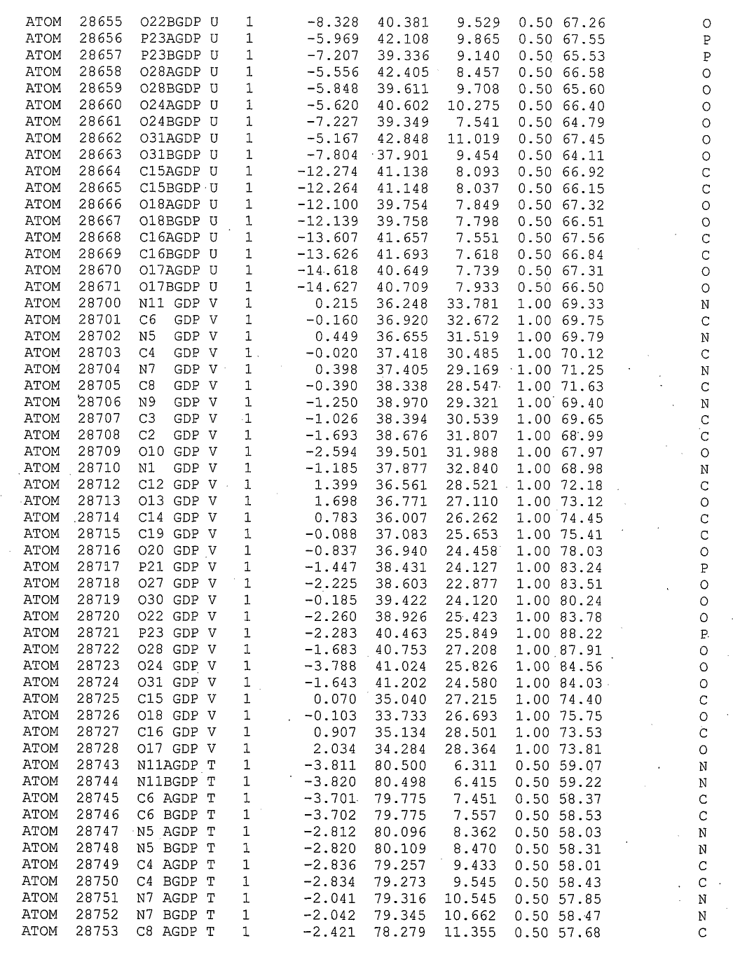

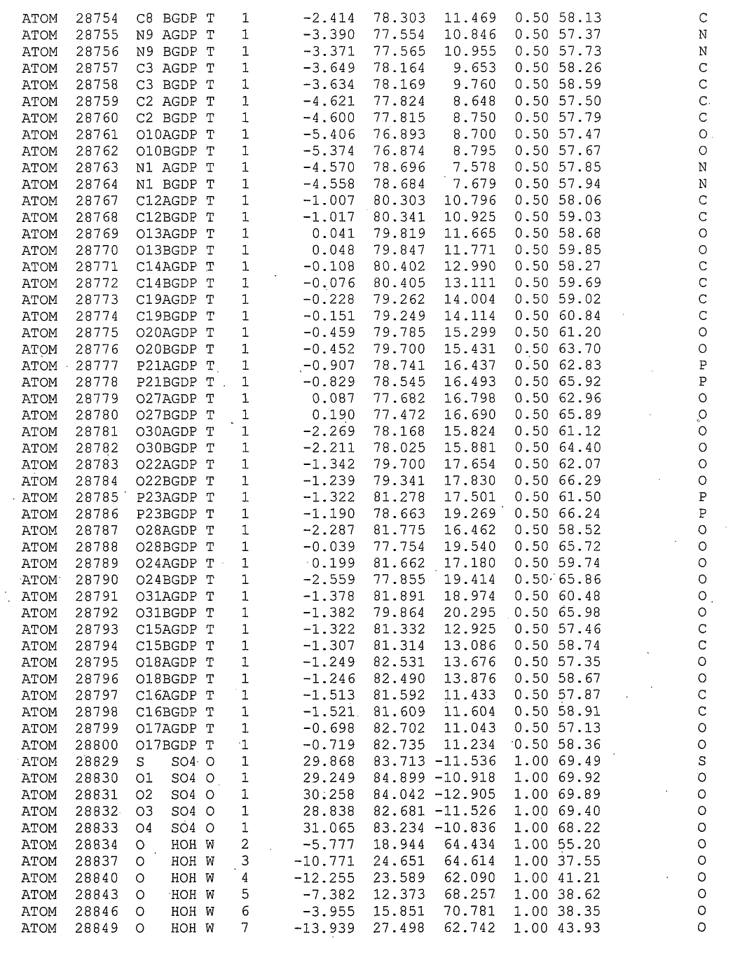

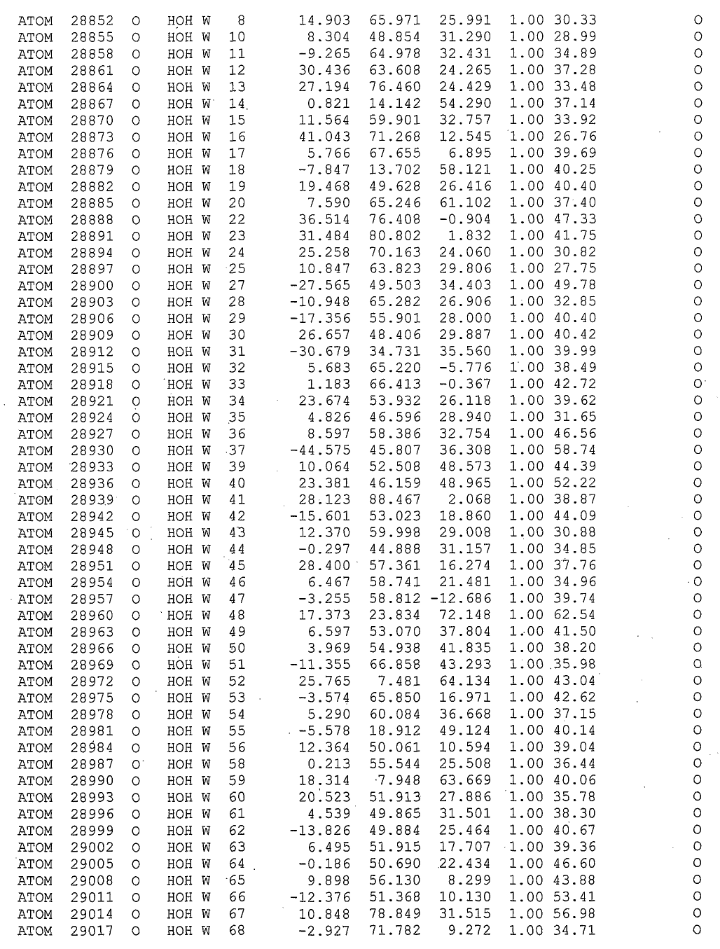

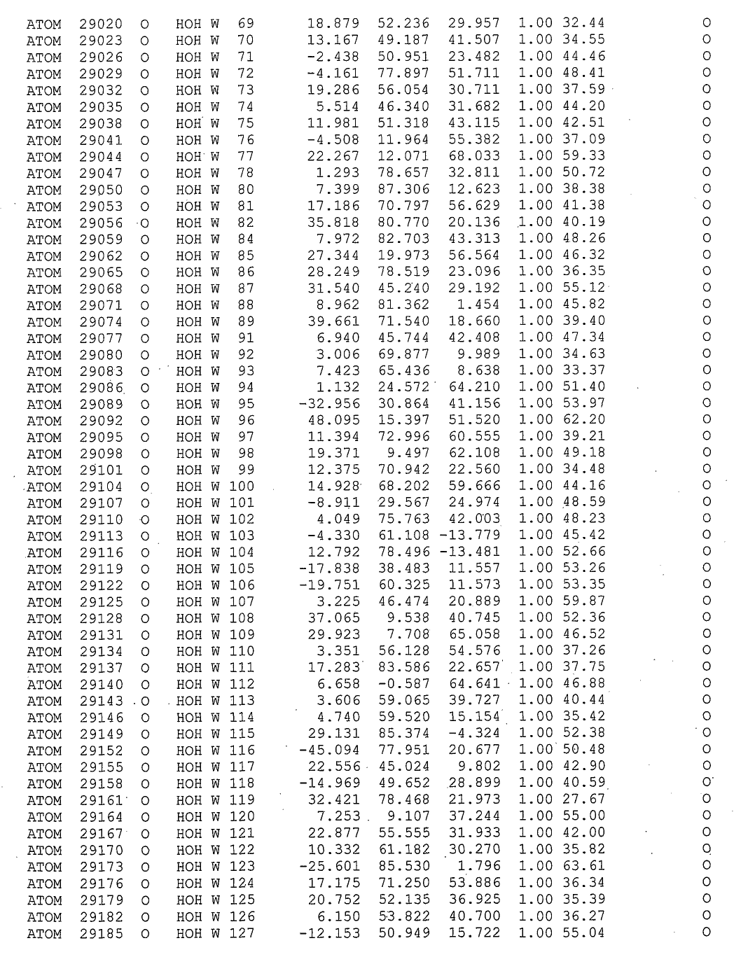

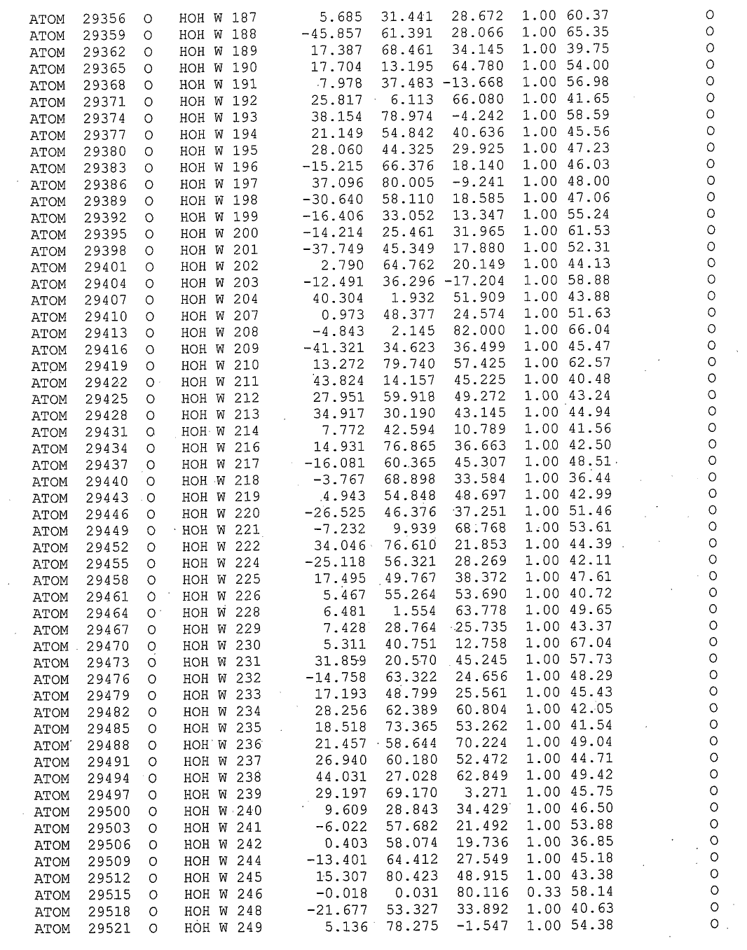

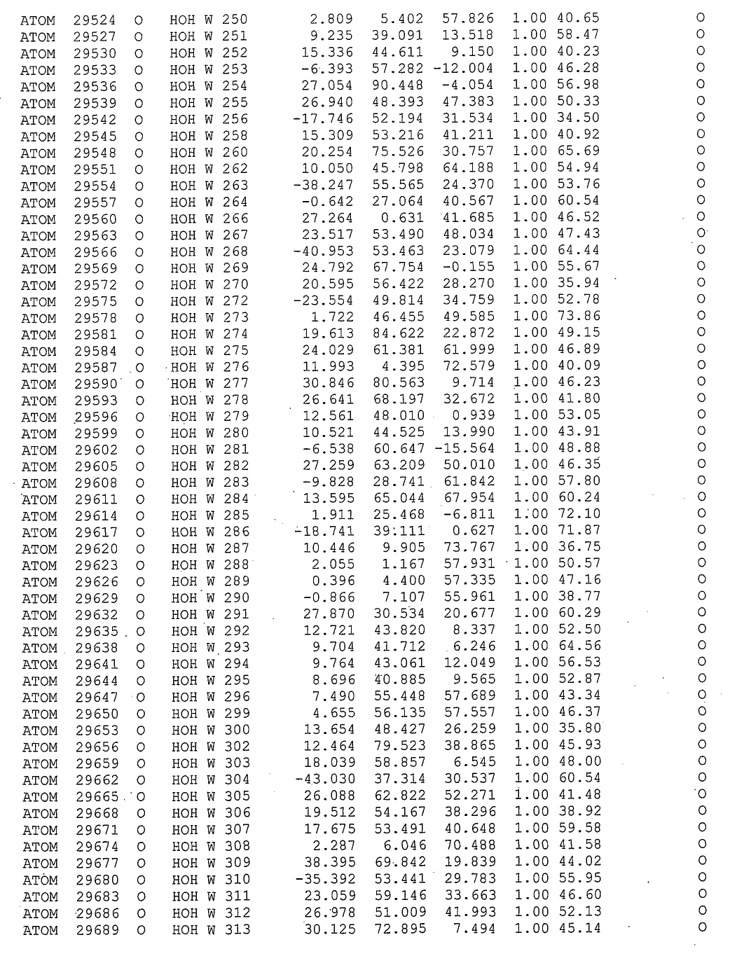

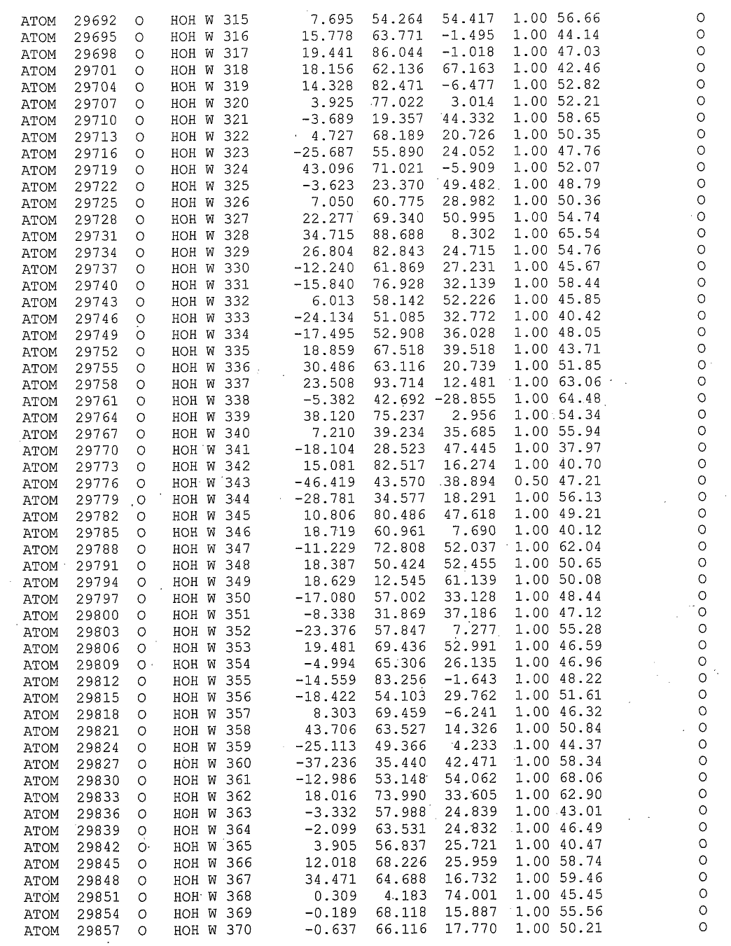

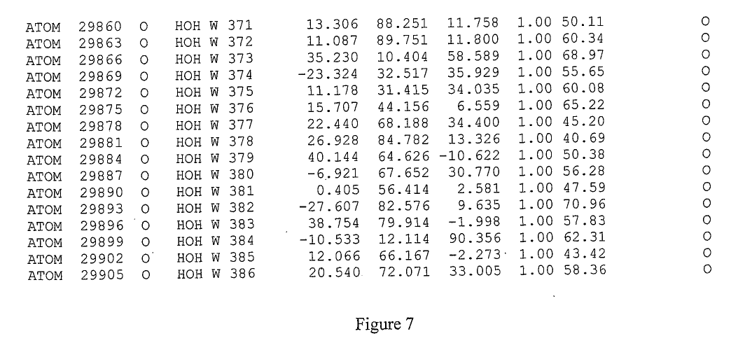

In another embodiment, the invention includes a crystallized H. influenzae pyrH complexed with phosphate and GTP, and characterized by the atomic coordinates presented in FIG. 7. In the present invention, the crystals can diffract to about 2.3 Å.

One example of the crystallized complex is characterized as belonging to the rhombohedral space group R32 and has unit cell parameters of a=b=c=(146.5+/−0.7) Å, and α=β=γ=(97.38+/−0.07)°. Those of skill in the art will recognise that it is convenient to work in the hexagonal equivalent of the rhombohedral space group wherein the pyrH crystal of the invention has unit cell parameters of a=b=(215+/−1.0) Å and c=(233.6+/−1.5) Å, and α=β=90°, and γ=120°.

The PyrH crystal of the invention possesses an asymmetric unit containing eight molecules of pyrH, comprising a homohexameric assembly, and an additional homodimeric assembly from which those of skill in the art will recognise that a hexameric assembly can be generated by applying the crystallographic symmetry operations inherent in the rhombohedral space group. Each homohexameric assembly can be described as a trimer of dimers. In one embodiment, each dimer comprises an intermolecular dimer interface comprising amino acid residues Ile25, Pro27, Leu30, Asp31, Phe57, Lys61, Leu62, Ala65, Gly66, Met67, Asn68, Arg69, Val71, His74, Met75, Gly76, Leu78, Ala79, Val81, Met82, Leu85, Ala86, Arg87, Asp88, Arg89, Phe104, Gln105, Leu106, Asn107, Gly108 and Ile109 of SEQ ID NO:14.

Methods of making crystals are known in the art. In one example, a crystallized complex, as described above, can be produced by the process of preparing a first solution containing H. influenzae pyrH of adequate purity, for example >95%, and in an appropriate buffer, for example 50 mM Tris-HCl pH8.5; preparing a second solution containing a suitable precipitant, for example a salt or polyethylene glycol; combining the first solution and the second solution, thereby producing a combination; and forming drops from the combination in a method of crystallization such that the pyrH is brought into a state of supersaturation, whereby, crystals of pyrH are produced.

### PyrH Crystal Structure

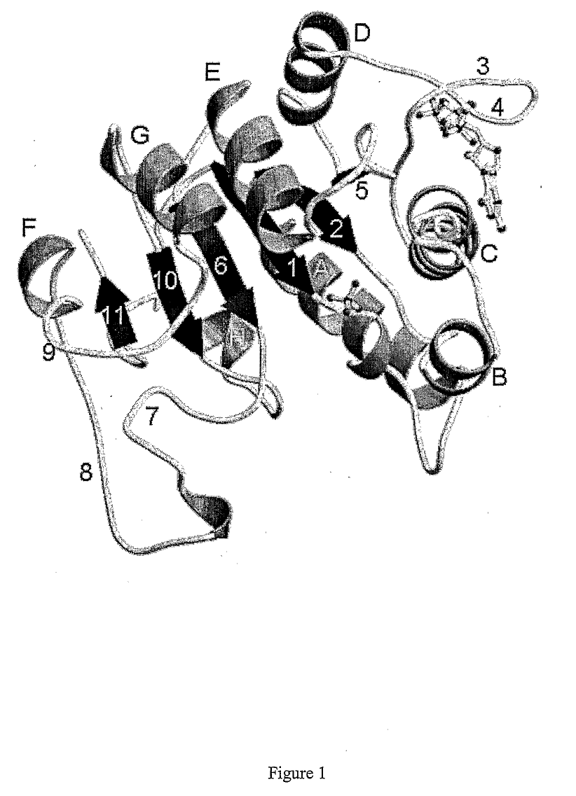

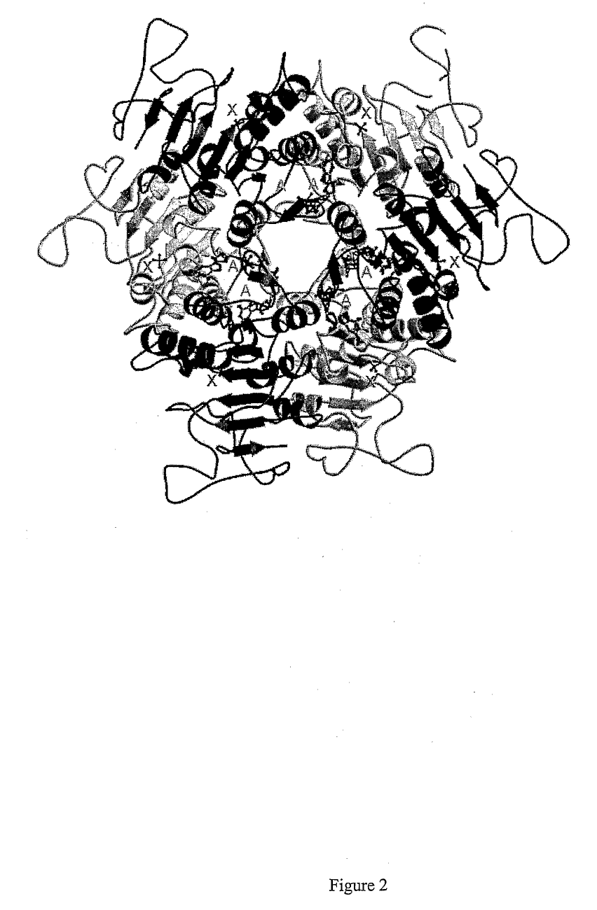

The asymmetric unit of the pyrH crystal consists of eight copies of the pyrH polypeptide chain, characterised by the atomic coordinates tabulated in FIG. 7, corresponding to a homohexameric assembly and a homodimer from which a hexameric assembly can be generated by the operation of crystallographic symmetry transformations. The active form of H. influenzae pyrH is a homohexamer. Each molecule of pyrH consists of a single domain comprising a central beta-sheet composed of nine beta-strands (labelled 1 to 6 and 9 to 11 in FIG. 1) flanked on both sides by four alpha-helices (those labelled D, E, F and G in FIG. 1 on one side, and those labelled A, B, C and H in FIG. 1 on the second side). The binding of a phosphate ion identifies the active site, which is located across the C-terminal edge of the beta-strands in a broad, solvent-exposed cavity bounded by loop regions (indicated with an X in FIG. 2). The six active sites are located on the outer face of the hexameric assembly. On a second face of the pyrH molecule (labelled with an A in FIG. 2), close to the centre of the hexameric assembly, at the interface of three pyrH molecules, is the binding site for an allosteric effector (for example an activator, such as GTP).

The H. influenzae pyrH hexameric assembly can be described as a trimer of dimers. The dimers are held together by a stable and conserved hydrophobic core, created by the interlocking of methionine residues along the faces of two extended antiparallel alpha-helices (residues 68 to 95, helix C, see FIG. 1). Additional interactions are made between the conserved beta3-beta4 loops of each pyrH molecule (comprising residues 104 to 109), which are oriented antiparallel. There are also stabilising contacts between residues 25, 27, 30 and 31 from the beta1-alphaA loop and the N-terminus of the alphaA helix (see FIG. 1) in one pyrH molecule and residues 57, 61, 62 and 65 from a short helical region (helix B, see FIG. 1) in the second molecule. The intermolecular dimer interface thus includes amino acid residues Ile25, Pro27, Leu30, Asp31, Phe57, Lys61, Leu62, Ala65, Gly66, Met67, Asn68, Arg69, Val71, His74, Met75, Gly76, Leu78, Ala79, Val81, Met82, Leu85, Ala86, Arg87, Asp88, Arg89, Phe104, Gln105, Leu106, Asn107, Gly108 and Ile109 of SEQ ID NO:14.

Three dimers come together to form a hexameric assembly through interactions involving residues Asn68, Arg69, Val70, Val71, His74, Arg88, Phe92, Lys99, Gln105, Leu106, Asn107, Gly108, Ile109, Cys110, Asp111, Thr112, Tyr113, Asn114, Trp115, Glu117, Thr134, Asn136, Pro137, Phe138, Phe139, Leu147, Arg148, Ile150, Glu151, Glu153, Leu198, Ser199, Thr202, Leu203 and His207 of a first molecule from a first dimer, residues Asn68, Arg69, Val70, Val71, His74, Thr134, Asn136, Pro137, Phe138, Phe139, Leu147, Arg148, Ile150, Glu151, Glu153, Leu198, Ser199, Thr202, Leu203 and His207 of a second molecule from a second dimer, residues Arg88, Phe92, Lys99, Asn107, Gly108, Ile109, Cys110 and Asp 111 of a third molecule from the second dimer, and residues Gln105, Leu106, Thr112, Tyr113, Asn114, Trp115 and Glu117 of a fourth molecule from a third dimer. Contacts were determined using a radius of 5 Å and the program Contact (CCP4, 1994, Acta Cryst., D50:760-763). The intermolecular dimer-dimer interface thus comprises amino acid residues Asn68, Arg69, Val70, Val71, His74, Arg88, Phe92, Lys99, Gln105, Leu106, Asn107, Gly108, Ile109, Cys110, Asp111, Thr112, Tyr113, Asn114, Trp115, Glu117, Thr134, Asn136, Pro137, Phe138, Phe139, Leu147, Arg148, Ile150, Glu151, Glu153, Leu198, Ser199, Thr202, Leu203 and His207 of SEQ ID NO:14.

### Binding Sites

The term “binding site” refers to a specific region (or atom) of pyrH that enters into an interaction with a molecule that binds to pyrH. A binding site can be, for example, a conserved structural element or a combination of several conserved structural elements, a substrate binding site, an activator binding site, an inhibitor binding site, an allosteric binding site, or an intermolecular interface.

A substrate binding site includes a specific region (or atom) of pyrH that interacts with a substrate, such as adenosine triphosphate (ATP) or uridine monophosphate (UMP). A substrate binding site may comprise, or be defined by, the three dimensional arrangement of one or more amino acid residues within a folded polypeptide. In the present invention, a substrate can be a compound such as ATP or UMP which pyrH reversibly converts to adenosine diphosphate (ADP) or uridine diphosphate (UDP), respectively. Thus, a substrate can also act as a product. The substrate can be a naturally-occurring or artificial compound. Two substrates, for example UMP and ATP, can be bound either simultaneously or individually to separate, but adjacent binding sites. A subset of the residues defining each of these adjacent binding sites may therefore also be encompassed in the definition of the other binding site.

In one embodiment of the invention, the substrate binding site for H. influenzae pyrH includes the amino acids Lys11, Leu12, Ser13, Gly14, Glu15, Ala16, Leu17, Val50, Leu51, Gly52, Gly53, Gly54, Asn55, Arg58, Thr80, Asn83, Thr140, Thr141, Asp142, Ser143, Ala145, Lys159, Ala160, Thr161, Lys162, Val163, Gly165, Val166, Tyr167, Asp168, Cys169, Asp170, Pro171, Lys173, Asp174, Ala177, Lys178, Tyr180, Lys191, Glu192, Leu193, Lys194, Val195, Met196, Asp197, Val213 and Phe214 of SEQ ID NO:14. In another embodiment of the invention, the substrate binding site for H. influenzae pyrH additionally includes the amino acids Phe57, Gly59, Arg69, Val70, Val71, Gly72, Asp73, His 74, Met75, Gly76, Met77, Leu78, Ala79, Ala103, Phe104, Ser131, Ala132, Gly133, Thr134, Gly135, Asn136, Pro137, Phe138, Phe139, Thr144, Leu147 and Arg148 of SEQ ID NO: 14. In yet another embodiment of the invention, the substrate binding site for H. influenzae pyrH includes the amino acids Lys11, Leu12, Ser13, Gly14, Glu15, Val50, Leu51, Gly52, Gly53, Gly54, Asn55, Phe57, Arg58, Gly59, Arg69, Val70, Val71, Gly72, Asp73, His 74, Met75, Gly76, Met77, Leu78, Ala79, Thr80, Asn83, Ala103, Phe104, Ser131, Ala132, Gly133, Thr134, Gly135, Asn136, Pro137, Phe138, Phe139, Thr140, Thr141, Asp142, Ser143, Thr144, Ala145, Leu147 and Arg148 of SEQ ID NO:14. In yet another embodiment, the substrate binding site for H. influenzae pyrH includes the amino acids Lys11, Leu12, Ser13, Gly14, Glu15, Ala16, Leu17, Val50, Leu15, Gly52, Gly53, Gly54, Asn55, Phe57, Arg58, Gly59, Arg69, Val70, Val71, Gly72, Asp73, His74, Met75, Gly76, Met77, Leu78, Ala79, Thr80, Asn83, Ala103, Phe104, Ser131, Ala132, Gly133, Thr134, Gly135, Asn136, Pro137, Phe138, Phe139, Thr140, Thr141, Asp142, Ser143, Thr144, Ala145, Leu147, Arg148, Lys159, Ala160, Thr161, Lys162, Val163, Gly165, Val166, Tyr167, Asp168, Cys169, Asp170, Pro171, Lys173, Asp174, Ala177, Lys178, Tyr180, Lys191, Glu192, Leu193, Lys194, Val195, Met196, Asp197, Val213 and Phe214.

An inhibitor binding site includes a specific region (or atom) of pyrH that interacts with an inhibitor, such as 5′-adenylylimidodiphosphate (AMP-PNP), that acts to prevent pyrH activity. An inhibitor binding site may comprise, or be defined by, the three dimensional arrangement of one or more amino acid residues within a folded polypeptide. In the present invention, an inhibitor can be a compound which can undergo a catalytic reaction, bind to the substrate binding site, or another site, such as the allosteric binding site, on pyrH and which may compete with substrate turnover of ATP and/or UMP, or otherwise prevents pyrH activity. Inhibitors of the present invention can be a compound such as AMP-PNP, which binds at the substrate binding site, or UTP which binds at an allosteric binding site. The inhibitor can be a naturally-occurring or artificial compound.

An “allosteric binding site” includes a specific region (or atom) of pyrH that interacts with an allosteric effector, such as GTP or UTP. An allosteric binding site may comprise, or be defined by, the three dimensional arrangement of one or more amino acid residues within a folded polypeptide. The allosteric effector can be an activator, such as GTP. The allosteric effector can be a naturally-occurring or an artificial compound.

In one embodiment of the invention, the allosteric binding site for H. influenzae pyrH includes the amino acids Arg7, Gly66, Met67, Asn68, Arg69, Val70, Val71, Gly72, His74, Gly84, Leu55, Ala86, Met87, Arg88, Asp59, Ser90, Leu91, Phe92, Arg93, Asp95, Val96, Asn97, Ala98, Lys99, Leu100, Met101, Ile109, Cys110, Asp111, Asn114, Trp115, Ser116, Glu117, Ala118, Ile119, Lys120, Met121, Arg123, Glu124, Arg126, Val127, Ile129, Glu151, Ile152 and Glu153 of SEQ ID NO:14.

### Machine Readable Data Storage Medium

The list of atomic coordinates defining the pyrH crystal structure can be stored electronically, for example on a machine readable storage medium, such as a disk, so that the coordinates may be accessed and manipulated by a computer. For example, using 3D-visualisation software it is possible to depict the structure represented by the atomic coordinates on a computer graphics screen and to study hypothetical interactions with candidate inhibitors. In this way, the atomic coordinates of this invention are a useful tool for the design of novel inhibitors that are candidates for new antibacterial agents.

Computer-Assisted Methods of Identifying pyrH Binding Agents

The present invention includes a computer-assisted method for identifying a potential pyrH binding agent such as a modifier, particularly a potential inhibitor of pyrH activity.

Those of skill in the art will understand that a set of atomic co-ordinates, such as those tabulated in FIG. 7, may be manipulated mathematically, for example by rotation or translation, such that an entirely different set of atomic co-ordinates from those presented in FIG. 7 define a similar or identical shape and thus represent the same invention.

The crystal structure of pyrH, and the binding sites described herein are useful for the design of agents, particularly selective inhibitory agents, which inhibit pyrH, and, thus, could act as antibacterial agents. In a related embodiment, the present invention encompasses a method for structure-based drug design of an agent that inhibits pyrH activity.

More particularly, the design of compounds that inhibit pyrH according to this invention generally involves consideration of two factors. First, the compound must be capable of physically and structurally associating with pyrH via covalent and/or non-covalent interactions. Non-covalent molecular interactions important in the association of pyrH with its substrates, allosteric effectors, or inhibitor, include hydrogen bonding, van der Waals and hydrophobic interactions.

Second, the compound must be able to assume a conformation that allows it to associate with pyrH. Although certain portions of the compound will not directly participate in this association with pyrH, those portions may still influence the overall conformation of the molecule. This, in turn, may have a significant impact on potency. Such conformational requirements include the overall three-dimensional structure and orientation of the chemical entity or compound in relation to all or a portion of a binding site, e.g., a substrate binding site, an allosteric binding site, an intermolecular interface of pyrH, or the spacing between functional groups of a compound comprising several chemical entities that directly interact with pyrH.

The potential inhibitory effect of a chemical compound on pyrH may be estimated prior to its synthesis and testing by the use of computer modeling techniques. If the theoretical structure of the given compound suggests insufficient interaction and association between it and pyrH, synthesis and testing of the compound is obviated. However, if computer modeling indicates a strong interaction, the molecule may then be synthesized and tested for its ability to bind to pyrH in a suitable assay. In this manner, synthesis of inactive compounds may be avoided.

One embodiment of the present invention relates to any computer-assisted method using known binding agents of pyrH, such as ATP, ADP, UTP, UMP, UDP, or GTP to determine the fit of a known agent for comparison to a candidate inhibitor.

In a specific embodiment, the computer-assisted method of identifying an agent that is a binding agent of pyrH comprises the steps of (1) supplying the computer modeling application the atomic coordinates of a known agent, such as an allosteric effector and/or a substrate of pyrH, that binds a binding site of pyrH; (2) supplying the computer modeling application the atomic coordinates of pyrH as provided in FIG. 7, or alternatively, atomic coordinates having a root mean square deviation from the atomic coordinates of FIG. 7 with respect to conserved backbone atoms of the listed amino acid sequence of not more than 1.0 Å, or a root mean square deviation of not more than 1.5 Å; (3) quantifying the fit of an agent that binds the binding site of pyrH to pyrH; (4) supplying the computer modeling application with a set of atomic coordinates of an agent to be assessed to determine if it binds a binding site of pyrH; (5) quantifying the fit of the test agent in the binding site using a fit function; (6) comparing the fit calculation for the known agent with that of the test agent; and (7) selecting a test agent that has a fit better than, or approximates, the fit of the known agent. For example, the atomic co-ordinates of the known binding agent used in the method above can be those of a GDP molecule bound to the pyrH of the invention as defined by the atomic coordinates tabulated in FIG. 7. The fit of the GDP molecule to the binding site of pyrH can be quantified by calculating the surface area on both the GDP molecule and the pyrH molecule which is removed from solvent (buried surface) upon binding of the GDP to the binding site, using, for example, a program such as Areaimol (CCP4, 1994, supra). The ratio of these two values provides an estimation of the surface or shape complementarity of GDP to the binding site of pyrH. The fit of a test agent, such as UTP, which may bind to the same or similar binding site of pyrH as GDP, can then be compared to the fit of GDP by, for example, docking of UTP into the binding site of pyrH where GDP is observed to bind, and again performing a calculation to compare the surface area on both the UTP and pyrH molecules that is removed from solvent upon binding of UTP. A ratio of the buried surface areas that is closer to unity may indicate a better fit.

Another approach made possible by this invention, is to screen computationally small molecule databases for chemical entities or compounds that can bind in whole, or in part, to a binding site of pyrH. In this screening, the quality of fit of such entities or compounds to the binding site may be judged either by shape complementarity (DesJarlais et al., 1988, J. Med. Chem. 31:722-729) or by estimated interaction energy (Meng et al., 1992, J. Comp. Chem., 13:505-524).

Methods to screen chemical entities or fragments for their ability to associate with pyrH and more particularly with the individual binding sites of pyrH are known in the art. Such methods can include the use of computers in a process known as docking. Docking may be accomplished using software such as Quanta and Sybyl, followed by energy minimization and molecular dynamics with standard molecular mechanics forcefields using software such as CHARMM and AMBER.

Specialized computer programs may also assist in the process of selecting fragments or chemical entities. These include:

1. GRID (Goodford, 1985, J. Med. Chem., 28:849-857). GRID is available from Oxford University, Oxford, UK;
2. MCSS (1991, Miranker and Karplus, Proteins: Structure, Function and Genetics, 11:29-34). MCSS is available from Molecular Simulations, Burlington, Mass.;
3. AUTODOCK (Goodsell and Olsen, 1990, Proteins: Structure, Function and Genetics, 8:195-202). AUTODOCK is available from Scripps Research Institute, La Jolla, Calif.; and
4. DOCK (Kuntz et al., 1982, J. Mol. Biol., 161:269-288). DOCK is available from University of California, San Francisco, Calif.

Additional commercially available computer databases for small molecular compounds include the Cambridge Structural Database and the Fine Chemical Database (Rusinko, 1993, Chem. Des. Auto. News, 8:44-47).

Once suitable chemical entities or fragments have been selected, they can be assembled into a single compound or inhibitor. Assembly may be proceeded by visual inspection of the relationship of the fragments to each other on the 3D image displayed on a computer screen in relation to the structure/atomic coordinates of pyrH. This would be followed by manual model building using software such as Quanta or Sybyl.

Useful programs to aid one of skill in the art in connecting the individual chemical entities or fragments include:

1. CAVEAT (Bartlett et al., 1989, in Molecular Recognition in Chemical and Biological Problems, Special Pub., Royal Chem. Soc., 78:182-196). CAVEAT is available from the University of California, Berkeley, Calif.;
2. 3D Database systems such as MACCS-3D (MDL Information Systems, San Leandro, Calif.) This area is reviewed in Martin, 1992, Med. Chem., 35:2145-2154; and
3. HOOK (available from Molecular Simulations, Burlington, Mass.).

Instead of proceeding to build a pyrH inhibitor in a step-wise fashion one fragment or chemical entity at a time as described above, inhibitory or other types of binding compounds may be designed as a whole or “de novo” using either an empty active site or optionally including some portion(s) of a known inhibitor(s). These methods include:

1. LUDI (Bohm, J. Comp. Aid. Molec. Design 6:61-78, 1992). LUDI is available from Biosym Technologies, San Diego, Calif.; and
2. LEGEND (Nishibata and Itai, Tetrahedron, 47:8985, 1991). LEGEND is available from Molecular Simulations, Burlington, Mass.
3. LeapFrog (available from Tripos Associates, St. Louis, Mo.)

The potential interference of the candidate inhibitor with the activity of pyrH is assessed and the candidate inhibitor is structurally modified as needed to produce a set of atomic coordinates for a modified candidate inhibitor. The modified candidate inhibitor is further assessed, using computer-assisted techniques and, optionally, in vitro and/or in vivo testing and modified further, if needed, to produce a modified candidate inhibitor with enhanced properties (e.g., greater inhibitory activity than the starting candidate inhibitor). A variety of conventional techniques may be used to carry out each of the above evaluations as well as the evaluations necessary in screening a candidate compound for ability to inhibit pyrH. Generally, these techniques involve determining the location and binding proximity of a given moiety, the occupied space of a bound inhibitor, the amount of complementary contact surface between the inhibitor and protein, the deformation energy of binding of a given compound and some estimate of hydrogen bonding strength and/or electrostatic interaction energies. Examples of techniques useful in the above evaluations include: quantum mechanics, molecular mechanics, molecular dynamics, Monte Carlo sampling, systematic searches and distance geometry methods (Marshall, Ann. Rev. Pharmacol. Toxicol., 27:193, 1987). Specific computer software has been developed for use in carrying out these methods. Examples of programs designed for such uses include: Gaussian 92 [M. J. Frisch, Gaussian, Inc., Pittsburgh, Pa. ©1993]; AMBER [P. A. Kollman, University of California at San Francisco, ©1993]; QUANTA/CHARMM, [Molecular Simulations, Inc., San Diego, Calif., ©1992]. Other molecular modeling techniques may also be employed to screen for inhibitors of pyrH. See, for example, Cohen et al., 1990, J. Med. Chem., 33:883-894; Navia & Murcko, 1992, Curr. Opin. Struct. Biol., 2:202-210. The model building techniques and computer evaluation systems described herein are not a limitation on the present invention, but all depend for their timely execution on the availability of the atomic coordinates of pyrH as provided in FIG. 7.

Other hardware systems and software packages will be known and of evident applicability to those skilled in the art.

Thus, using these computer evaluation systems, a large number of compounds may be quickly and easily examined and expensive and lengthy biochemical testing avoided. Moreover, the need for actual synthesis of many compounds is effectively eliminated.

In another embodiment, the present invention relates to a method of making a candidate modifier of pyrH by chemical, enzymatic or other synthetic methods. Candidate modifiers identified or designed as described herein can be made using techniques known to those of skill in the art.

Once identified by the modeling techniques described herein, the inhibitor may be tested for pyrH binding and inhibitory bioactivity using standard techniques. For example, pyrH may be used in binding assays using conventional formats to screen inhibitors. Suitable assays for use include, but are not limited to, the enzyme-linked immunosorbant assay (ELISA) or a fluorescence quench assay. Other assay formats may be used, for example a coupled assay in which generation of product may be spectrophotometrically detected; these assay formats are not a limitation on the present invention.

### In Vitro and In Vivo Binding Analysis

Methods of the invention include methods for identifying inhibitors of pyrH using the crystal structure and novel binding sites described herein. Inhibitors included in the invention include any inhibitor that can bind to all, or a binding site, of pyrH, and may be competitive or non-competitive inhibitors. Once identified and screened for biological activity, these inhibitors may be used therapeutically or prophylactically to block bacterial growth and spread.

One design approach is to probe the pyrH of the invention with molecules composed of a variety of different chemical entities to determine optimal sites for interaction between candidate pyrH binding agents and pyrH. For example, high resolution X-ray diffraction data collected from crystals soaked with a solution containing a compound(s) of interest allows the determination of where each type of molecule binds. As used herein, the term “soaked” refers to a process in which the crystal is transferred to a solution containing the compound of interest, for example an organic solvent, an inhibitor, a substrate or an allosteric modulator. Small molecules that bind tightly to those sites can then be designed, synthesized and tested for their pyrH inhibitory activity (Bugg et al., 1993, Scientific American, December:92-98; West et al., 1995, TIPS, 16:67-74).

The pyrH of the invention may also be used to confirm the binding, and provide information on the binding mode of agents identified by, for example, any of the computer modelling methods described herein, in vitro binding assays, or high throughput screening. For example, high resolution diffraction data collected from crystals of pyrH grown in the presence of the proposed binding agent can be used in combination with the pyrH atomic coordinates tabulated in FIG. 7, to obtain the structure of the complex between pyrH and the proposed binding agent using the method of molecular replacement as described below. Alternatively, the atomic coordinates of the pyrH molecules listed in FIG. 7 may be used directly in combination with the experimental X-ray diffraction data to generate a difference Fourier electron density map from which the binding of the agent can be identified. Pre-existing crystals of pyrH may alternatively be transferred to a solution containing the proposed binding agent for a length of time sufficient to allow the agent to diffuse through the crystal lattice and bind to a binding site of pyrH. X-ray diffraction data can then be collected from these crystals and used as described above to determine the nature of the binding of the agent to pyrH. These methods provide confirmation of the binding of the agent to pyrH, and additionally elucidate the nature of any interactions between pyrH and the binding agent, thus permitting further rounds of optimisation of the binding agent.

The pyrH of the invention may also be used in combination with, for example, data from NMR spectroscopic experiments, to confirm the binding of agents identified by any of the computer modelling methods described above or by any other methods, for example in vitro binding assays, or high throughput screening. For example, measurement of changes in NMR chemical shifts for samples of pyrH analysed in the presence and absence of the binding agent allows determination of the binding affinity of the agent (K_{D}) for pyrH. Further, mapping of the residues giving rise to the changes in chemical shift onto the structure of the pyrH of the invention allows identification of the binding site for the agent of interest. See for example, Otting, 1993, Current Opinion in Structural Biology, 3:760-768; Hensmann et al., 1994, Protein Science, 3:1020-1030; Craik et al., 1990, Annual Reports on NMR Spectroscopy, 22:61-128.

The present invention encompasses an in vitro biological assay to identify an inhibitor of pyrH. The assay can be a single or double enzyme activity assay as described in detail in PCT/GB2004/002158, filed May 19, 2004, which is incorporated herein by reference in its entirety, or an equivalent in vitro assay system wherein small molecules, proteins, or fragments thereof are added to bacteria prior to the addition of allosteric effector and/or substrate. When growth of the bacteria is inhibited compared to the control (which lacks inhibitor) an inhibitor of pyrH has been identified.

The present invention also includes an in vivo analysis of the antibacterial activity of the test binding agents. The method includes infecting a mammalian subject (preferably a non-human primate or a rodent) with a clinically relevant amount of bacteria sufficient to establish an infection in the subject. After the bacterium has established an infection, the test inhibitor (e.g., antibacterial binding agent) can be administered to the subject. A separate control group can be administered with a placebo. Tissue, blood, and blood products can be collected at various time points to determine the course of infection, and agents that reduce (partially or totally) the extent of infection are determined to be effective inhibitors of infections (see for example, Sande et al., 1988, Reviews of Infectious Diseases, 10 Suppl. 1:S113-6 or Jacobs, 2003, International Journal of Infectious Diseases, 7 Suppl 1:S13-20).

### Homology Modelling

In certain embodiments the present invention relates to a method for generating 3-D atomic coordinates of a protein homologue or a variant of H. influenzae pyrH using the atomic coordinates of H. influenzae pyrH described in FIG. 7, comprising,

a. identifying one or more polypeptide sequences homologous to H. influenzae pyrH;

b. aligning the sequences with the sequence of H. influenzae pyrH which comprises a polypeptide with the amino acid sequence of SEQ ID NO:14;

c. identifying structurally conserved and structurally variable regions between the homologous sequence(s) and H. influenzae pyrH;

d. generating 3-D atomic coordinates for structurally conserved residues of the homologous sequence(s) from those of H. influenzae pyrH using atomic coordinates of H. influenzae pyrH, such as those listed in FIG. 7;

e. generating conformations for helices, strands, loops, and/or turns in the structurally variable regions of the homologous sequence(s);

f. building side-chain conformations for the homologous sequence(s); and

g. combining the 3-D atomic coordinates of the conserved residues, loops and side-chain conformations to generate full or partial 3-D atomic coordinates for the homologous sequence(s).

Thus the pyrH structure described herein allows the modelling of structures of homologous proteins for which experimental structural information cannot be easily obtained.

### Molecular Replacement

PyrH may crystallize in more than one form. Therefore, the atomic coordinates of pyrH as described herein are particularly useful to solve the structure of additional crystal forms of pyrH, or binding domains of additional crystal forms of pyrH. Portions of pyrH of the present invention function as the active site (substrate binding site). They may also be used to solve the structure of pyrH mutants, pyrH complexes, pyrH isozymes or of the crystalline form of other proteins with significant amino acid sequence homology or structural homology to a functional domain of pyrH. In one embodiment, significant amino acid sequence homology comprises at least 40%, 45%, 55%, 60%, 65%, 70%, 75%, 80%, 85%, 90%, 95%, 98%, or 99% identity to any functional domain of pyrH.

One method that may be employed for this purpose is molecular replacement. In this method, the unknown crystal structure, whether it is another crystal form of H. influenzae pyrH, a pyrH isozyme, or a pyrH co-complex, or the crystal of some other protein with significant amino acid sequence homology to any functional domain of pyrH, may be determined using the pyrH atomic coordinates of this invention. This method will provide an accurate structural form for the unknown crystal more quickly and efficiently than attempting to determine such information ab initio.

Examples of programs that may be used to carry out the steps of molecular replacement include MOLREP (Vagin and Teplyakov, 1997, J. Appl. Cryst., 30:1022-1025), AMoRe (Navaza, 2001, Acta Cryst., D57(10):1367-1372), Beast (Read, 2001, Acta Cryst., D57(10):1373-1382), GLRF (Tong & Rossmann, 1990, Acta Cryst., A46:783-792), COMO (Jogl et al., 2001, Acta Cryst., D57(8):1127-1134), EPMR (Kissinger et al., 1999, Acta Cryst., D55(2):484-491). The MOLREP, AMoRe and Beast software are distributed as part of the CCP4 software package (CCP4, Acta Cryst., D50:760-763, 1994). As an example, MOLREP is an integrated molecular replacement program that finds molecular replacement solutions using a two-step procedure: (1) rotation function (RF) search to identify the orientation of the model and (2) cross translation function (TF) and packing function (PF) search to identify the position of the oriented model. The translation function checks several peaks of the rotation function by computing a correlation coefficient for each peak and sorting the result. The packing function is important in removing incorrect solutions that correspond to overlapping symmetry. MOLREP can be set to search for any number of molecules per asymmetric unit and will automatically stop when no further improvement of the solution can be achieved by adding additional molecules.

In another aspect, the present invention provides a method involving molecular replacement to obtain structural information about a molecule or molecular complex of unknown structure using the software programs described above, or equivalent programs known to those skilled in the art, and the atomic coordinates described herein and tabulated in FIG. 7.

### Practice of the Invention

The practice of the present invention employs, unless otherwise indicated, conventional techniques of cell biology, cell culture, molecular biology, microbiology and recombinant DNA manipulation, X-ray crystallography, NMR spectroscopy and molecular modeling which are within the skill of the art. Such techniques are explained fully in the literature. See, for example, Molecular Cloning: A Laboratory Manual, 2nd Ed., ed. Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Crystallography made crystal clear: a guide for users of macromolecular models (Gale Rhodes, 2nd Ed. San Diego: Academic Press, 2000).

### EQUIVALENTS

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with the true scope and spirit of the invention being indicated by the claims.

The invention is further illustrated by way of the following examples, which are intended to elaborate several embodiments of the invention. These examples are not intended to, nor are they to be construed to, limit the scope of the invention.

### EXAMPLES

### Example 1

### Cloning, Purification and Characterization of H. influenzae pyrH

The H. influenzae pyrH UMP kinase gene (HI-1065) was cloned from genomic DNA extracted from the sequenced Rd strain KW20 (Fleischmann et al., 1995 Science, 269: 496-512). Cloning was accomplished using specifically designed primers to amplify the protein coding sequence of HI-1065. An NcoI restriction enzyme (RE) site was engineered into the 5′ primer and a SalI RE site was engineered into the 3′ primer, to facilitate subcloning into pET28b for over-expression. The 5′ primer included 9 bp upstream of the ATG start codon and the 3′ primer included 16 bp (including SalI) downstream of the TAG stop codon. When introducing the NcoI site, the first nucleotide of the second codon of pyrH was changed from an A(GC) to a G(GC) resulting in a serine (in the HI1065 encoded protein sequence) to glycine (in the pSM143 encoded protein sequence) substitution.

The two primers used for amplification were as follows:

5′ HI1065-NcoI

SEQ ID NO: 11

5′ GAAAAAACCATGGGCCAACCAATTTATAAACG 3′
3′ HI1065R-SalI

SEQ ID NO: 12

5′ TTCTTGTCGACATTCACTAACAAATAGTGGTGCC 3′

After PCR amplification of pyrH using the above primers, the resulting DNA was gel purified and cloned into pGEM-T (Promega). The DNA insert in this clone (PSM143) was analysed to confirm that the correct sequence was present without unintended errors. Upon sequencing, it was discovered that the 3′ SalI RE site was not present as designed (most likely due to an error in the primer synthesis). The protein coding sequence was, however, correct, so the pyrH gene was first subcloned from pSM143 into pUC128 (Keen et al., 1988, Gene, 70(1): 191-197) as a PstI-SacII fragment (pSM144), and subsequently cloned into pET28b as a NcoI-EcoRI fragment to make pSM145. pSM145 was transformed into E. coli strain BL21 (DE3) for over-expression. Expression was carried out at 30° C. The growing culture was induced with 1 mM IPTG when the cell density reached an OD₆₀₀ between 0.35 and 0.5. The cells were collected by centrifugation, and chilled to 4° C., after growing for 2 additional hours. The resulting cell paste was stored frozen. Production of seleno-methionine (SeMet) labelled pyrH was achieved by growth of E. coli BL21(DE3)/pSM145 cells in 2 L M9 minimal medium supplemented with 0.05 mg/ml SeMet and 40 μg/ml Kanamycin at room temperature for 5.5 hours, followed by induction of pyrH expression overnight at room temperature with 1 mM IPTG. Production of ²H¹³C¹⁵N labeled pyrH was achieved by growth of E. coli BL21(DE3)/pSM145 cells in 1 L Silante's E. coli OD2 CDN medium (VLI Research) supplemented with 40 μg/ml Kanamycin at 30° C. for 6.5 hours, followed by induction of pyrH expression overnight at 30° C. with 1 mM IPTG.

Nucleotide sequence of cloned insert in pSM145:

(SEQ ID NO: 13)

ATGGGCCAACCAATTTATAAACGTATTTTATTGAAATTAAGCGGTGAAGC
ATTACAAGGAGAAGATGGTCTTGGTATCGATCCTGCGATTCTCGATCGTA
TGGCTGTTGAAATTAAAGAATTAGTGGAGATGGGTGTGGAAGTCAGTGTC
GTTCTCGGTGGTGGCAACTTATTCCGTGGCGCAAAACTAGCAAAAGCGGG
GATGAATCGCGTGGTGGGCGATCATATGGGAATGCTTGCTACTGTGATGA
ATGGTTTGGCAATGCGTGATTCTTTATTCCGTGCTGATGTGAACGCAAAA
TTAATGTCCGCTTTCCAATTAAATGGTATTTGCGATACTTATAACTGGTC
TGAAGCTATCAAAATGTTACGCGAAAAACGCGTAGTCATTTTCTCTGCGG
GAACGGGAAATCCATTCTTTACCACTGATTCTACCGCTTGTTTGCGTGGT
ATTGAAATTGAAGCTGATGTTGTGTTGAAAGCGACTAAAGTTGATGGTGT
GTATGATTGTGATCCTGCGAAAAATCCTGATGCAAAACTTTATAAAAATT
TAAGTTATGCAGAAGTGATCGATAAAGAATTAAAAGTGATGGACTTATCG
GCGTTTACTTTAGCTCGCGATCATGGCATGCCGATTAGAGTGTTCAATAT
GGGTAAACCTGGAGCATTACGTCAAGTAGTGACTGGTACTGAAGAAGGCA
CCACTATTTGTTAG

Amino acid sequence of cloned insert in pSM145:

(SEQ ID NO: 14)

MGQPIYKRILLKLSGEALQGEDGLGIDPAILDRMAVEIKELVEMGVEVSV
VLGGGNLFRGAKLAKAGMNRVVGDHMGMLATVMNGLAMRDSLFRADVNAK
LMSAFQLNGICDTYNWSEAIKMLREKRVVIFSAGTGNPFFTTDSTACLRG
IEIEADVVLKATKVDGVYDCDPAKNPDAKLYKNLSYAEVIDKELKVMDLS
AFTLARDHGMPIRVFNMGKPGALRQVVTGTEEGTTIC

Purification of H. influenzae pyrH for use in Crystallisation

The frozen cell paste was suspended in 40 ml of Lysis Buffer [50 mM Tris-HCl, pH 8.0, 2 mM EDTA, 2 mM DTT, 2 mM UTP, 1 mM PMSF, 1 Protease inhibitor cocktail tablet (Roche Molecular Biochemical)]. Cells were disrupted by passing them twice through a French press operated at 18,000 psi, and the crude extract was centrifuged at 25,000 rpm (45Ti rotor, Beckman) for 30 min at 4° C. The supernatant was loaded at a flow rate of 1.5 ml/min onto a 20 ml Q-Sepharose HP(HR16/10) column (Amersham Biosciences) pre-equilibrated with Buffer A (50 mM Tris-HCl, pH 8.0, 2 mM EDTA, 2 mM DTT, 2 mM UTP). The column was then washed with Buffer A, and the protein was eluted by a linear gradient from 0 to 1 M NaCl in Buffer A. Fractions containing PyrH were pooled, and solid (NH₄)₂SO₄ (0.4 g/ml) was added, to precipitate all the proteins, and mixed on ice for 1 hour. The sample was centrifuged at 11,000 rpm for 30 min at 4° C. (JA12 rotor, Beckman). The pellet was then dissolved in 7 ml of Buffer A. The 7 ml sample was applied at a flow rate of 1.0 ml/min to a 320 ml Sephacryl S-300 (HR 26/60) column (Amersham Biosciences) pre-equilibrated with Buffer B (50 mM Tris-HCl, pH 8.0, 2 mM EDTA, 2 mM DTT, 2 mM UTP, 150 mM NaCl). The fractions containing PyrH were pooled and dialyzed against 1 L Storage Buffer (50 mM Tris-HCl, pH 8.0, 0.1 mM EDTA, 1 mM UTP, 150 mM NaCl, 2 mM DTT, 20% glycerol).

The protein was characterized by SDS-PAGE analysis and analytical LC-MS. The determined mass of the protein indicated that the N-terminal methionine of the polypeptide predicted from the DNA sequence was not present [expected MW=25569.0 Da (—N-terminal Met), observed=25565 Da (—N-terminal Met)]. The protein was stored at 193K.

Purification of SeMet labelled H. influenzae PyrH for use in Crystallisation

The frozen cell paste was suspended in 40 ml of Lysis Buffer [50 mM Tris-HCl, pH 8.0, 2 mM EDTA, 2 mM DTT, 2 mM UTP, 1 mM PMSF, 1 Protease inhibitor cocktail tablet (Roche Molecular Biochemical)]. Cells were disrupted by passing them twice through a French press operated at 18,000 psi, and the crude extract was centrifuged at 25,000 rpm (45Ti rotor, Beckman) for 30 min at 4° C. The supernatant was loaded at a flow rate of 1.5 ml/min onto a 20 ml Q-Sepharose HP(HR16/10) column (Amersham Biosciences) pre-equilibrated with Buffer A (50 mM Tris-HCl, pH 8.0, 2 mM EDTA, 2 mM DTT, 2 mM UTP). The column was then washed with Buffer A, and the protein was eluted by a linear gradient from 0 to 1 M NaCl in Buffer A. Fractions containing PyrH were pooled, and solid (NH₄)₂SO₄ (0.4 g/ml) was added to precipitate all the proteins and mixed on ice for 1 hour. The sample was centrifuged at 11,000 rpm for 30 min at 4° C. (JA12 rotor, Beckman). The pellet was then dissolved in 6 ml of Buffer A. The 6 ml sample was applied at a flow rate of 1.0 ml/min to a 320 ml Sephacryl S-300 (HR 26/60) column (Amersham Biosciences) pre-equilibrated with Buffer B (50 mM Tris-HCl, pH 8.0, 2 mM EDTA, 2 mM DTT, 2 mM UTP, 150 mM NaCl). The fractions containing PyrH were pooled and dialyzed against 1 L Storage Buffer (50 mM Tris-HCl, pH 8.0, 0.1 mM EDTA, 1 mM UTP, 150 mM NaCl, 2 mM DTT, 20% glycerol).

The protein was characterized by SDS-PAGE analysis and analytical LC-MS. The determined mass of the protein indicated that the N-terminal methionine of the polypeptide predicted from the DNA sequence was not present, and that all other methionines were correctly substituted with seleno-methionine (SeMet) [expected MW=26127.6 Da (—N-terminal SeMet), observed=26126.0 Da (—N-terminal SeMet)]. The protein was stored at 193K.

Purification of Isotopically Labelled H. influenzae pyrH for use in NMR Spectroscopy Experiments

Purification of ²H¹³C¹⁵N labeled H. influenzae pyrH was carried out as described for the SeMet labelled pyrH.

### Example 2

### Crystallisation of H. influenzae pyrH

Purified H. influenzae pyrH was subjected to sparse matrix crystallisation screening, using a protein concentration of about 10 mg/ml at a temperature of 288K in the presence of nucleotides (1 mM UMP, 1 mM UTP, 1 mM GTP and 1 mM ATP). Screening leads were optimised using standard techniques.

Crystals having the atomic coordinates of FIG. 7 were obtained by vapor diffusion using the hanging drop method (see, for example, “Protein Crystallization”, Terese M. Bergfors (Ed.), International University Line, pp 7-15, 1999). Purified SeMet labelled H. influenzae pyrH had been stored at 193K at a concentration of 93 mg/ml in Storage Buffer (50 mM Tris-HCl pH 8.0, 0.1 mM EDTA, 1 mM UTP, 150 mM sodium chloride, 2 mM DTT, 20% glycerol). Single aliquots containing approximately 2.3 mg of protein were thawed from storage, and were extensively washed in the following buffer: 50 mM Tris-HCl pH 8.5, 50 mM NaCl, 10 mM DTT, 0.1 mM EDTA, 1 mM ATP; 1 mM GTP, imM UMP, 1 mM UTP. The final protein concentration was adjusted to 10 mg/ml. Crystals were also obtained using a final protein concentration of from about 5 to about 20 mg/ml. AMP-PNP, 2′-BrATP or 2′-IATP were all successfully substituted for ATP in the final protein solution. The reservoir solution typically contained 22-27% (w/v) polyethylene glycol (PEG) 1000, 100 mM lithium sulphate, and 100 mM phosphate-citrate buffer pH 5.0. Phosphate-citrate buffer was prepared by titrating sodium hydrogen phosphate with citric acid to achieve the required pH. The concentration of PEG1000 in the reservoir solution could be varied from about 15% (w/v) to about 30% (w/v), and crystals obtained by corresponding adjustment of the pyrH concentration in the protein solution, or the ratio of protein solution to reservoir solution in the hanging drop. Crystals were also obtained using reservoir solutions in which a variety of different molecular weight PEGs (PEG600 to PEG8000) were substituted for PEG1000, and in which a number of other salts (for example, sodium sulphate, sodium chloride, lithium chloride) were substituted for lithium sulphate. The concentration of lithium sulphate in the reservoir solution could be varied from about 50 mM to about 400 mM, with optimal results observed from about 100 to about 250 mM lithium sulphate.

Hanging drops were set up by mixing 2 microliters of protein solution with 2 microliters of the reservoir solution and suspending the drop over 500 microliters of reservoir solution. Crystals of dimensions up to 400×200×100 microns were observed to grow within 5 days at an ambient temperature of 288 K. Crystals were also obtained over a wider temperature range (from about 285 K to about 295 K). The size of the hanging drop, and the ratio of protein to reservoir solution may also be varied. Unlabelled pyrH was crystallised under similar conditions as for the SeMet labelled protein, with the exception that 2 mM DTT was substituted for 10 mM DTT in the protein solution.

Cryoprotectant was introduced into drops containing selected crystals in the following manner. Stock solutions containing the reservoir solution corresponding to the selected drop supplemented with 5, 10, 15 and 20% (v/v) ethylene glycol were prepared. A nominal drop volume of 2 microliters was assumed for fully equilibrated drops containing crystals set up as described above (i.e., by mixing 2 microliters protein solution with 2 microliters reservoir solution). One nominal drop volume (i.e., 2 microliters) of the reservoir solution supplemented with 5% (v/v) ethylene glycol was added to the selected drop containing the crystals, mixed gently and allowed to equilibrate for 30 seconds. One nominal drop volume (i.e., 2 microliters) of the reservoir solution supplemented with 10% (v/v) ethylene glycol was then added to the drop, mixed gently and allowed to equilibrate for 30 seconds. Two nominal drop volumes (i.e., 4 microliters) of the reservoir solution supplemented with 15% (v/v) ethylene glycol were then added to the drop containing the crystals, mixed gently and allowed to equilibrate for 30 seconds. A sample of the drop solution was then taken up in a crystal mounting loop (Hampton Research, California, USA) and tested by flash cooling in a cold (100K) nitrogen stream. If the solution formed a clear glass, then selected crystals were taken from the drop and similarly flash cooled. If the solution formed an opaque ice, then two nominal drop volumes (i.e., 4 microliters) of the reservoir solution supplemented with 20% (v/v) ethylene glycol were added to the drop containing the crystals, mixed gently and allowed to equilibrate for 30 seconds, before removing the selected crystals from the drop using a crystal mounting loop, and flash cooling in a cold (100 K) nitrogen stream. Flash-cooled crystals were typically tested on an in-house Mar345 detector (MarResearch, Hamburg, Germany) prior to data collection at a synchrotron radiation source.

### Example 3

### X-ray Diffraction Data Collection

Crystals diffracted to about 3.2 Å resolution using an in-house X-ray source (MarResearch 345 mm image-plate detector system with X-rays generated on a Bruker-Nonius FR591 rotating anode operated at 45 kV and 90 mA). Crystals belonged to the rhombohedral space group R32 and had cell parameters of a=b=c=(146.5+/−0.7) Å, and α=β=γ=(97.38+/−0.07)°. Those of skill in the art will recognise that it is convenient to work in the hexagonal equivalent of the rhombohedral space group, wherein the pyrH crystal of the invention had unit cell parameters of a=b=(215+/−1.0) Å and c=(233.6+/−1.5) Å, and α=β=90°, and γ=120°. This crystal form is encompassed by the atomic coordinates of FIG. 7. A complete data set was collected at PX14.2, SRS, Daresbury, UK to 2.3 Å resolution (see Table 2, peak 1).

MAD (Multiwavelength Anomalous Dispersion) data were collected from a single crystal of SeMet labelled H. influenzae pyrH at PX14.2, SRS, Daresbury, UK. Four datasets were collected at three different wavelengths (0.9600 Å, 0.9794 Å, and 0.9797 Å). The data were autoindexed and integrated, scaled and merged, and truncated using the programs MOSFLMv6.2.3 (Leslie, Jnt CCP4/ESF EACMB Newslett. Protein Crystallogr., 26, 1992; Powell, 1999, Acta Cryst. D55(10):1690-1695), SCALA and TRUNCATE (Collaborative Computational Project, Number 4 (CCP4), Acta Cryst., D50:760-763, 1994), respectively. Statistics of the MAD data collection are shown in Table 2.

TABLE 2
MAD data collected at SRS, PX14.2; maximum resolution 2.3 Å

| | Wave- | | | | | | | |
|---|---|---|---|---|---|---|---|---|
| | length | Resolution | | | % | Multi- | | |
| Dataset | (Å) | limit (Å) | Nₘₑₐₛ | N_{ref} | poss | plicity | R_{fac} | Rₐₙₒₘ |
| peak1 | 0.9794 | 2.3 | 475345 | 77376 | 90.9 | 5.9 | 0.053 | 0.037 |
| (native) | | | | | | | | |
| peak2 | 0.9794 | 2.6 | 401878 | 60497 | 98.9 | 6.5 | 0.084 | 0.049 |
| inflection | 0.9797 | 2.4 | 480691 | 73231 | 95.1 | 6.4 | 0.052 | 0.029 |
| remote | 0.9600 | 2.7 | 398160 | 55667 | 99.9 | 7.1 | 0.074 | 0.058 |

R_{fac} = Sum|<I> − Ij|/Sum |Ij|
Rₐₙₒₘ = Sum|<I⁺> − <I⁻>|/Sum|<I⁺> − <I⁻>|

### Example 4

### Phase Determination using MAD Data

The program Xprep (Bruker, Madison, USA) was used to analyse, scale together and calculate deltaF values (where deltaF=|F⁺−F⁻|) from the four datasets described in Table 1.

Analysis of the crystal unit cell contents indicated that the crystallographic asymmetric unit could contain between 6 and 12 molecules of pyrH. Given that H. influenzae pyrH is known to exist as a homo-hexamer in solution, we initially assumed that the asymmetric unit would contain a single hexameric assembly (i.e., six pyrH molecules and thus 72 selenium atoms (Se)). The deltaF values output by Xprep were used as input to the program ShelxD (Schneider et al., 2002, Acta Cryst., D58: 1772-1779), which was asked to find 72 Se sites. The top-ranking solution (CC=59.4%) contained 101 potential Se sites, with a clear drop in occupancy after the 70^{th} site. The top 72 Se sites were verified, and the correct enantiomorph determined using the program ShelxE (Sheldrick, 2002, Zeitschrift fur Kristallographie, 217(12):644-650): high values for contrast (0.46, this value provides an indication of the presence within the experimental electron density map of regions of large fluctuations (within the protein envelope) and of little fluctuation (within the solvent) in the value of the electron density) and connectivity (0.91, this value is the fraction of adjacent pixels in the electron density map that are either both classed as solvent or as not solvent) indicated a correct solution. These 72 Se sites were refined, phases were calculated and improved by solvent flipping (a method of density modification), and a partial model for the polypeptide backbone was automatically built and refined using the programs SHARP (La Fortelle et al., 1997, Methods Enzymol., 276:472-494), SOLOMON (Abrahams et al., 1996. Acta Cryst., D52:30-42, 1996; CCP4, 1994, supra), ARP/wARP (Perrakis et al., 1999, Nature Struct. Biol., 6(5):458-463) and Refmac (Murshudov et al., 1997, Acta Cryst., D53(3):240-255; CCP4, 1994, supra) as implemented in the AutoSHARP program suite (Bricogne et al., 2003, Acta Cryst., D59(11):2023-2030). Three of the datasets—peak1 (used as native), inflection and remote (see Table 2), were used as input for AutoSHARP. The partial model output by ARP/wARP contained 1236 residues grouped into 70 separate chains. Inspection of this partial model, and the associated electron density map, confirmed the presence of a hexameric assembly of pyrH molecules in the crystallographic asymmetric unit. However, an additional dimer of pyrH molecules was also identified. This dimer lies close to the crystallographic three-fold axis with the result that a hexamer can be generated from the dimer within the crystal lattice by operation of crystallographic symmetry. The crystal, therefore, has eight molecules of pyrH in the asymmetric unit.

A repeat run of ShelxD located eighty-seven of the ninety-six possible Se sites. These sites were again verified, and the correct enantiomorph determined using the program ShelxE. High values for contrast (0.60) and connectivity (0.93) indicated a correct solution. Five additional Se sites were located, a total of 92 Se positions were refined, phases were calculated and improved by solvent flipping, and a partial model for the polypeptide backbone was automatically built and refined as before using programs from the AutoSHARP program suite. The partial model output by ARP/wARP contained 1244 of a possible 1888 residues grouped into 65 separate chains. Phasing statistics using SHARP, SOLOMON and DM are given in Table 3.

TABLE 3
Phasing statistics using SHARP, SOLOMON and DM (2.3 Å)

| | | Peak1 | | Inflection | | remote | |
|---|---|---|---|---|---|---|---|
| | | Phasing | | Phasing | | Phasing | |
| | | power | R_{cullis} | power | R_{cullis} | power | R_{cullis} |
| isomorphous | acentric | | | 0.26 | 0.915 | 0.211 | 0.907 |
| | centric | | | 0.397 | 0.682 | 0.247 | 0.666 |
| anomalous | acentric | 0.826 | 0.89 | 0.586 | 0.935 | 0.48 | 0.874 |

FOM (before density modification): acentric 0.314, centric 0.147
FOM (after solvent flattening (SOLOMON)): 0.710
FOM (after density modification with ncs averaging (DM)): 0.769

### Example 5

### Model Building and Refinement of the H. influenzae pyrH Crystal Structure

The electron density map output by AutoSHARP was marginally improved by density modification with eight-fold non-crystallographic symmetry (ncs) averaging as implemented in the program DM (CCP4, 1994, supra). The polypeptide chain was easily traced using the solvent flattened maps from AutoSHARP in combination with the density-modified maps from DM, and guided by the partial model and the 92 SeMet sites. An initial model was built for a single molecule (monomer) of pyrH: the entire main-chain and the majority of the side-chains were well defined, with the exception of two loop regions (amino acid residues 18 to 24, and amino acid residues 168 to 179 of SEQ ID NO:14). Electron density for the latter loop region, consisting of residues 168 to 179, was visible in a subset of the molecules in the crystallographic asymmetric unit allowing a model for this region to be built with some degree of confidence. A significant peak in the difference Fourier (mFo-DmFc) electron density map consistent with a bound phosphate ion was observed close to a conserved glycine-rich region (Gly52-Gly53-Gly54 of SEQ ID NO: 14). The phosphate was most likely derived from the phosphate-citrate buffer present in the crystallisation reservoir solution.

Non-crystallographic symmetry operations derived from the partial model were then used to generate the eight molecules in the crystallographic asymmetric unit from the initial monomer model.

This model, containing eight molecules, was subjected to 20 cycles of rigid body refinement (each molecule was defined as a single rigid body) using the program Refmac5 (CCP4, 1994, supra). In this way an improved set of ncs operators was obtained for use in subsequent rounds of ncs-constrained refinement. Refinement was continued using the program CNX (Accelrys), applying bulk solvent and overall anisotropic B-factor corrections. Two rounds of ncs constrained simulated annealing with torsion angle dynamics (starting temperature 2500 K) followed by 20 cycles of energy minimisation and finally 15 cycles of individual isotropic B-factor refinement were performed against phases calculated from the model. A round of interactive model building was then carried out using the program QUANTA (Accelrys; Oldfield, Acta Cryst., D57(1):82-94, 2001). At this point, a second set of significant peaks in the difference Fourier electron density map that did not belong to any of the protein side-chains was found close to residue Arg88 near the C-terminii of the long helices that define the dimer interface (alphaC, see FIGS. 1-3). This density was interpreted as arising from bound nucleotide. In the later stages of refinement, the electron density in this region became sufficiently clear to place seven molecules of the allosteric effector, GTP, into the eight potential binding sites that exist in the asymmetric unit. The triphosphate moiety of the bound GTP molecules is poorly ordered, with the beta-phosphates adopting at least three distinct conformations. The position of the gamma-phosphate is not well determined, and it has therefore been omitted from the final model. After a further round of interactive rebuilding, the monomer model was subjected to a further round of ncs constrained refinement using the program CNX. The entire contents of the asymmetric unit were then regenerated from the refined monomer model by application of ncs operators as before. Further rounds of interactive model building using the program QUANTA were alternated with ncs restrained refinement using the program CNX (progressively releasing the ncs restraints as the quality of the model improved) to produce a model with an R-value of 0.25 and an R-free value of 0.28. Two further rounds of refinement using the program Refmac5, refining TLS (translation, libration and screw), positional and isotropic B parameters without applying ncs restraints, gave a final model with an R-value of 0.22 and an R-free value of 0.25.

The R-value describes the discrepancy between the observed data and synthetic data calculated from the model. The R-free is the same, but calculated from a test set of reflections, usually 5% of total, that are set aside at the beginning of the refinement and serve as an unbiased reference to avoid over-fitting of the data. The R-value is resolution dependent but should typically be equal to or less than 0.25, and the Rfree typically not more than 0.05 higher than the R-value.

The final model consists of eight polypeptide chains of up to 236 amino acids (amino acid residues 1 to 20, 23 to 168 and 179 to 236 of molecule A, amino acid residues 1 to 18 and 24 to 236 of molecule B, amino acid residues 1 to 236 of molecules C, D, F, G, and H, and amino acid residues 1 to 18 and 23 to 236 of molecule E as defined in SEQ ID NO:14 and FIG. 7), seven molecules of GDP, eight phosphate ions, one sulphate ion and 358 ordered water molecules. Despite the presence of 1 mM ATP, 11 mM UMP and 1 mM UTP in the crystallisation solution, these nucleotides were not observed in the H. influenzae pyrH crystal structure. Statistics of the final model are given in Table 4.

TABLE 4
Refinement statistics for H. influenzae pyrH final model

| Resolution limits (Å) | | 500-2.3 |
|---|---|---|
| Number of | Total | 83469 |
| reflections | Free set | 4195 |
| R-value (%) | | 21.6 |
| Free R value (%) | | 25.3 |
| Number of | Protein atoms | 14215 |
| atoms/molecules | Ligand molecules | 16 |
| in the model | Water molecules | 358 |
| RMSD from | Bond length (Å) | 0.013 |
| ideal values | Bond angle (°) | 1.366 |
| Ramachandran | Most favoured regions (%) | 92.3 |
| plot | Additional allowed regions | 7.2 |
| | (%) | |
| | Generously allowed regions | 0.3 |
| | (%) | |
| | Disallowed regions (%) | 0.2 |

| | Molecule | A | B | C | D | E | F | G | H |
|---|---|---|---|---|---|---|---|---|---|
| Averaged B- | overall | 66 | 56 | 55 | 45 | 47 | 53 | 69 | 71 |
| values (Å²) | Main-chain | 65 | 55 | 54 | 43 | 46 | 52 | 69 | 69 |
| | Side-chain | 67 | 57 | 56 | 46.5 | 48 | 54 | 70 | 72 |
| | phosphate | 65 | 62 | 43 | 56 | 46 | 54 | 66 | 59 |
| | GDP | R | Q | | P | V | S | T | U |
| | | 67 | 72 | | 57 | 75 | 61 | 60 | 68 |

| | |
|---|---|
| water | 47 |

FIG. 7 is a listing of the three-dimensional atomic coordinates of the crystal structure of pyrH from H. influenzae complexed with GTP and phosphate. The atomic coordinates presented are those of all eight polypeptide chains of the crystallographic asymmetric unit. Chains C, D, E, F, G and H constitute one hexameric assembly. Crystallographic symmetry operations can be used by one skilled in the art to generate a similar hexameric assembly from the dimeric assembly comprised of chains A and B. In the figures, the atom listing is preceded by the heading CRYST1, which is followed by the 3 dimensions of the crystallographic unit cell. The next three values define a matrix that converts atomic co-ordinates from orthogonal Angstrom coordinates to fractional coordinates of the unit cell. Each row labeled ATOM gives the (arbitrary) atom number, the label given to each amino acid main chain, each atom type, the amino acid residue type, the protein chain label (A comprises the first molecule (chain), B comprises the second molecule (chain), C comprises the third molecule (chain), D comprises the fourth molecule (chain), E comprises the fifth molecule (chain), F comprises the sixth molecule (chain), G comprises the sixth molecule (chain), and H comprises the sixth molecule (chain)), and the amino acid residue number. The first three numbers in the row give the orthogonal X, Y, Z coordinates of the atom. The next number is an occupancy number and is less than 1.0 if the atom was seen in more than one position (the amino acid could be seen in more than one orientation). The final number is a temperature factor that relates to the thermal amplitude of vibrations of the atom. At the end of the listing, there are lines of data indicating the bound GTP molecules (GDP), phosphate ions (PO₄), sulphate ion (SO₄) and ordered water molecules (HOH) included in the model.

### Example 6

### Defining the Binding Sites of H. influenzae pyrH

The asymmetric unit of the pyrH crystal consists of eight copies of the pyrH polypeptide chain, corresponding to a homohexameric assembly and a homodimer from which a hexameric assembly can be generated by the operation of crystallographic symmetry transformations. The H. influenzae pyrH hexameric assembly can be described as a trimer of dimers. The dimers are held together by a stable and conserved hydrophobic core, created by the interlocking of methionine residues along the faces of two extended antiparallel alpha-helices (residues 68 to 95, helix C, see FIG. 1). Additional interactions are made between the conserved beta3-beta4 loops of each pyrH molecule (comprising residues 104 to 109), which are oriented antiparallel. There are also stabilising contacts between residues 25, 27, and 31 from the beta1-alphaA loop and the N-terminus of the alphaA helix (see FIG. 1) in one pyrH molecule and residues 57, 61, 62 and 65 from a short helical region (helix B, see FIG. 1) in the second molecule. The intermolecular dimer interface thus comprises amino acid residues Ile25, Pro27, Leu30, Asp31, Phe57, Lys61, Leu62, Ala65, Gly66, Met67, Asn68, Arg69, Val71, His74, Met75, Gly76, Leu78, Ala79, Val81, Met82, Leu85, Ala86, Arg87, Asp88, Arg89, Phe104, Gln105, Leu106, Asn107, Gly108 and Ile109 of SEQ ID NO: 14.

Three dimers come together to form a hexameric assembly through interactions involving residues Asn68, Arg69, Val70, Val71, His74, Arg88, Phe92, Lys99, Gln105, Leu106, Asn107, Gly108, Ile109, Cys110, Asp111, Thr112, Tyr113, Asn114, Trp115, Glu117, Thr134, Asn136, Pro137, Phe138, Phe139, Leu147, Arg148, Ile150, Glu151, Glu153, Leu198, Ser199, Thr202, Leu203 and 1 is 207 of a first molecule (chain) from a first dimer, residues Asn68, Arg69, Val70, Val71, His74, Thr134, Asn136, Pro137, Phe138, Phe139, Leu147, Arg148, Ile150, Glu151, Glu153, Leu198, Ser199, Thr202, Leu203 and His207 of a second molecule (chain) from a second dimer, residues Arg88, Phe92, Lys99, Asn107, Gly108, Ile109, Cys110 and Asp 111 of a third molecule (chain) from the second dimer, and residues Gln105, Leu106, Thr112, Tyr113, Asn114, Trp115 and Glu117 of a fourth molecule (chain) from a third dimer. All contacts were determined using a radius of 5 Å and the program Contact (CCP4, 1994, supra). The intermolecular dimer-dimer interface thus comprises amino acid residues Asn68, Arg69, Val70, Val71, His74, Arg88, Phe92, Lys99, Gln105, Leu106, Asn107, Gly108, Ile109, Cys110, Asp111, Thr112, Tyr113, Asn114, Trp115, Glu117, Thr134, Asn136, Pro137, Phe138, Phe139, Leu147, Arg148, Ile150, Glu151, Glu153, Leu198, Ser199, Thr202, Leu203 and His207 of SEQ ID NO:14.

### Allosteric Binding Site

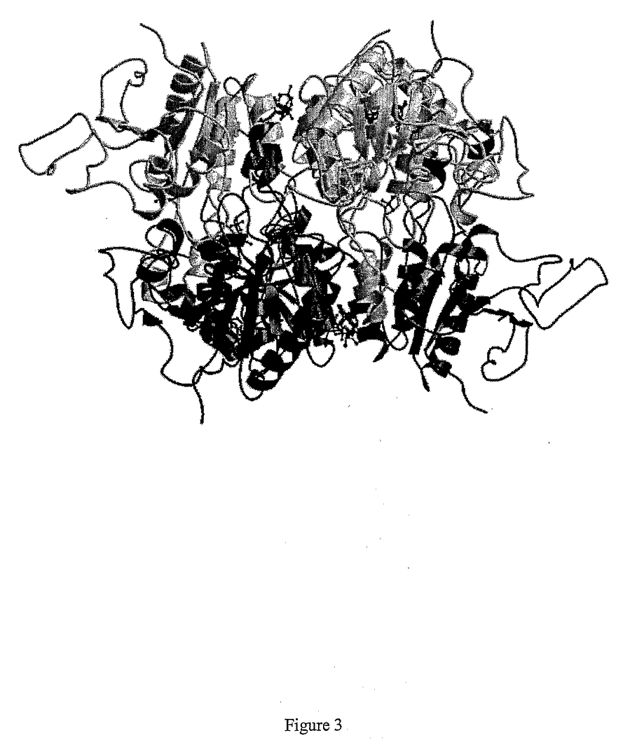

The allosteric effector, GTP, binds at the interface of two dimers, close to the centre of the hexameric assembly. Each bound GTP molecule is in contact with residues from three pyrH molecules (see FIGS. 2-3), binding at a site in sequence corresponding to residues 88-99 and 126 of a first molecule from one dimer, 68-71 of a second molecule from the same dimer and 115-123 of a third molecule from a second, adjacent dimer. GTP binding may therefore be both dependent on, and contribute to, hexamer formation. The hexamer is stabilised in the presence of UTP, which may bind at the interface of adjacent dimeric units at the same site as GTP.

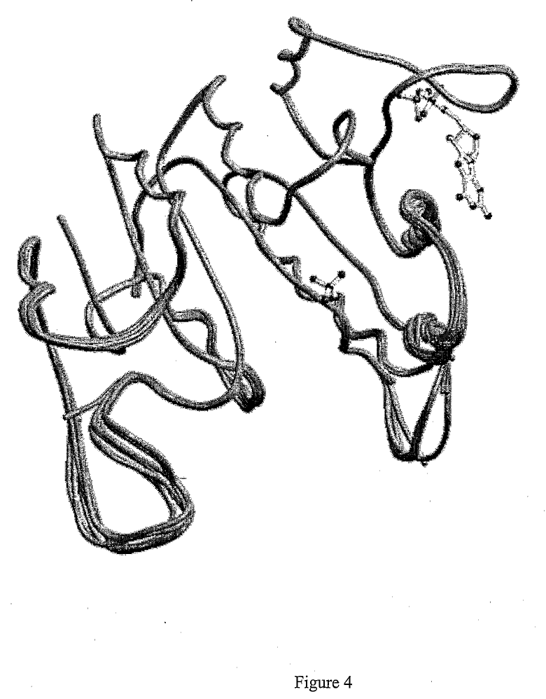

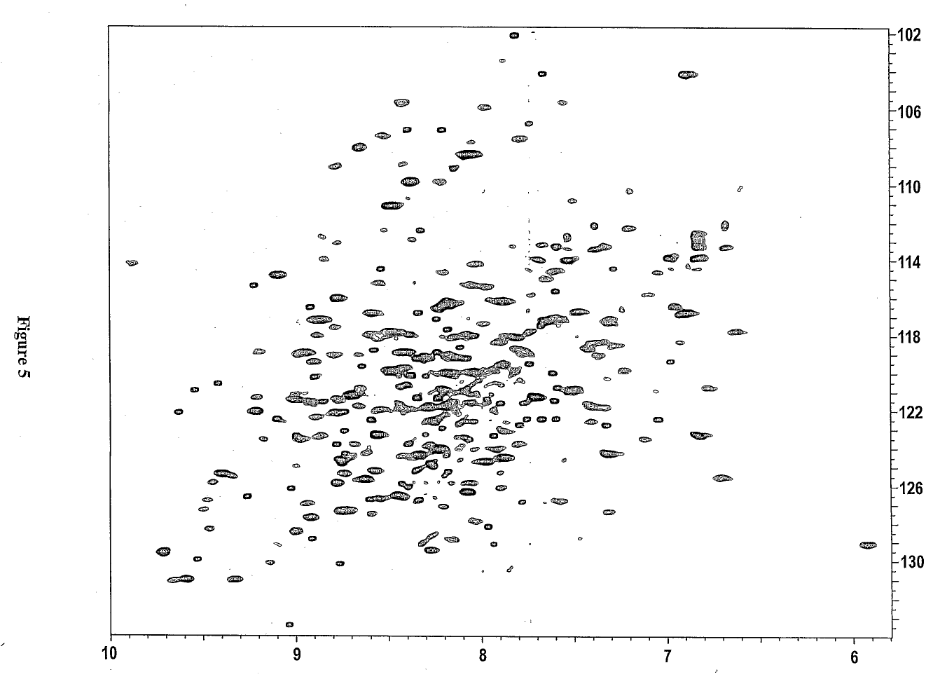

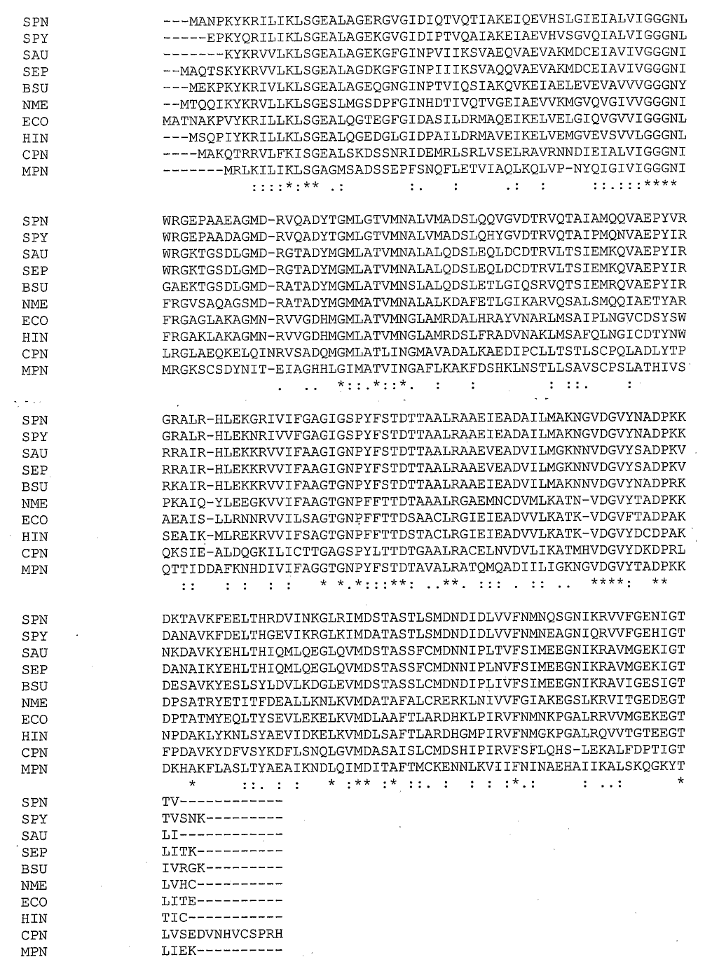

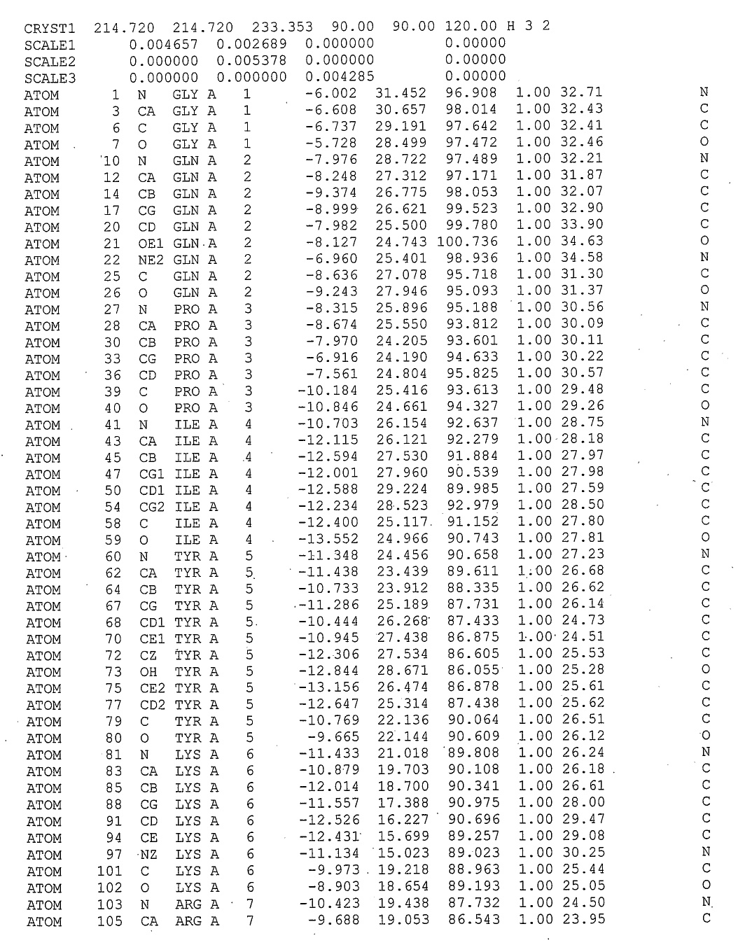

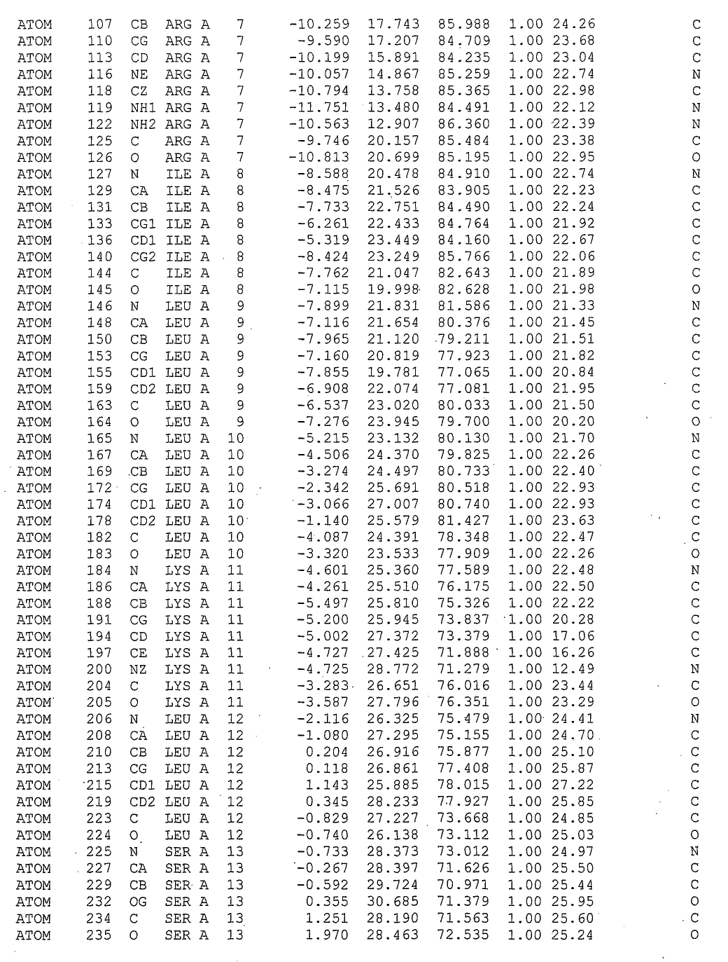

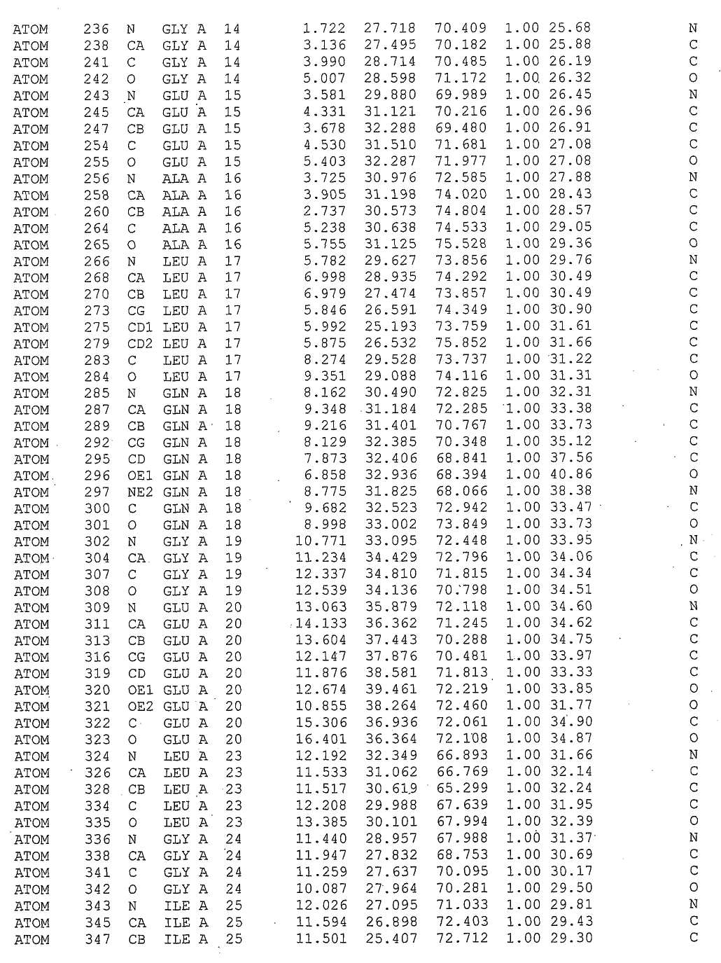

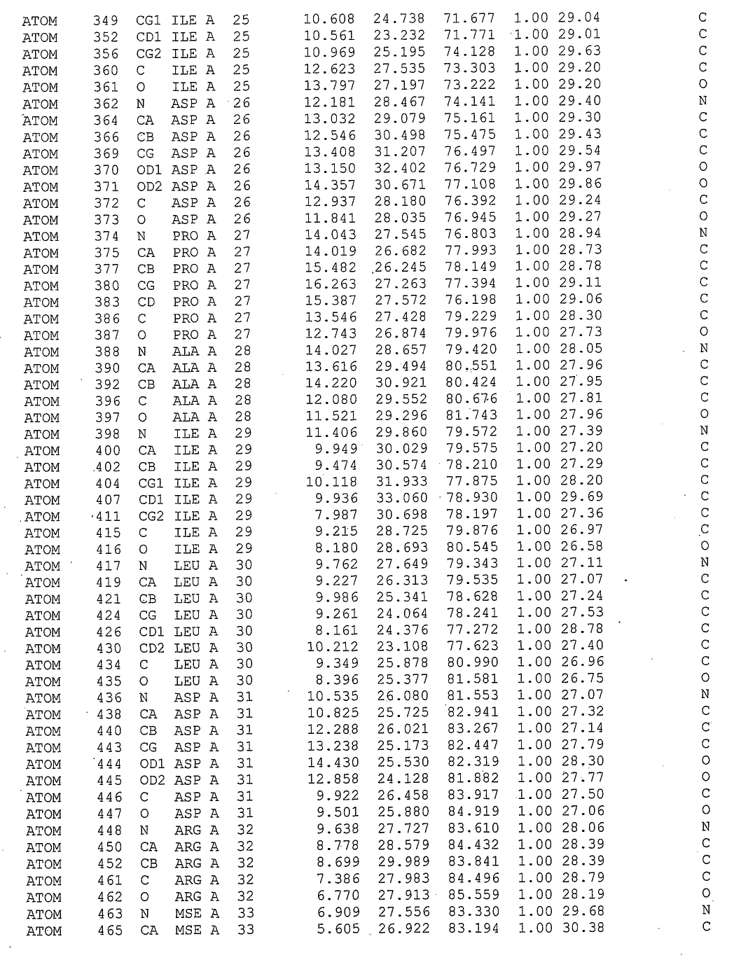

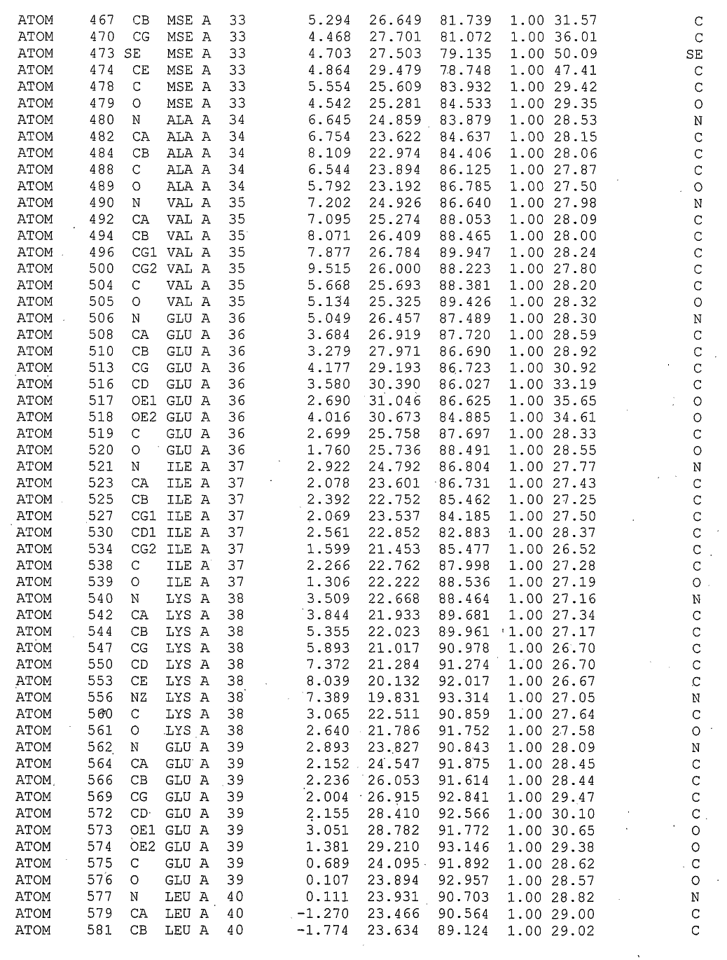

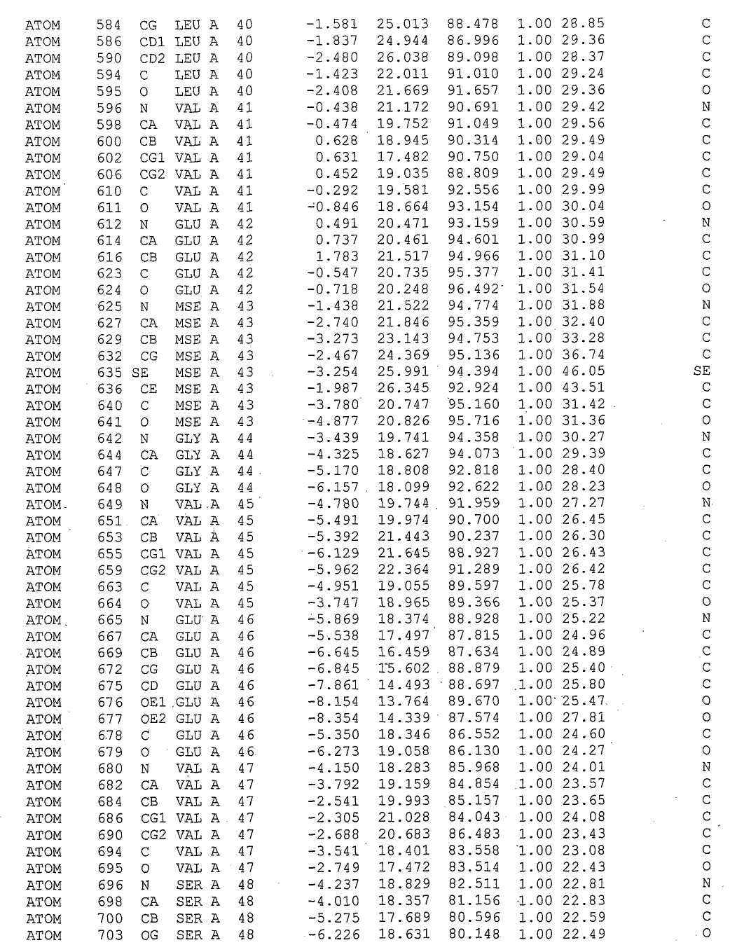

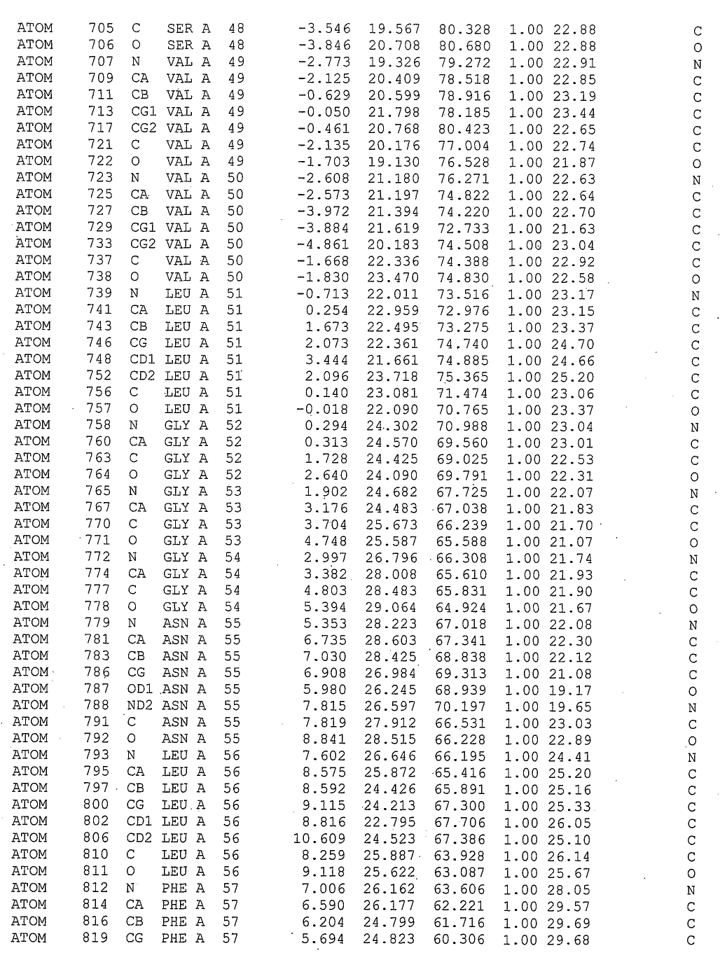

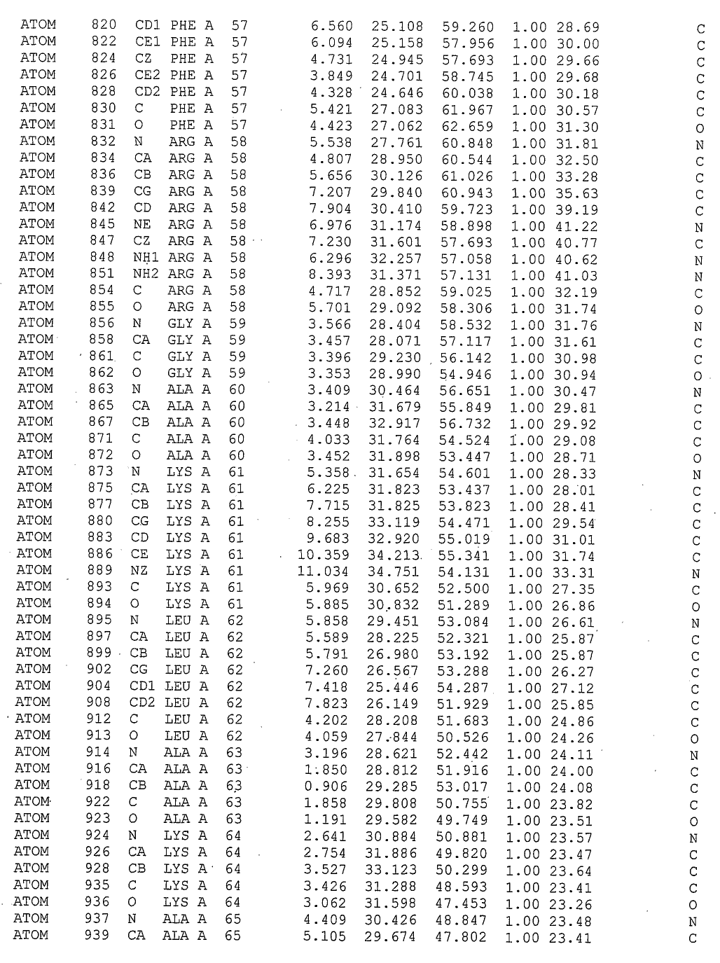

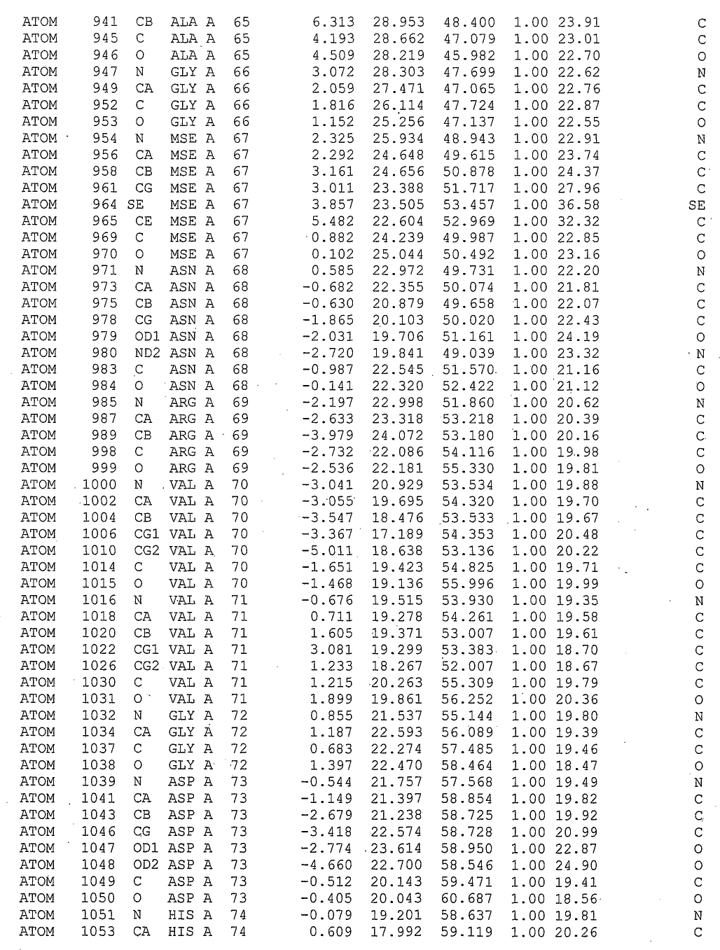

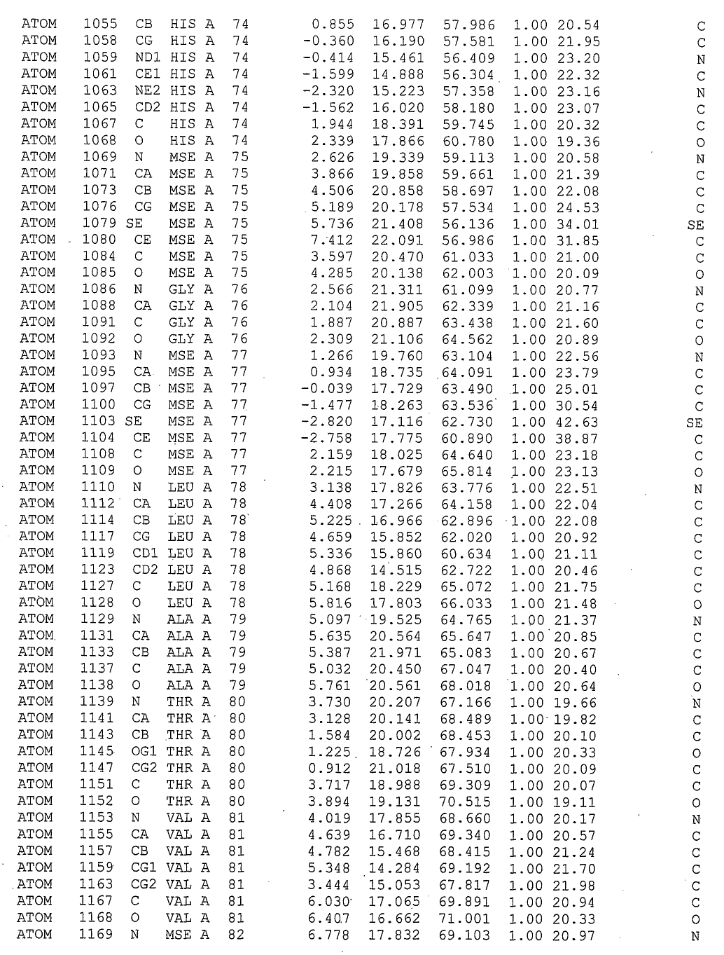

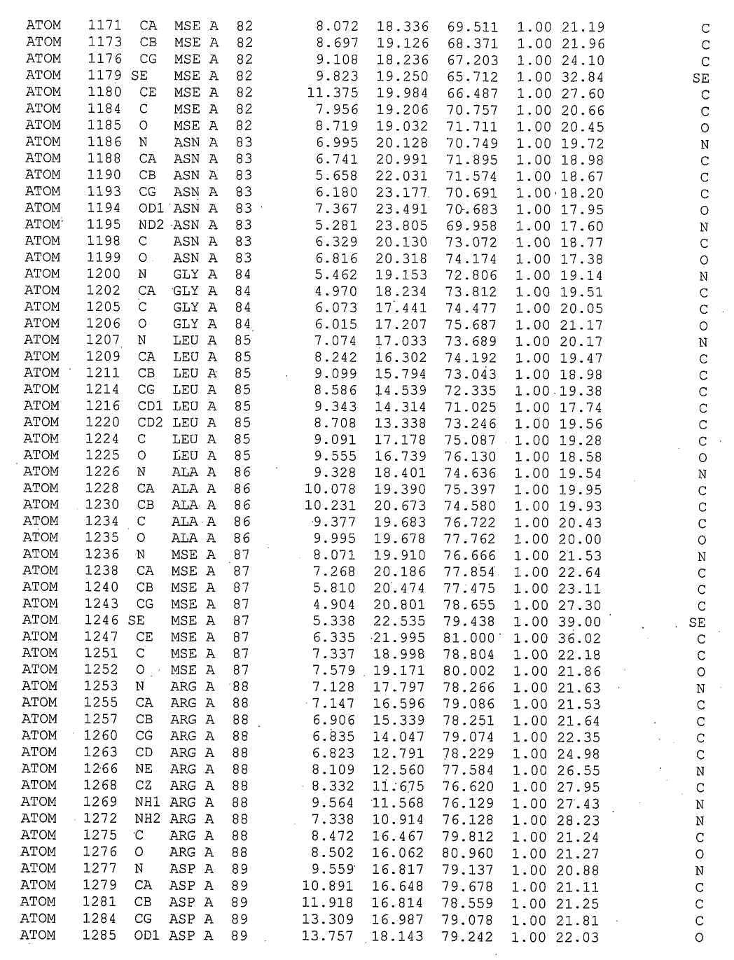

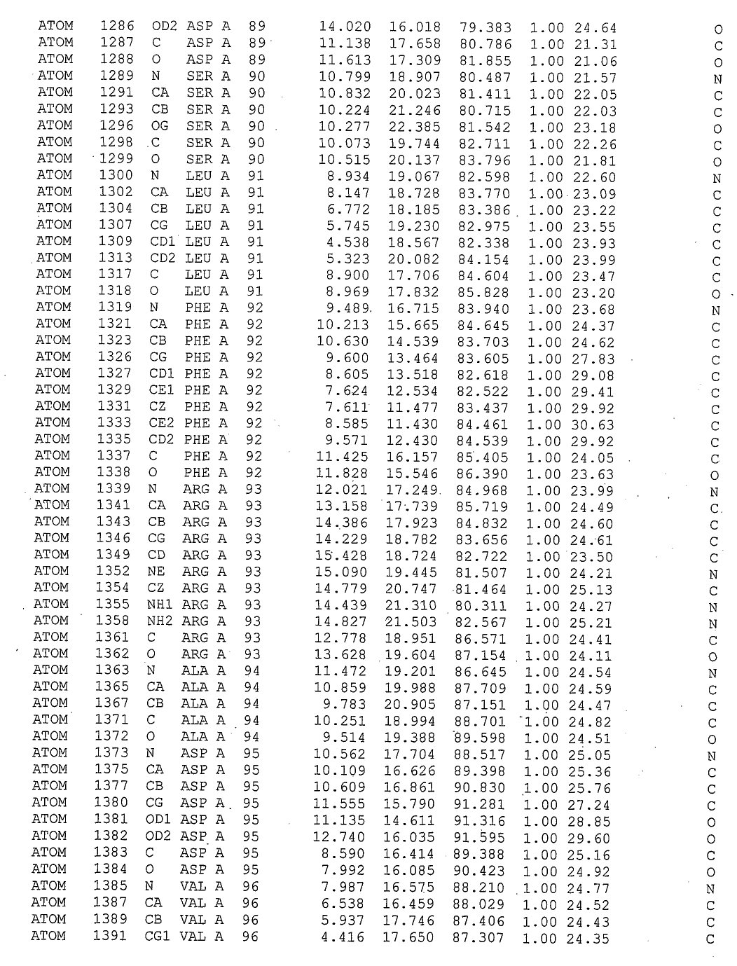

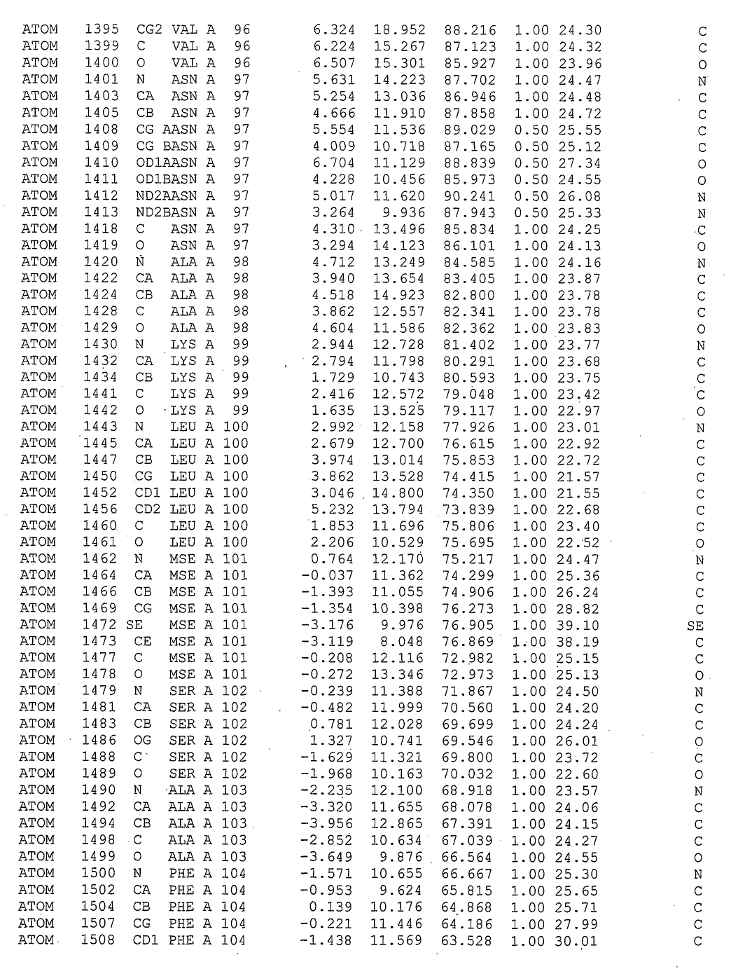

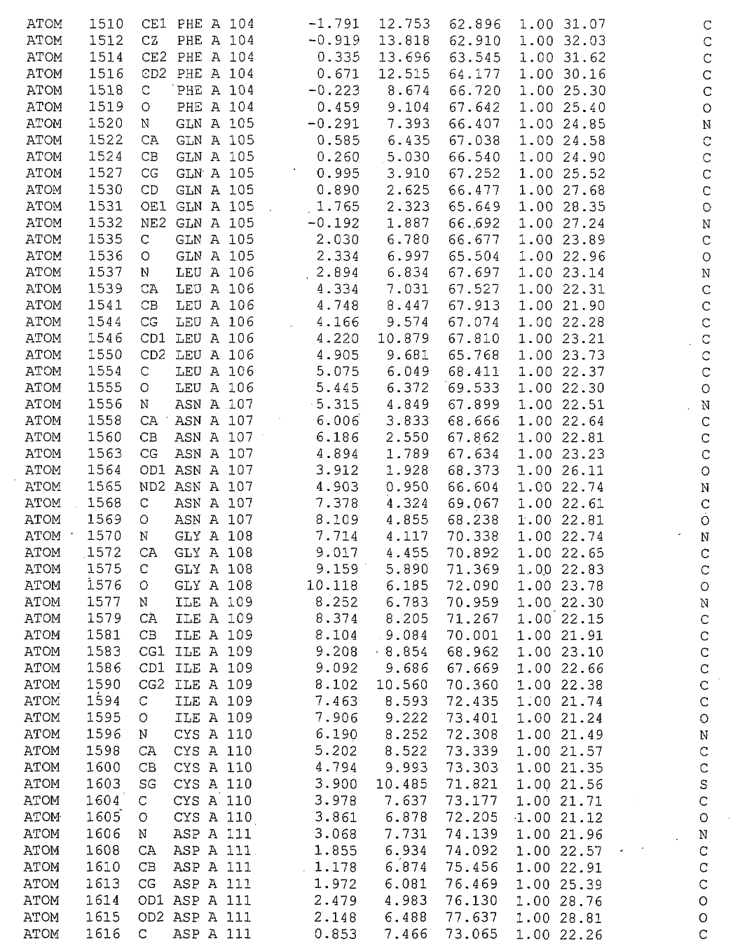

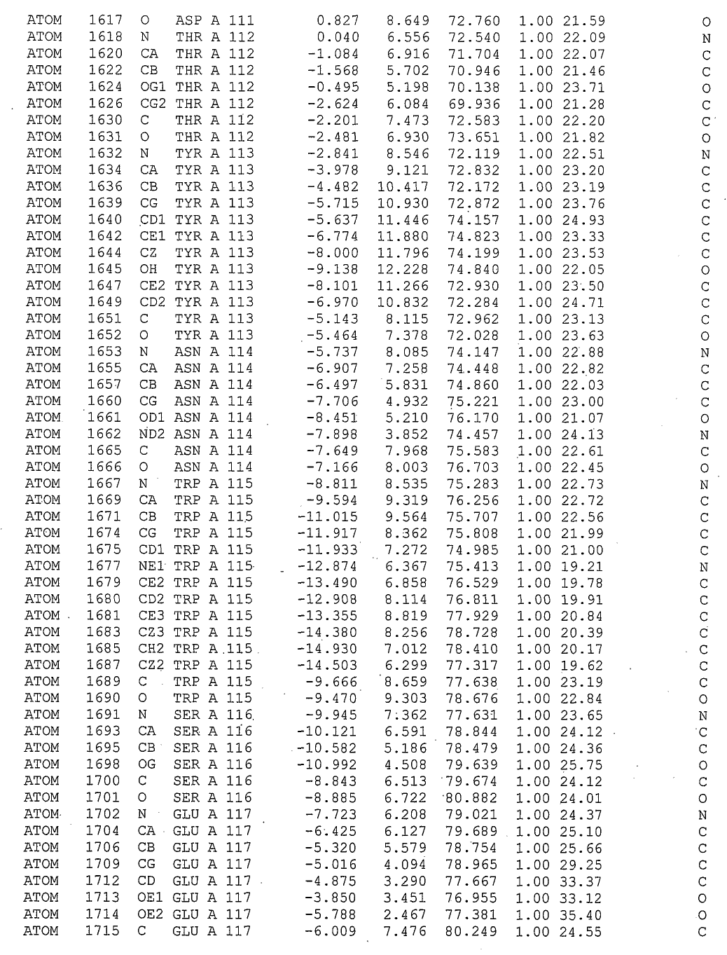

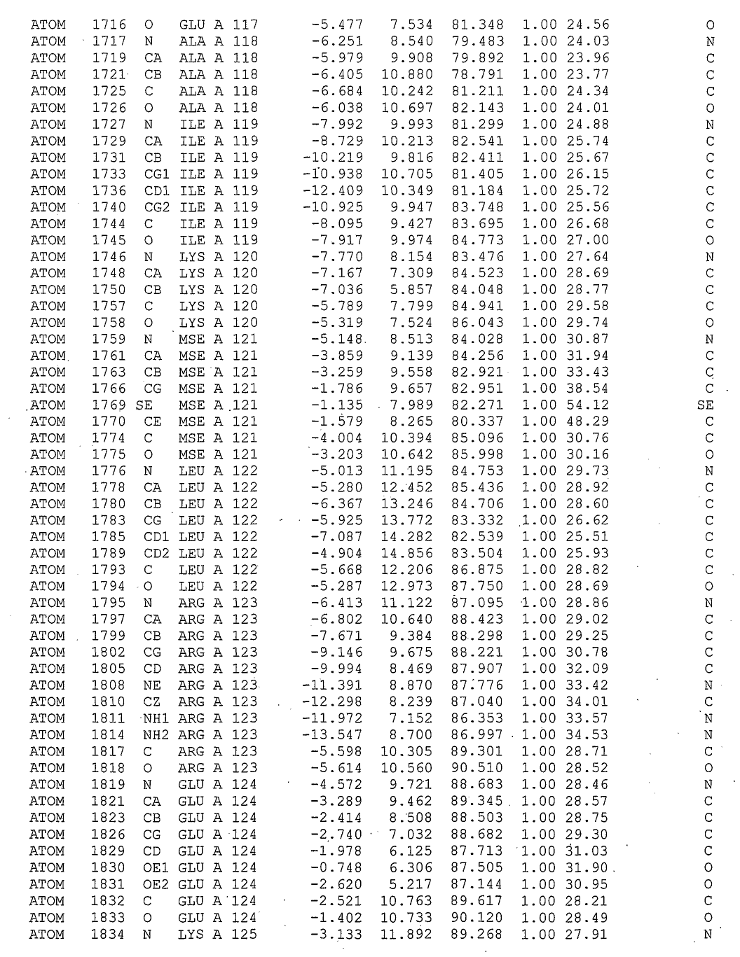

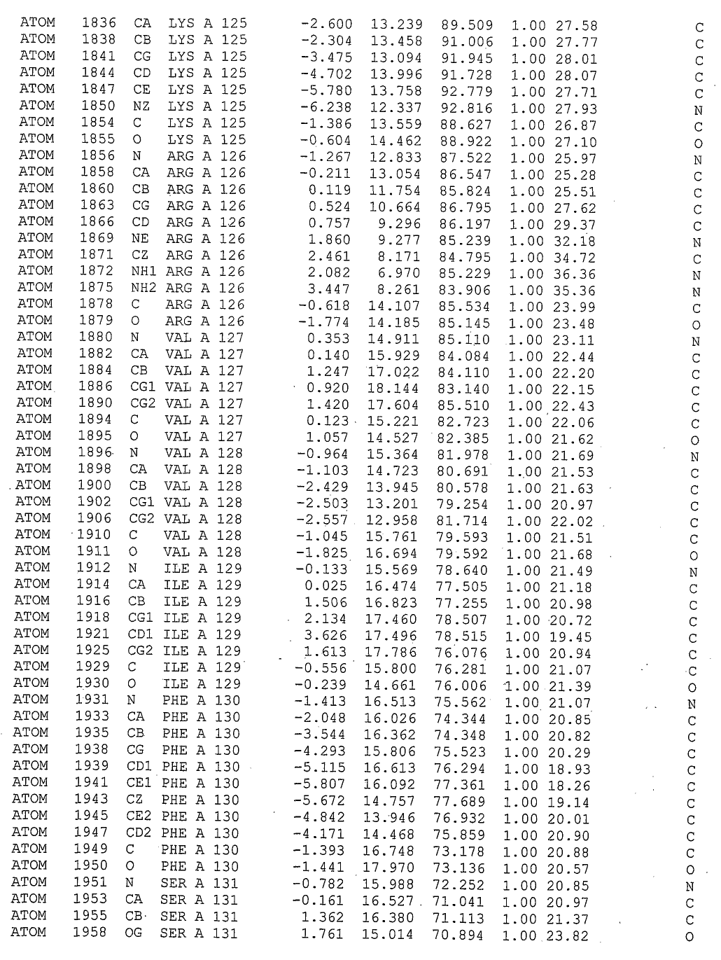

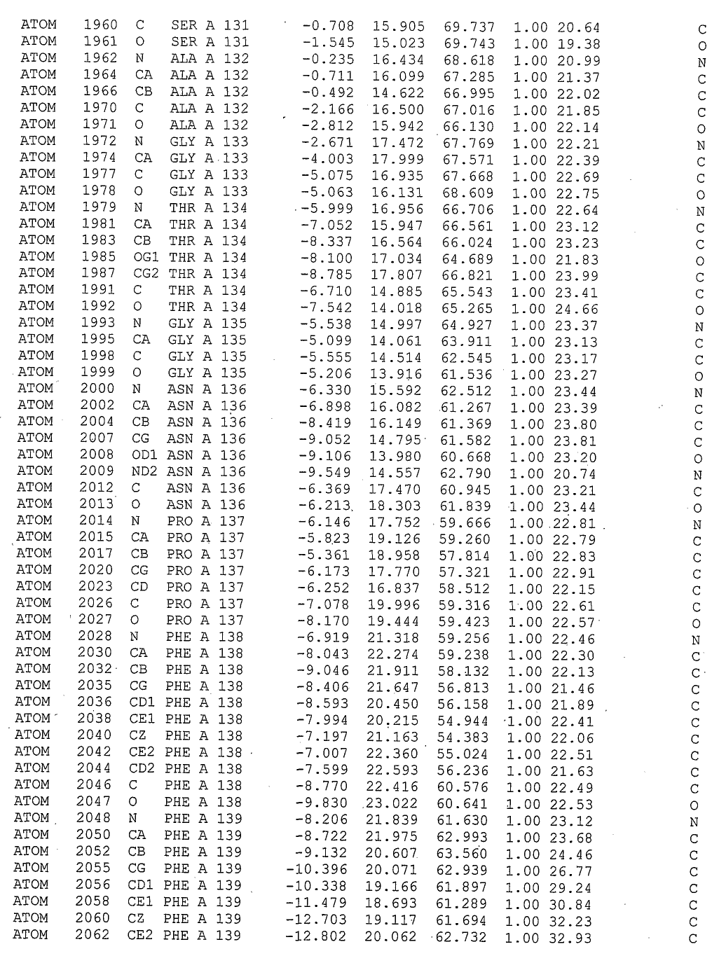

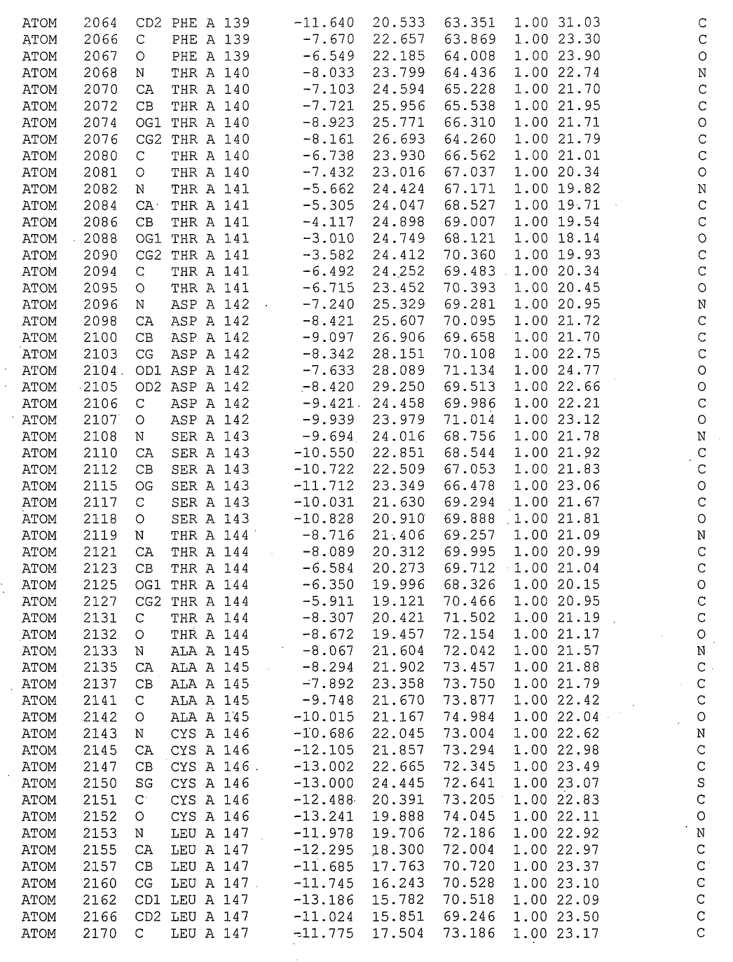

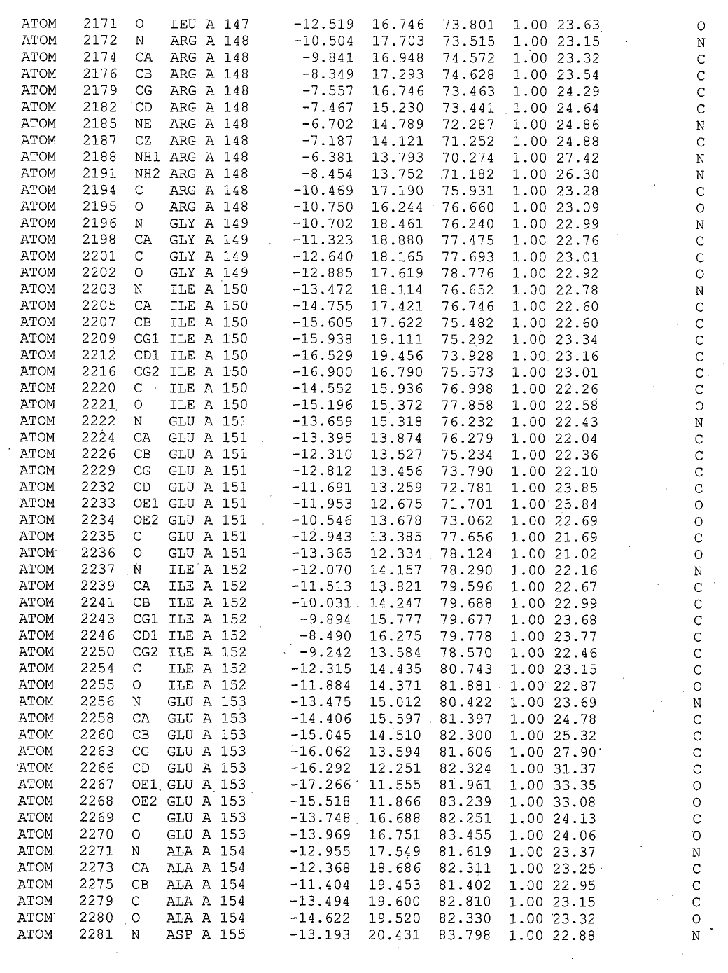

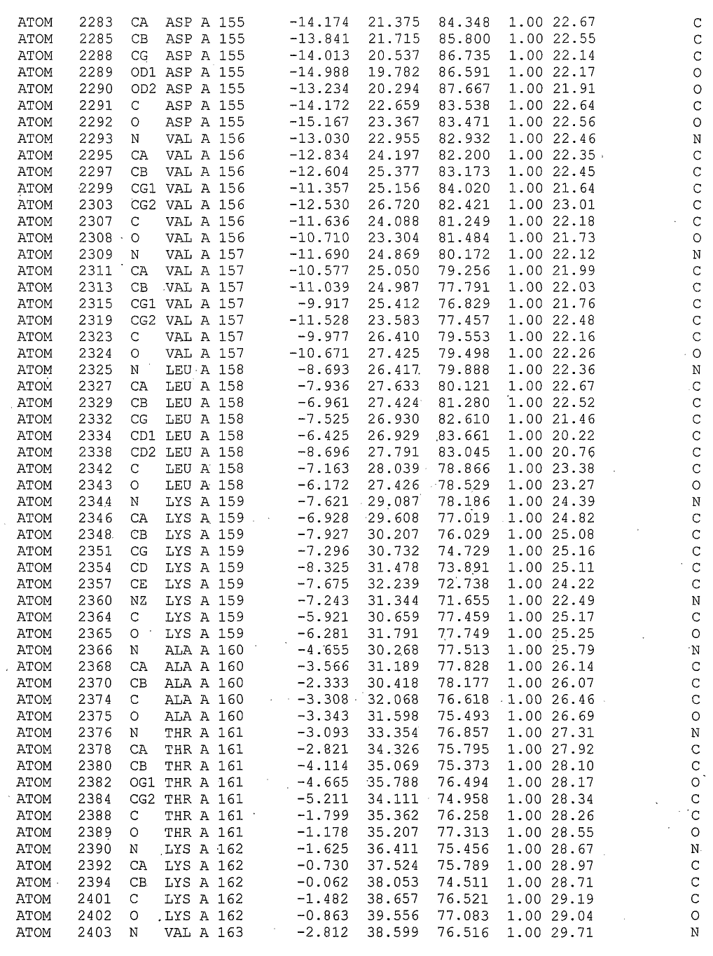

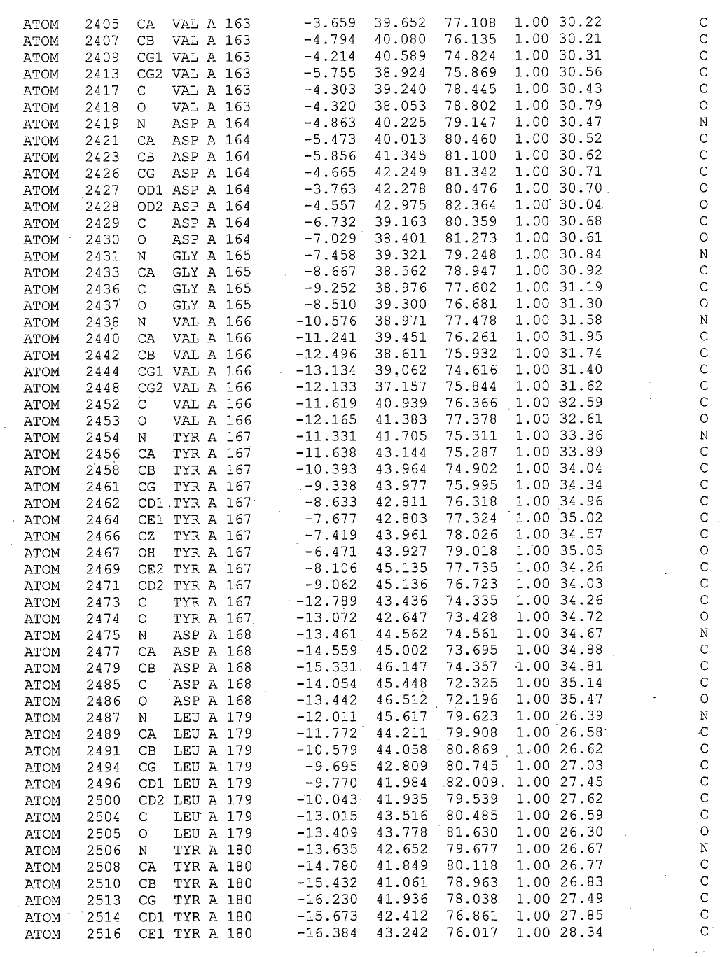

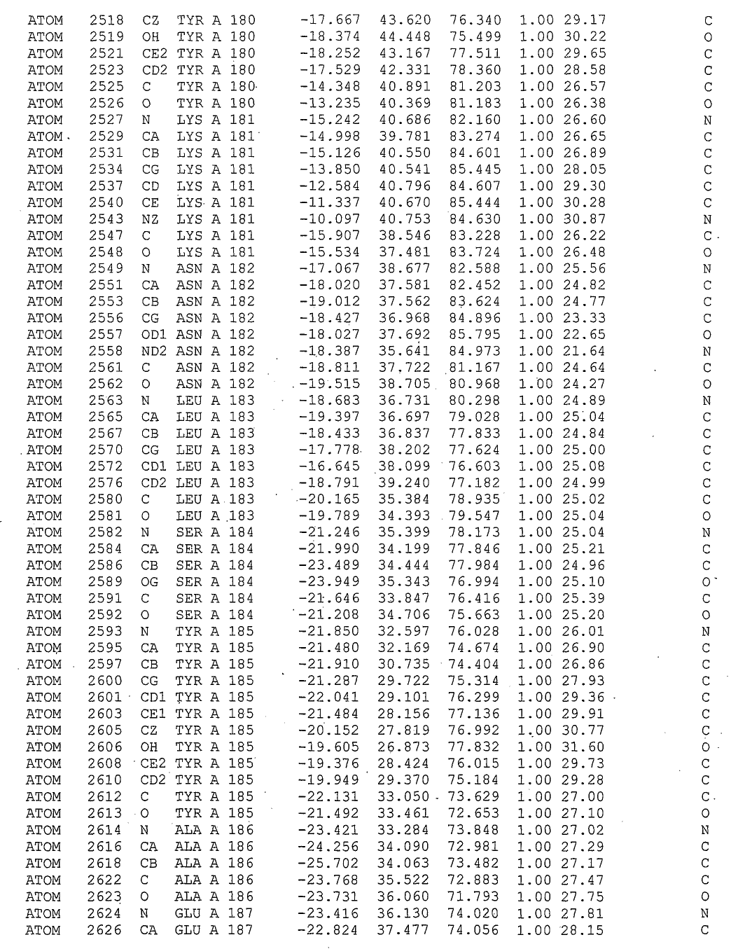

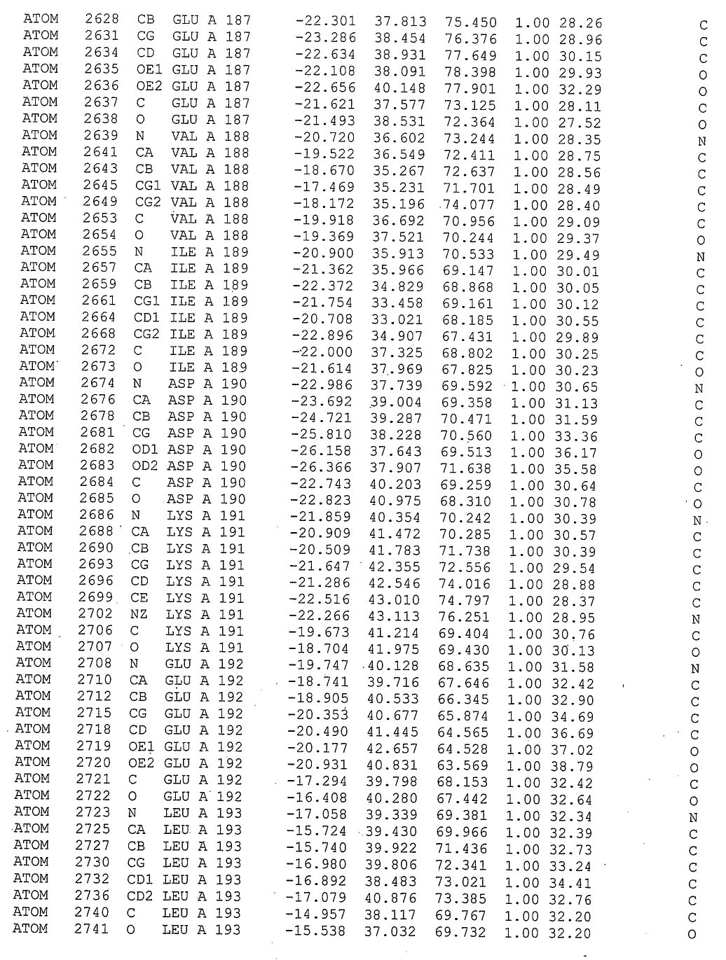

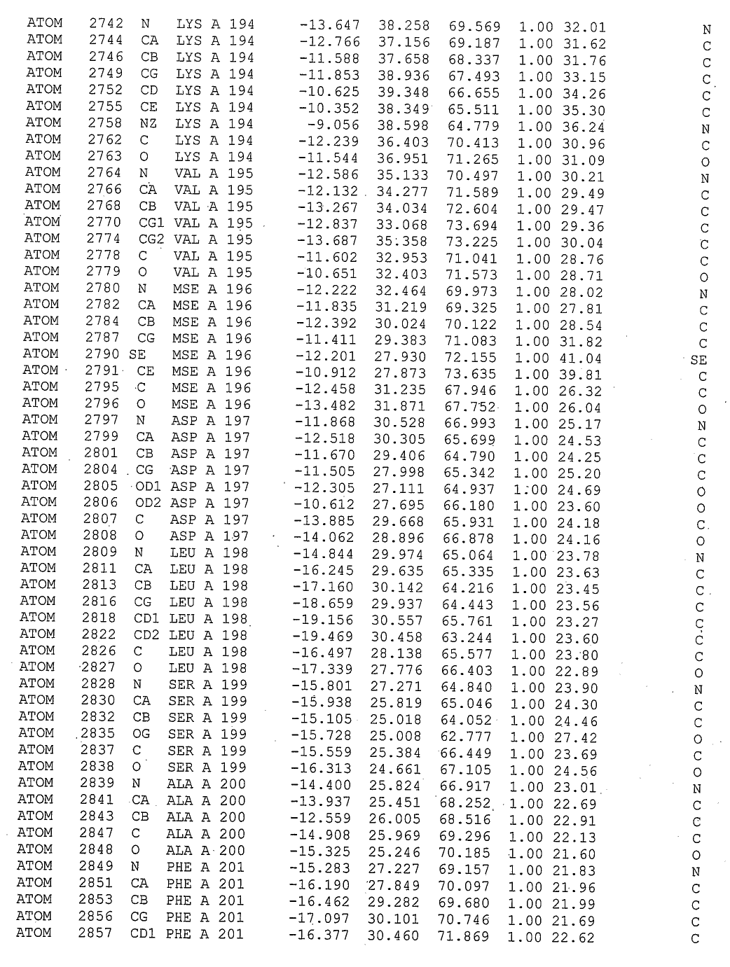

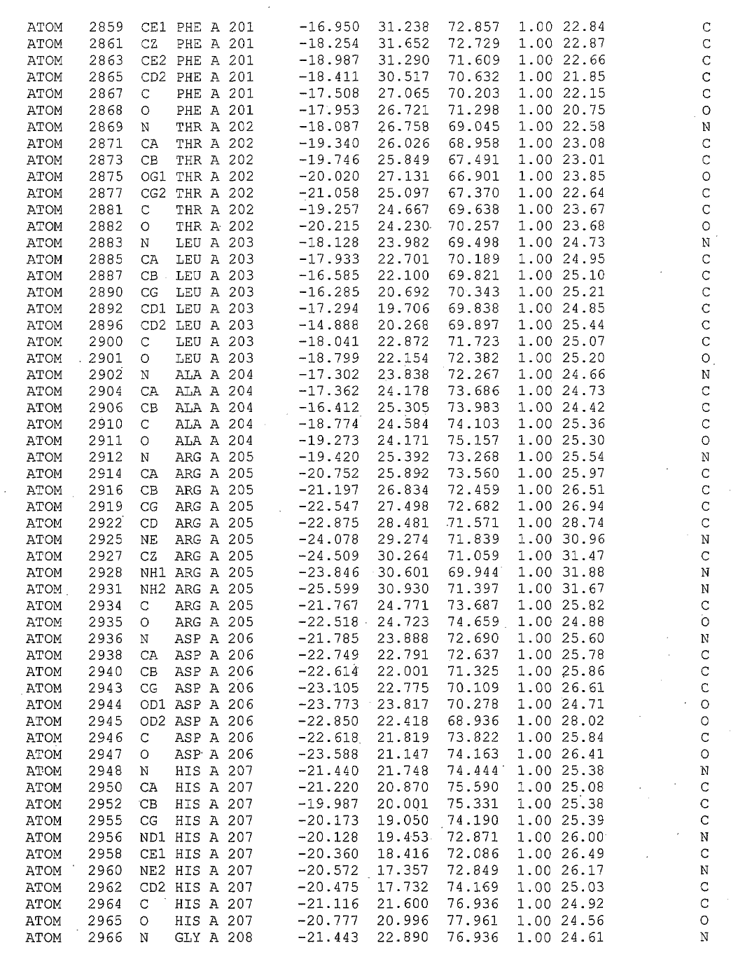

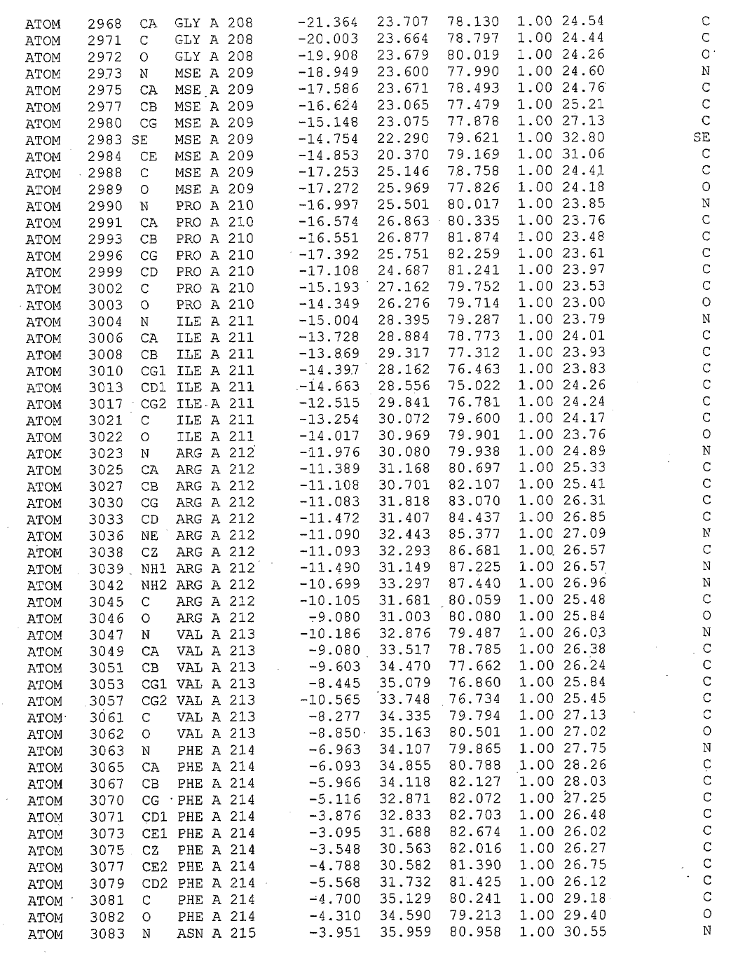

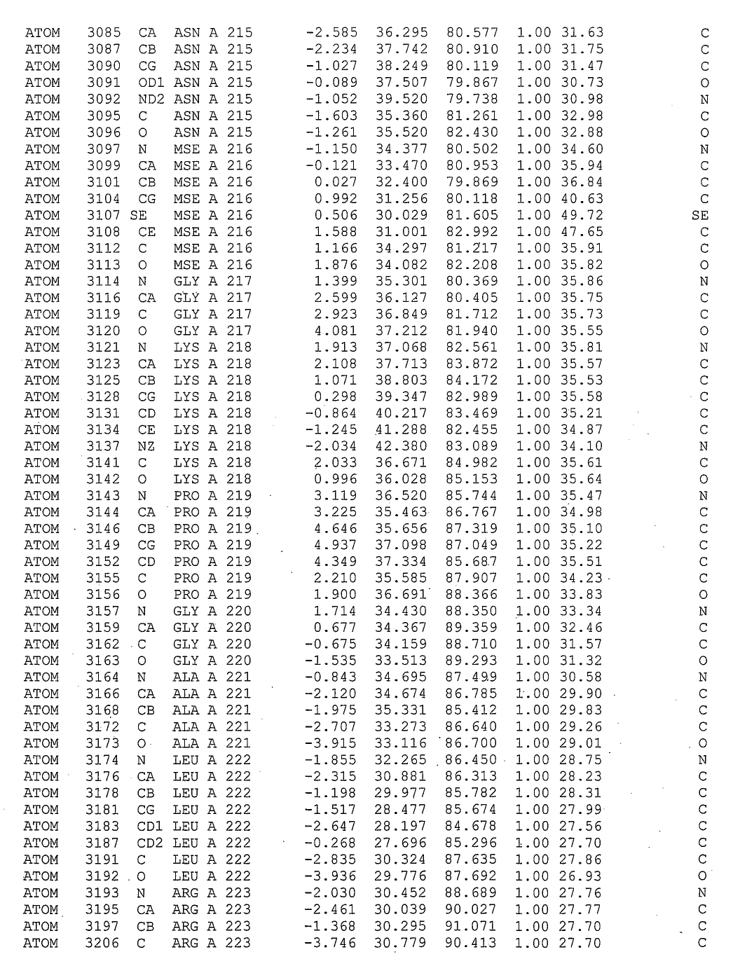

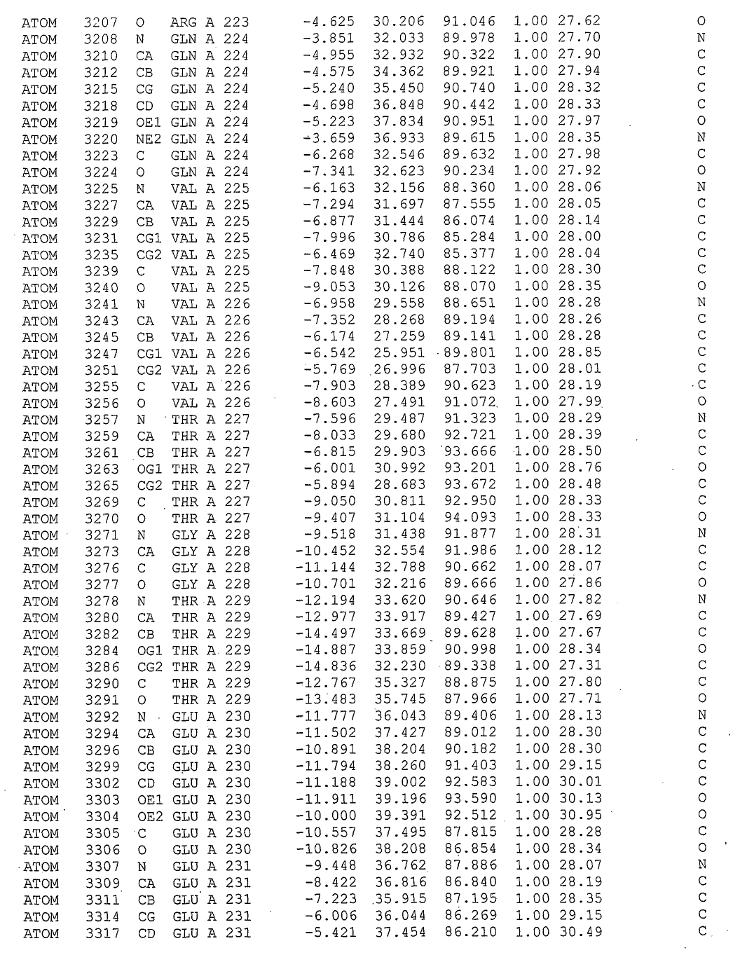

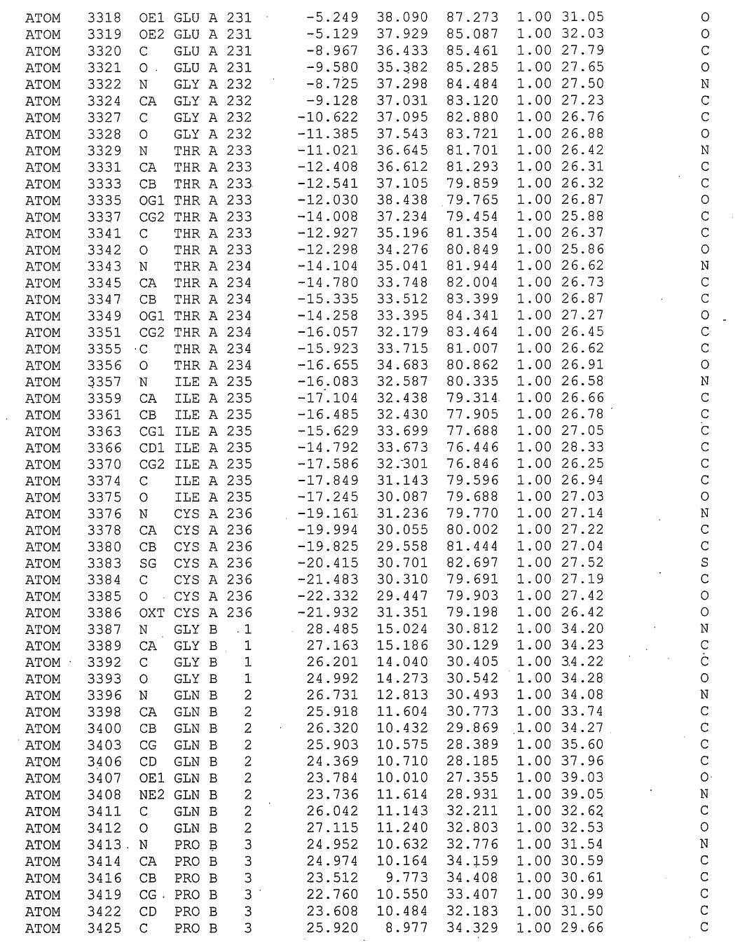

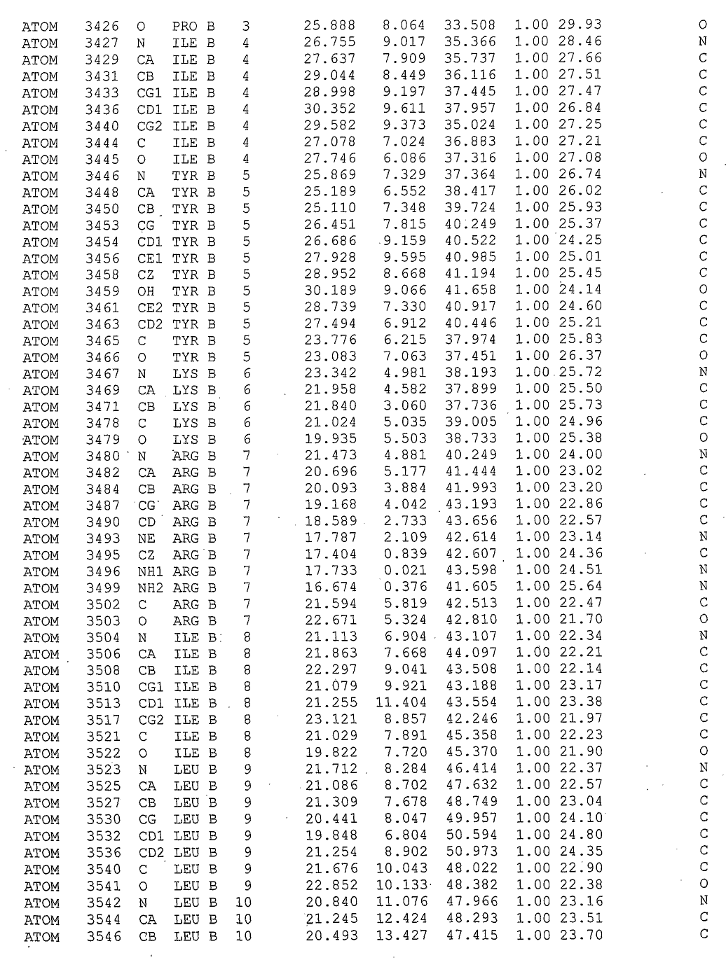

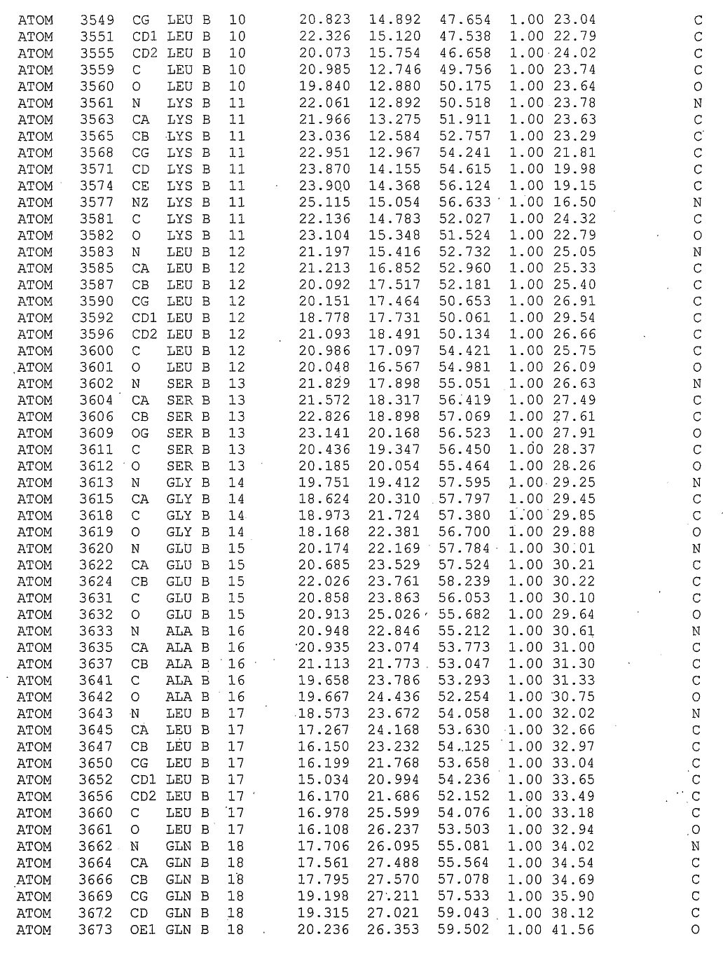

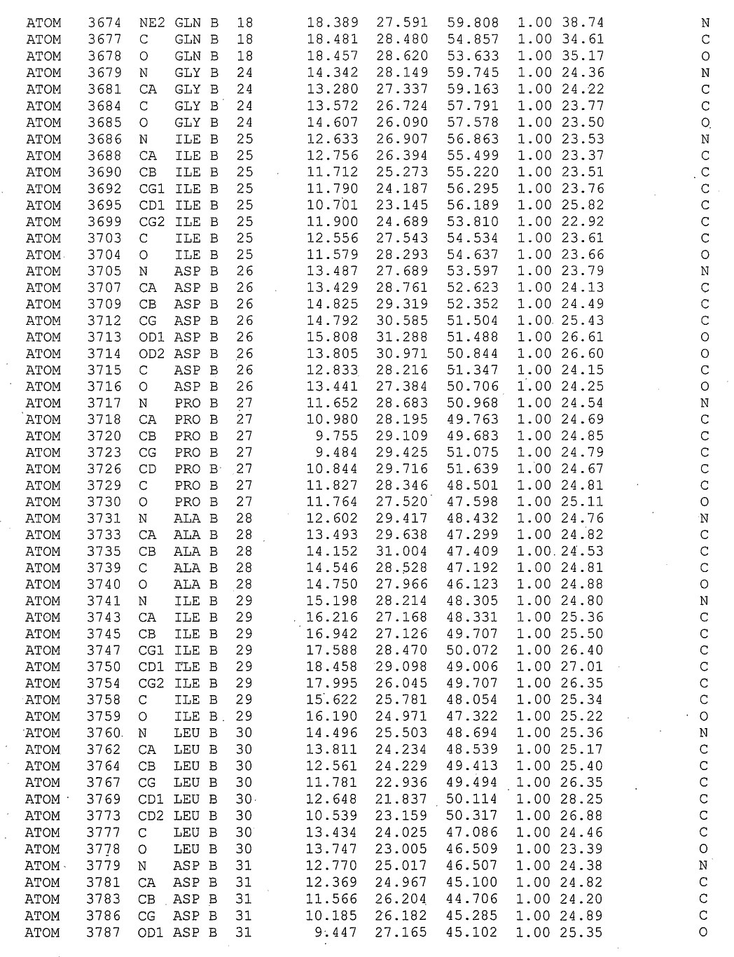

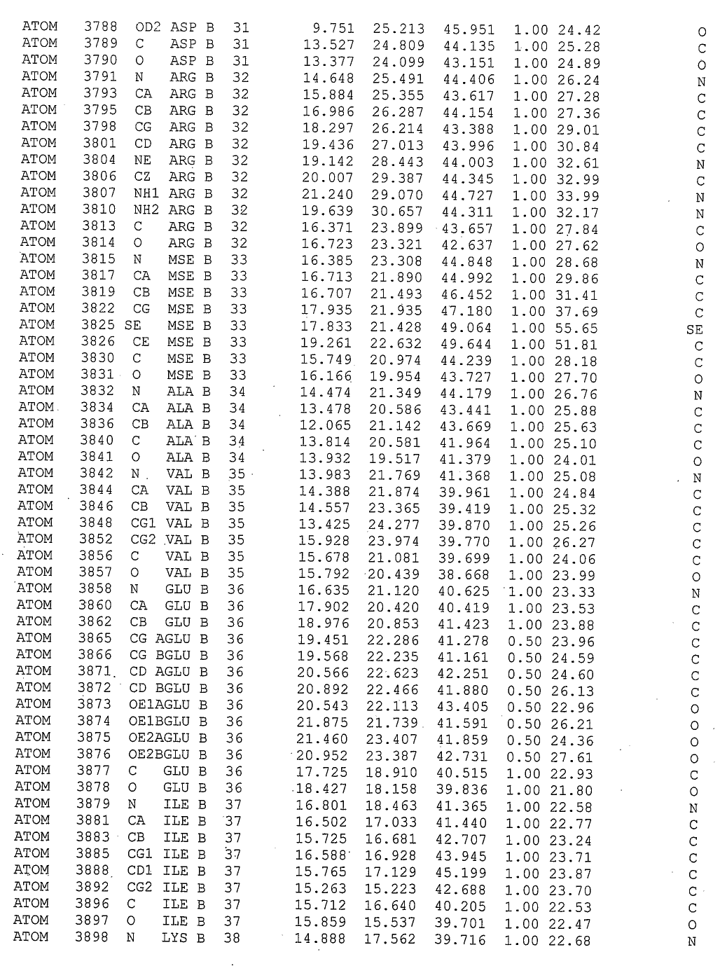

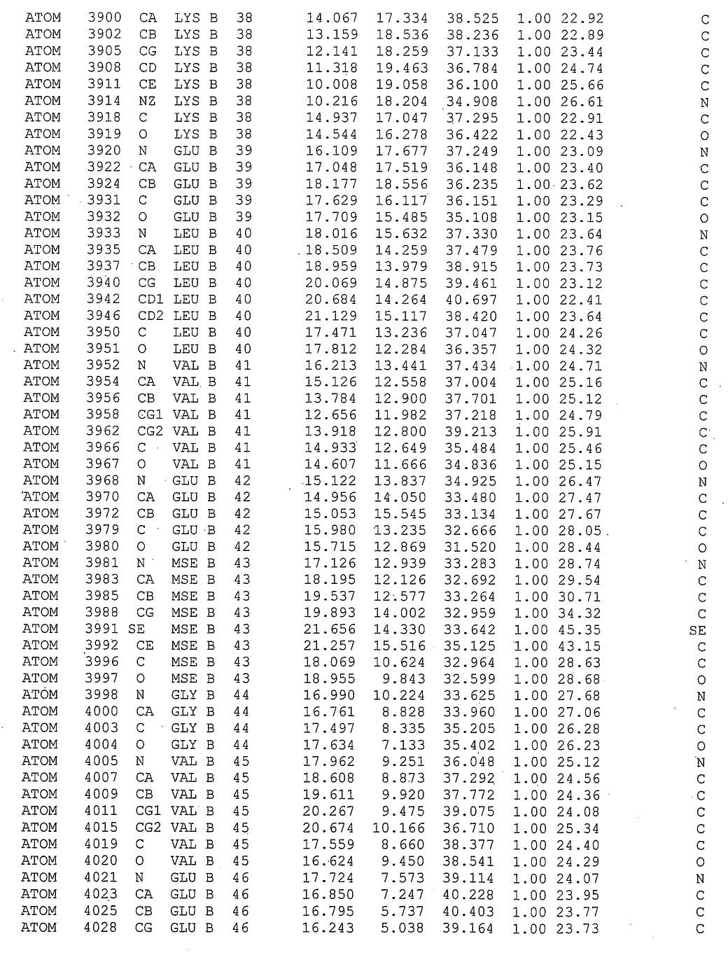

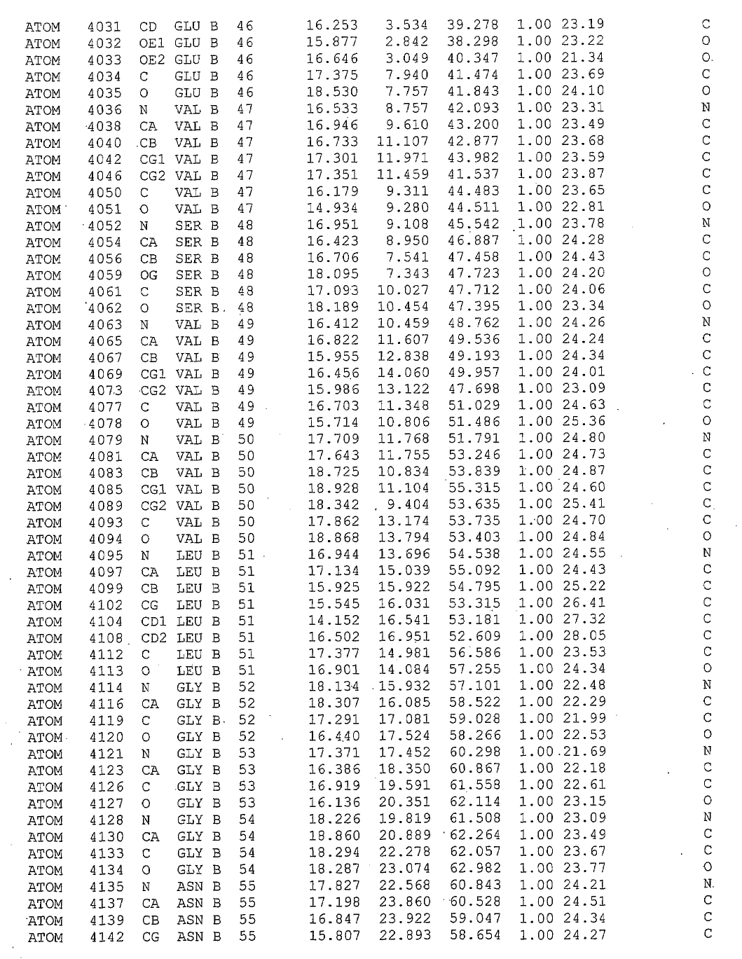

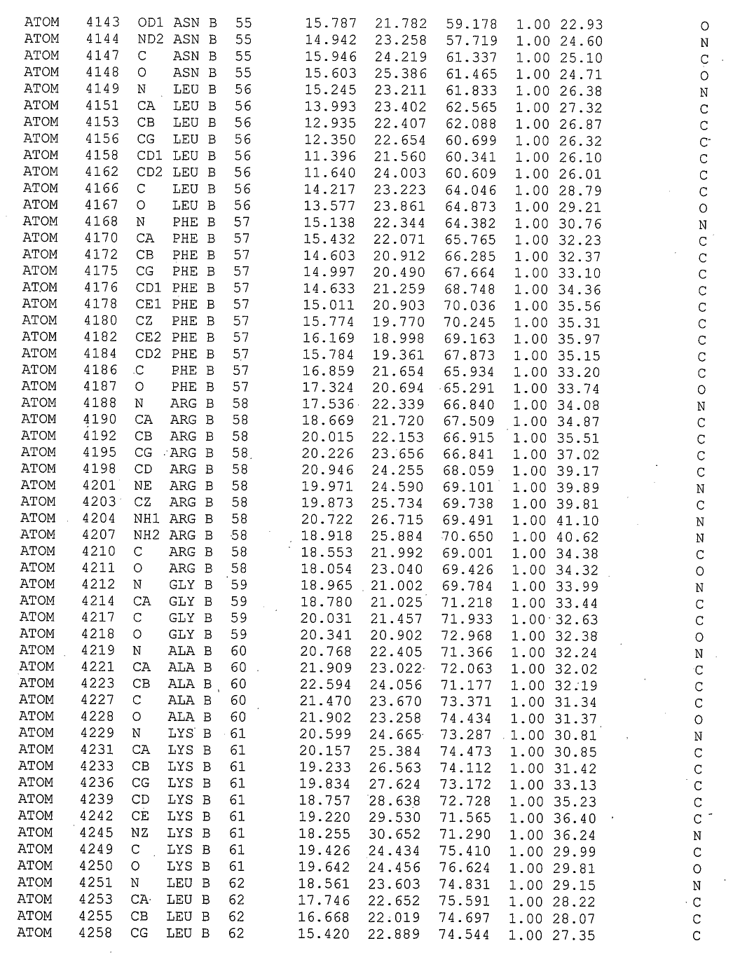

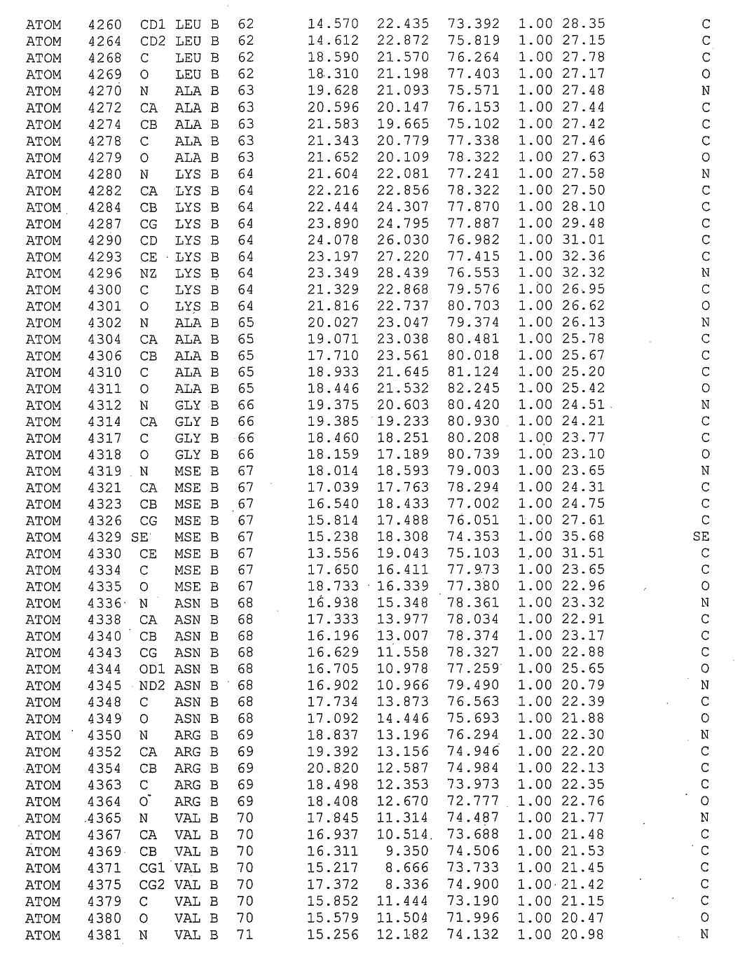

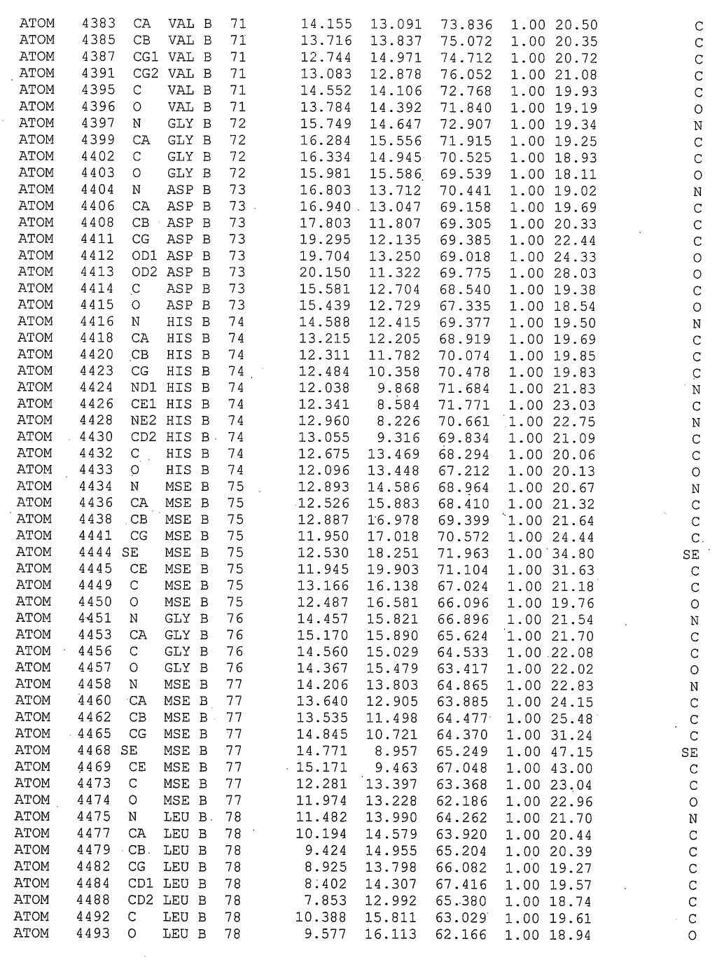

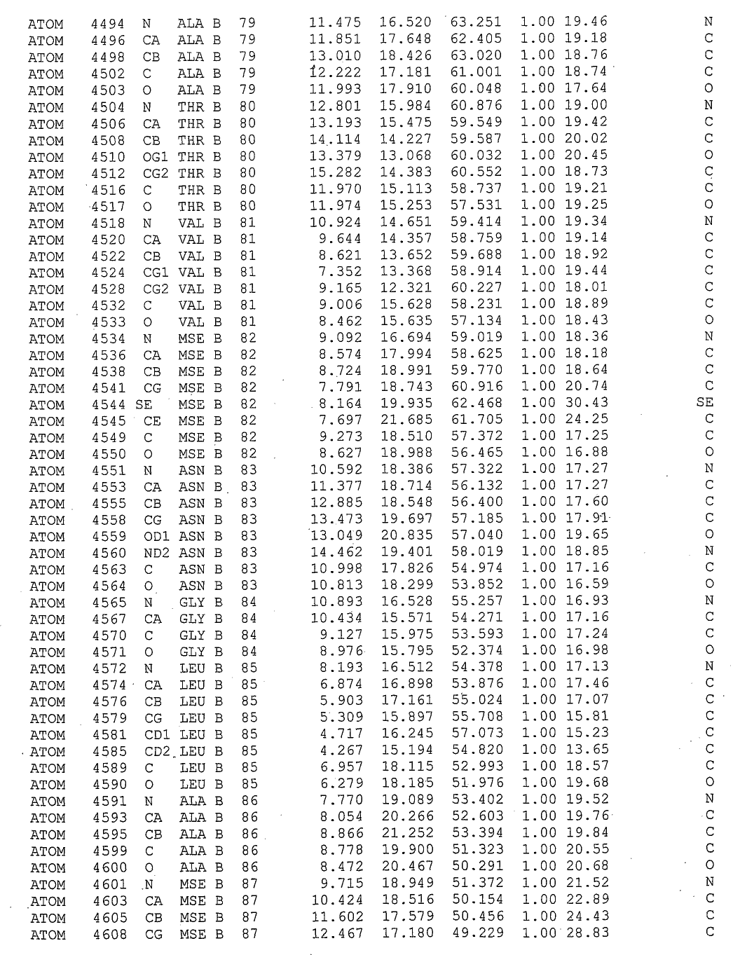

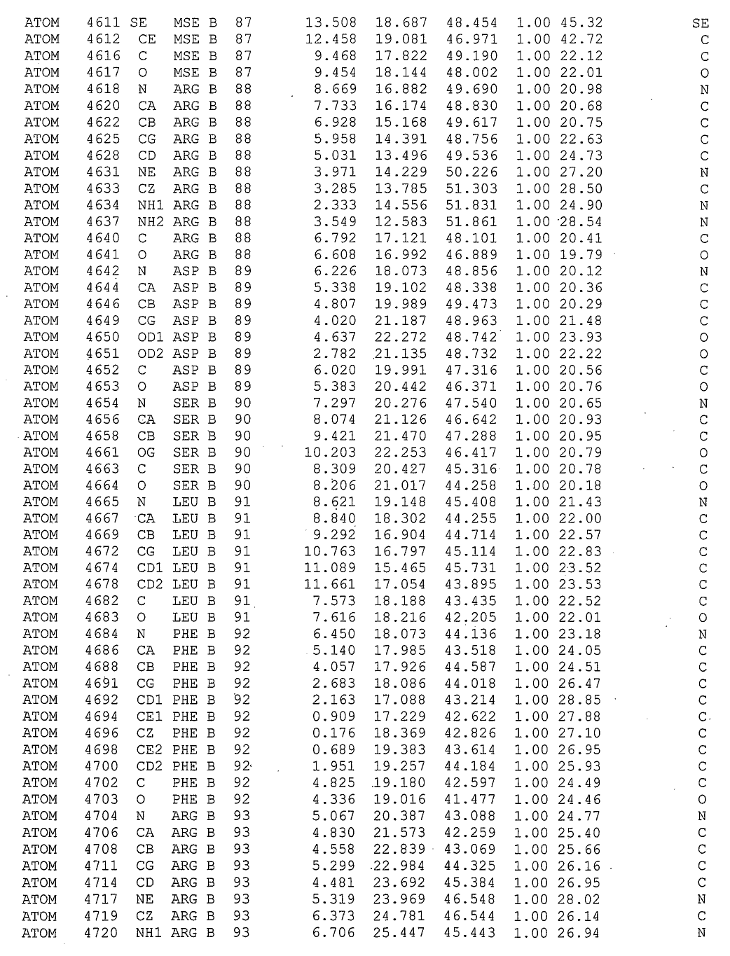

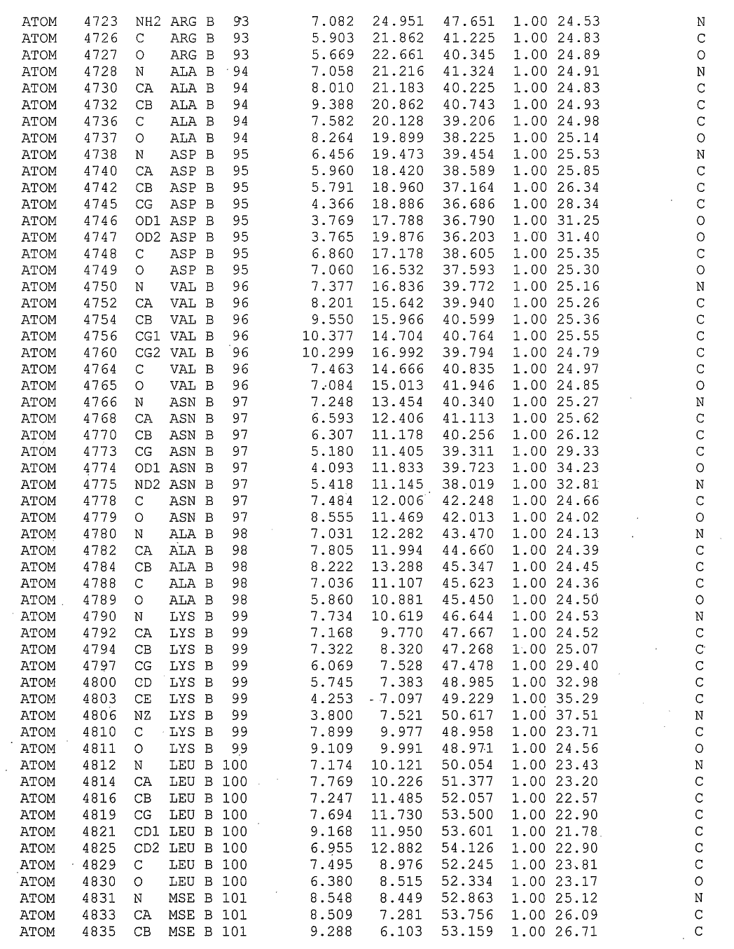

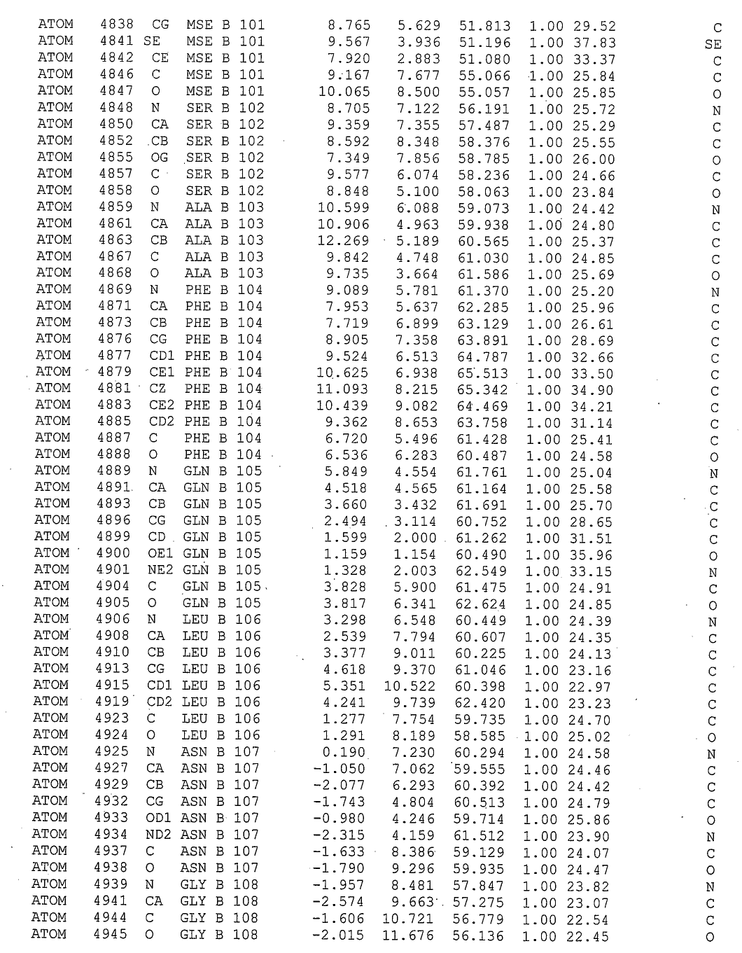

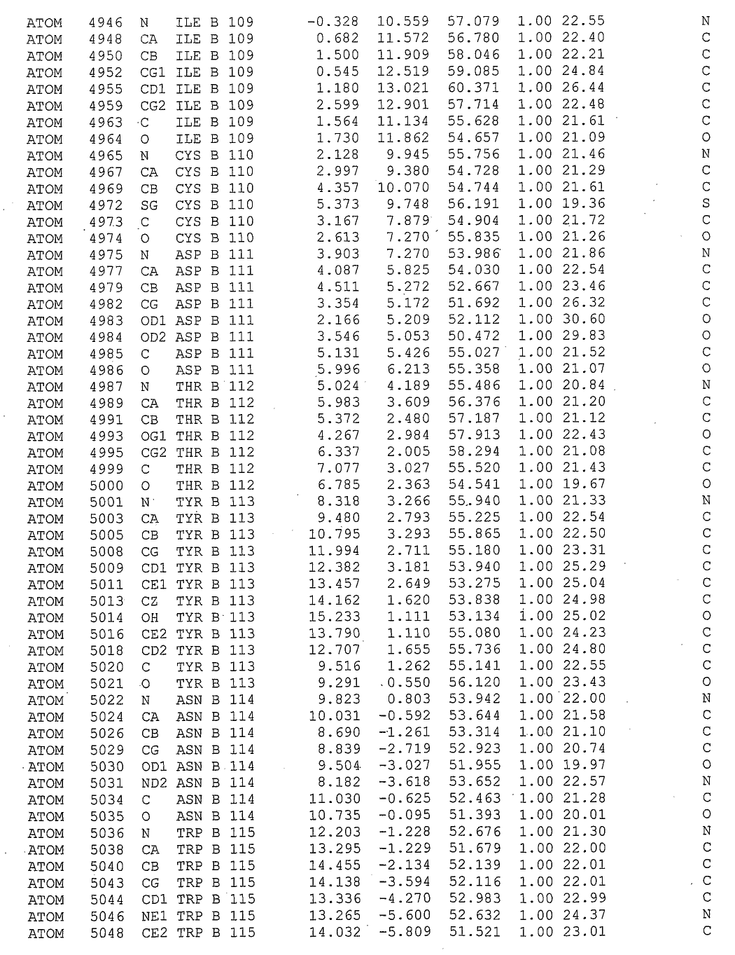

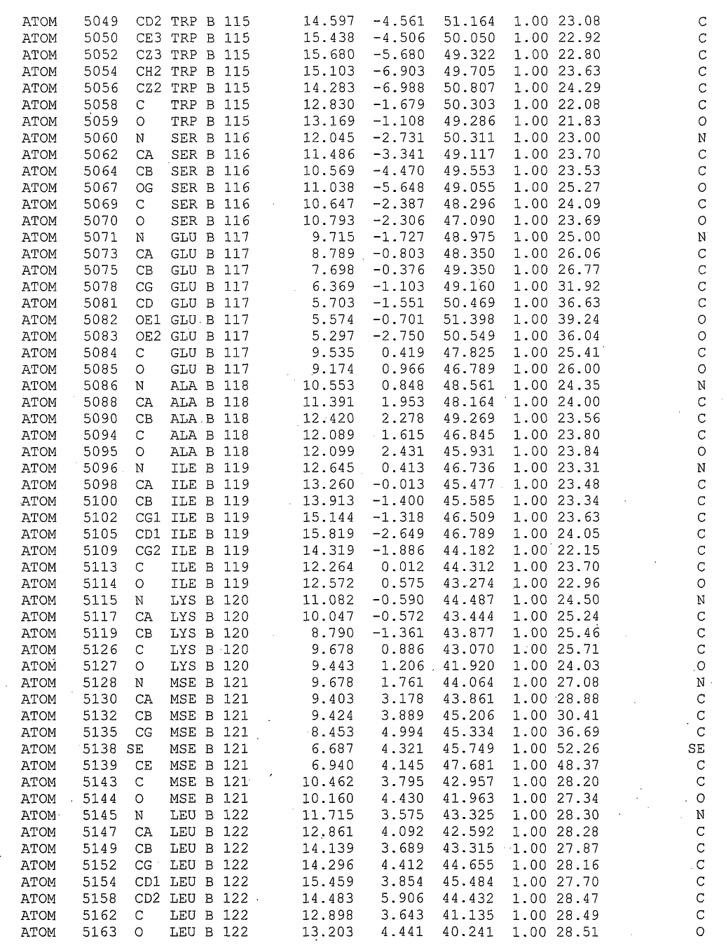

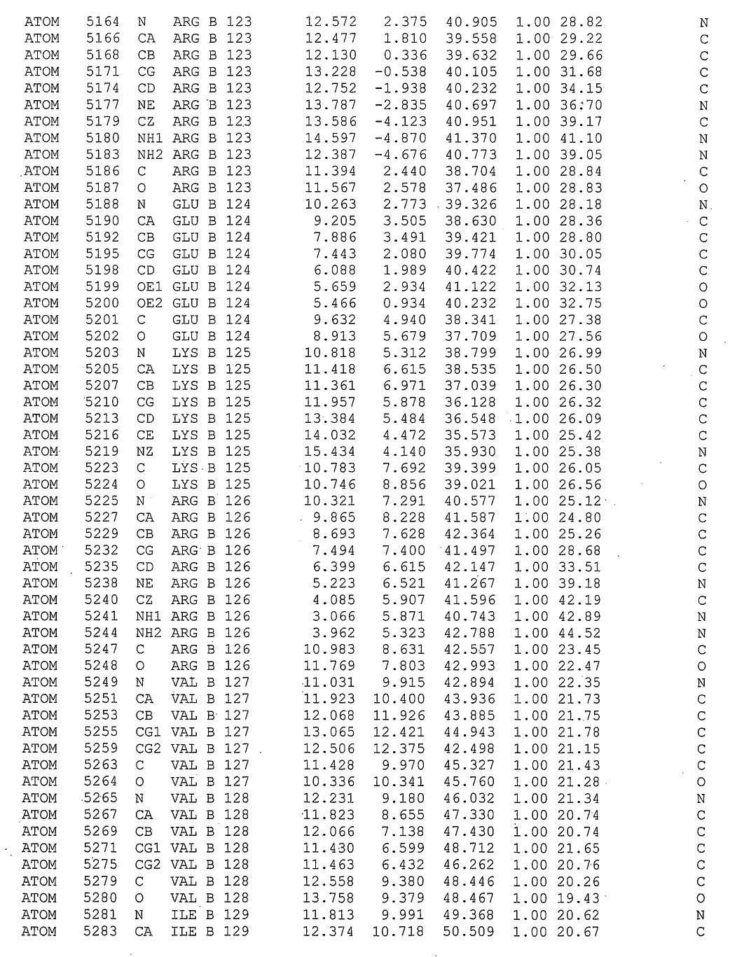

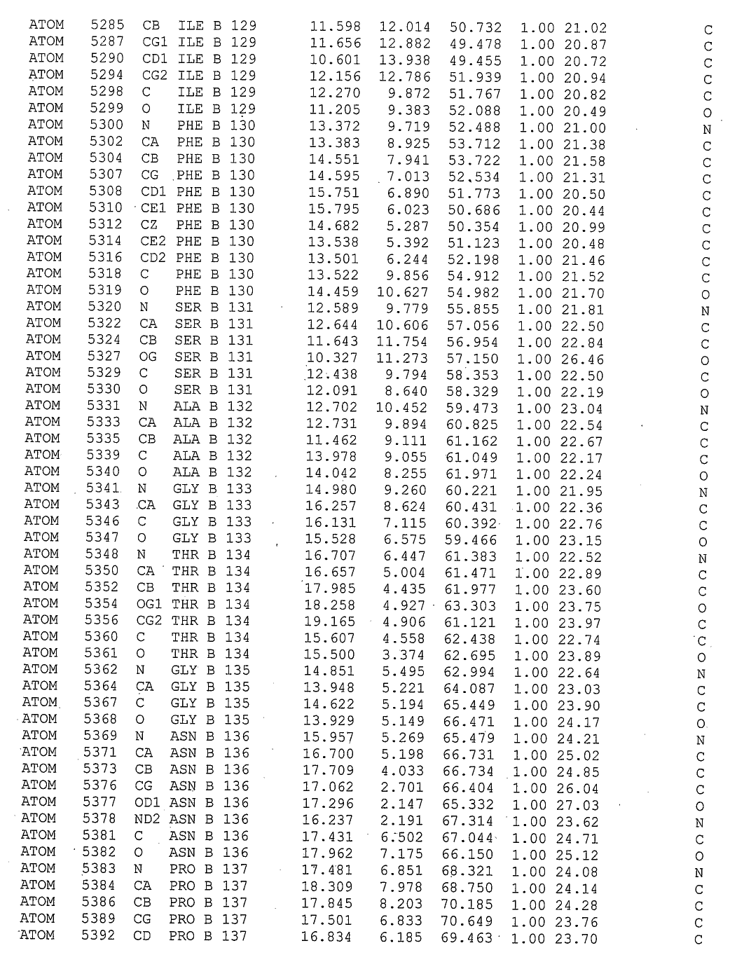

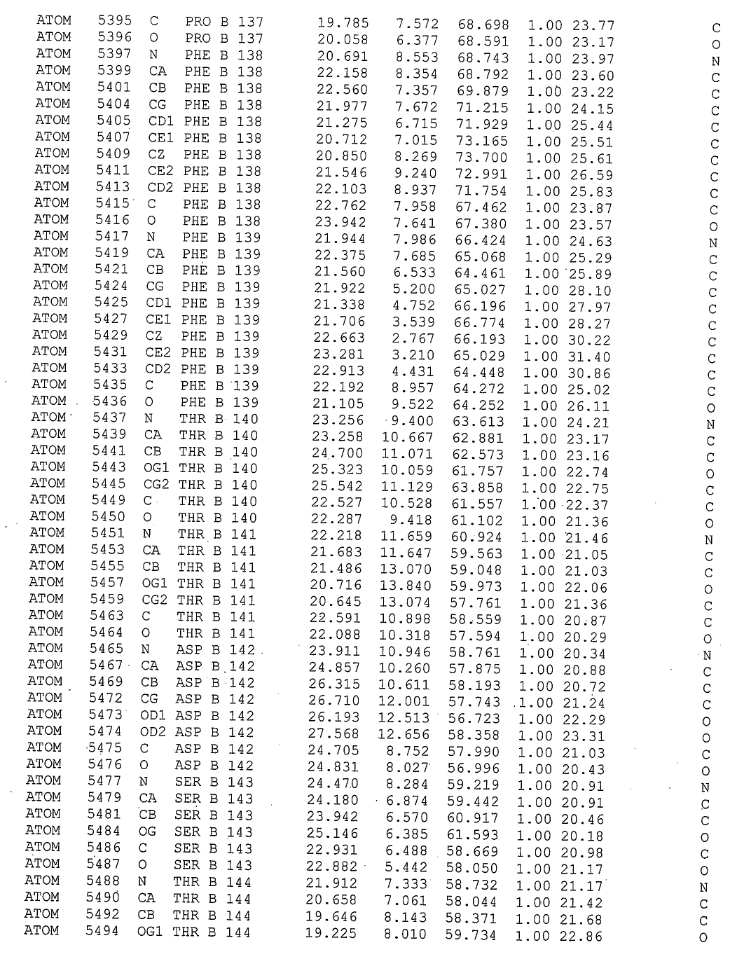

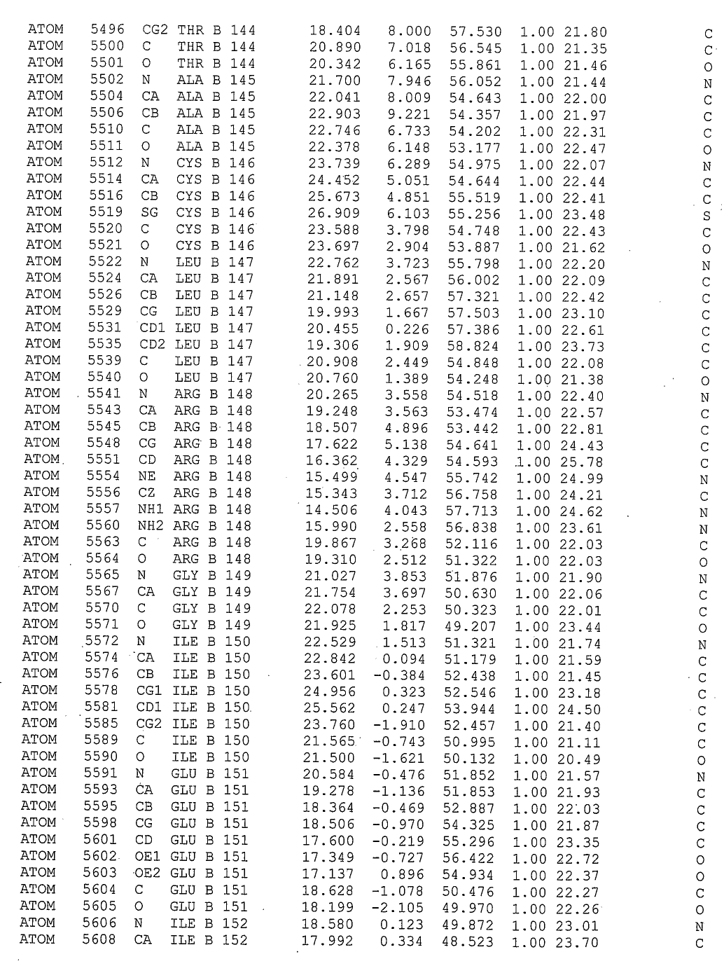

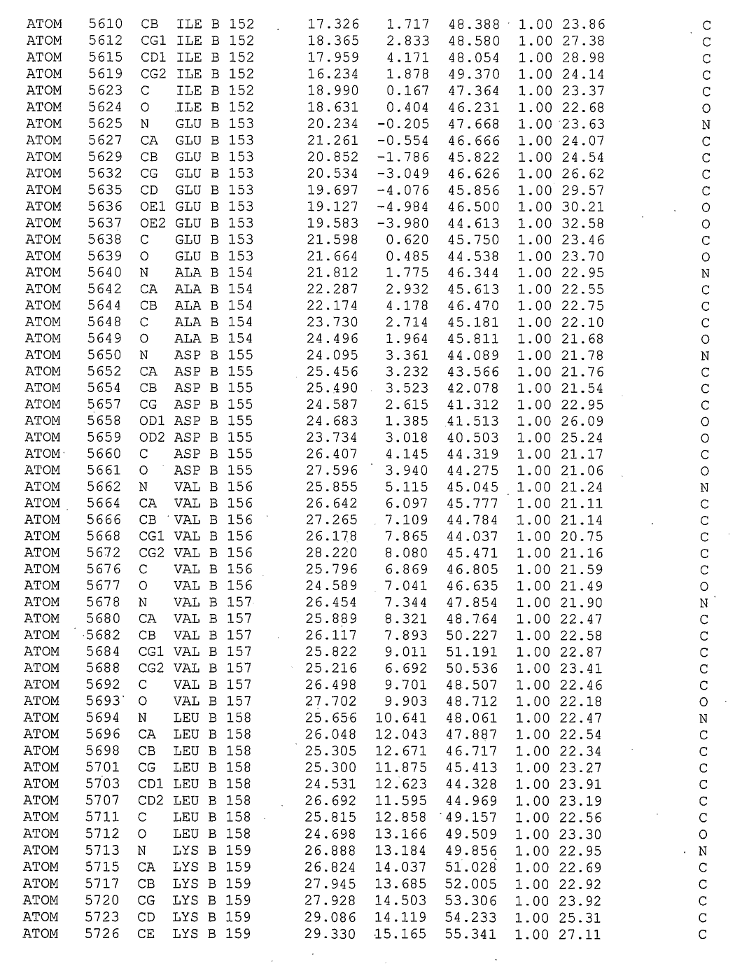

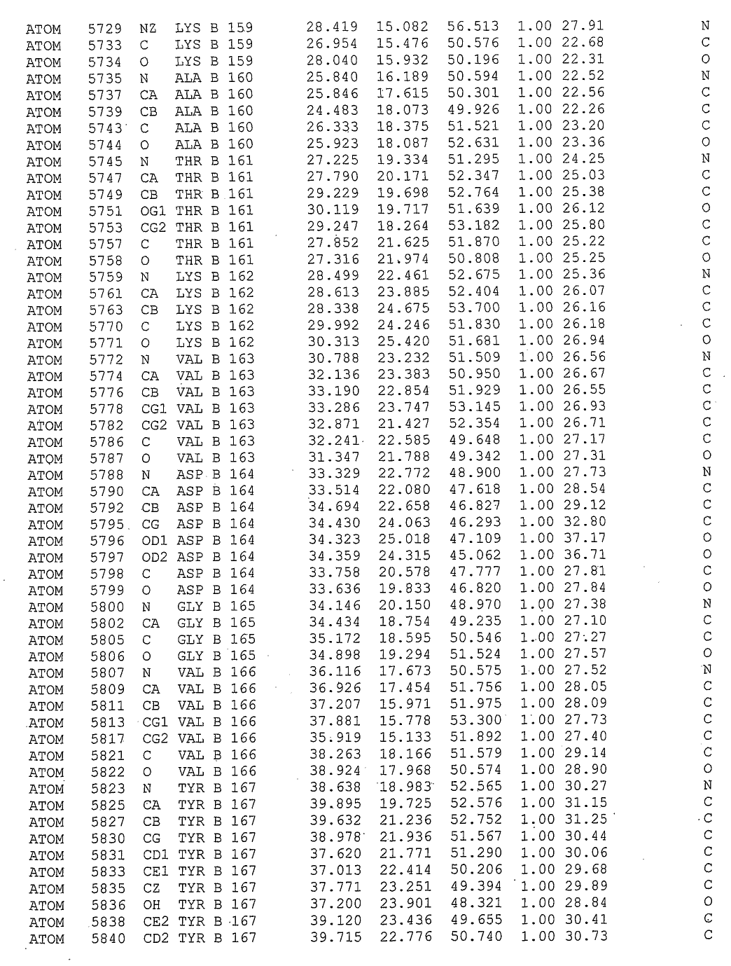

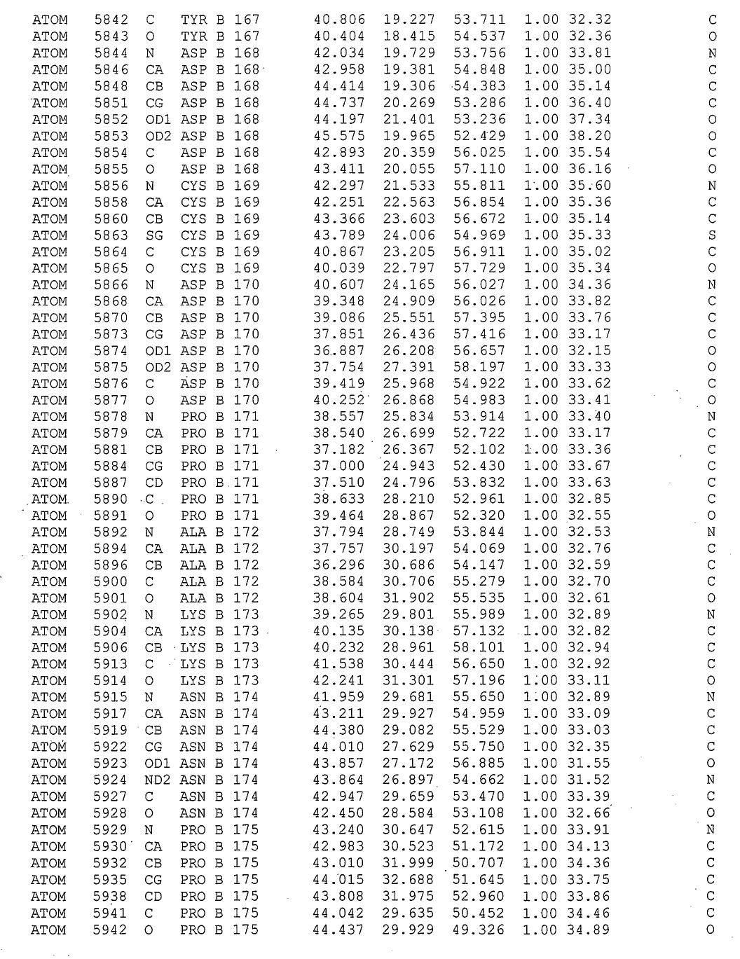

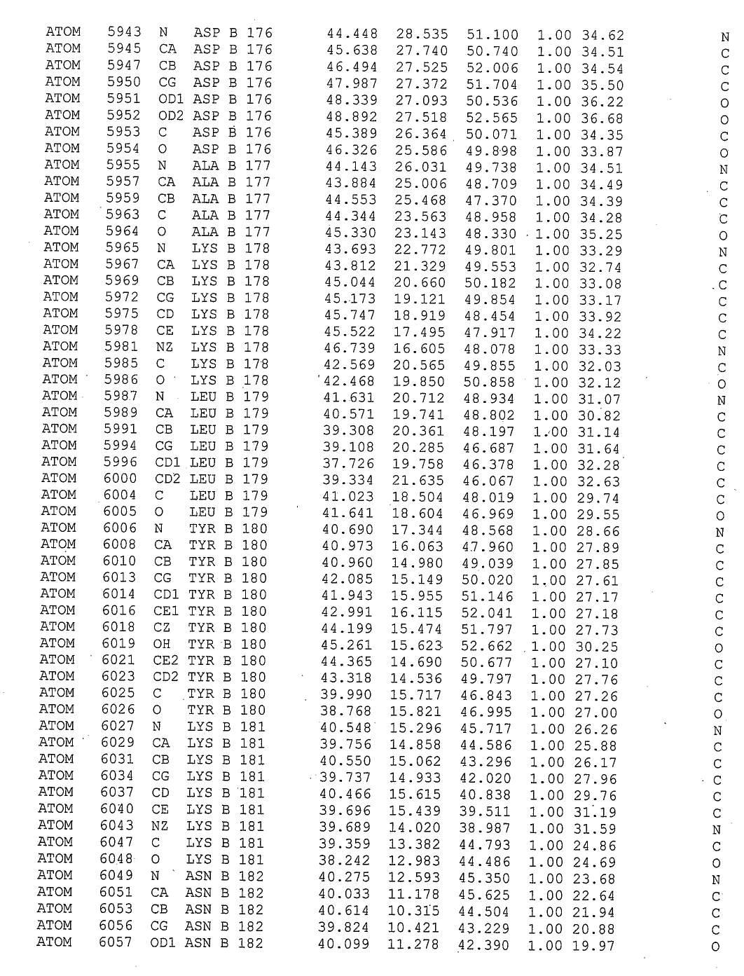

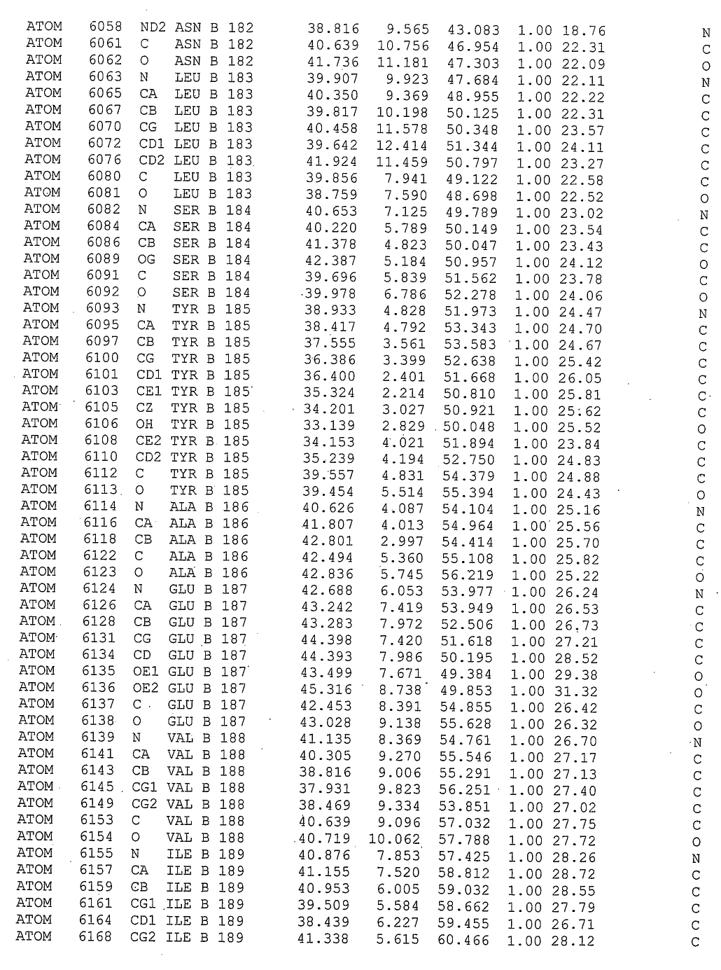

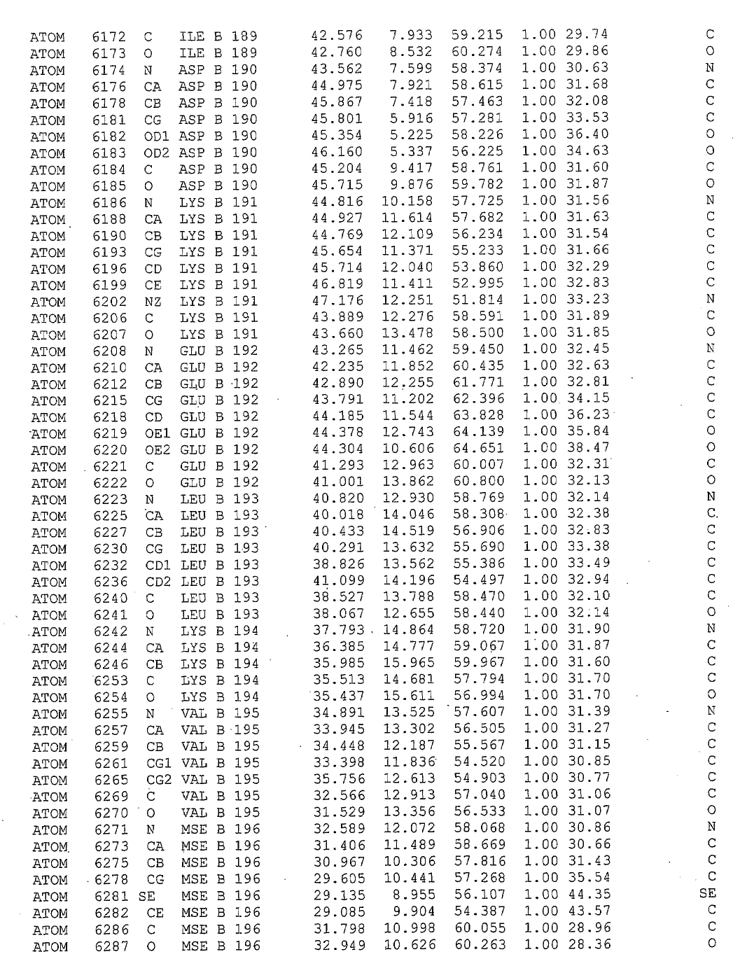

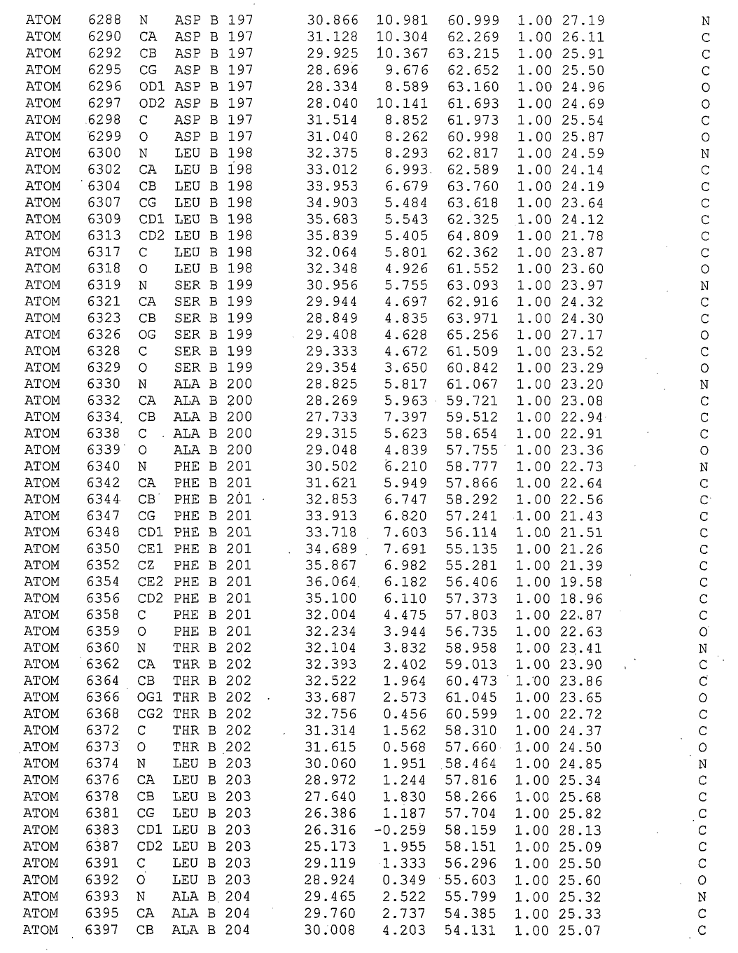

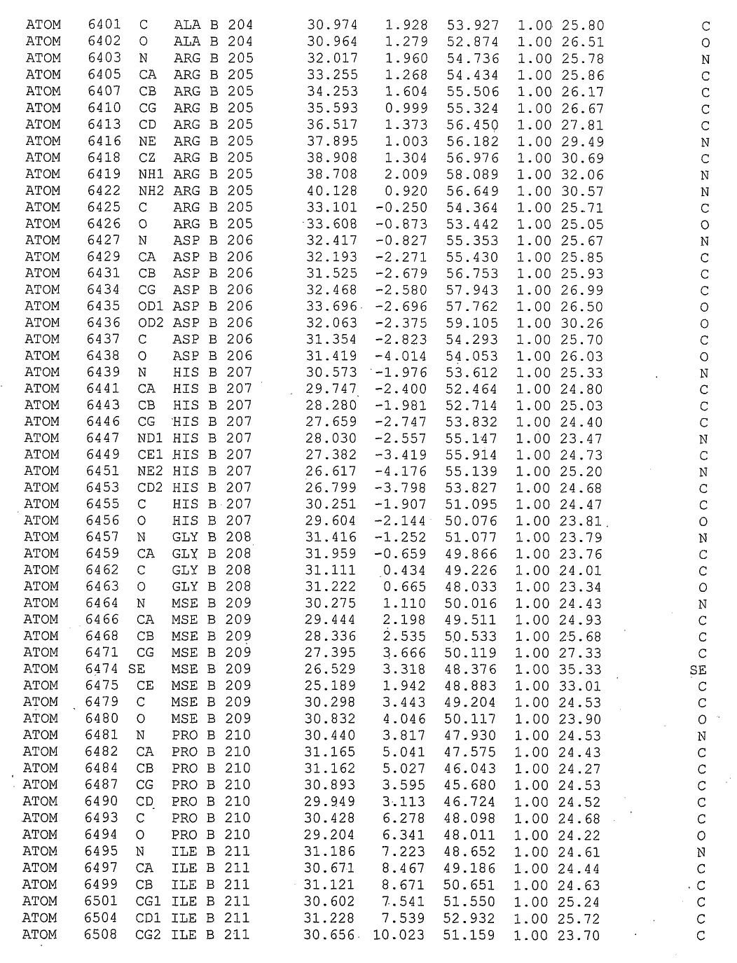

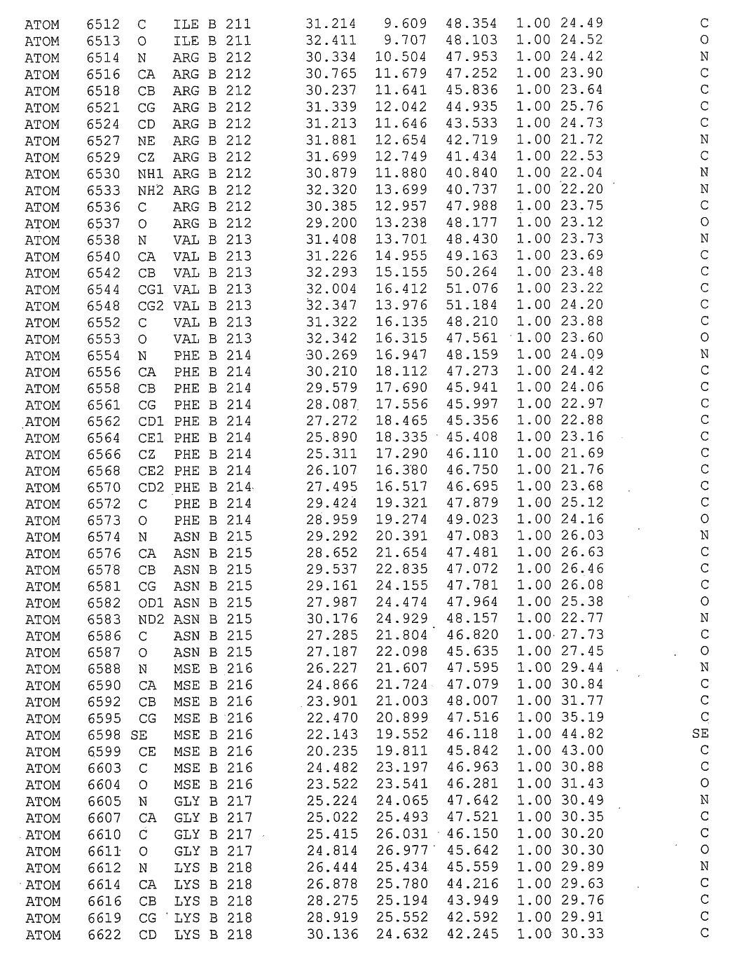

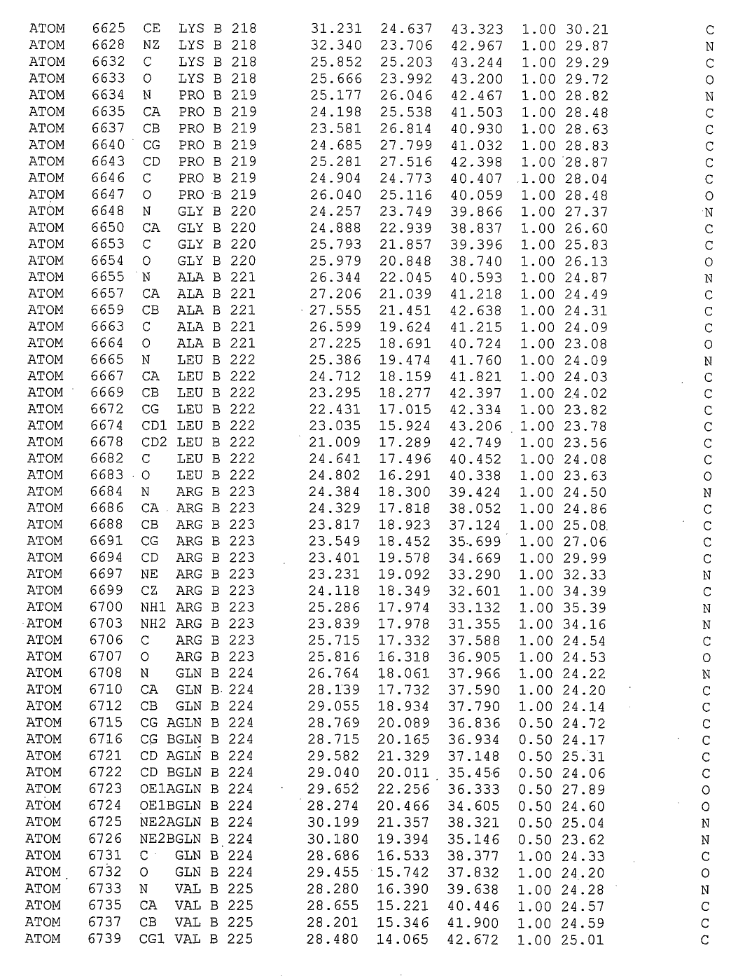

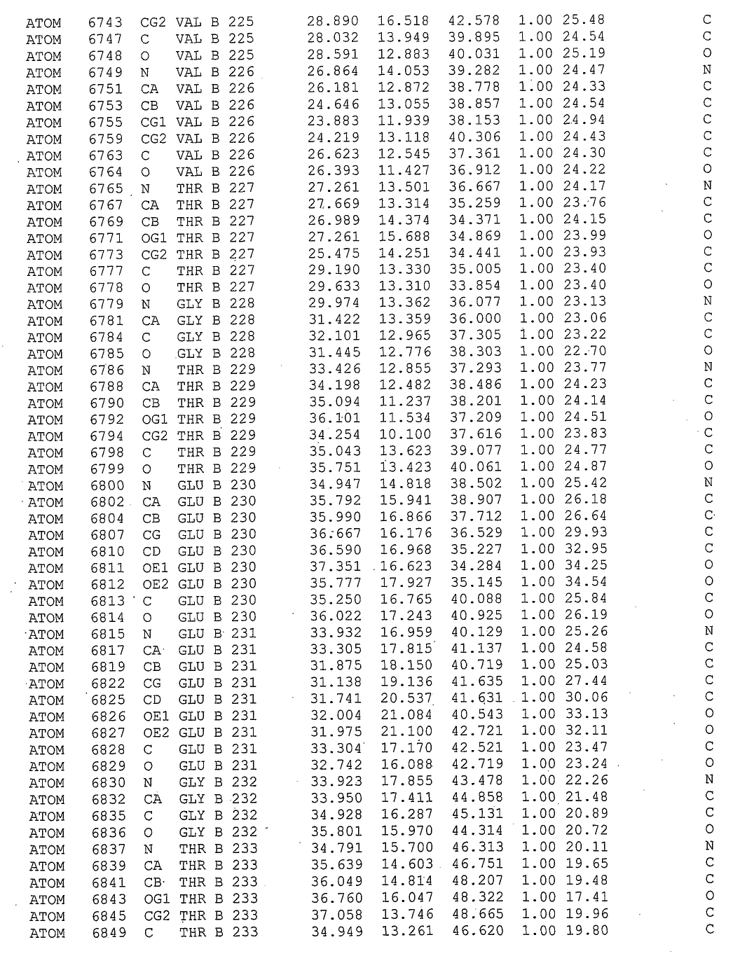

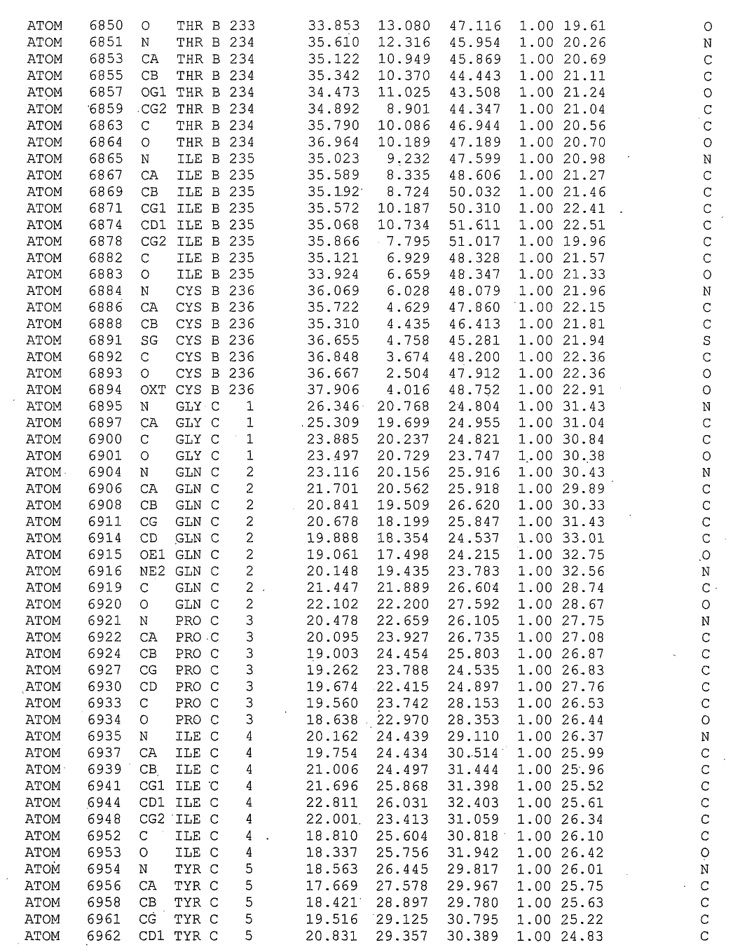

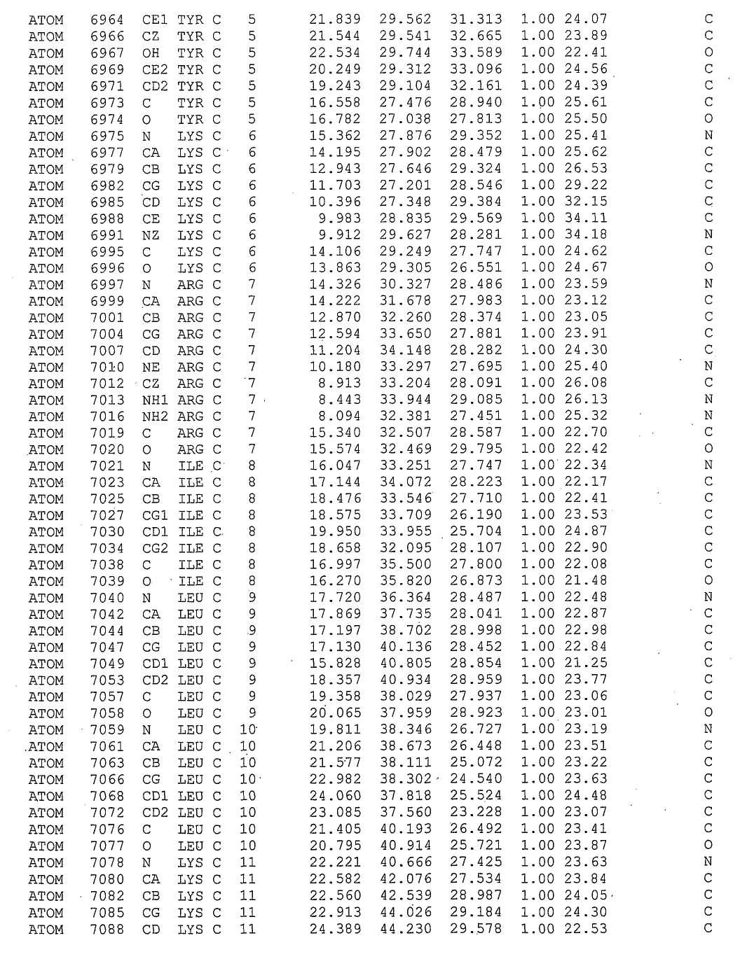

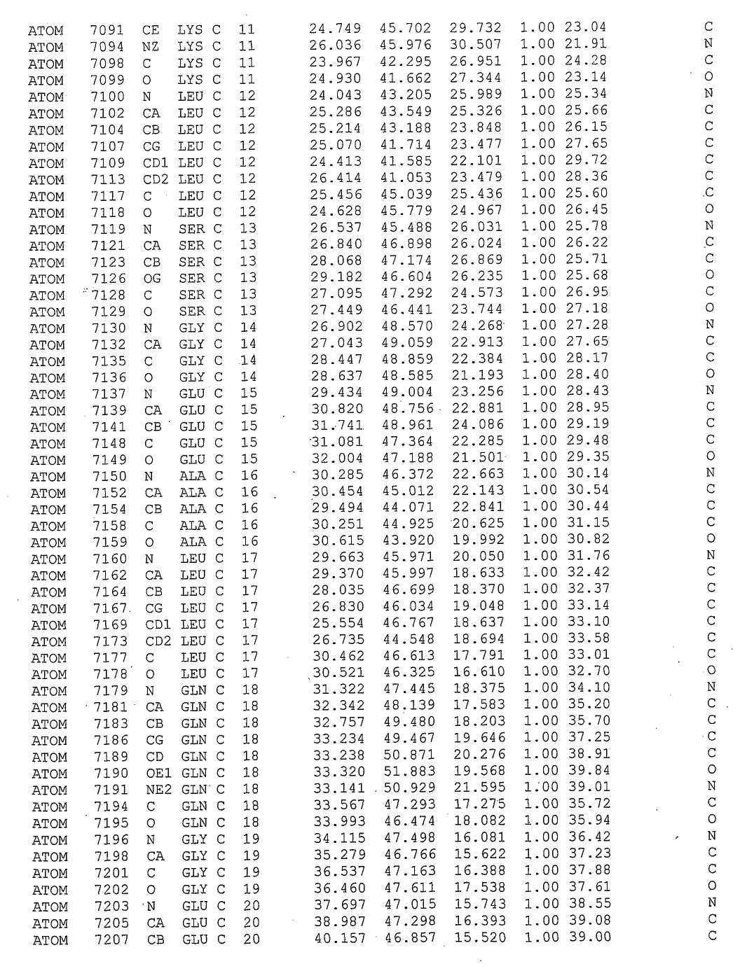

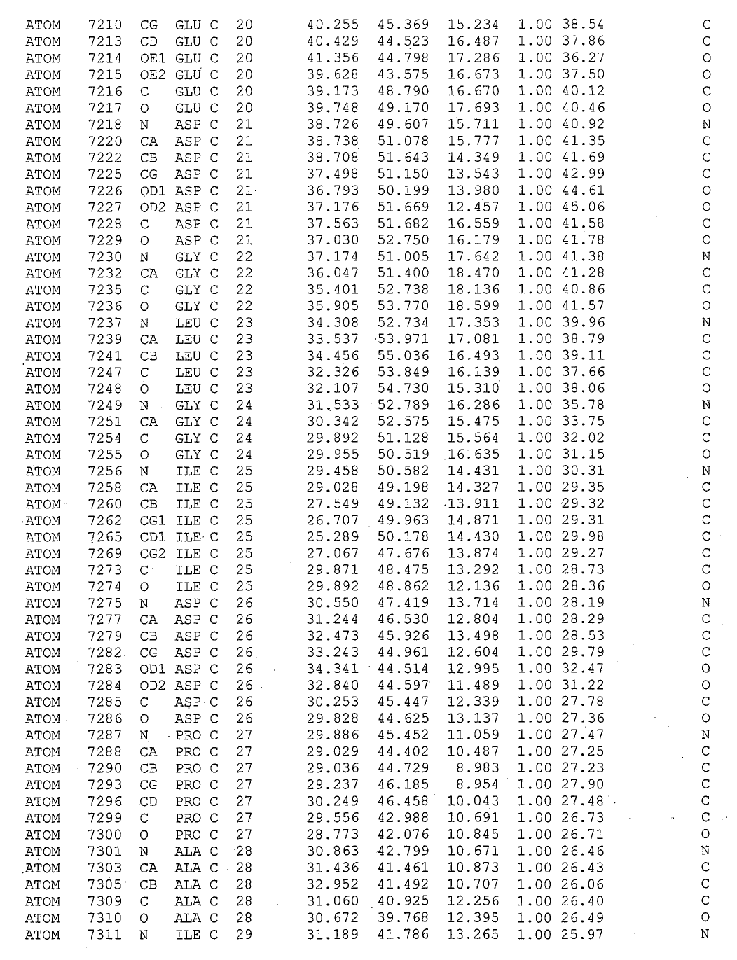

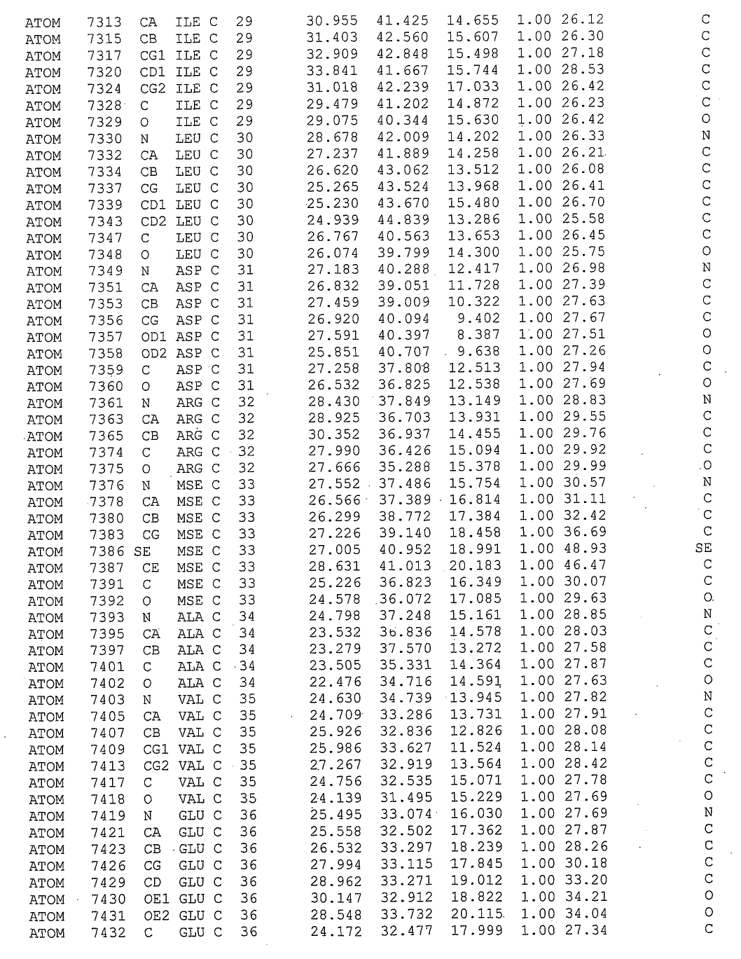

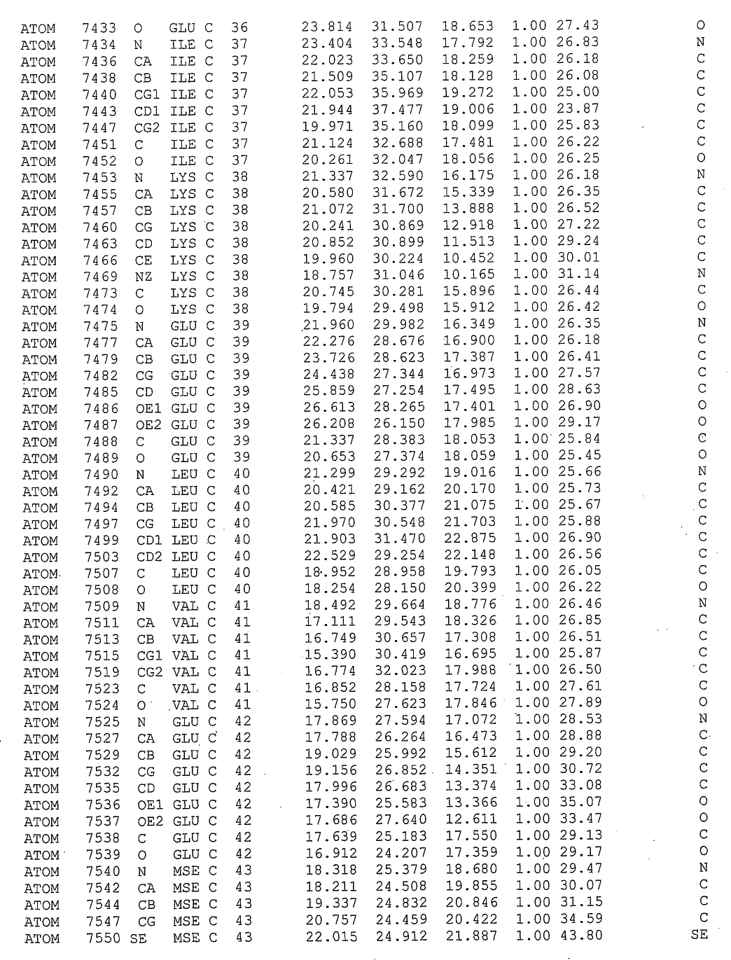

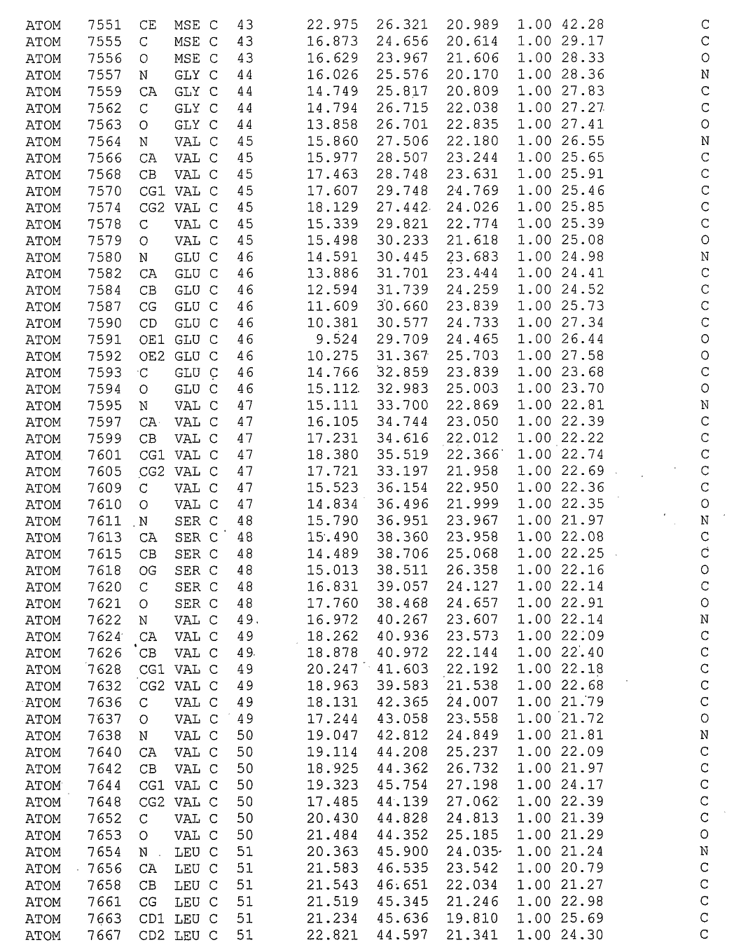

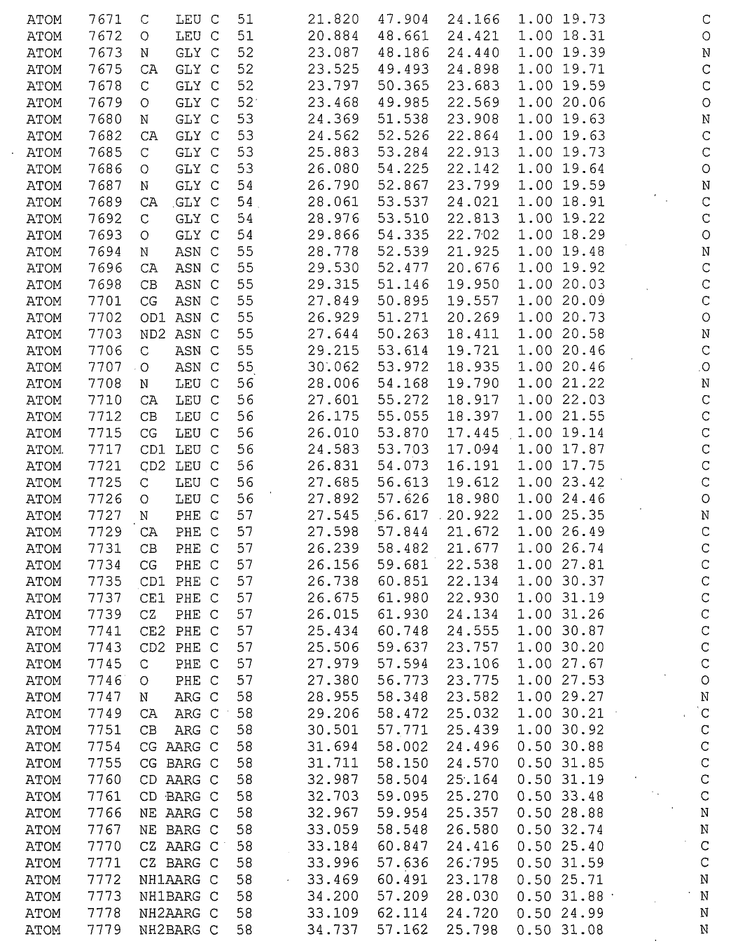

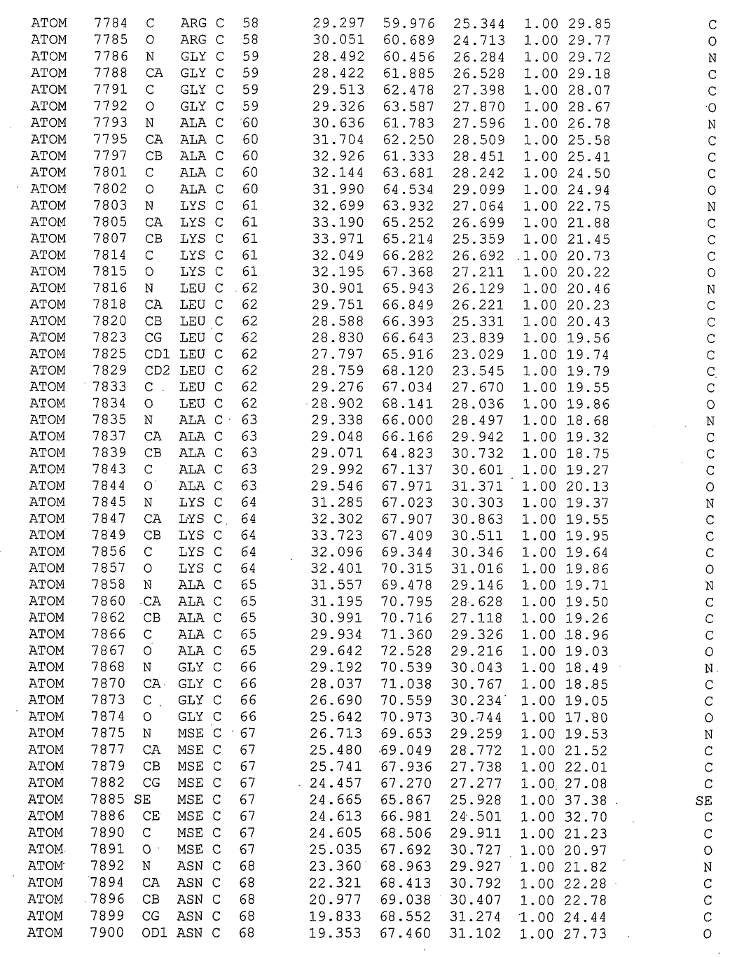

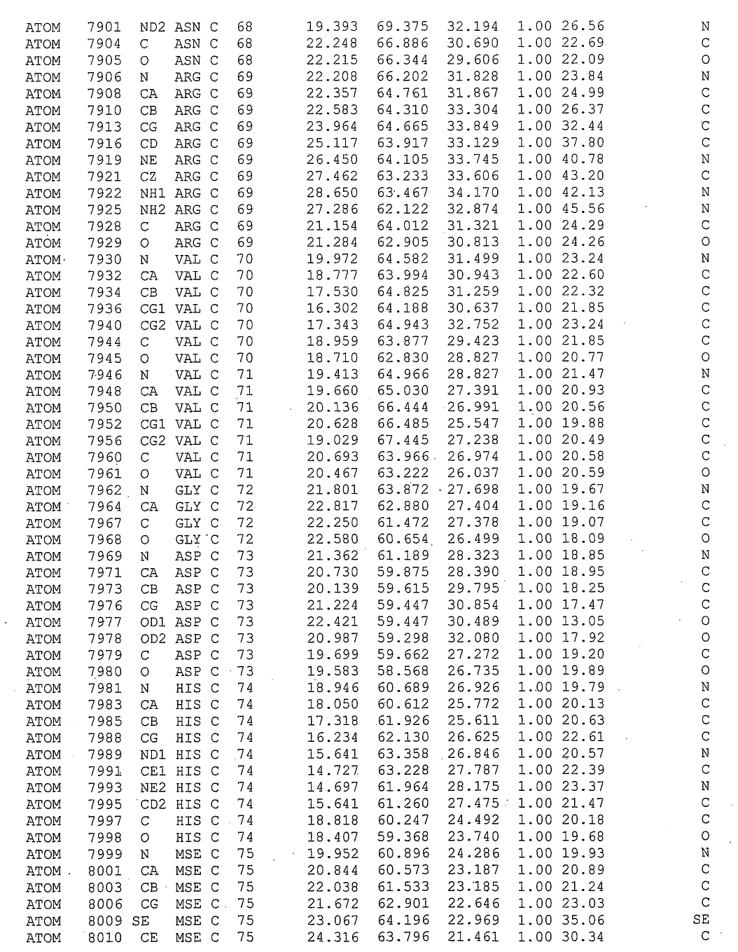

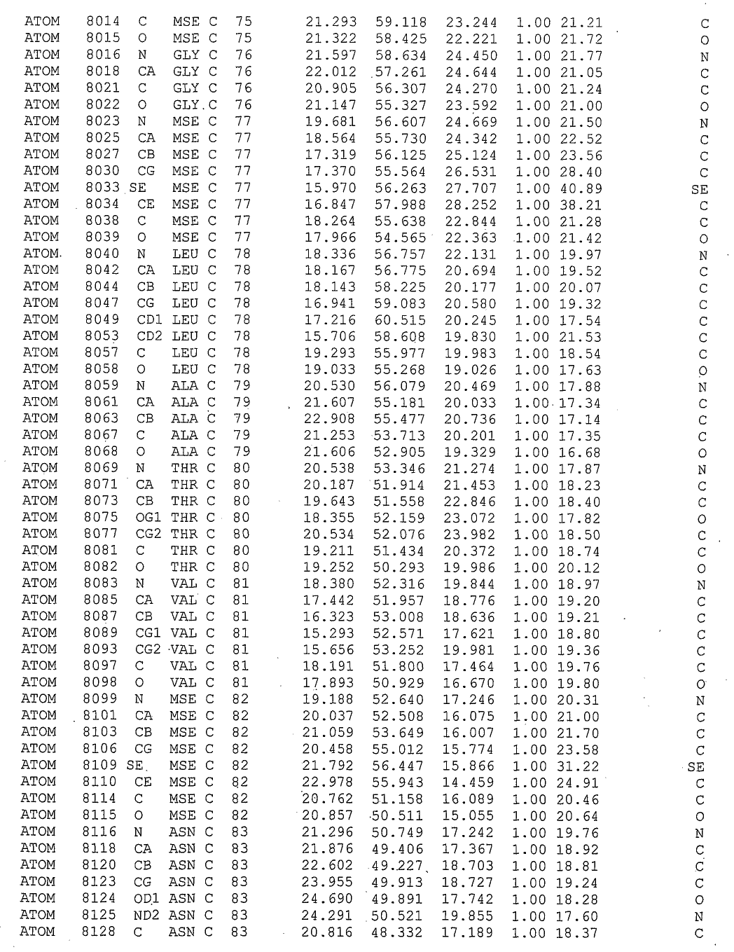

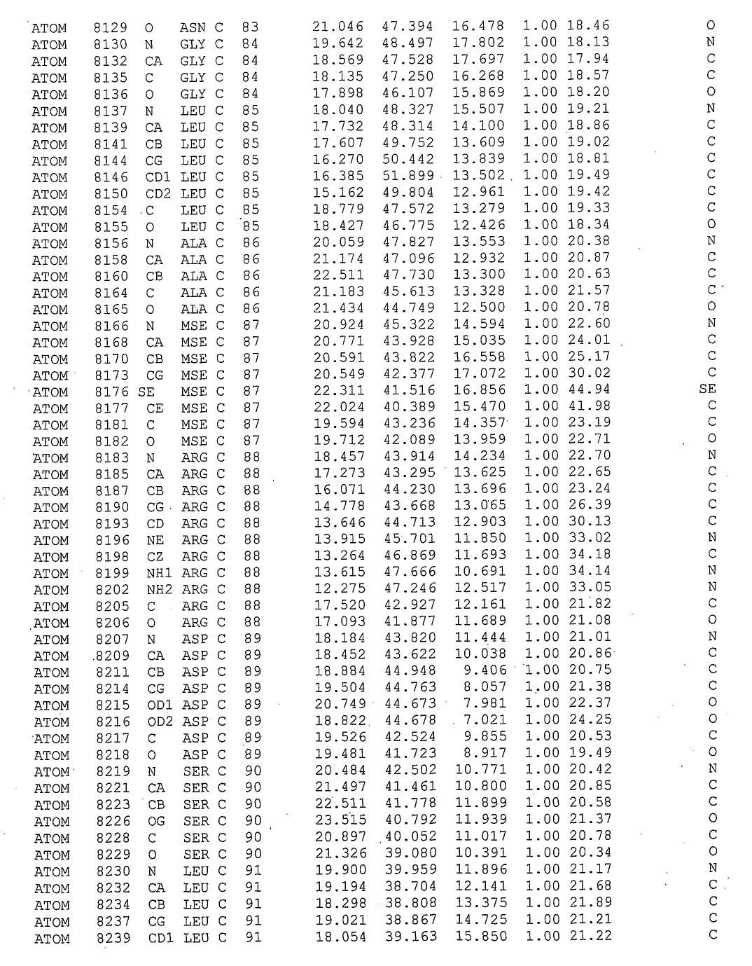

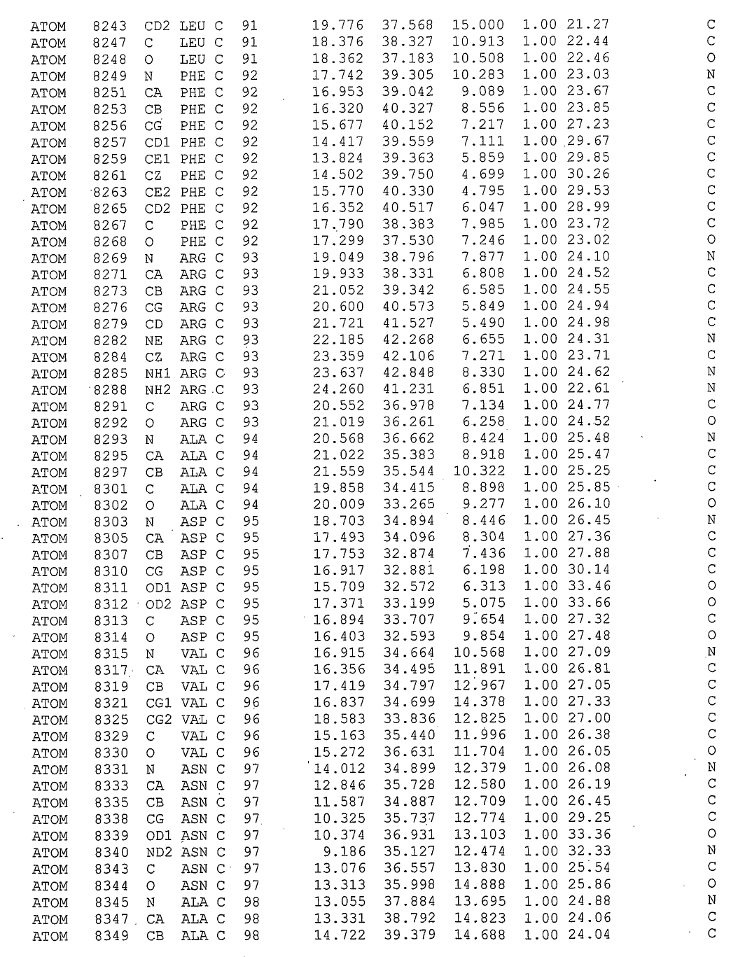

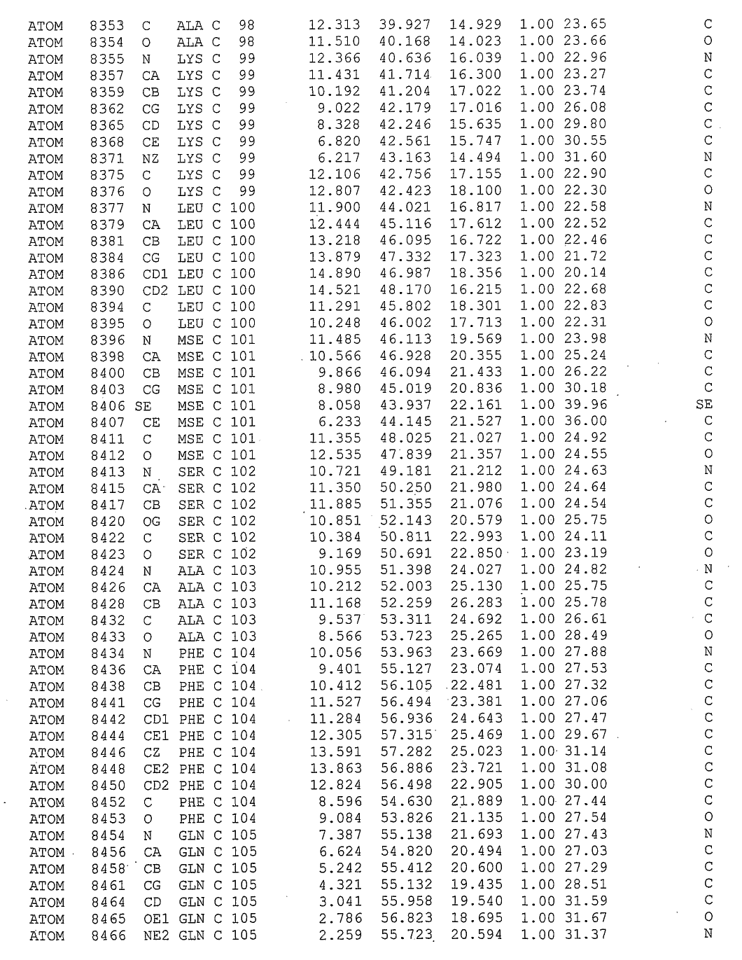

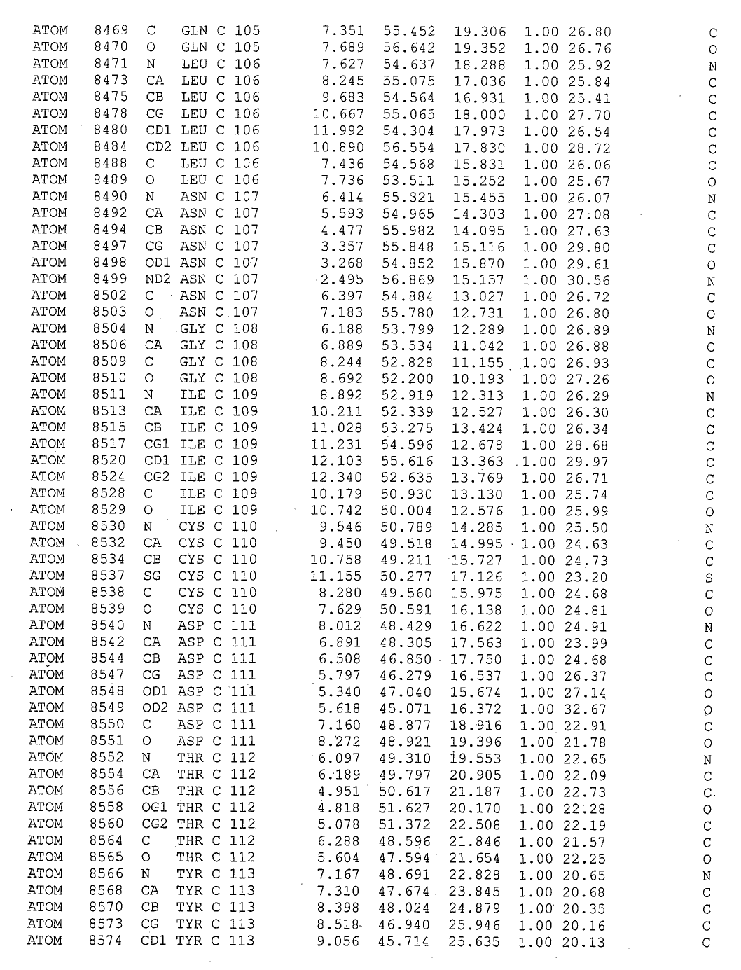

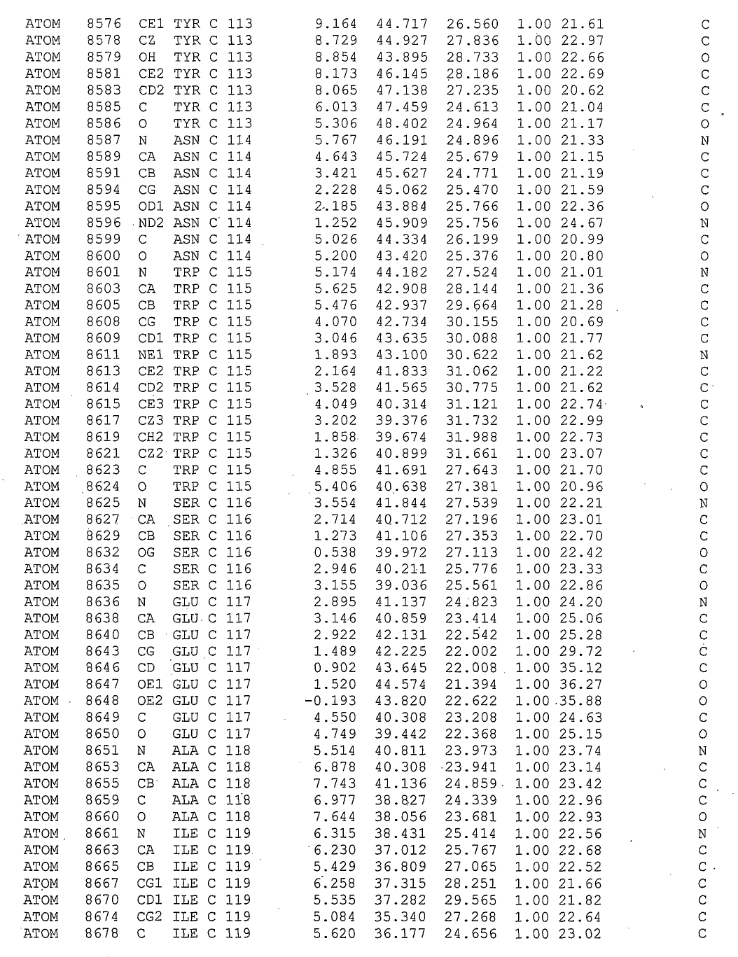

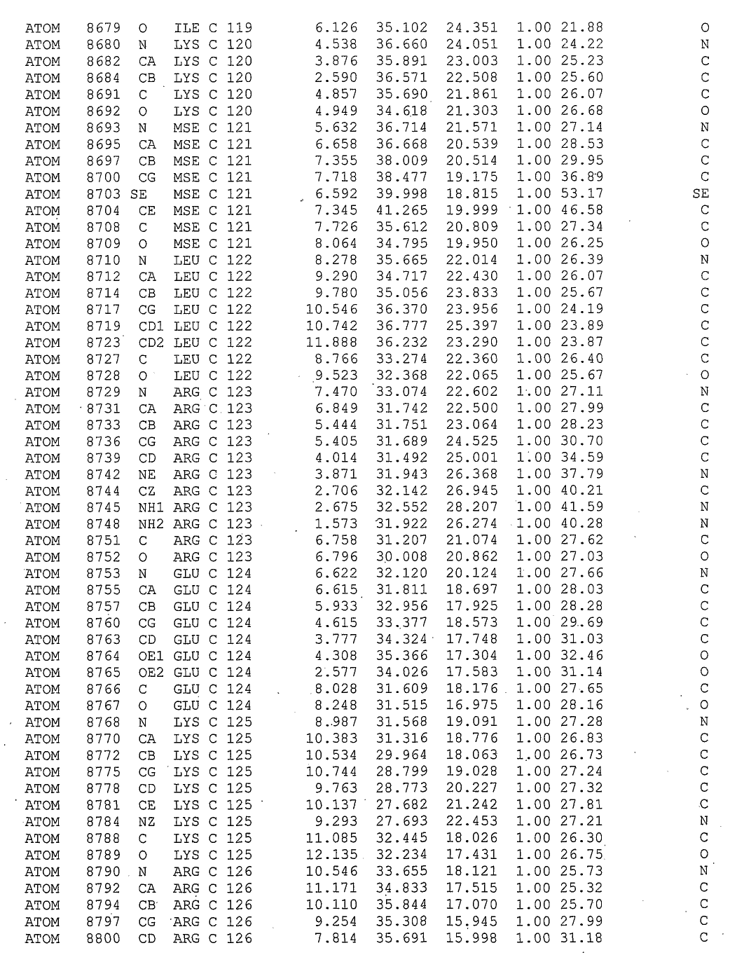

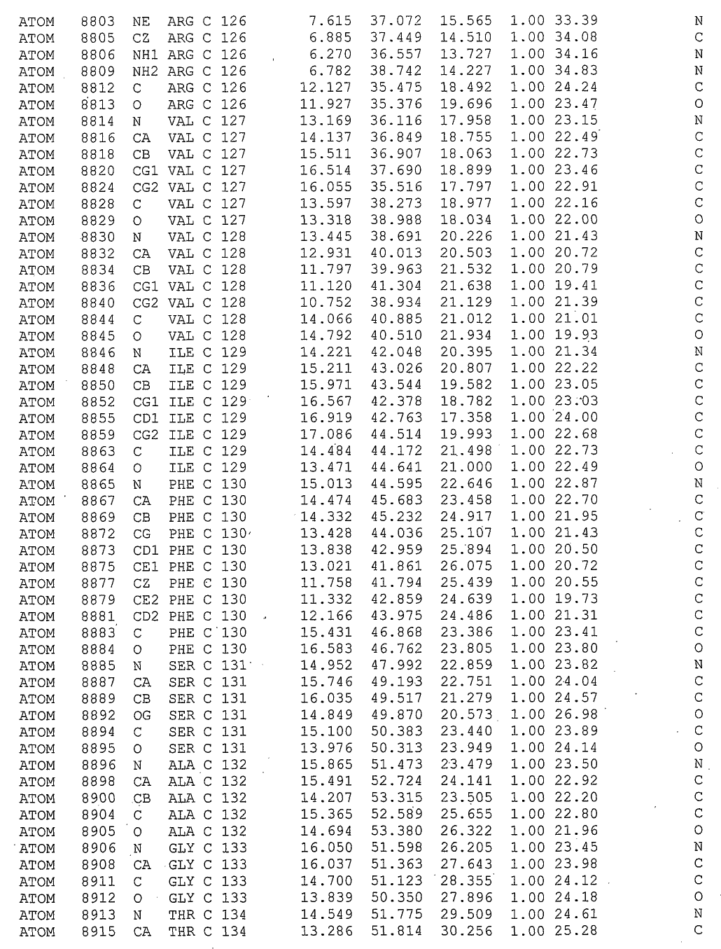

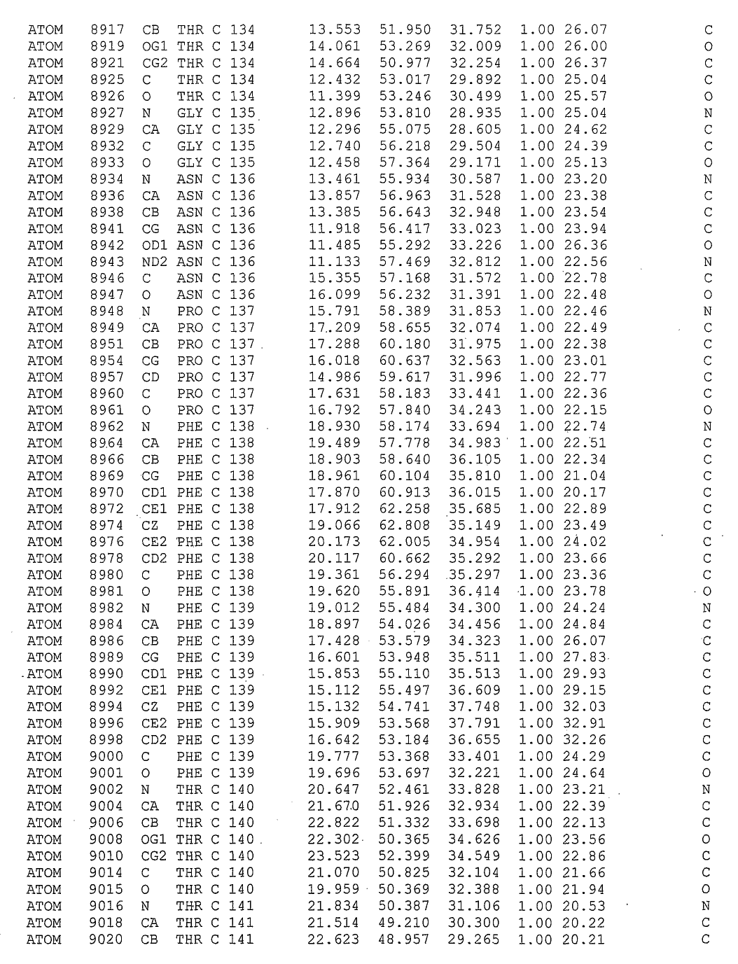

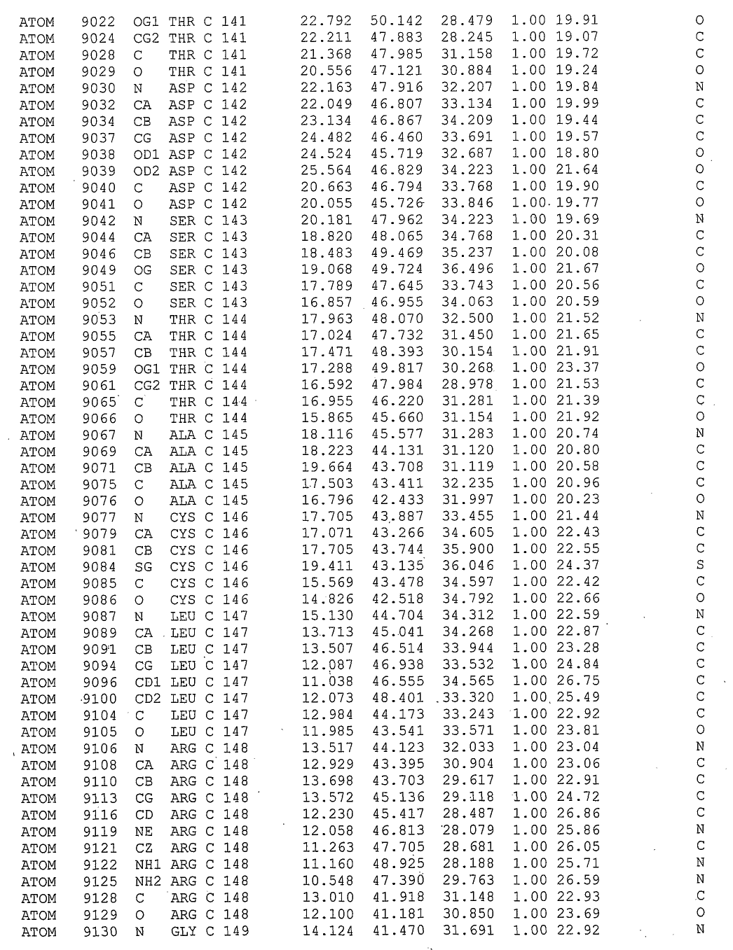

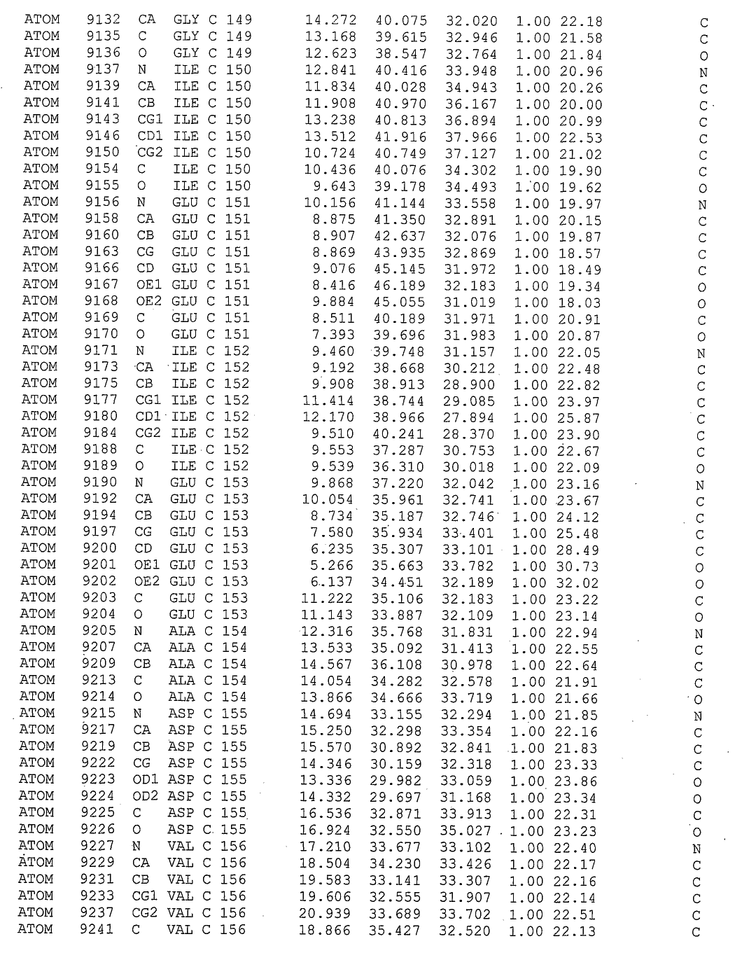

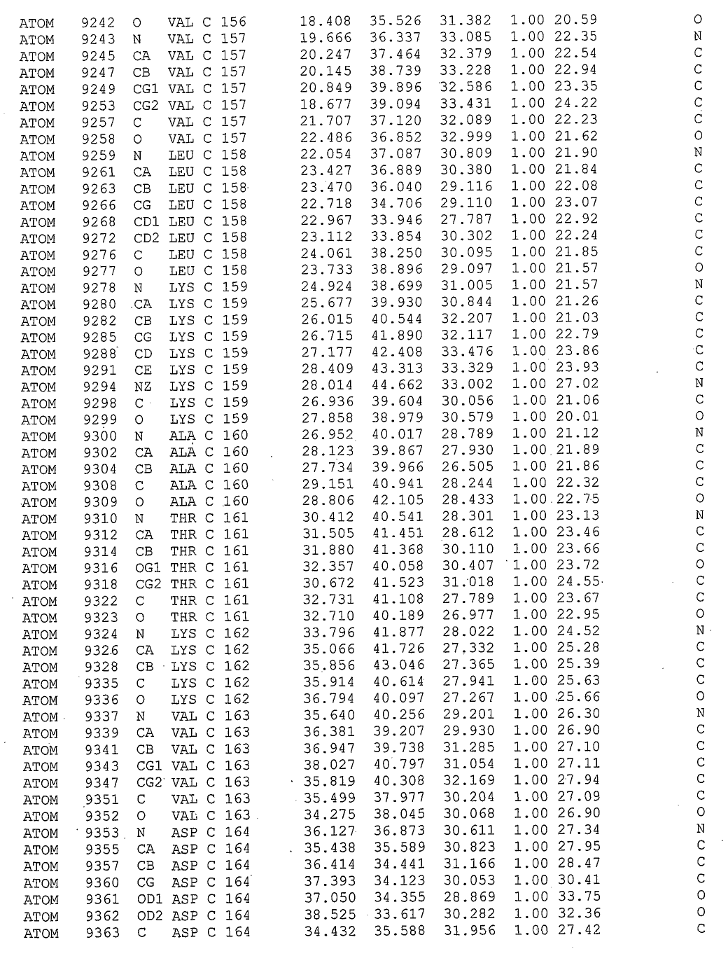

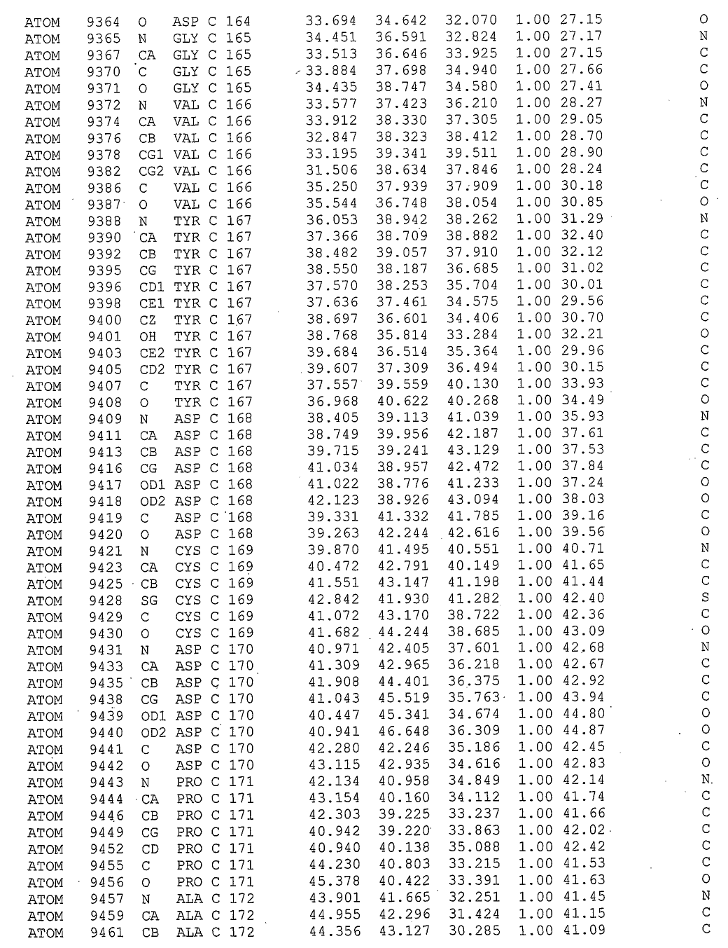

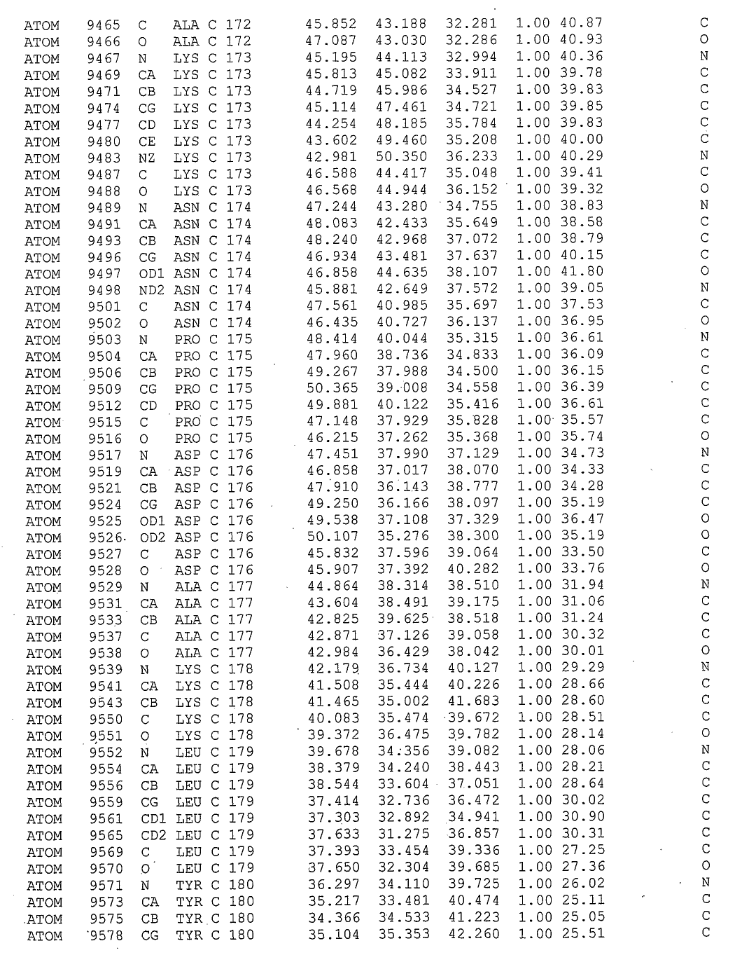

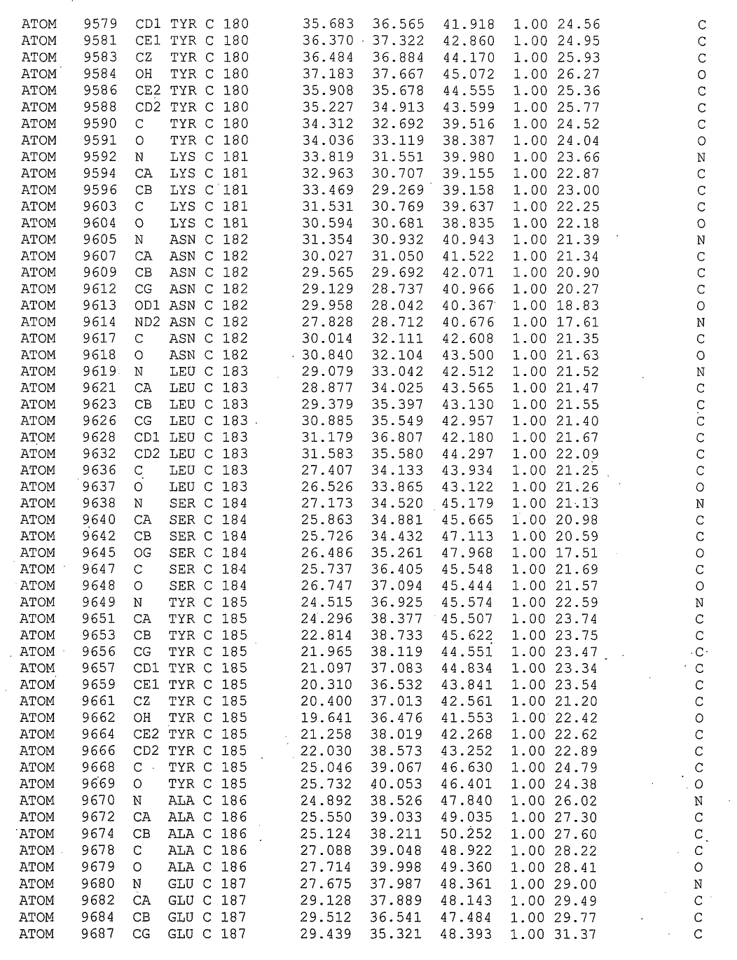

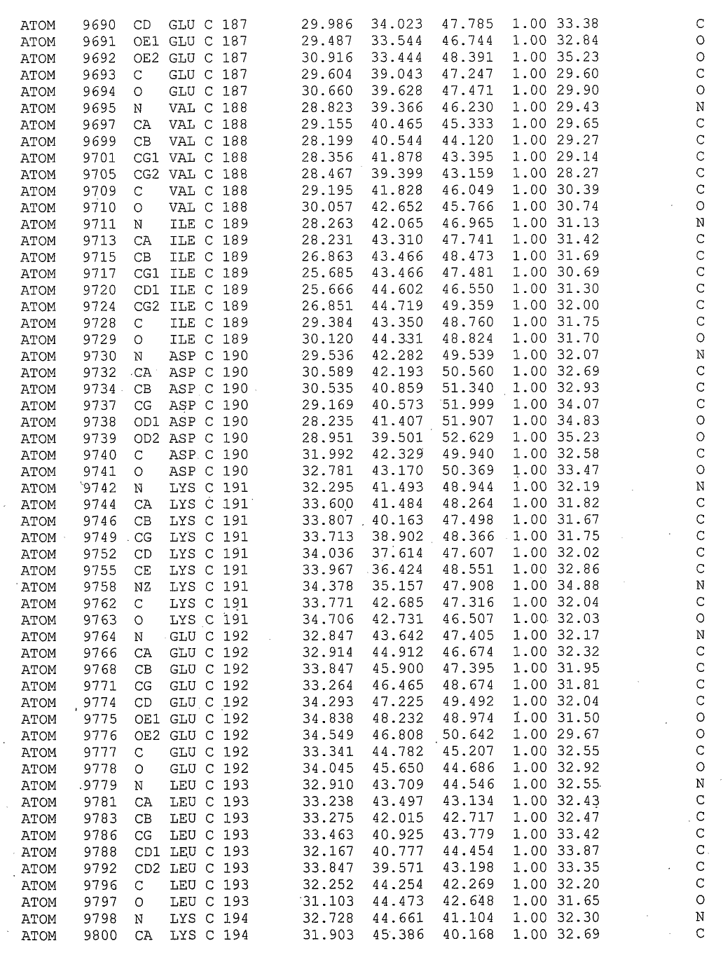

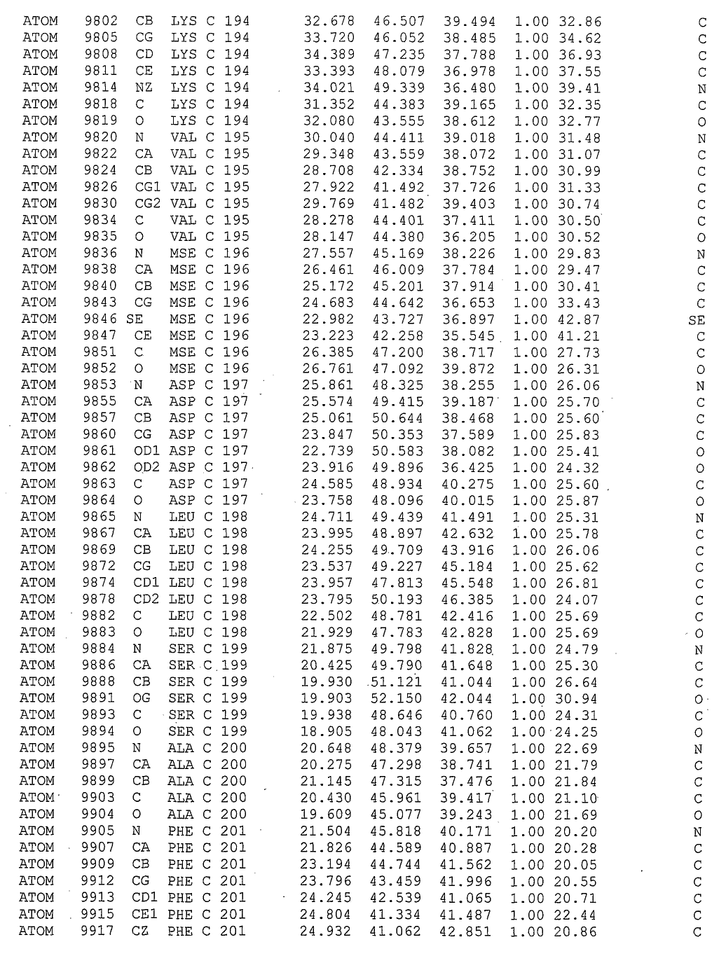

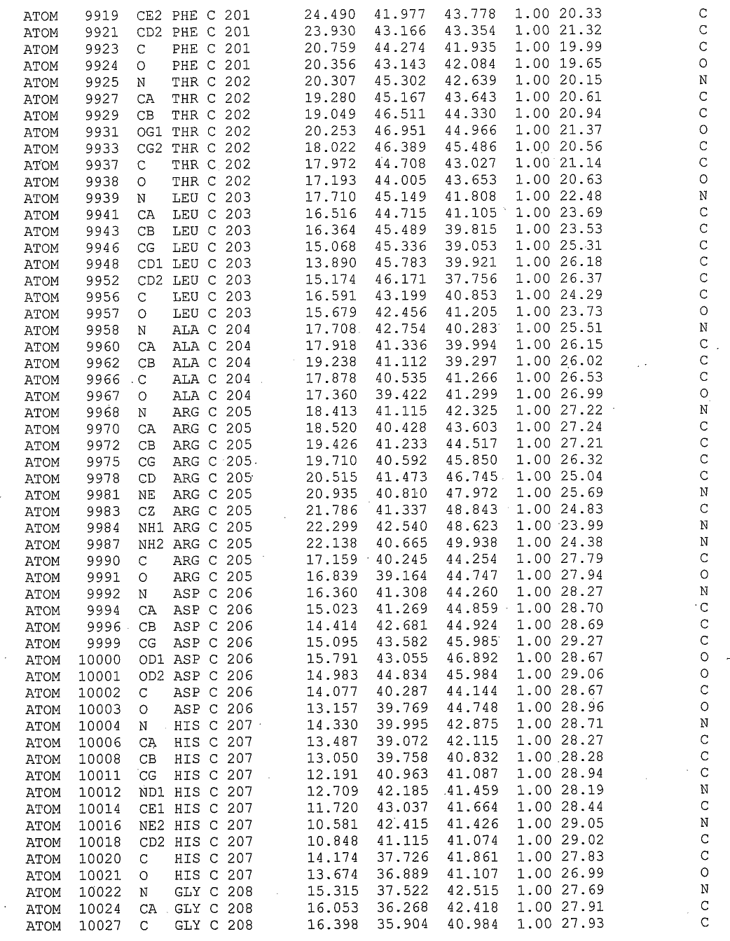

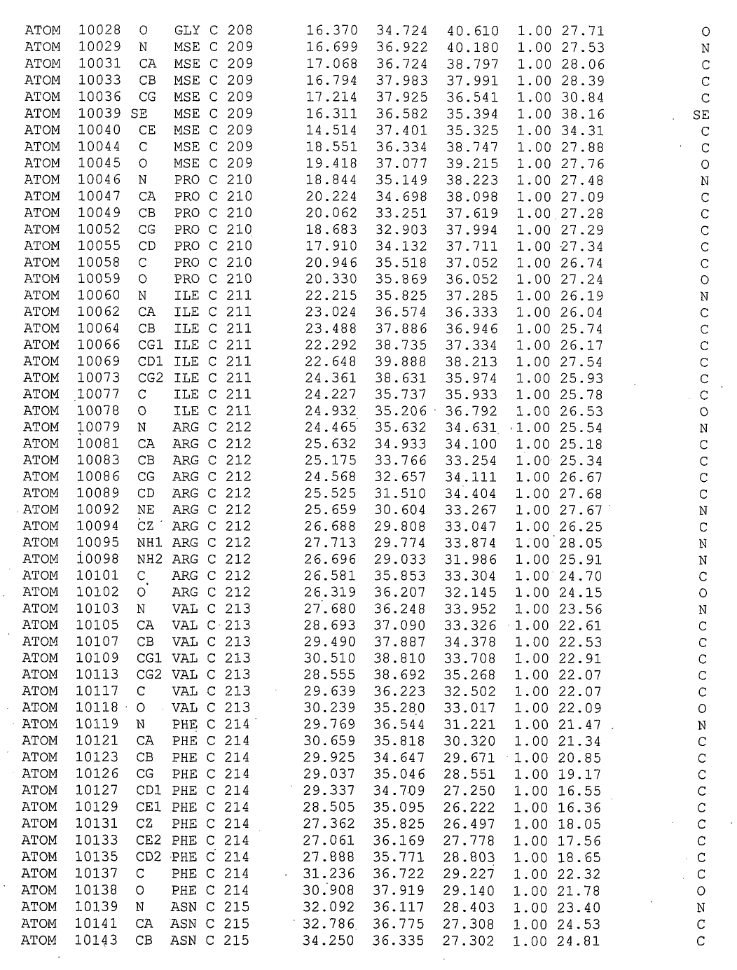

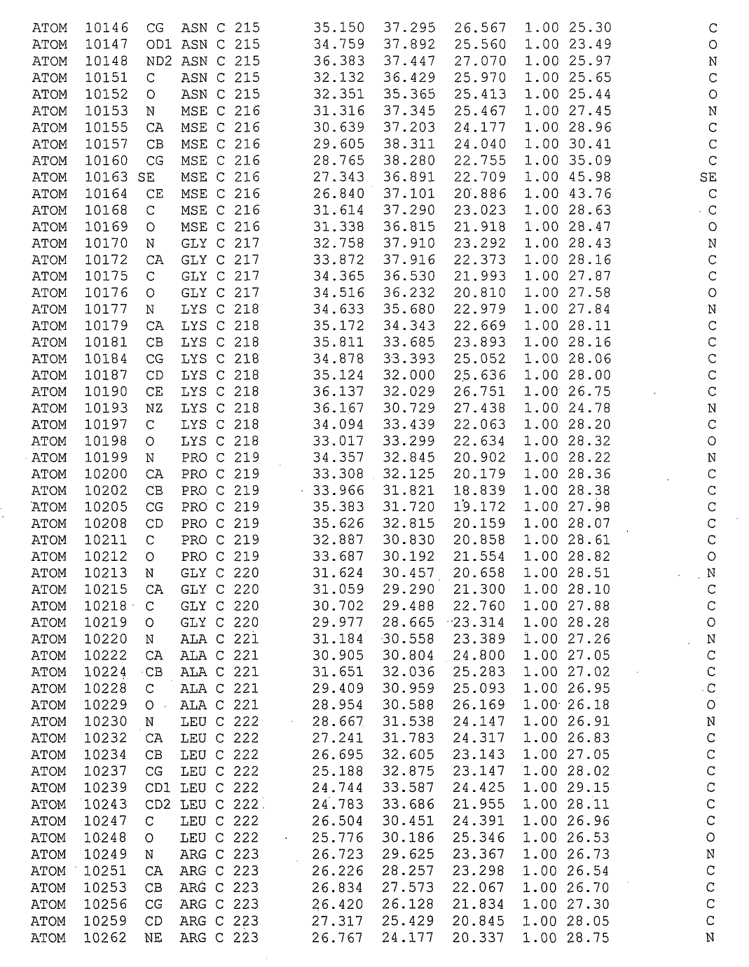

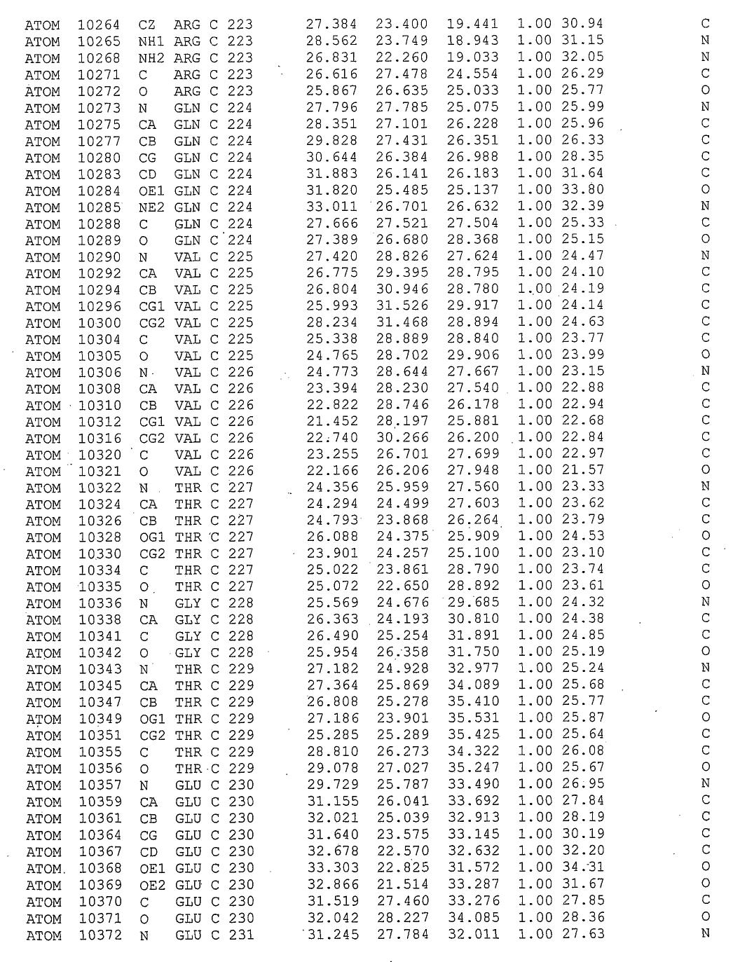

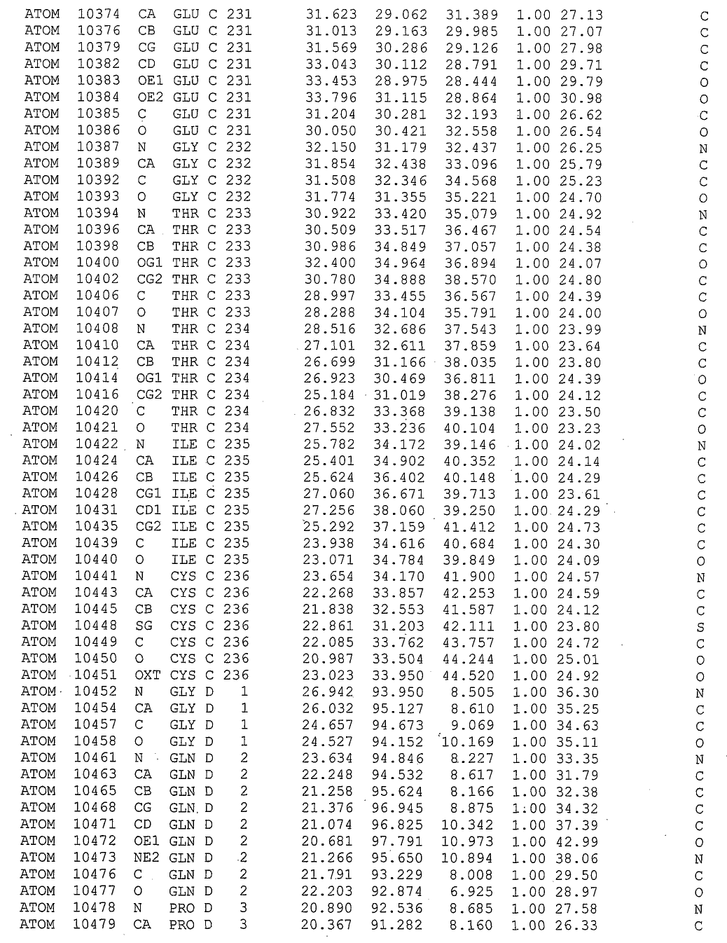

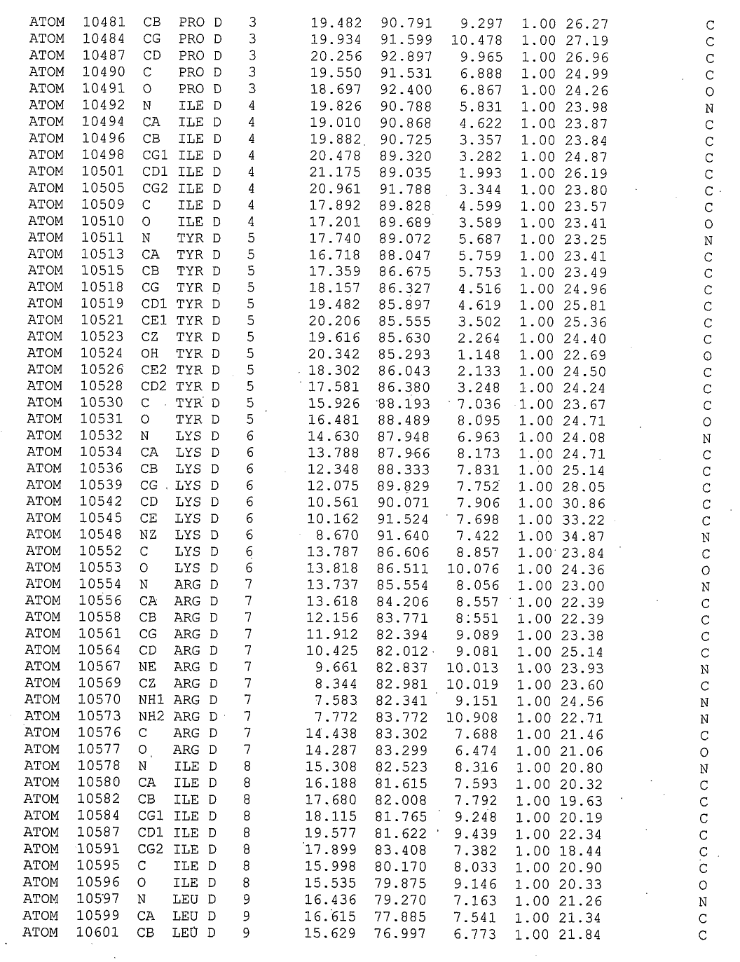

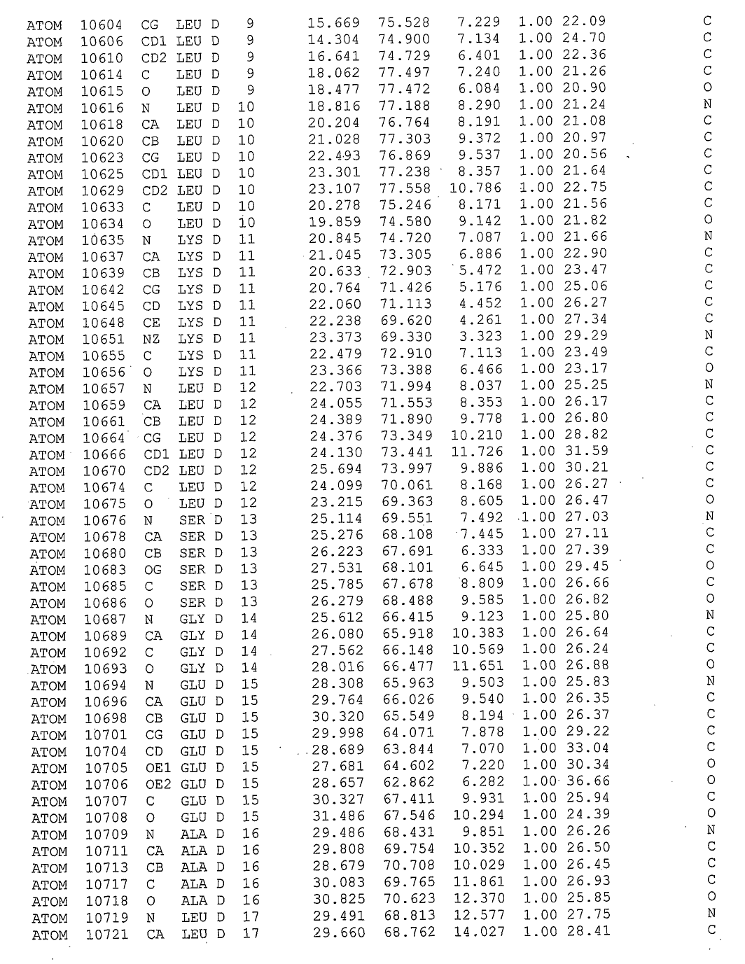

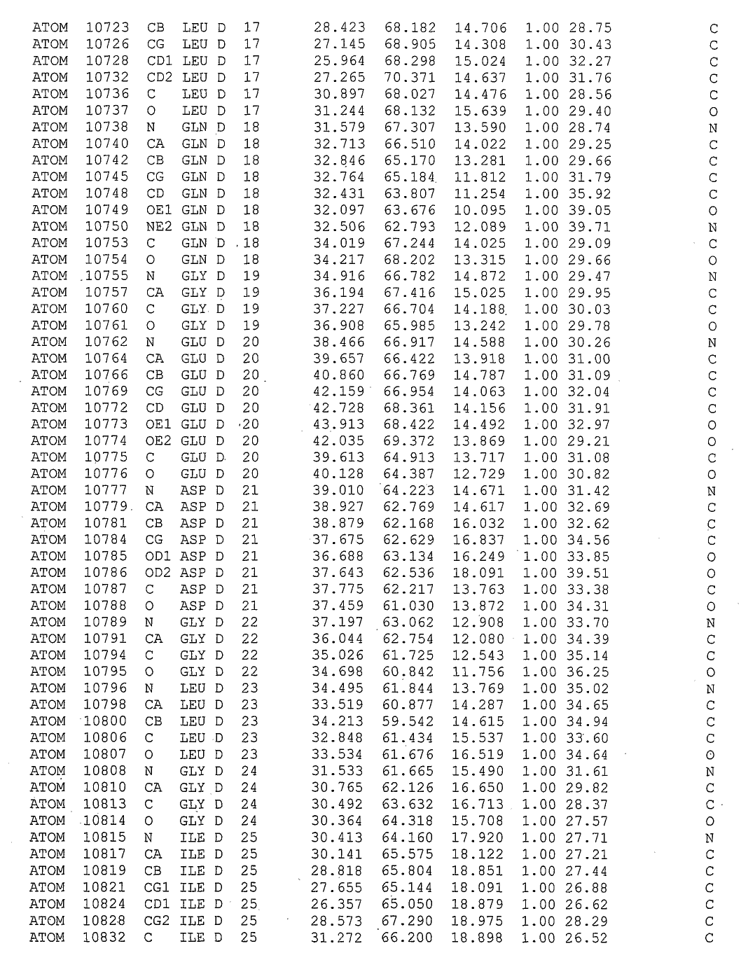

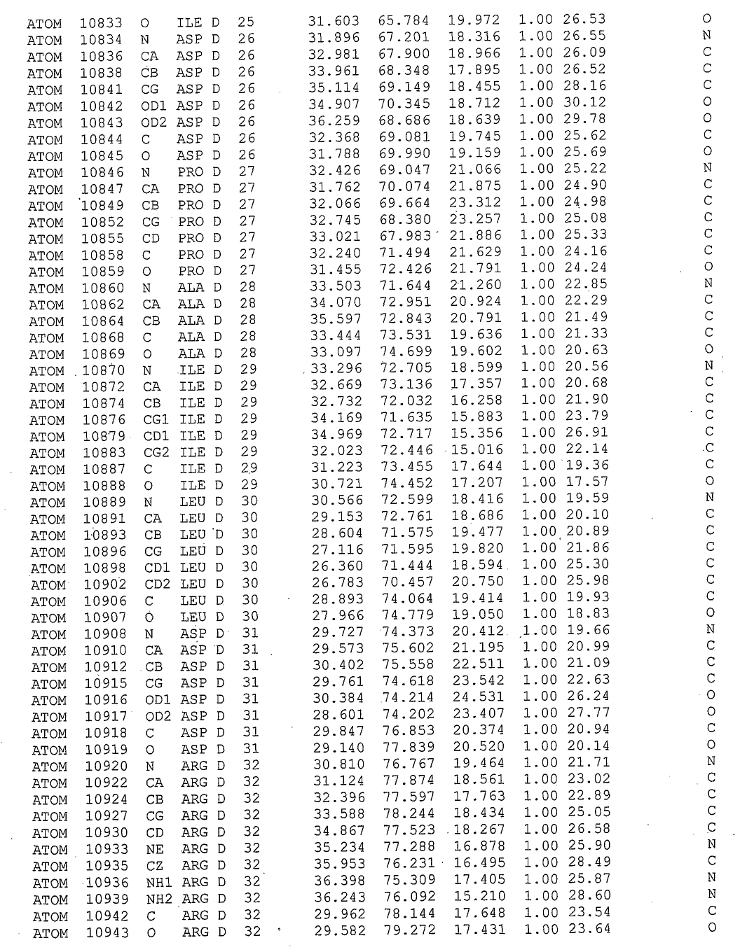

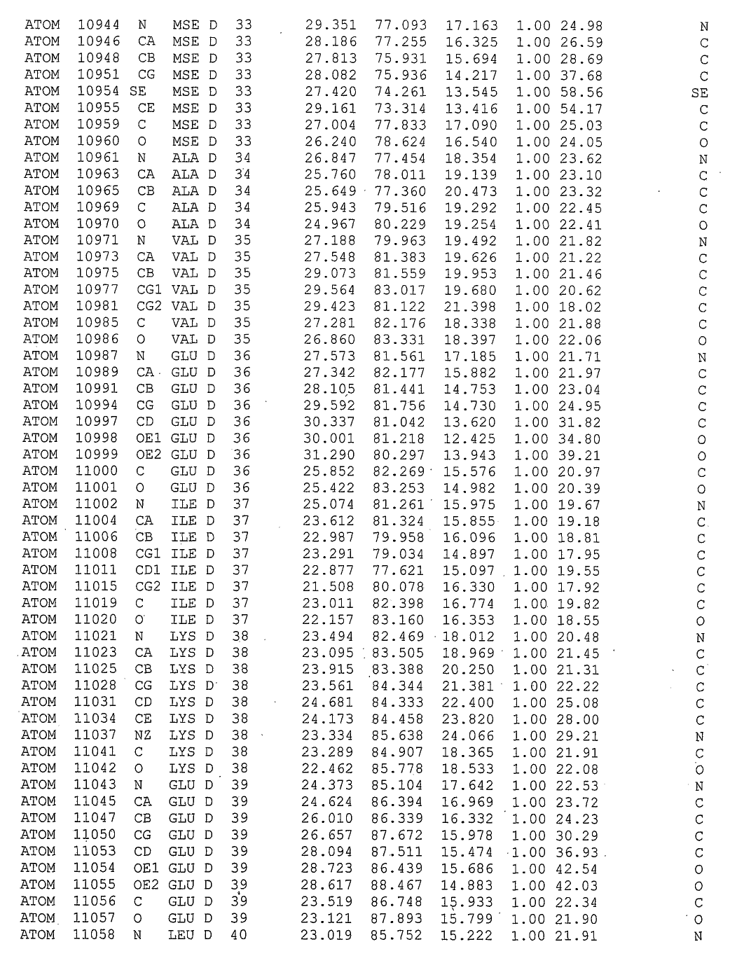

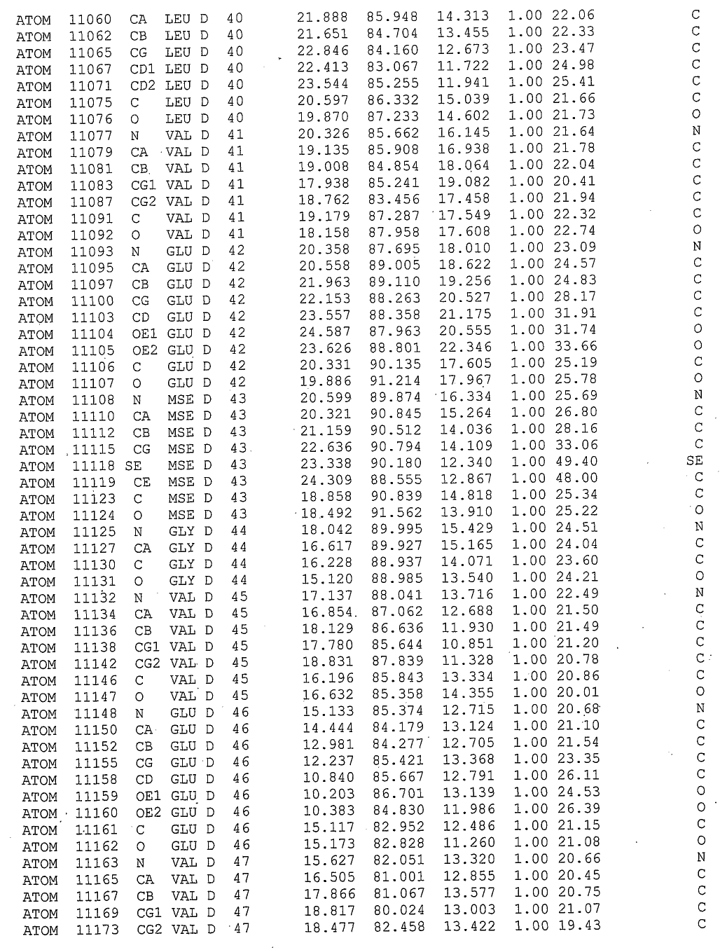

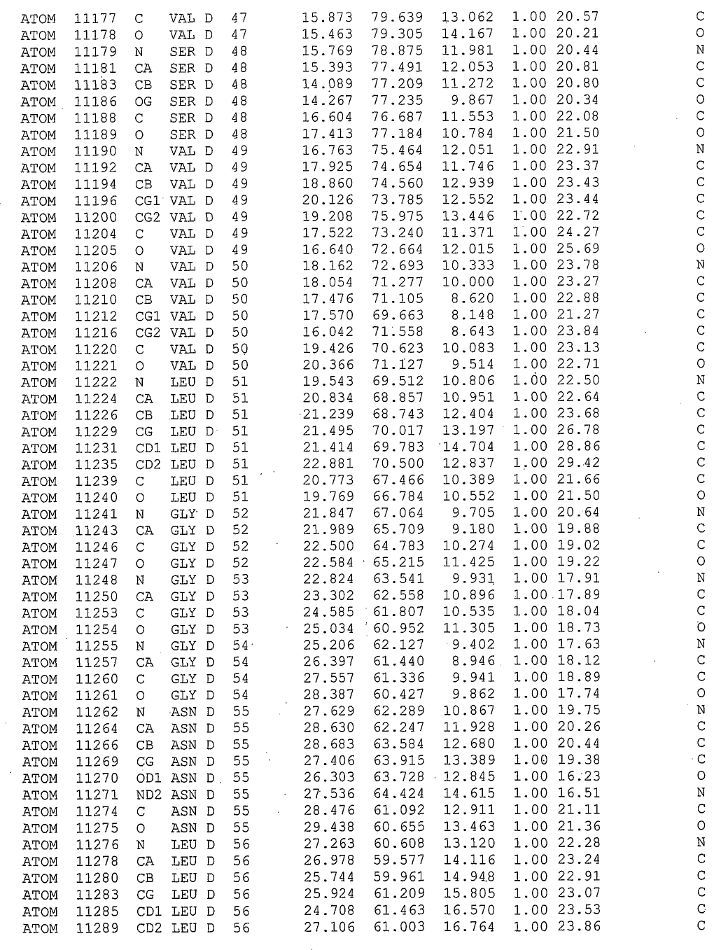

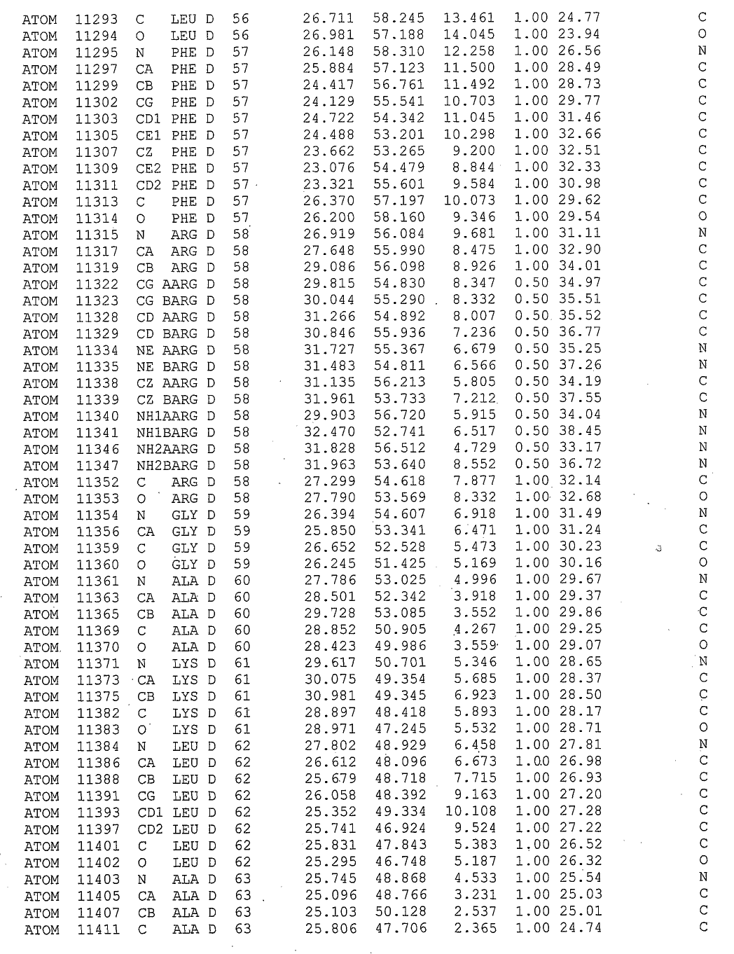

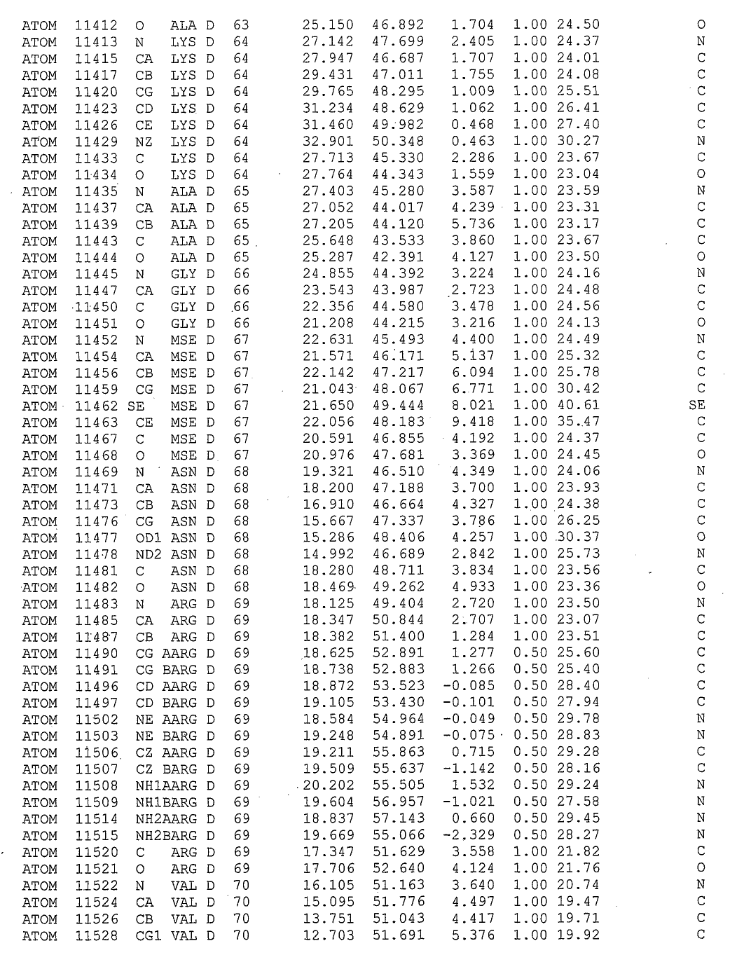

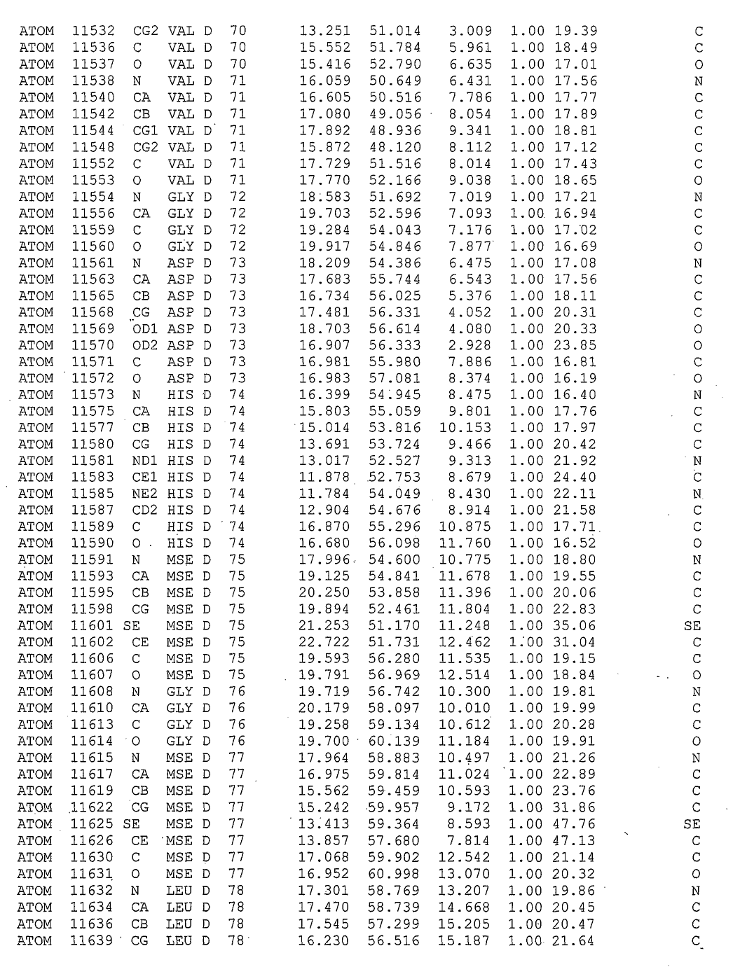

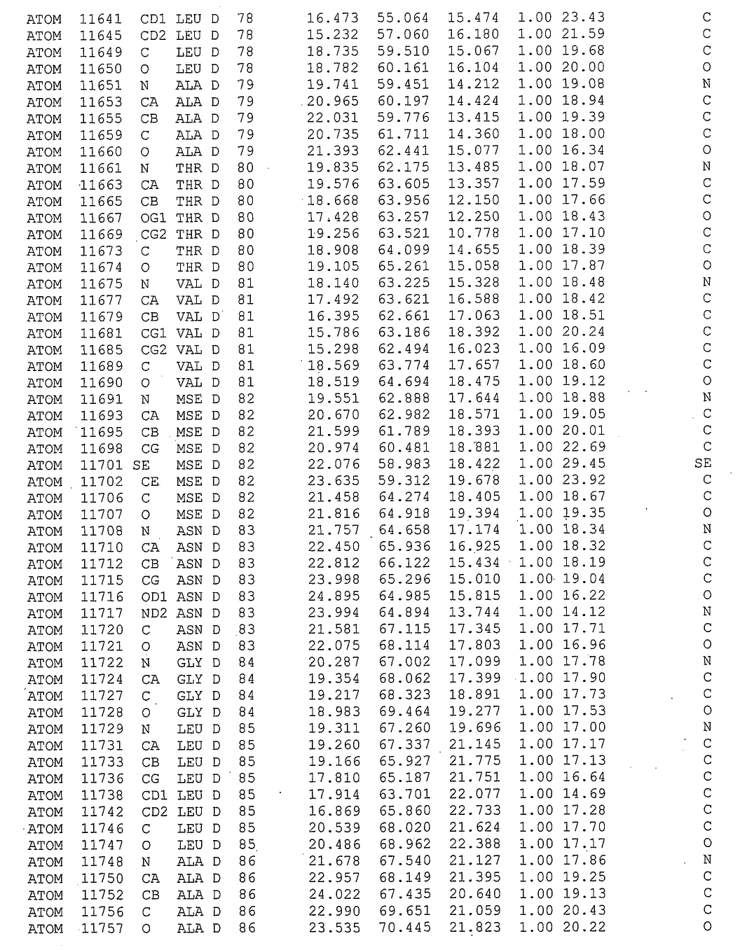

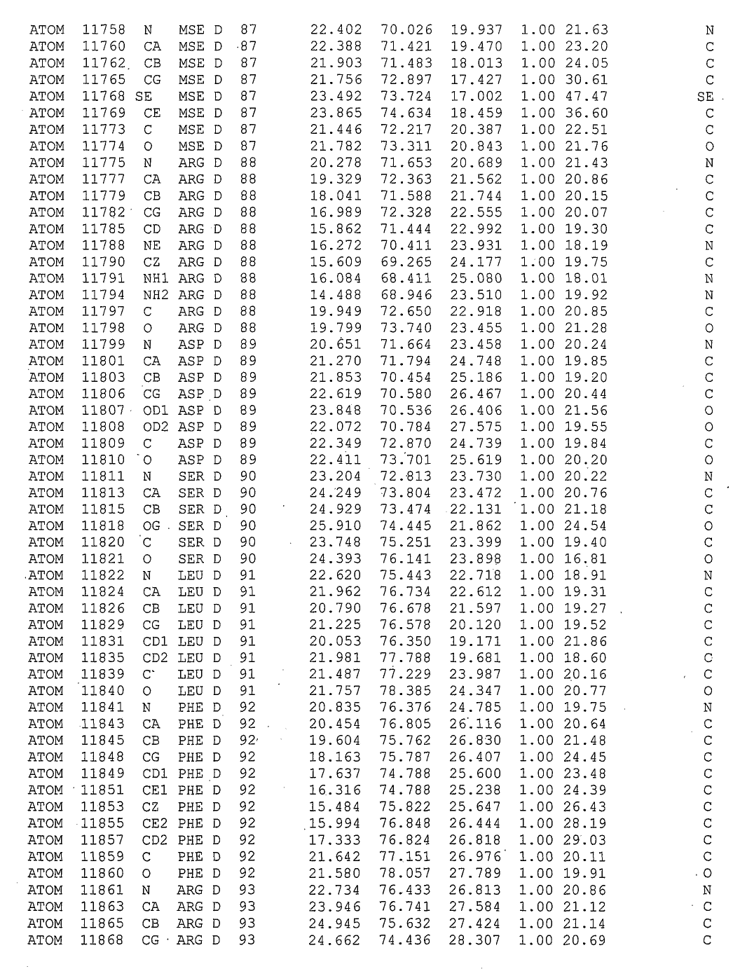

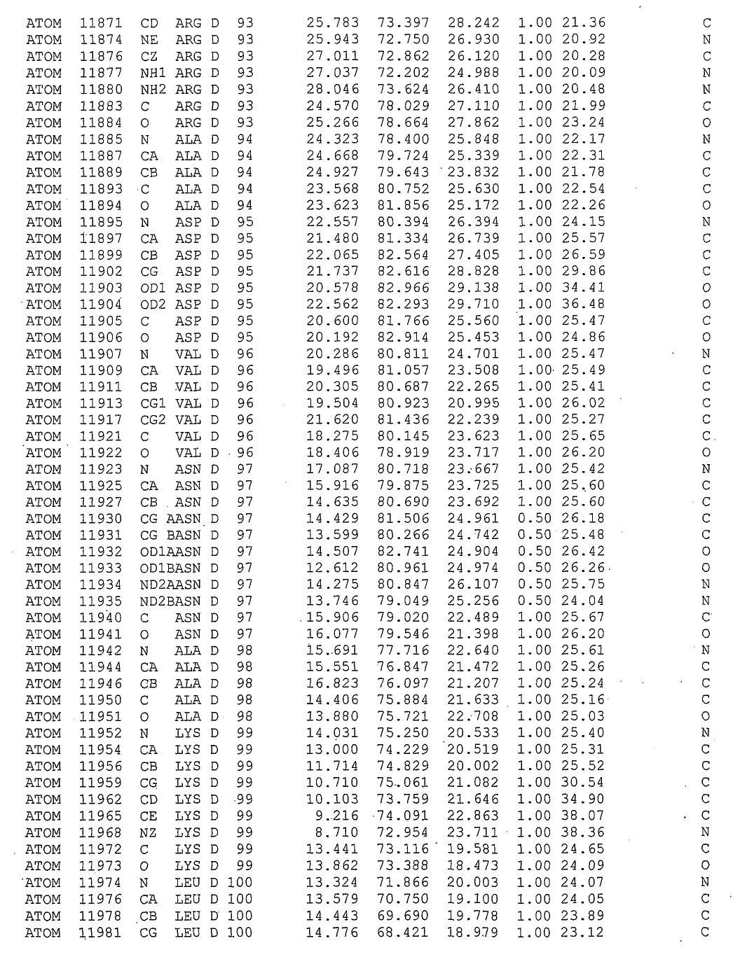

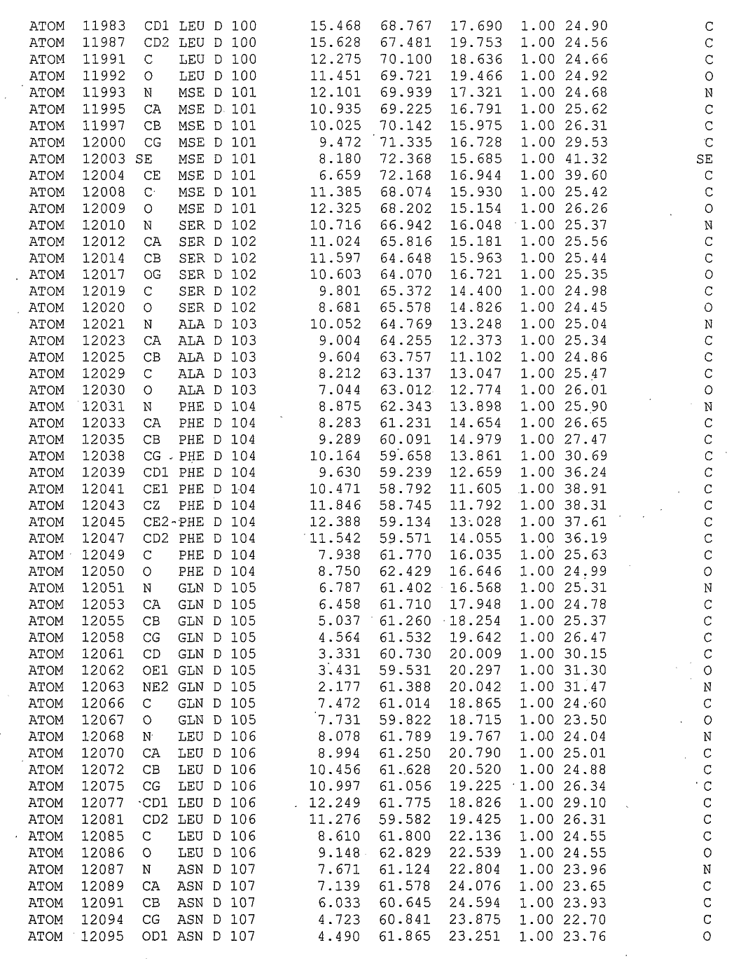

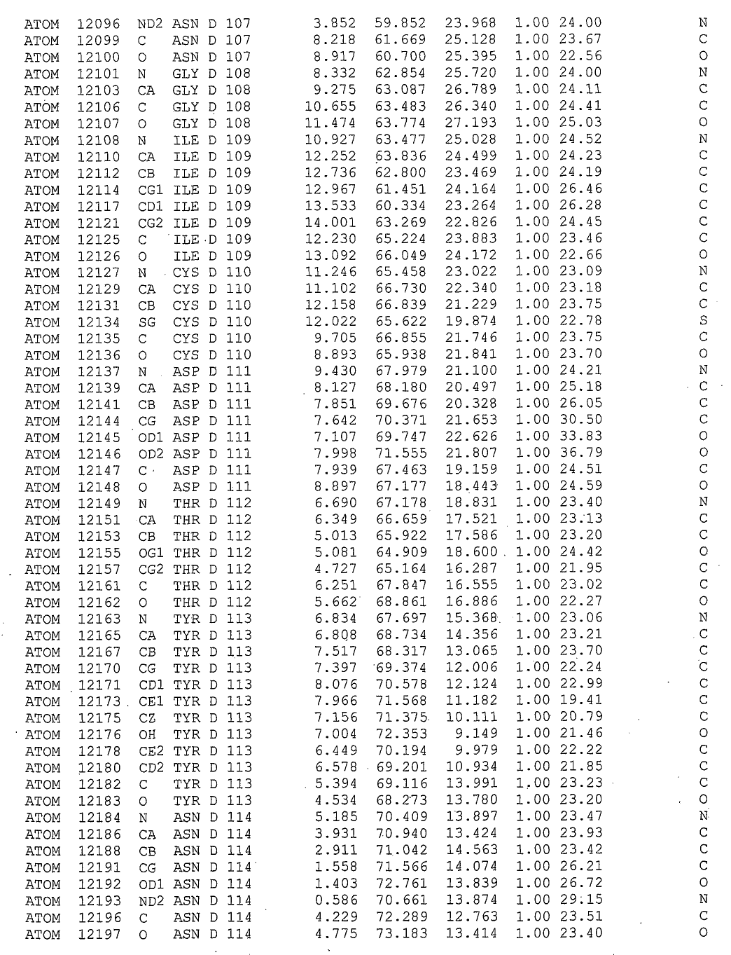

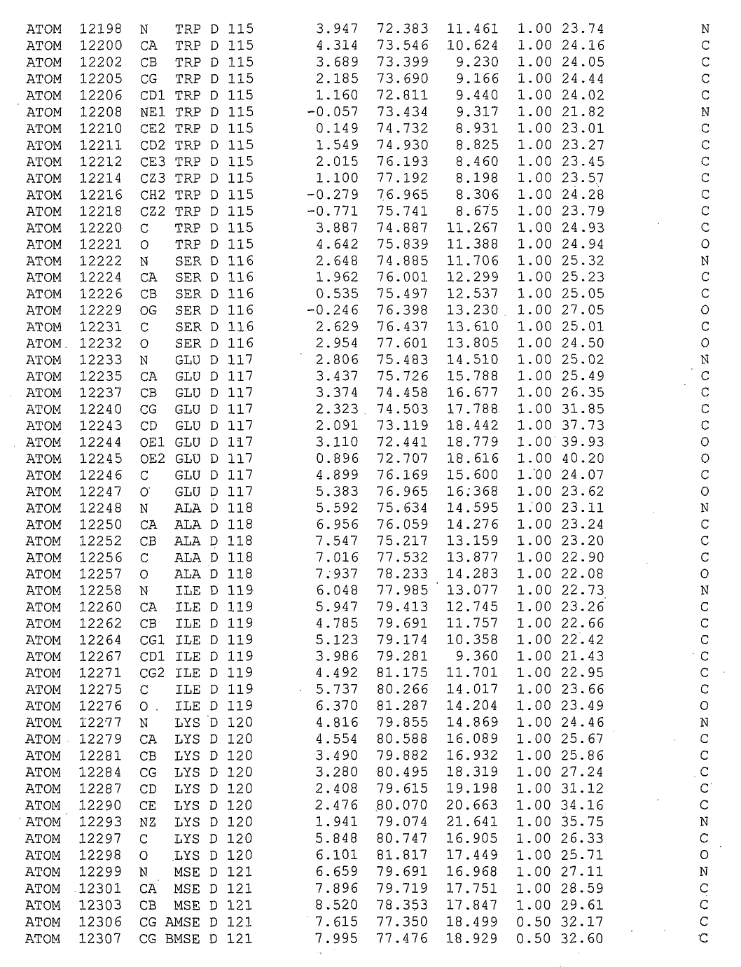

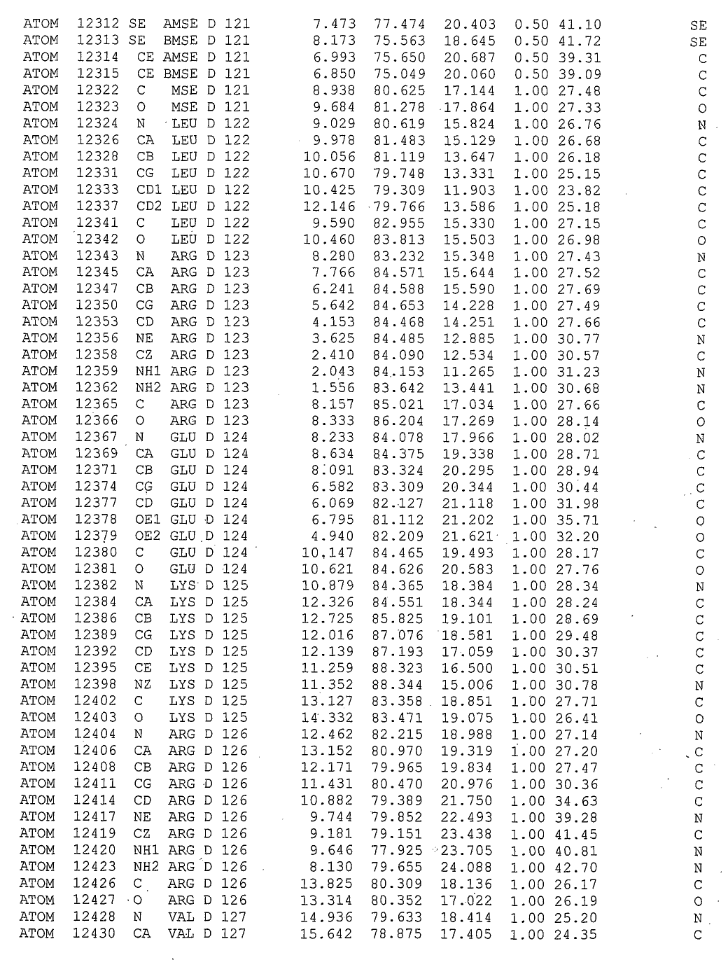

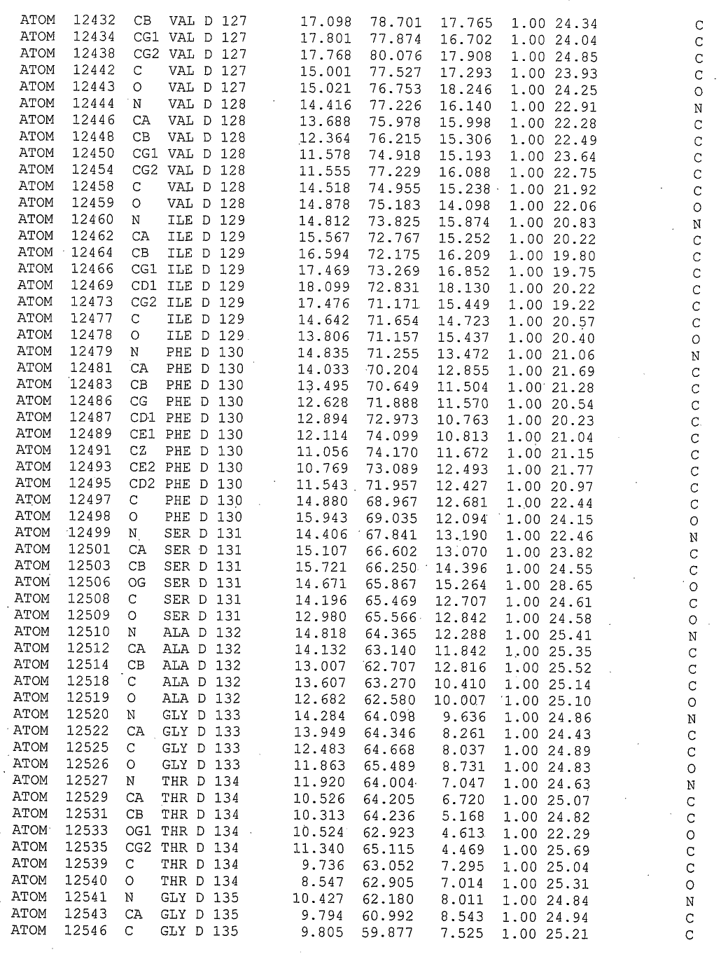

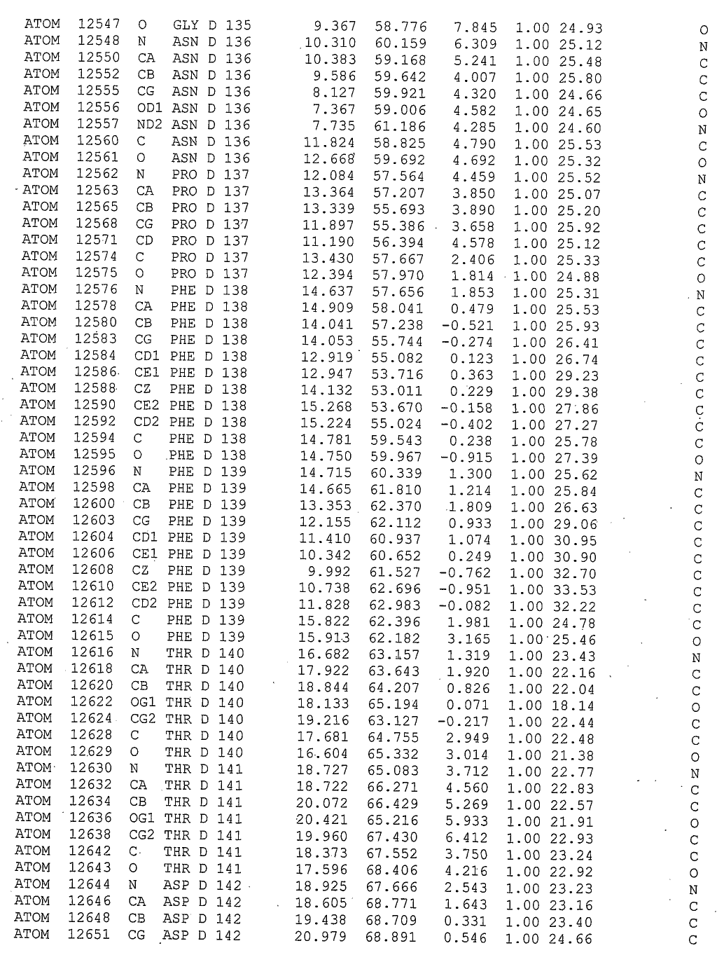

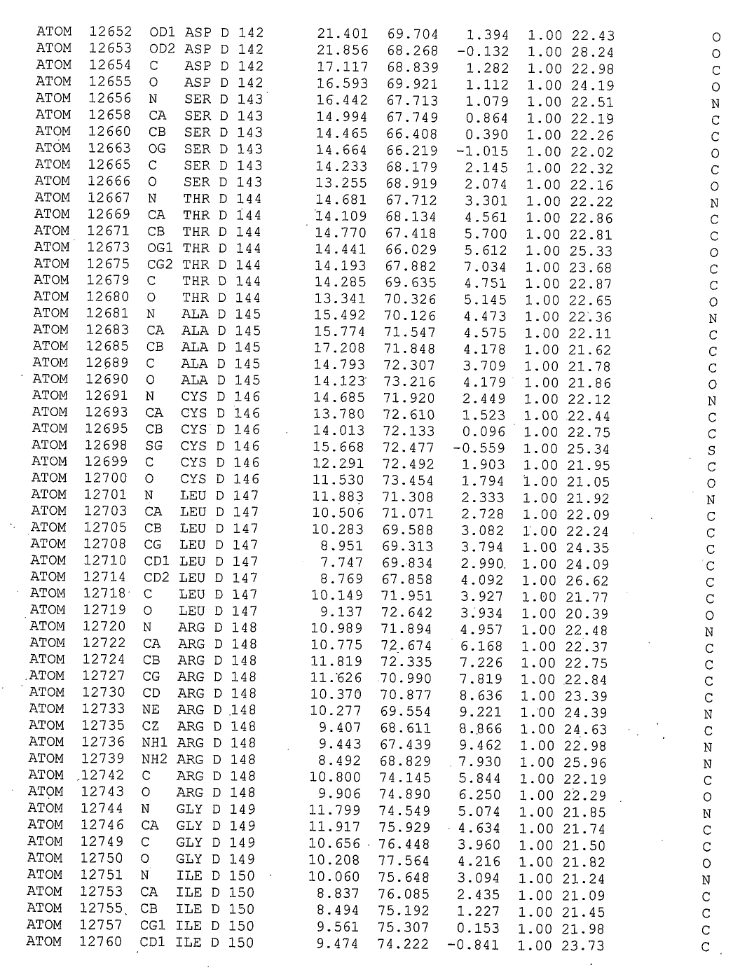

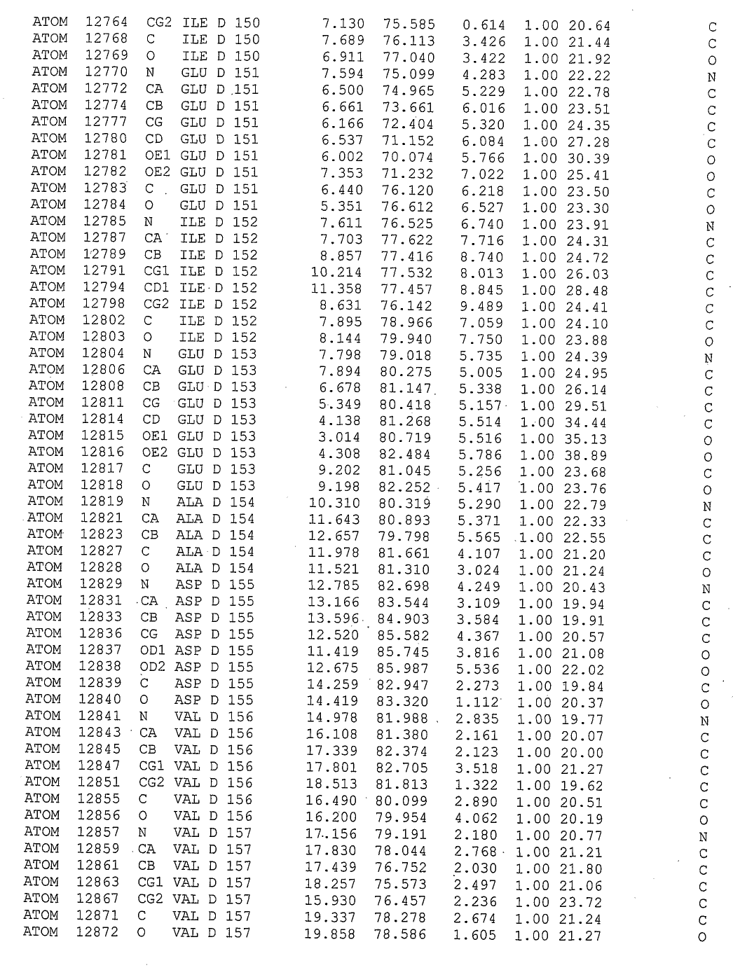

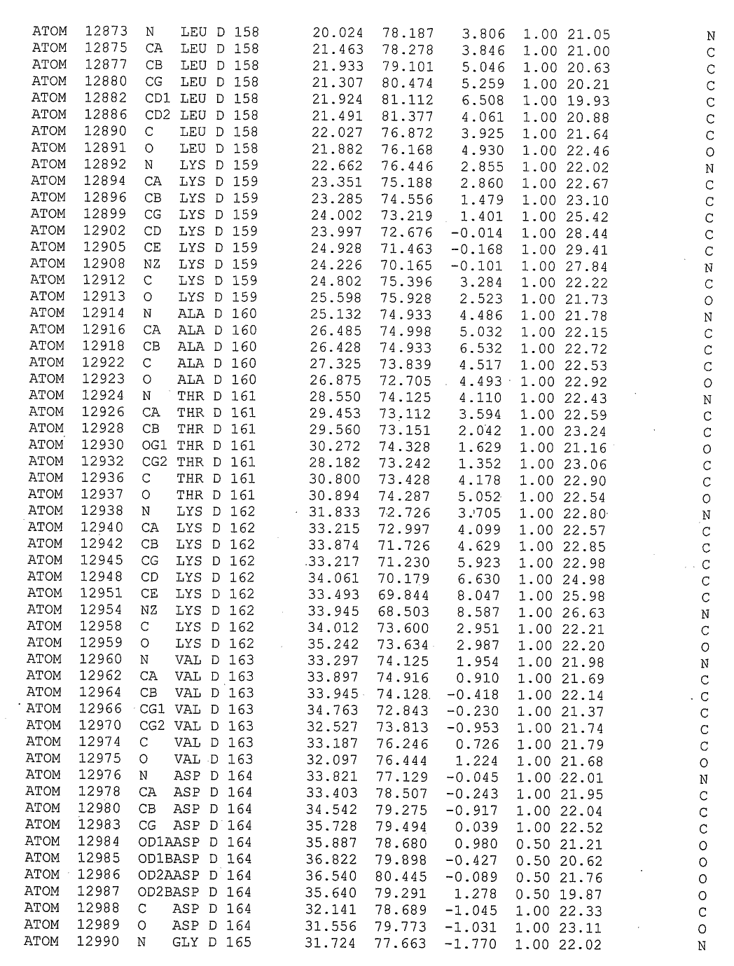

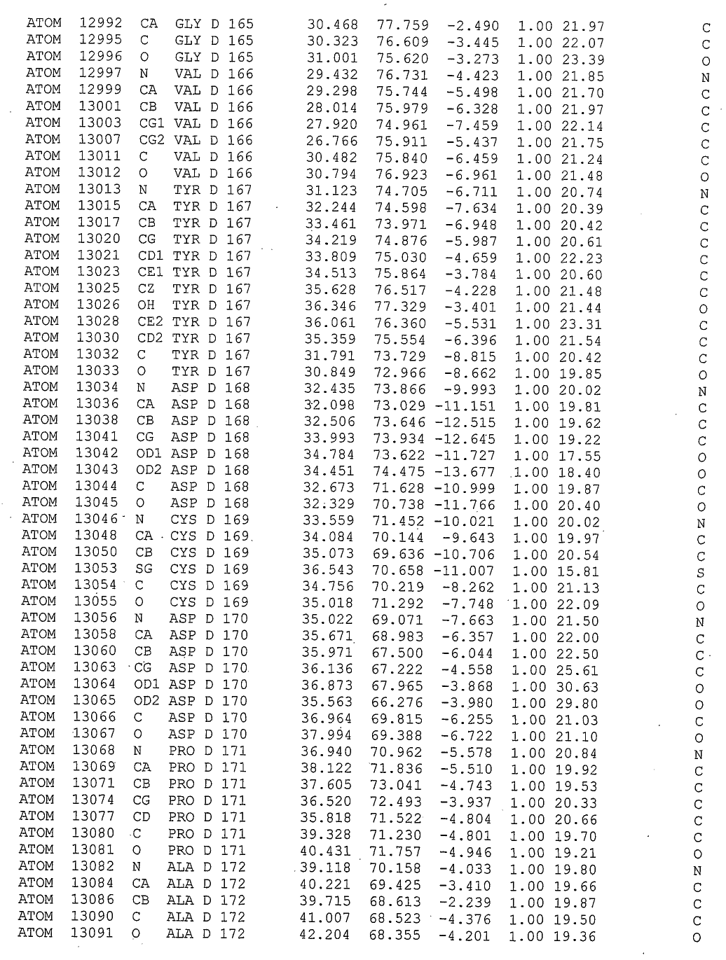

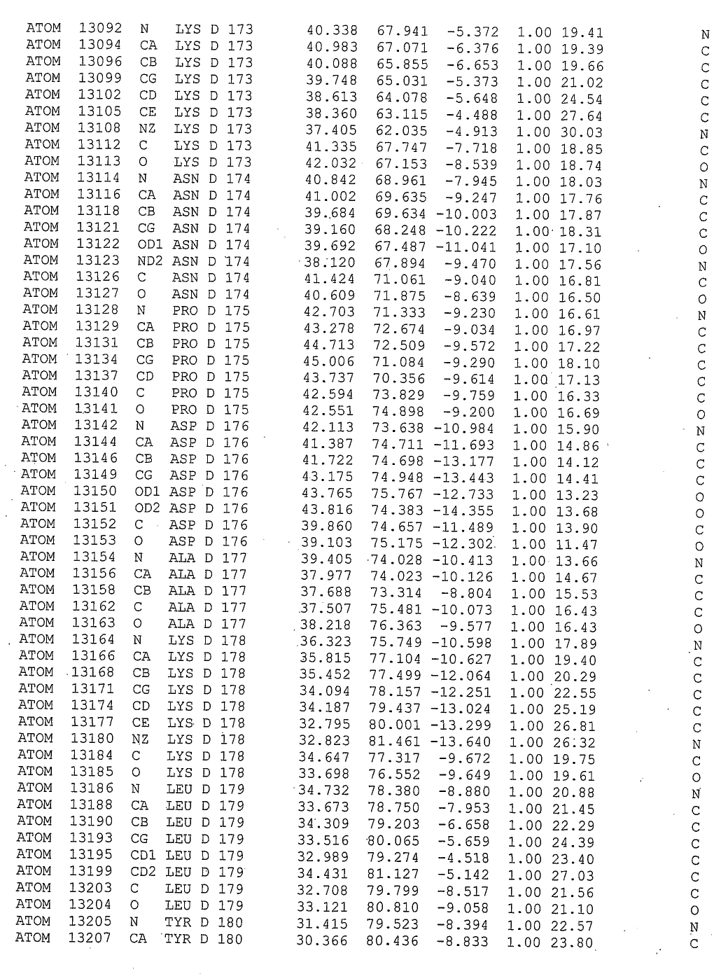

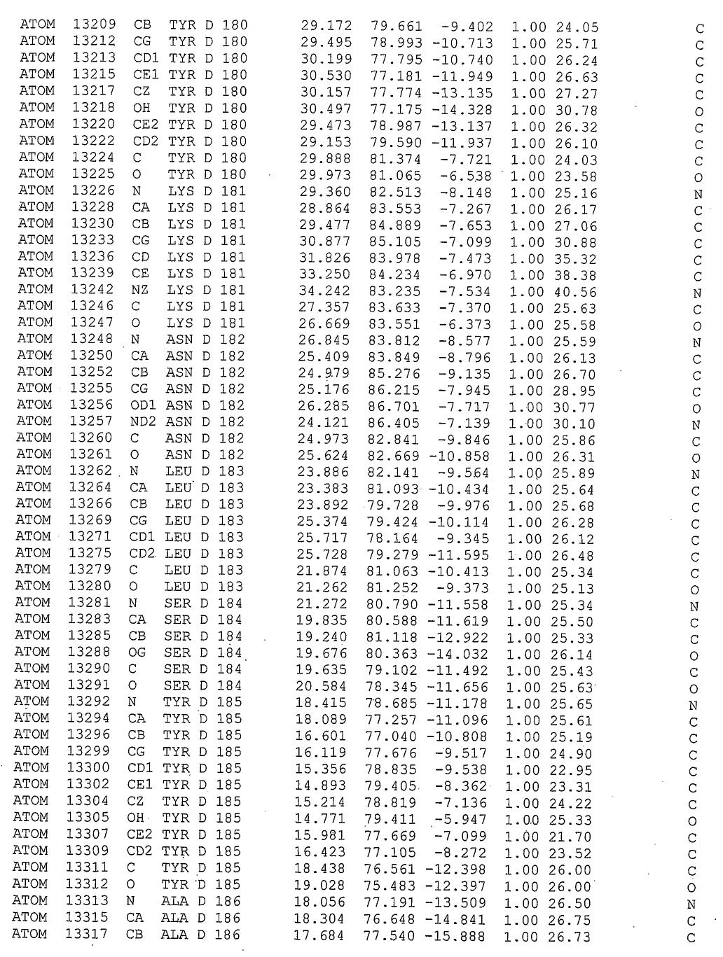

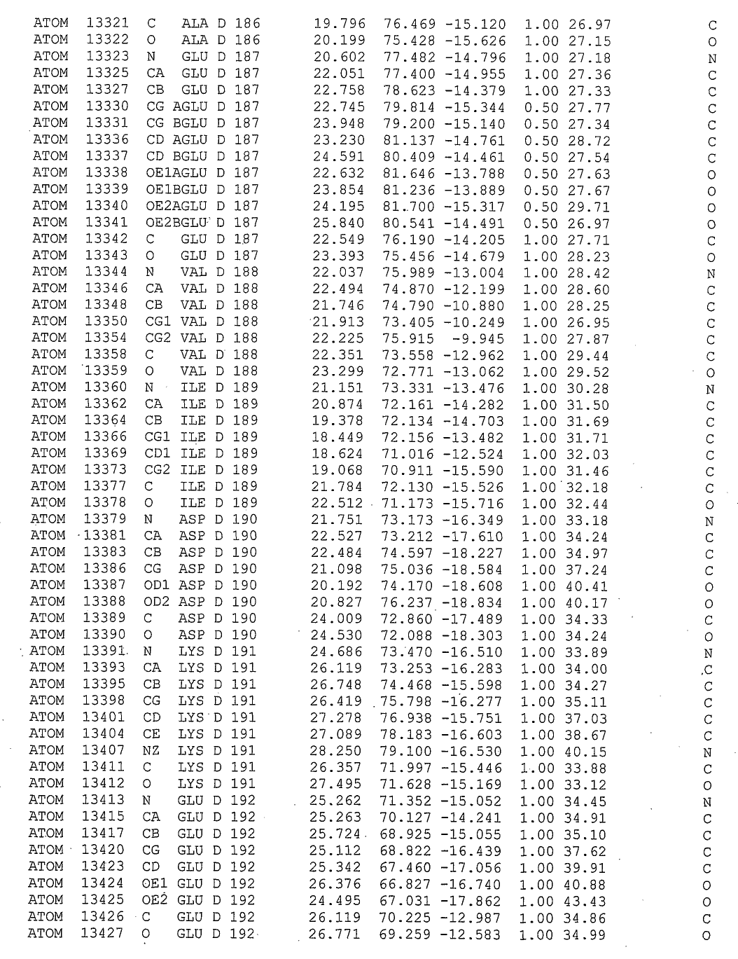

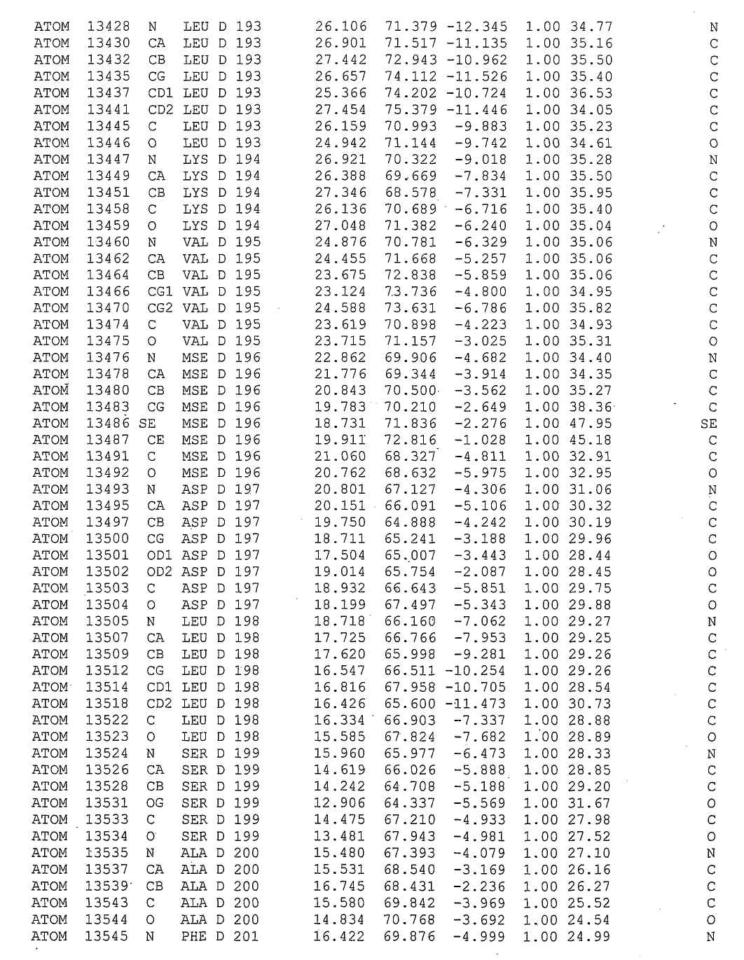

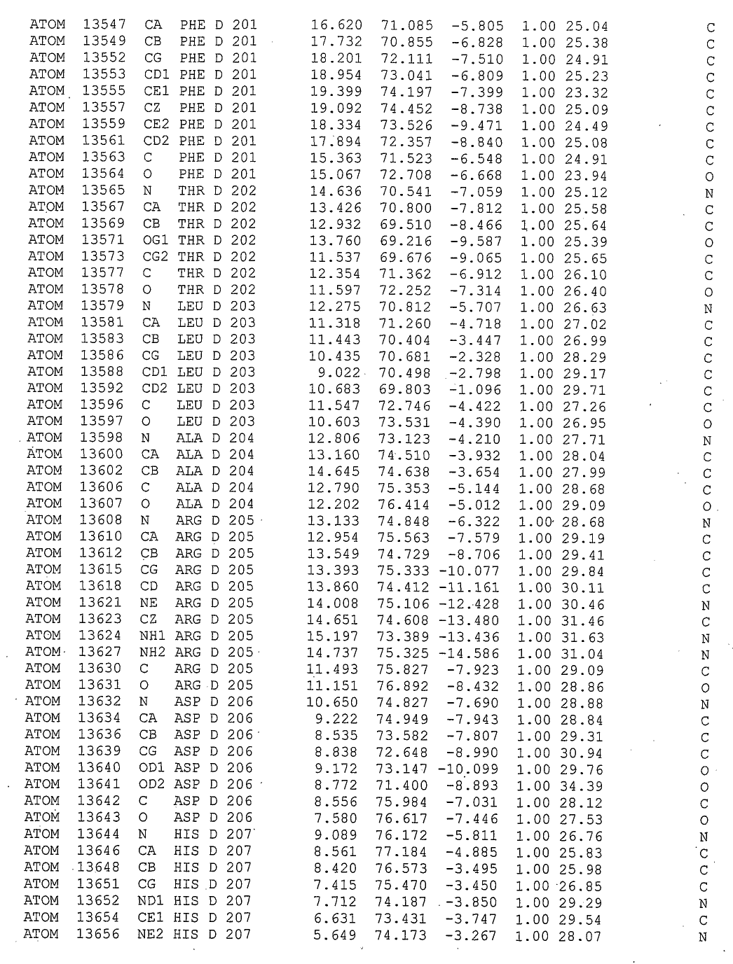

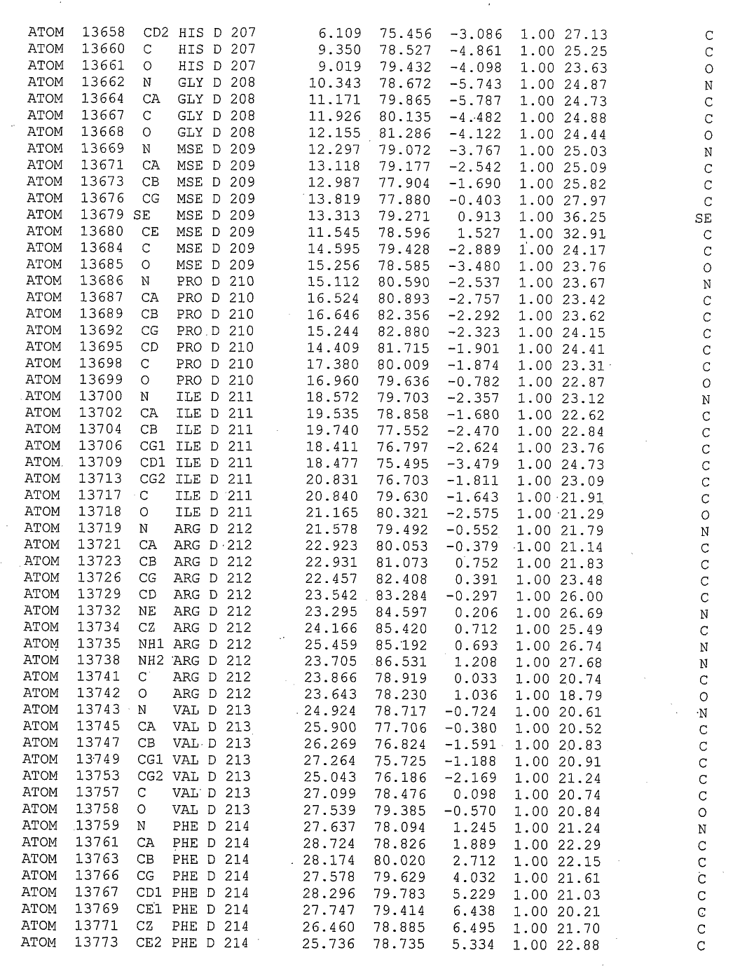

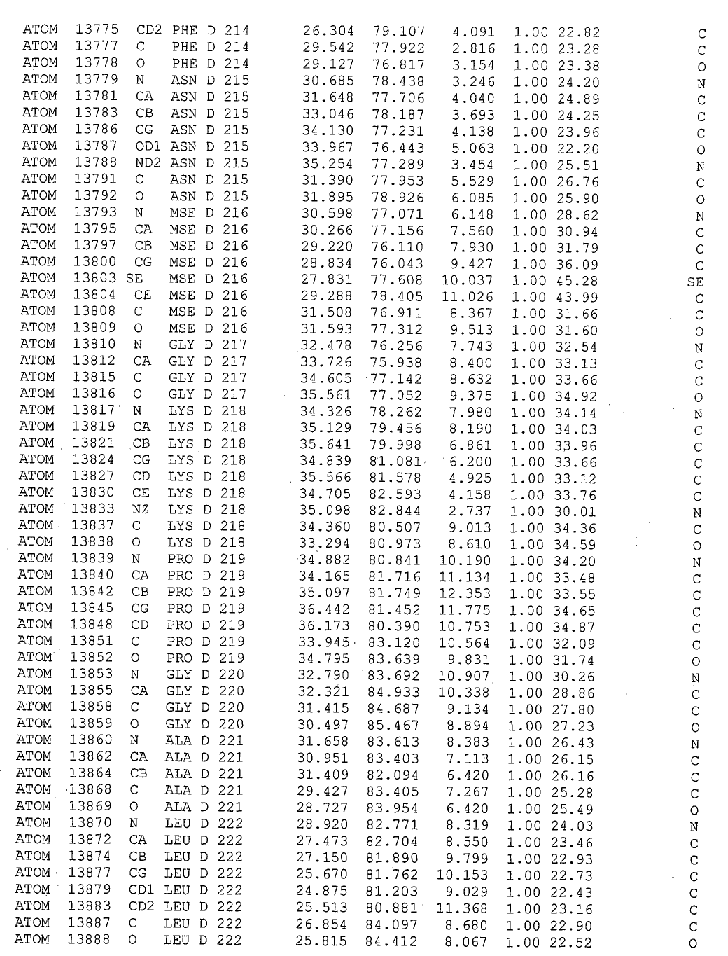

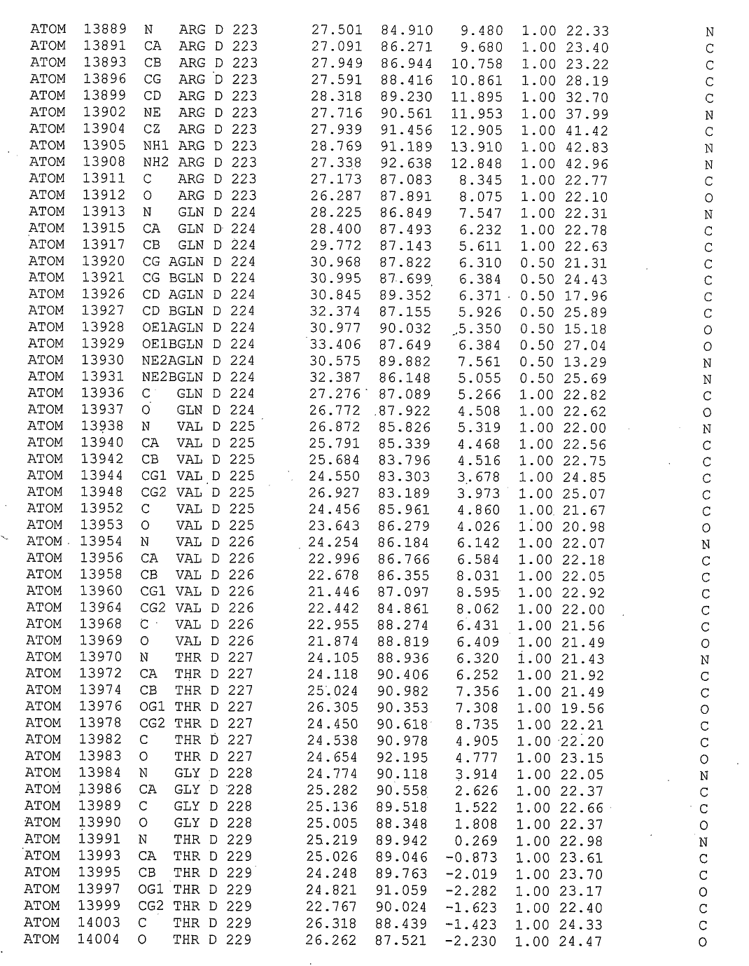

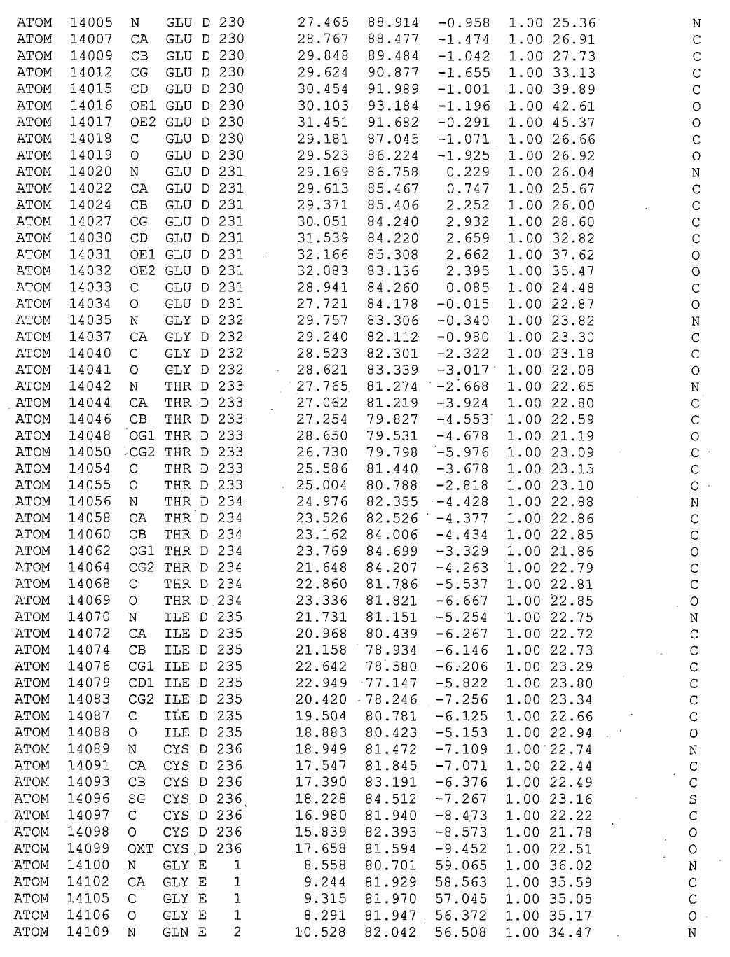

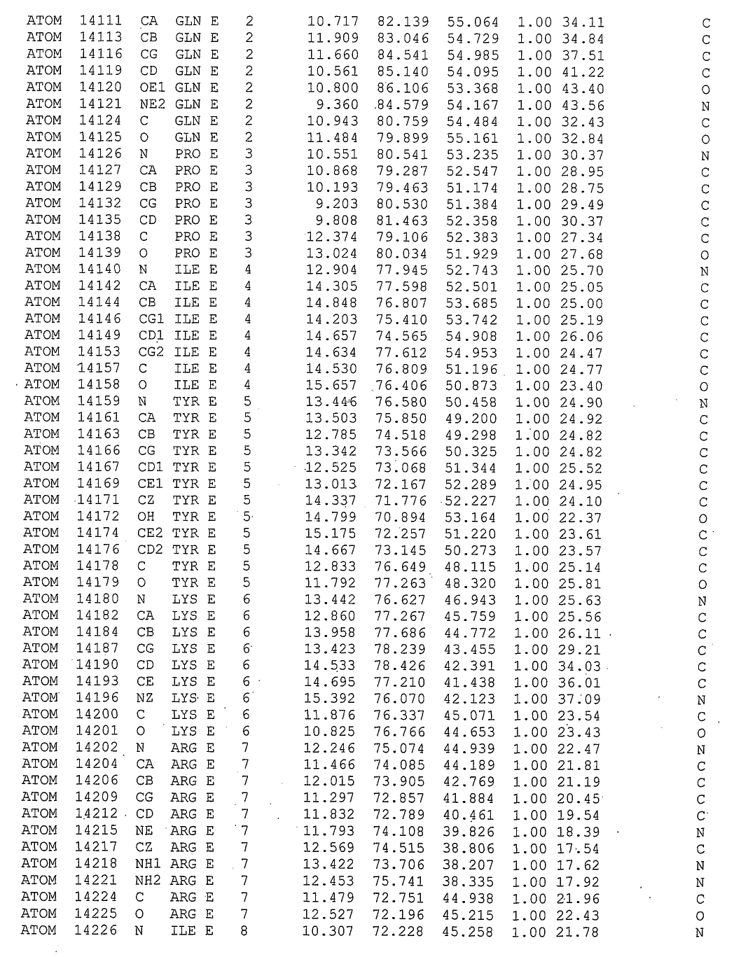

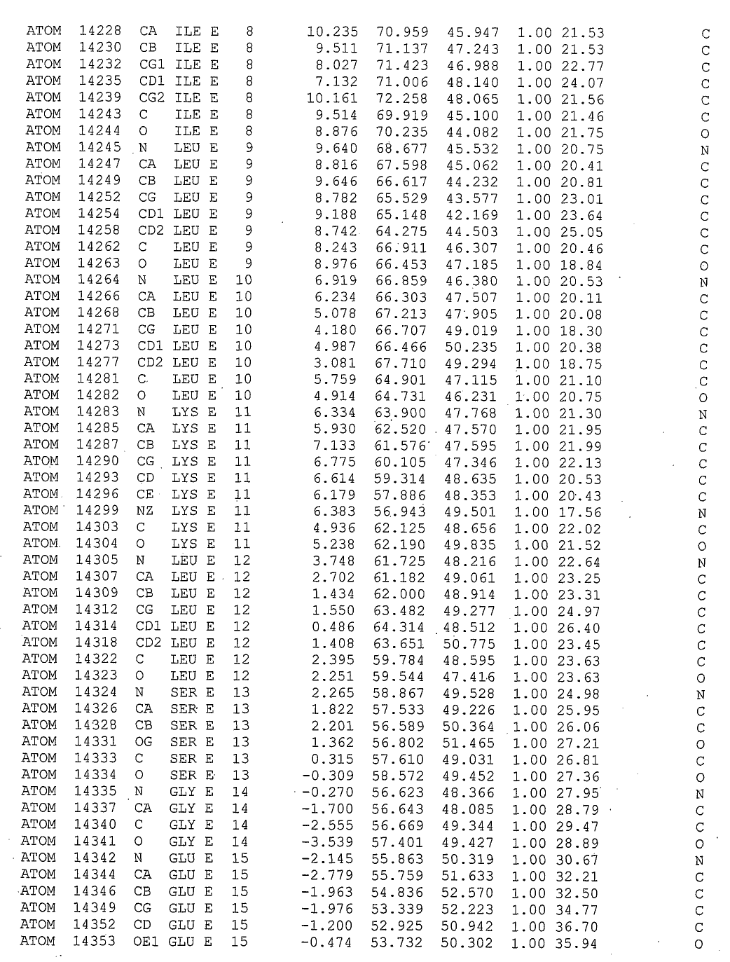

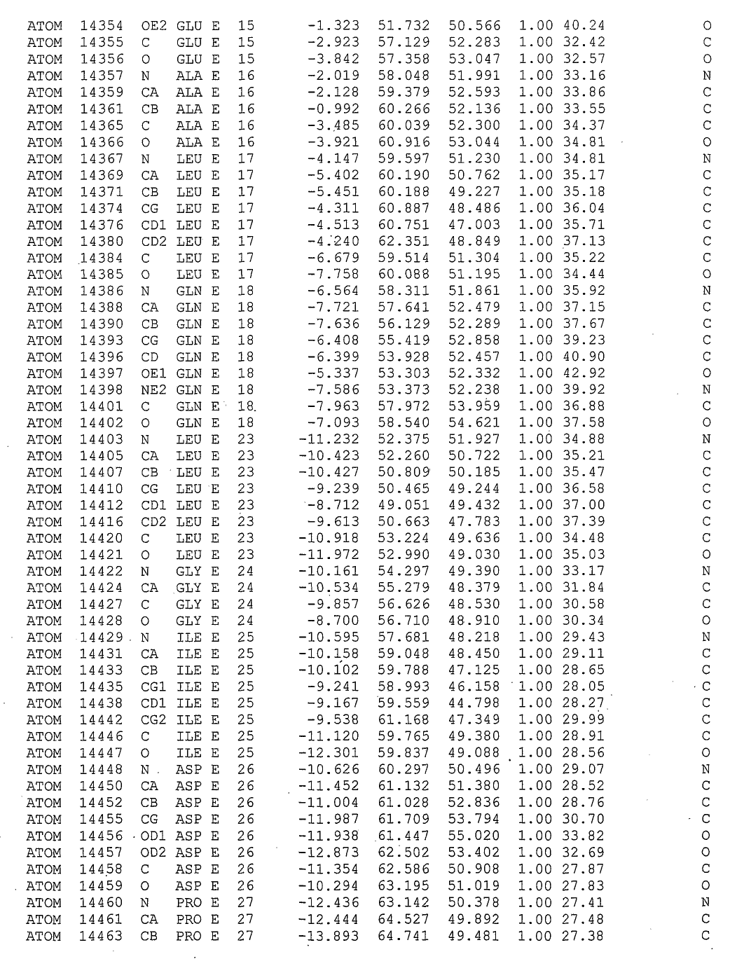

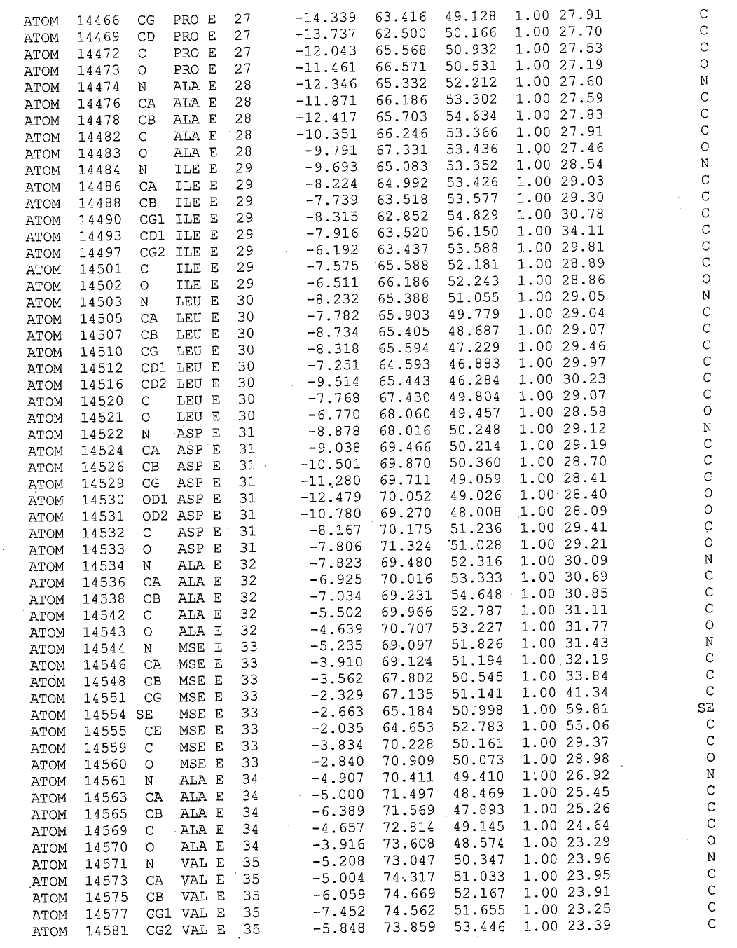

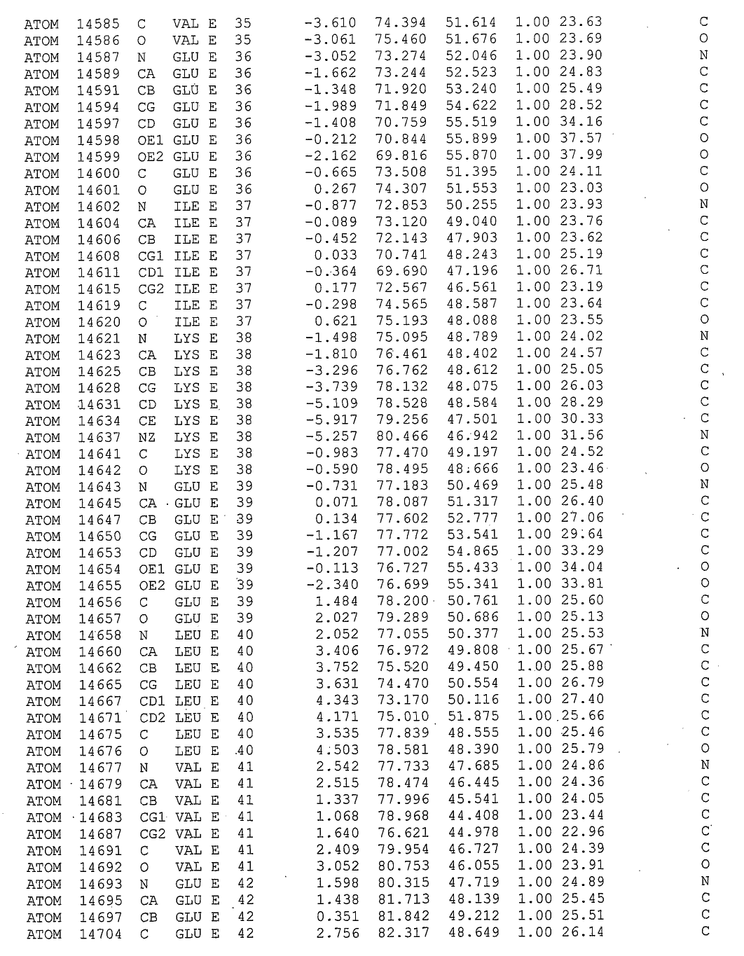

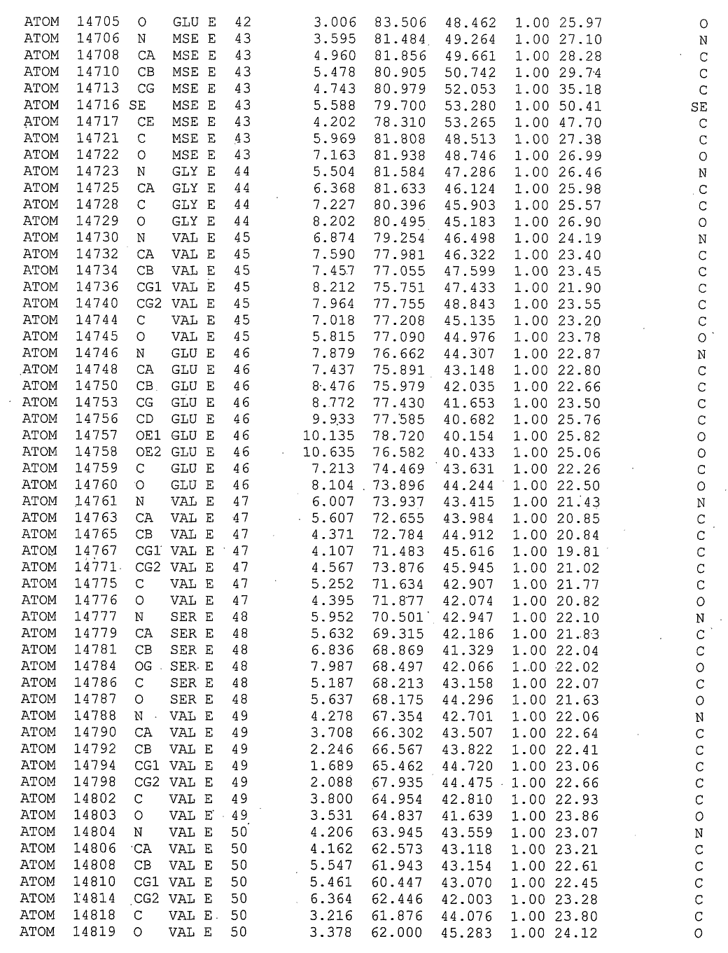

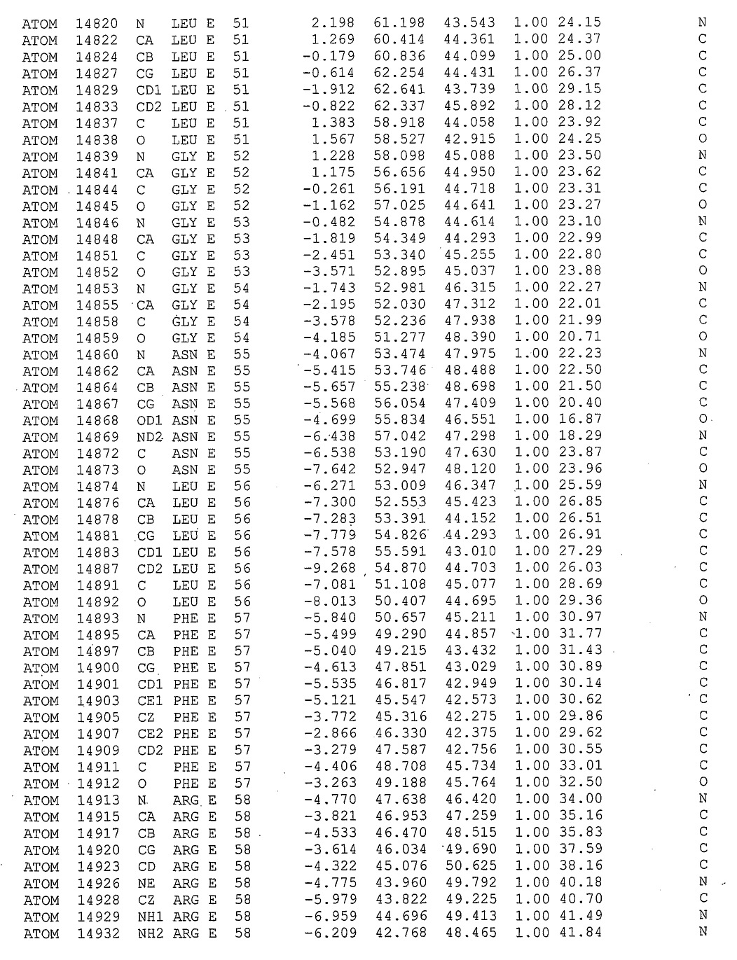

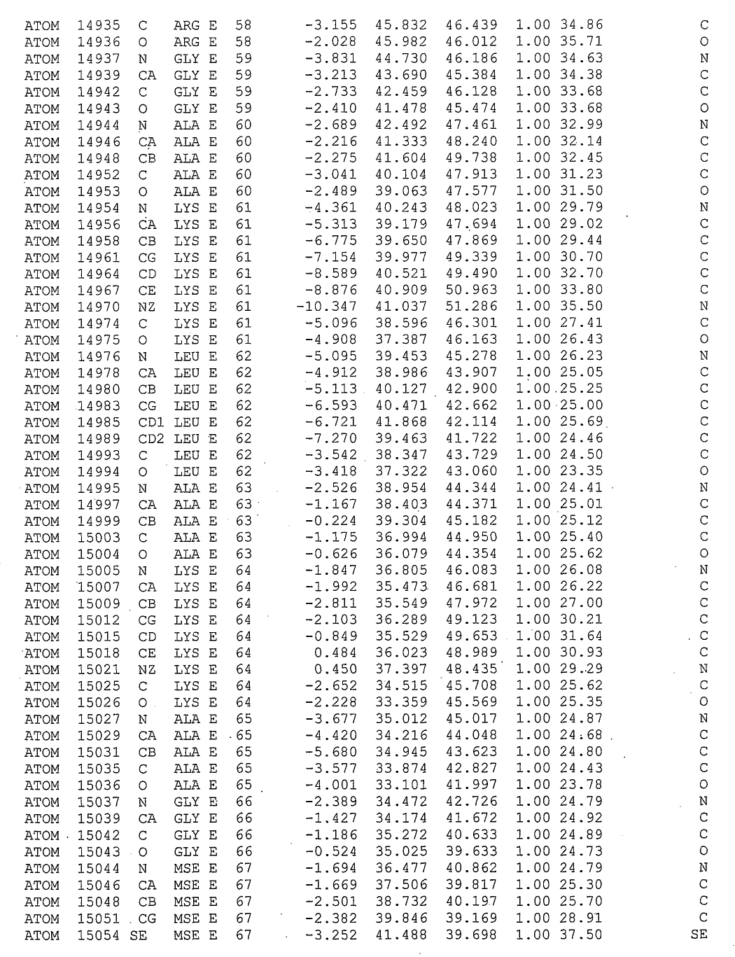

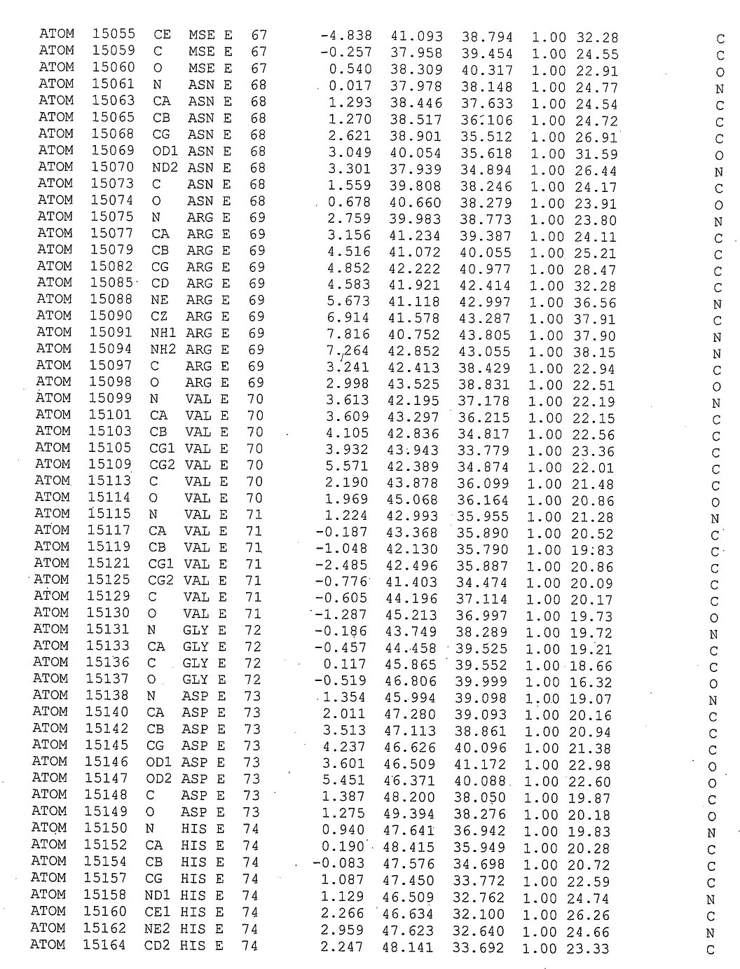

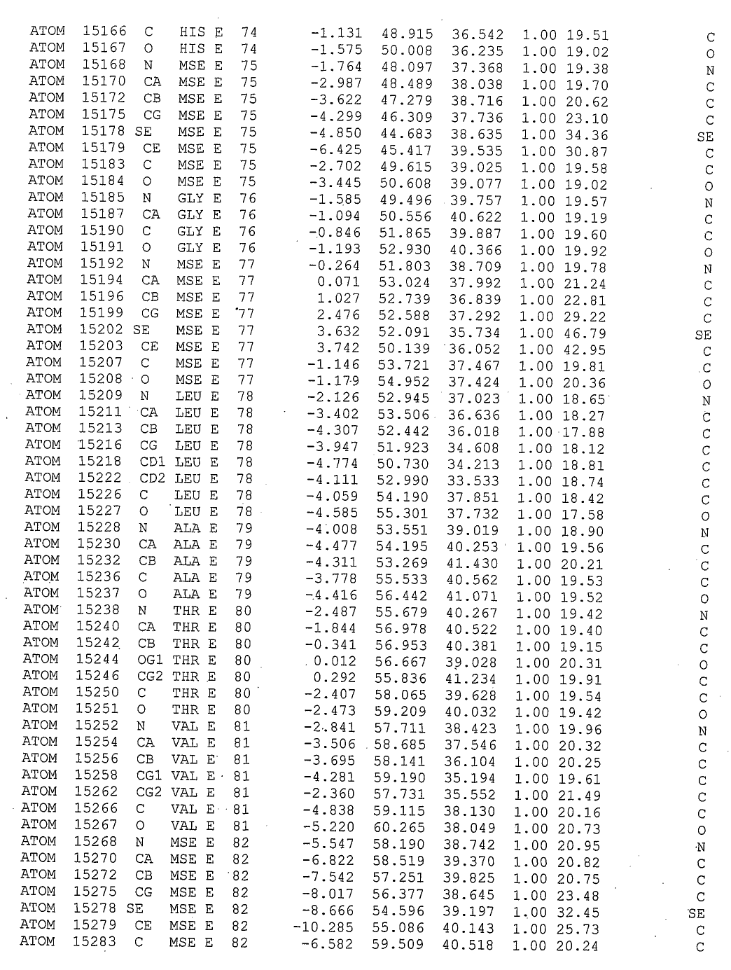

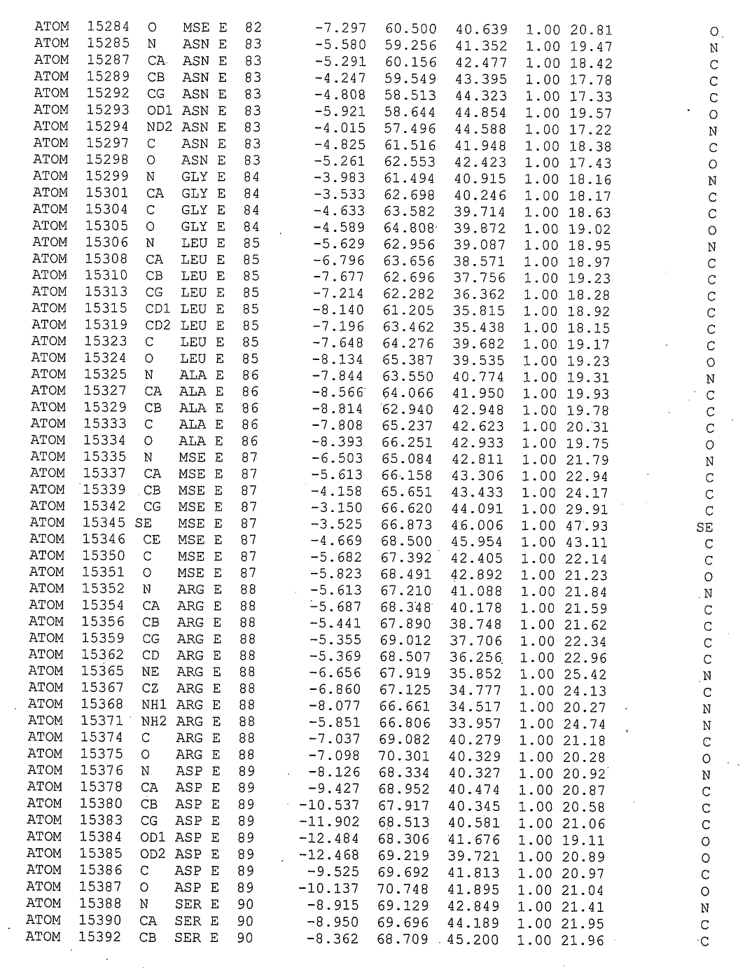

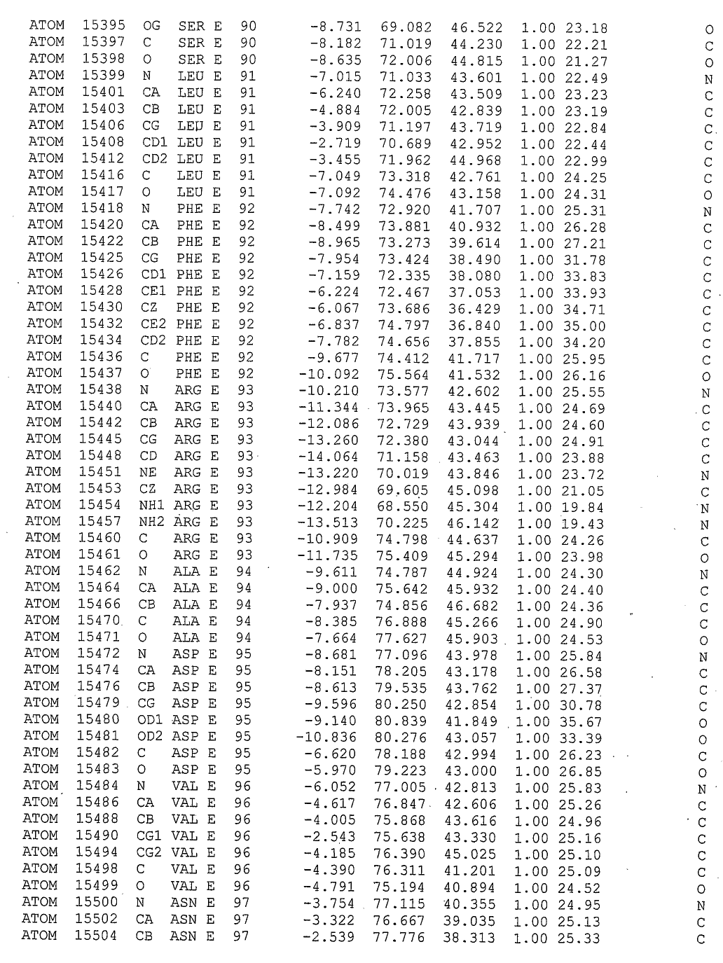

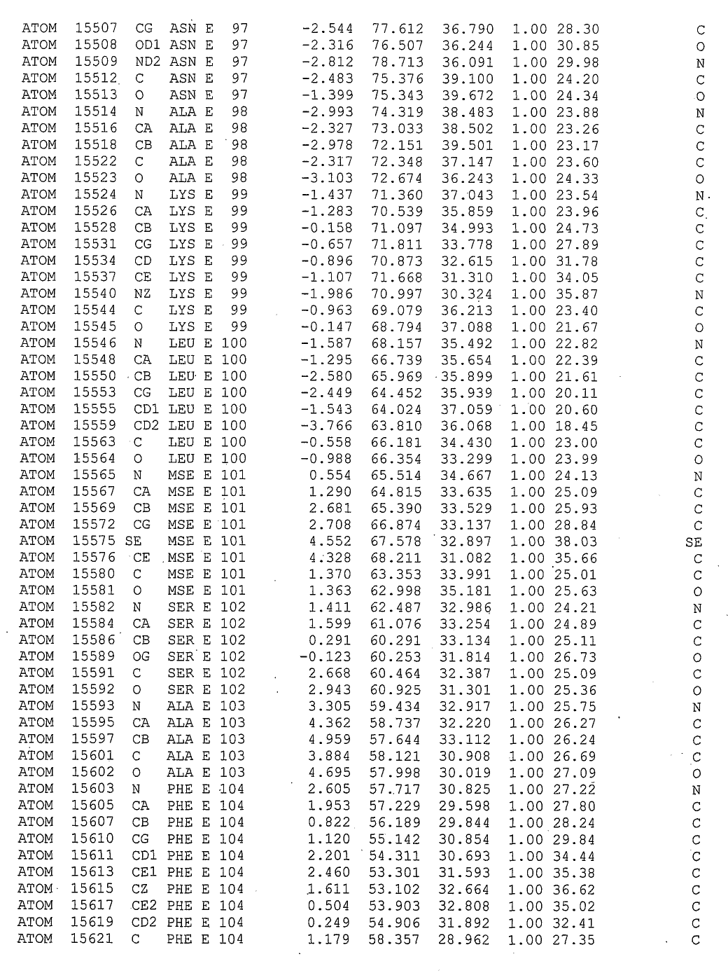

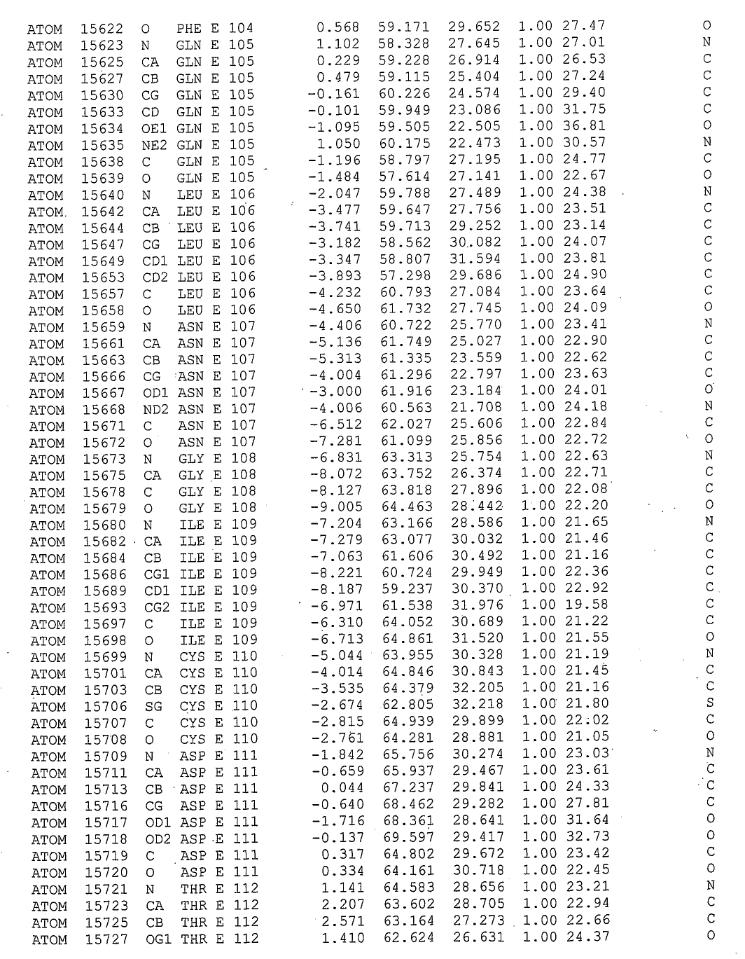

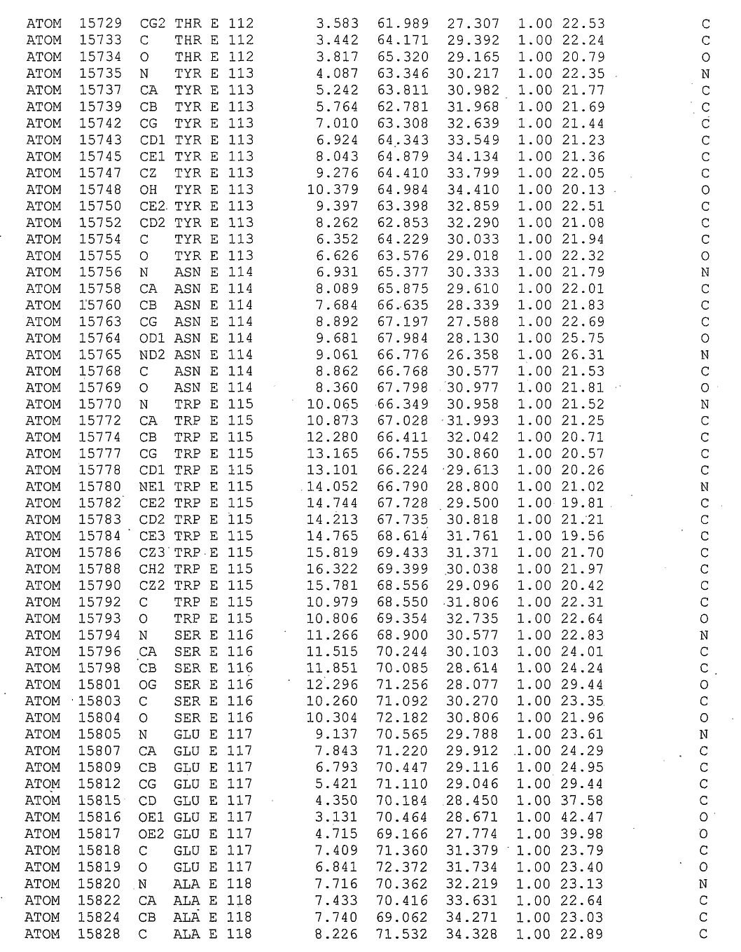

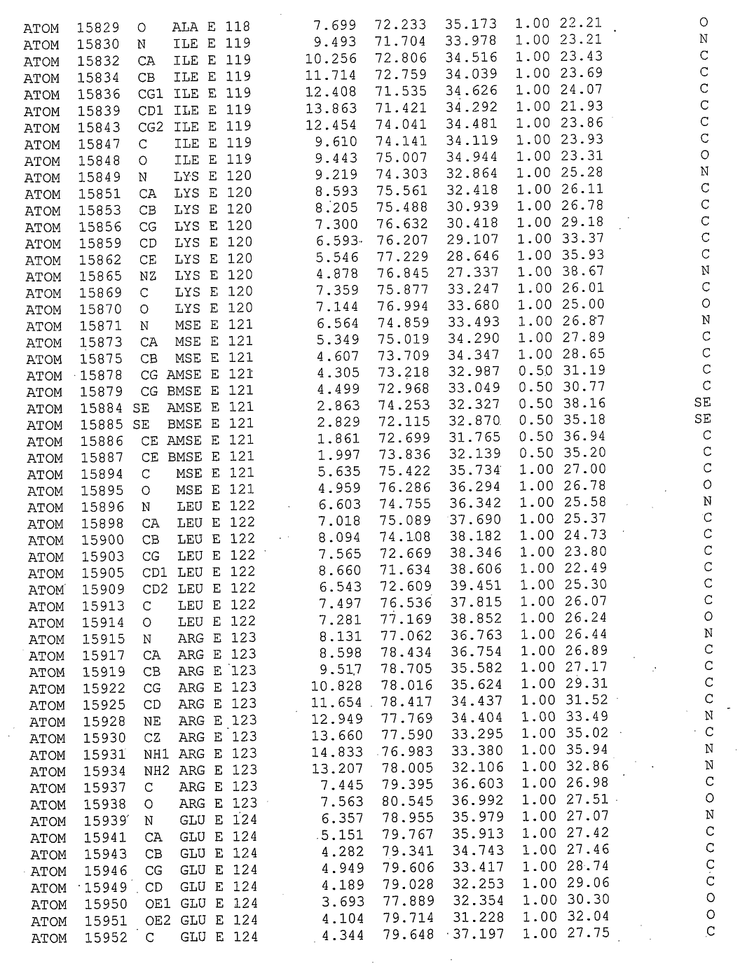

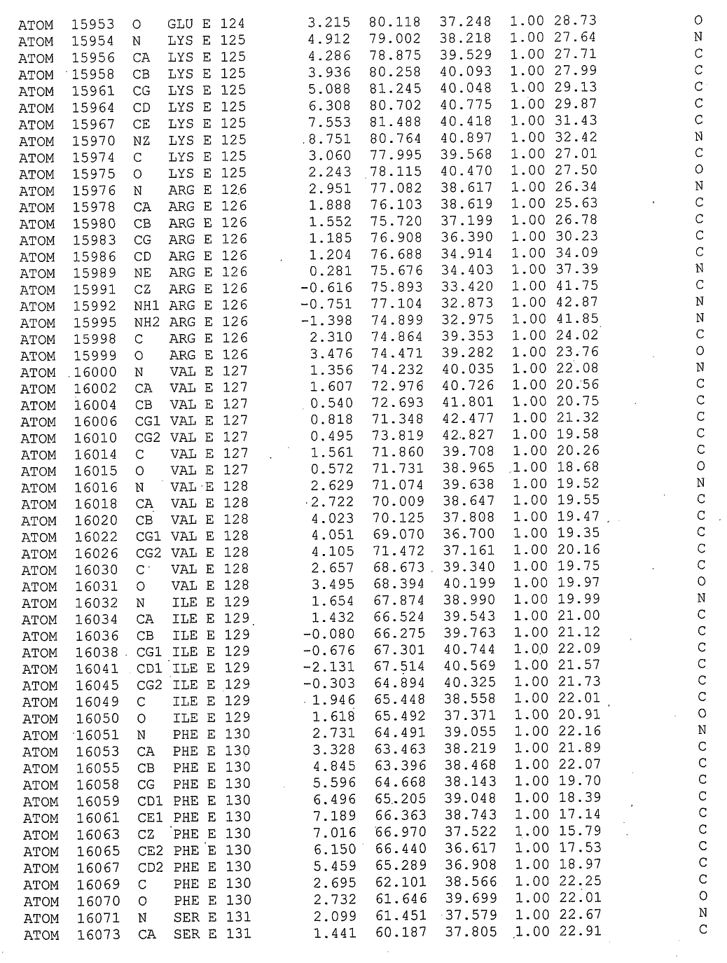

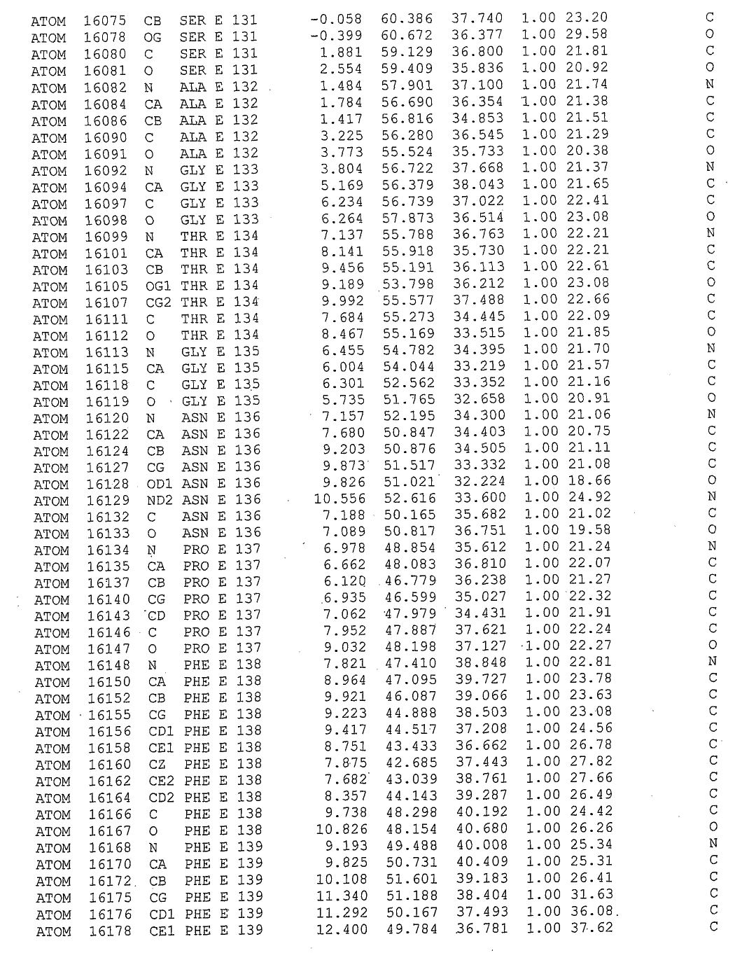

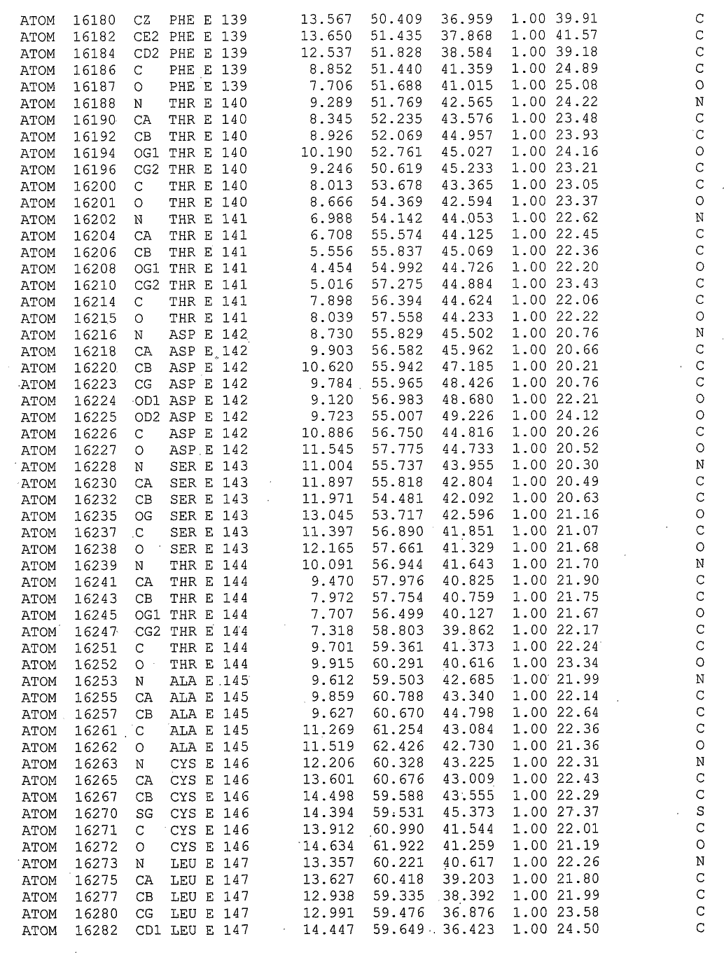

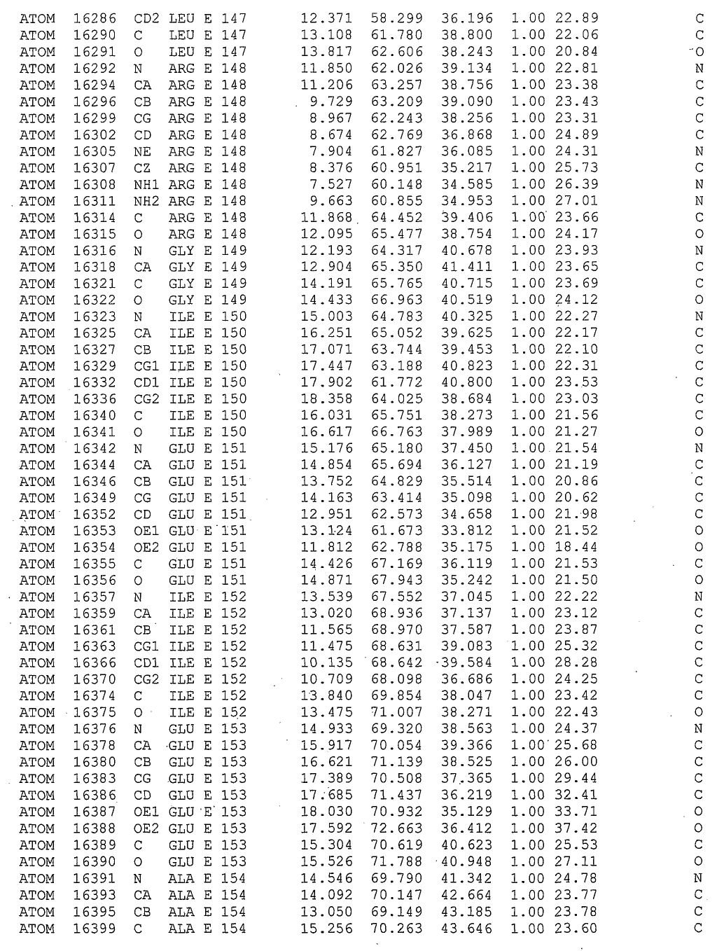

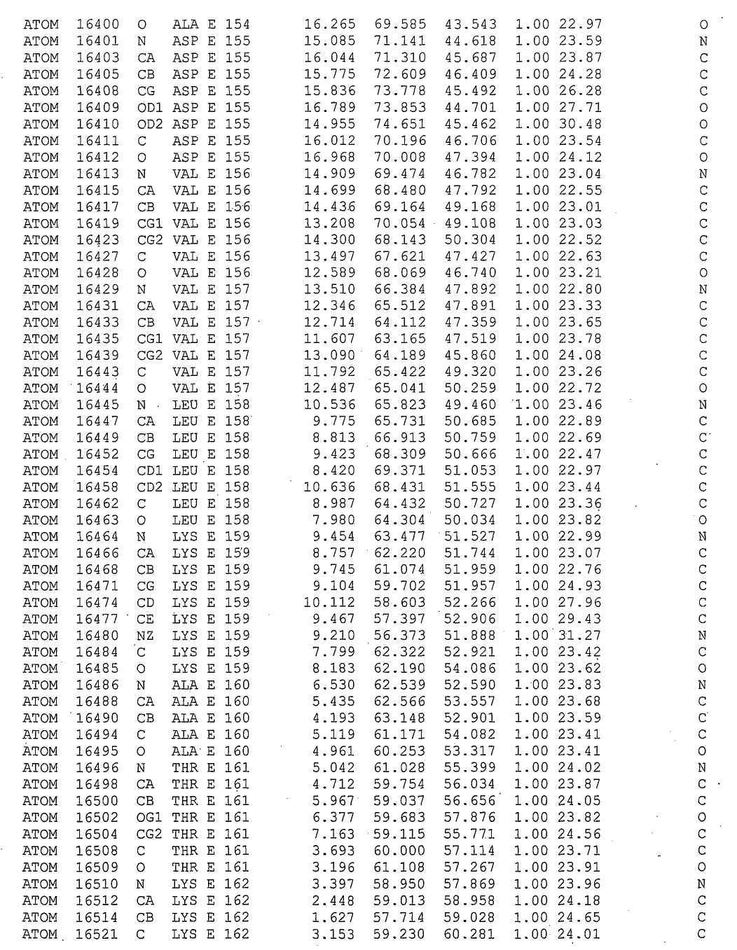

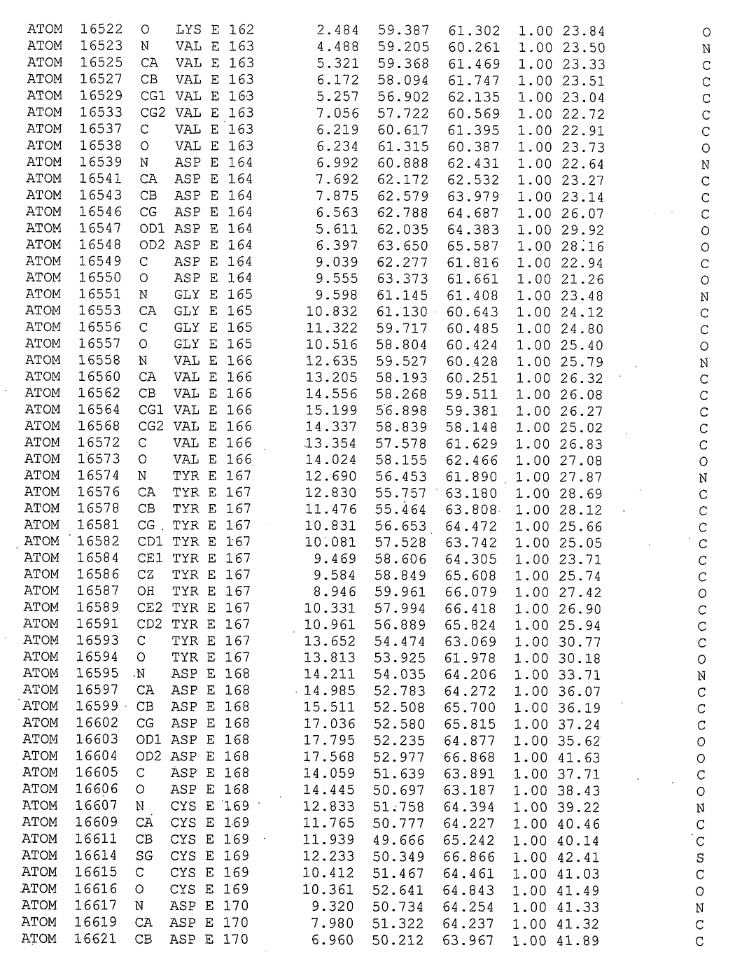

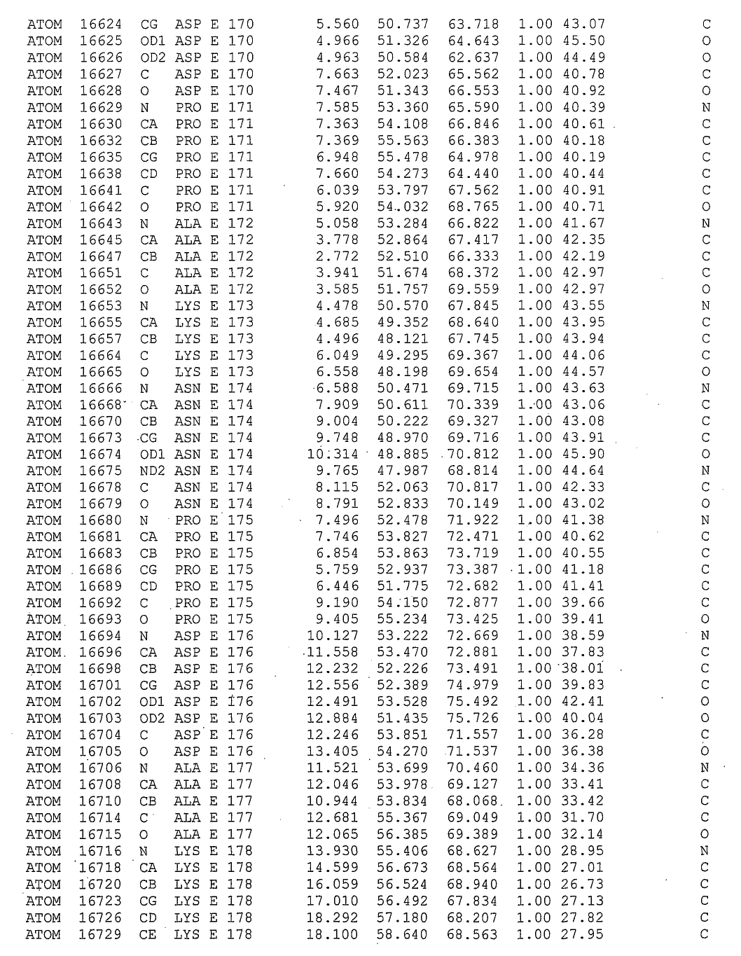

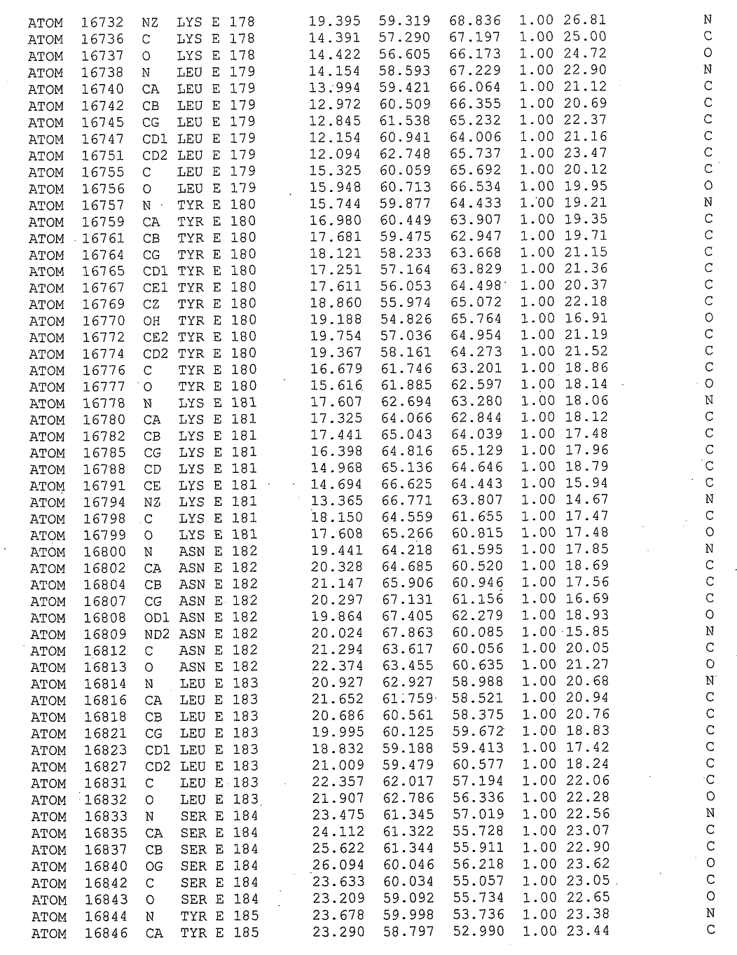

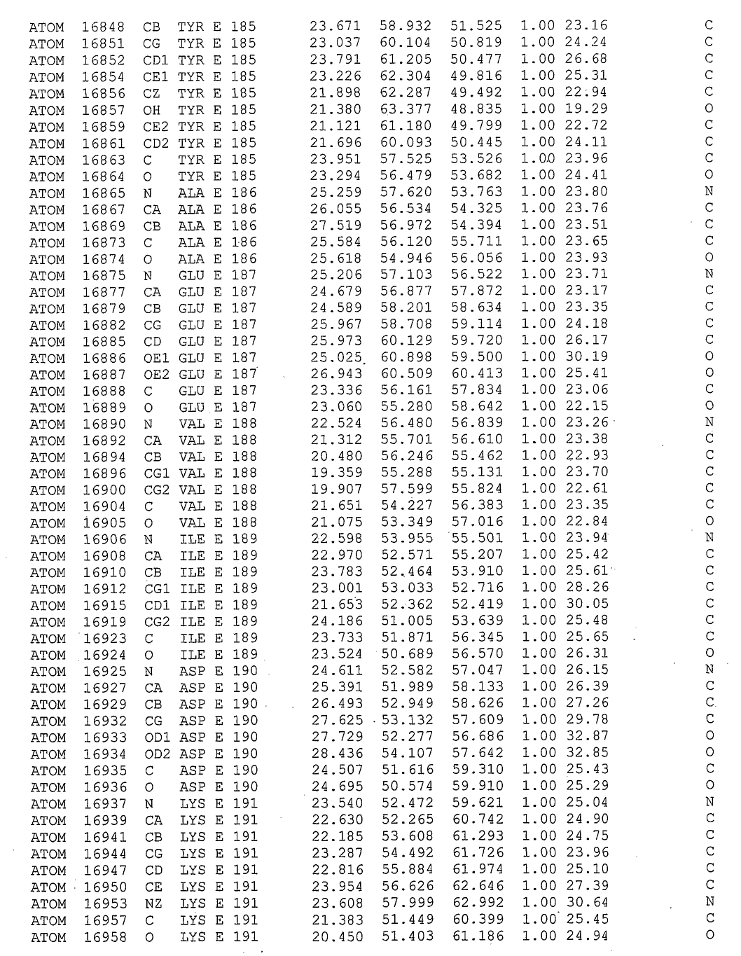

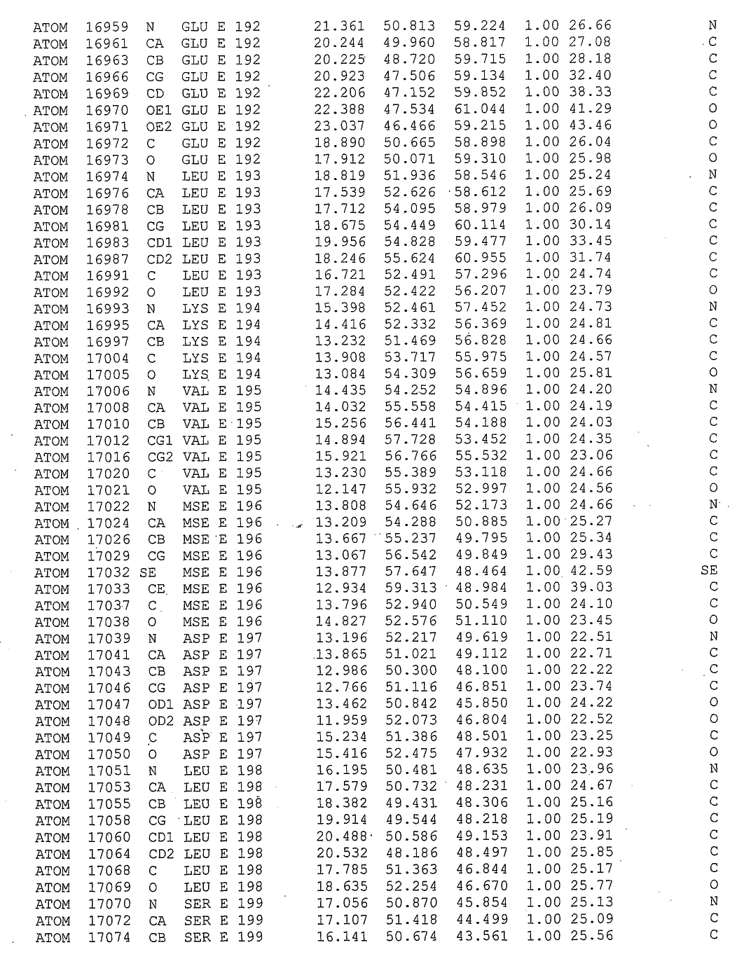

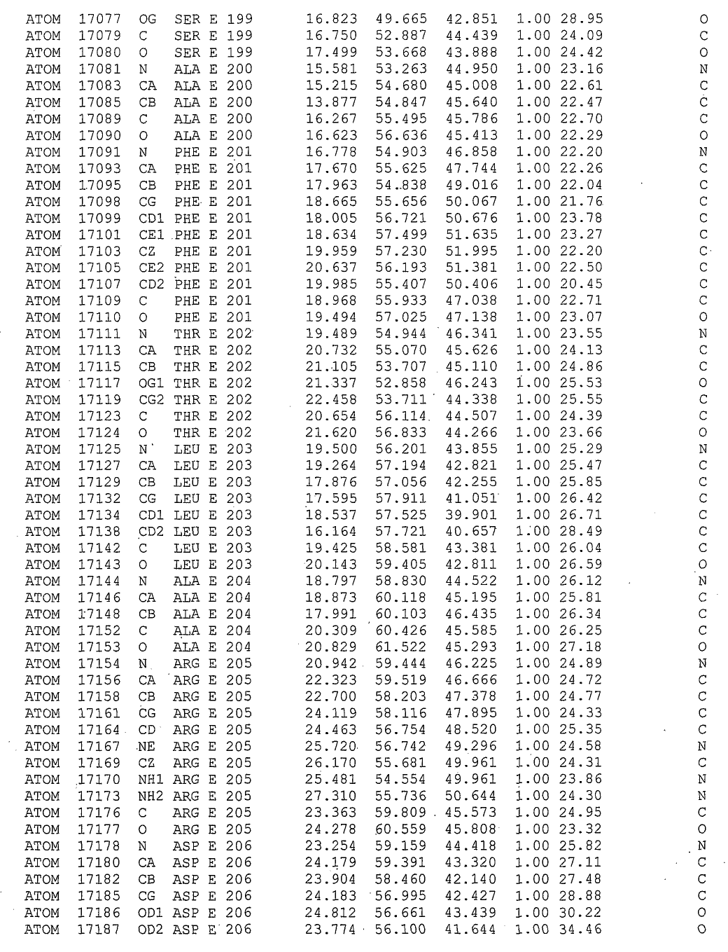

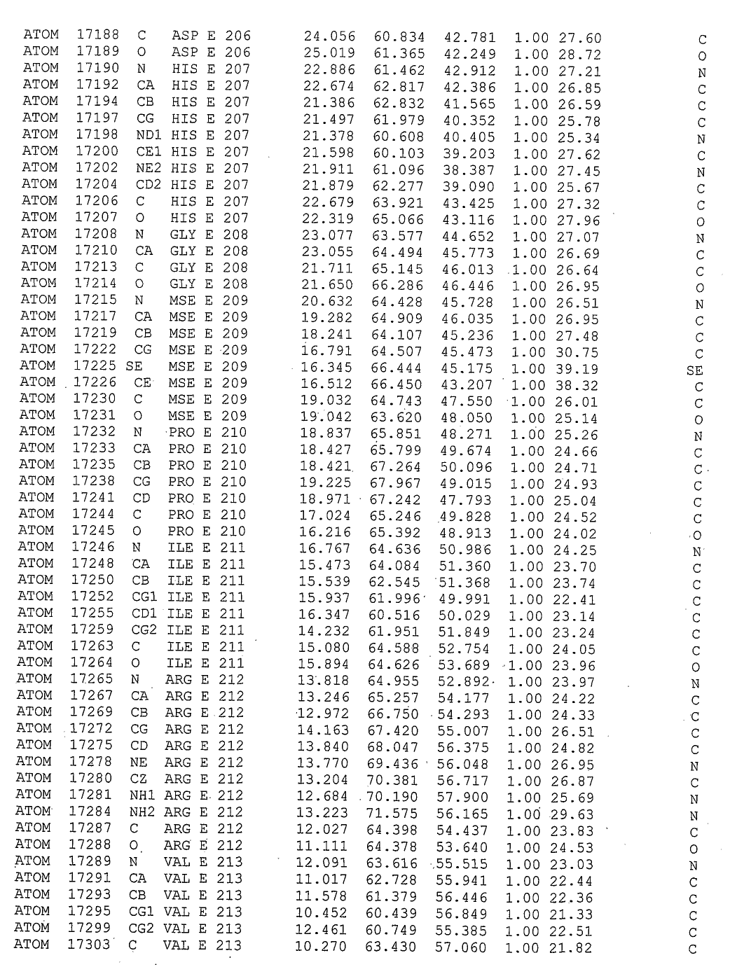

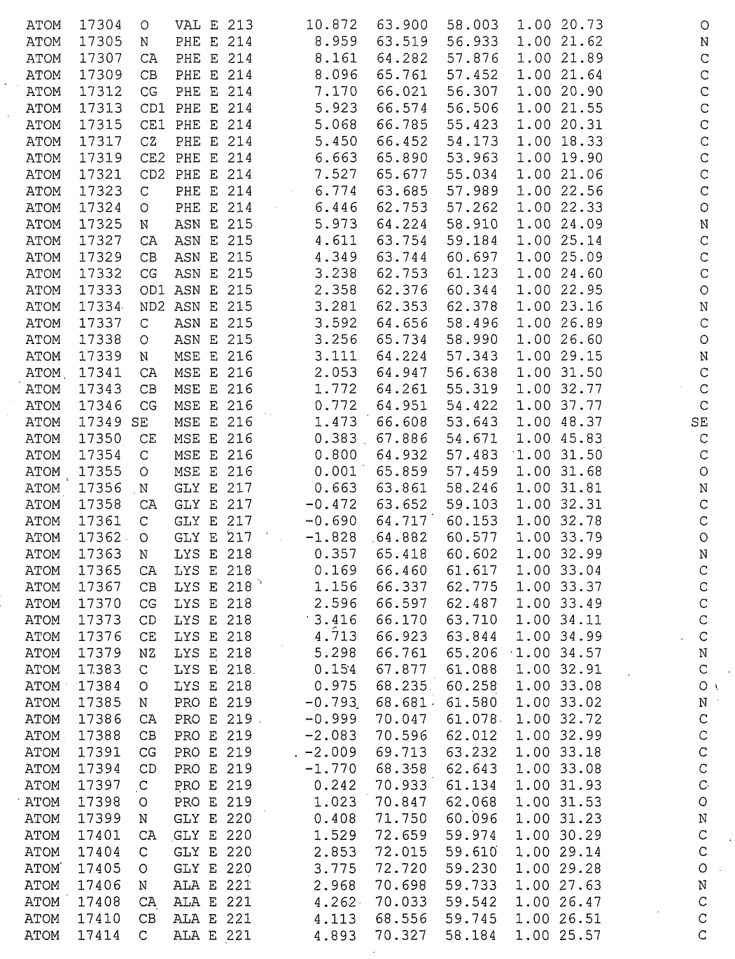

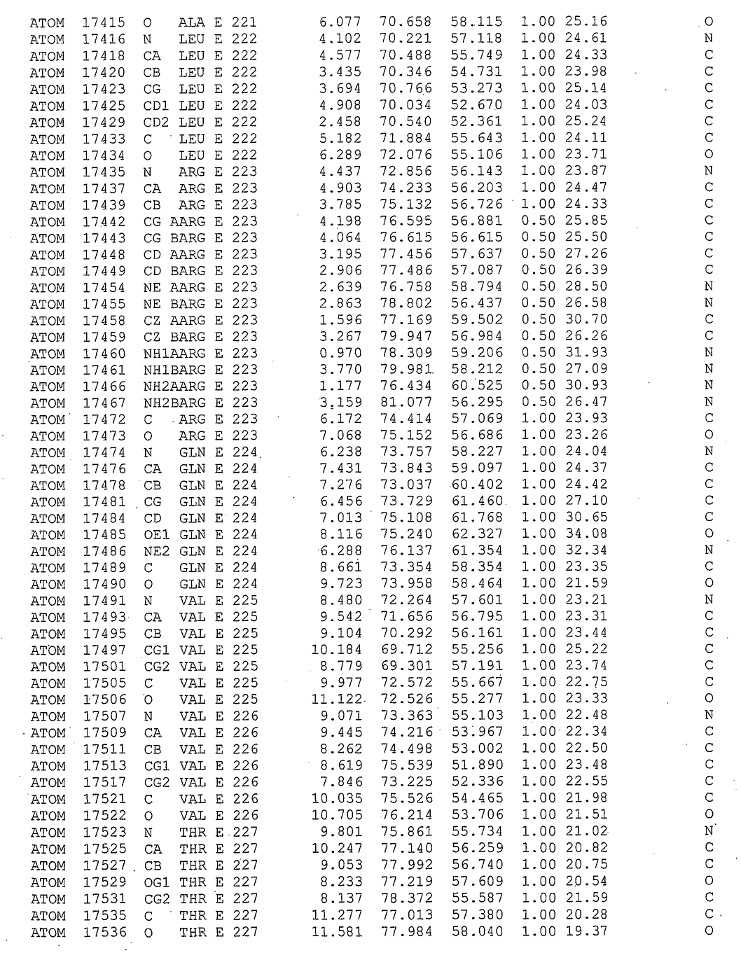

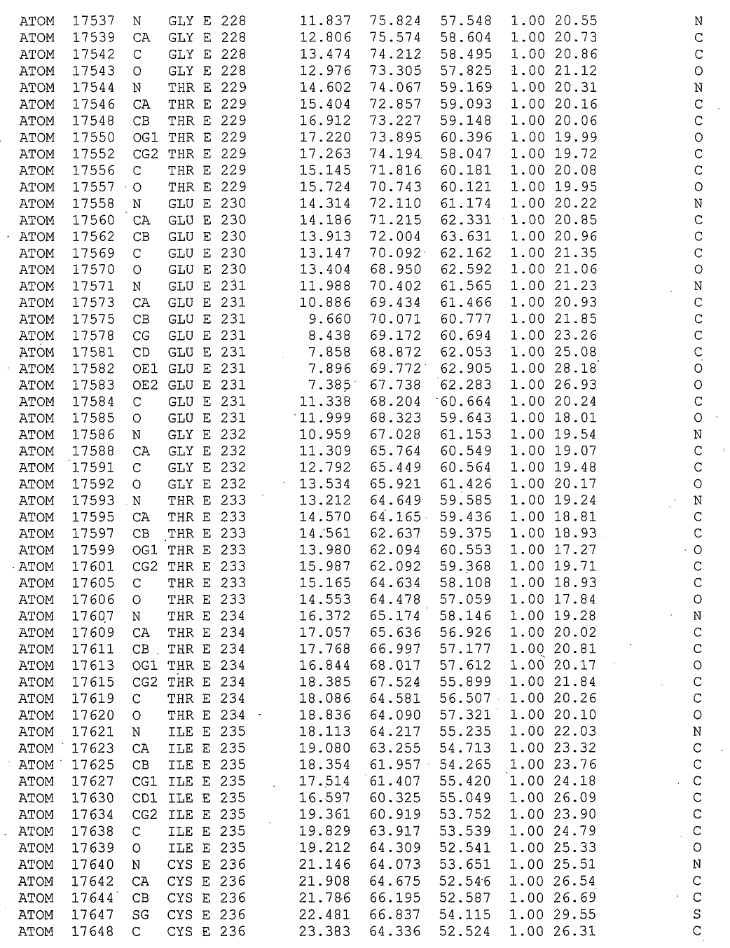

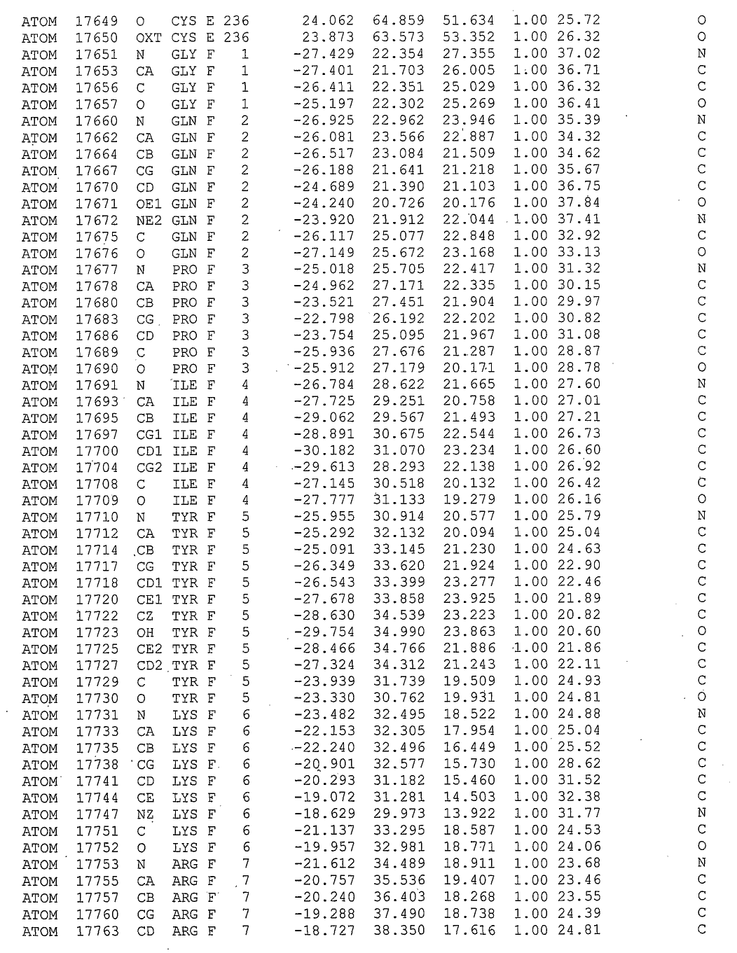

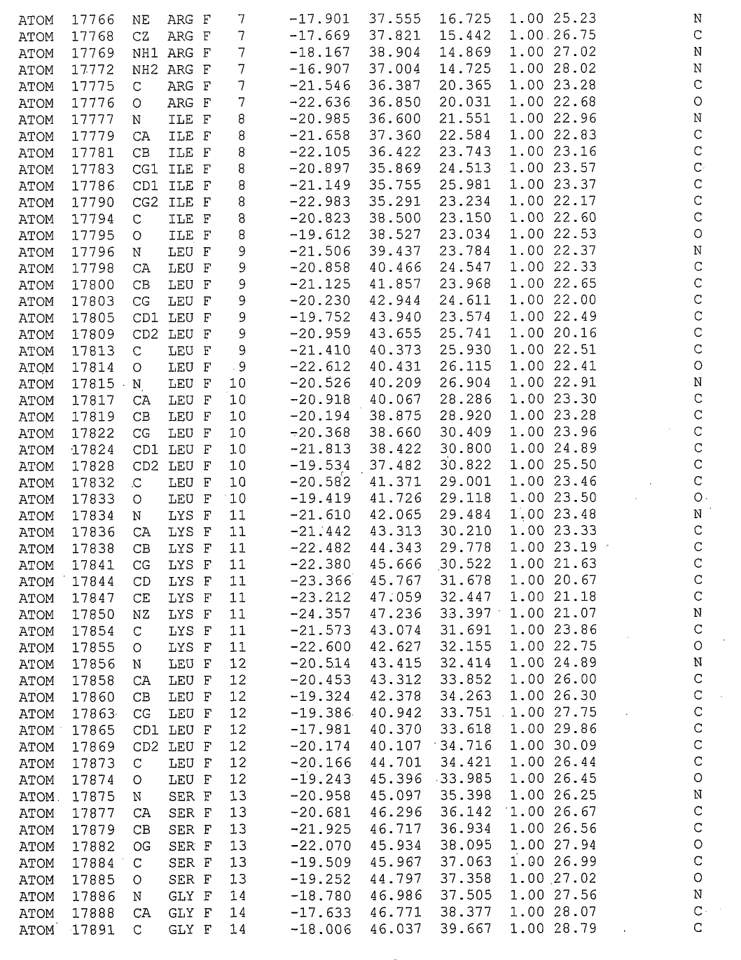

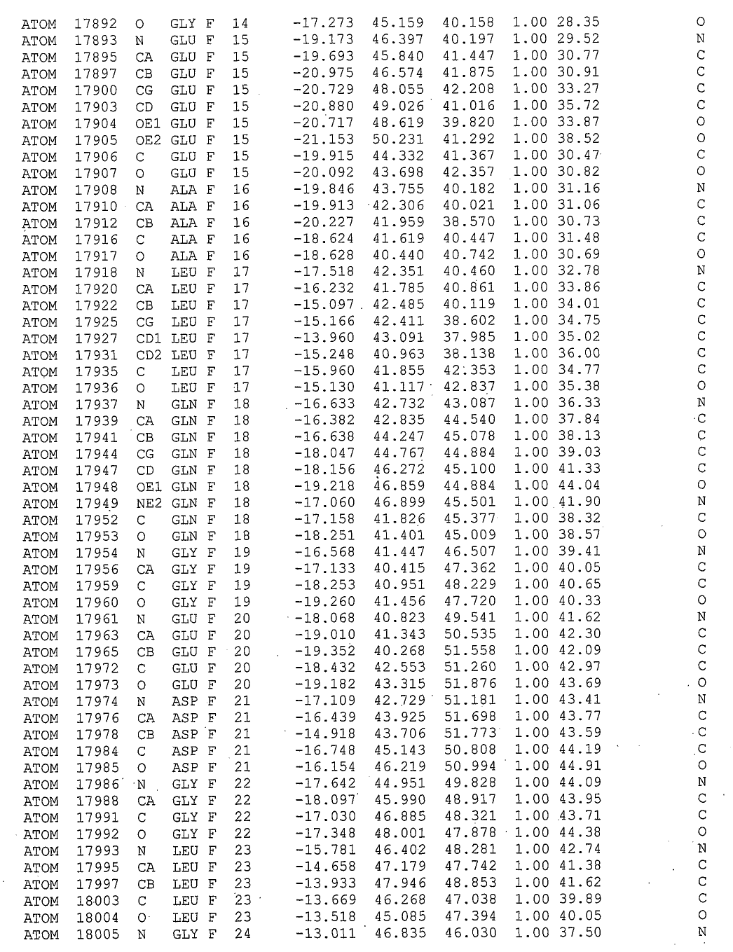

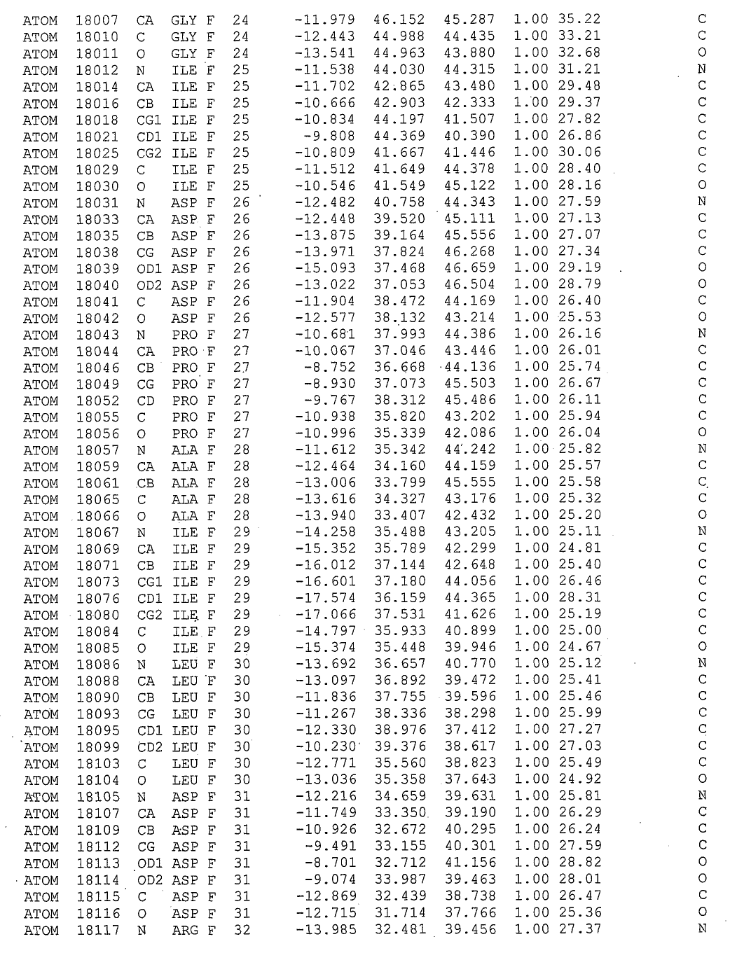

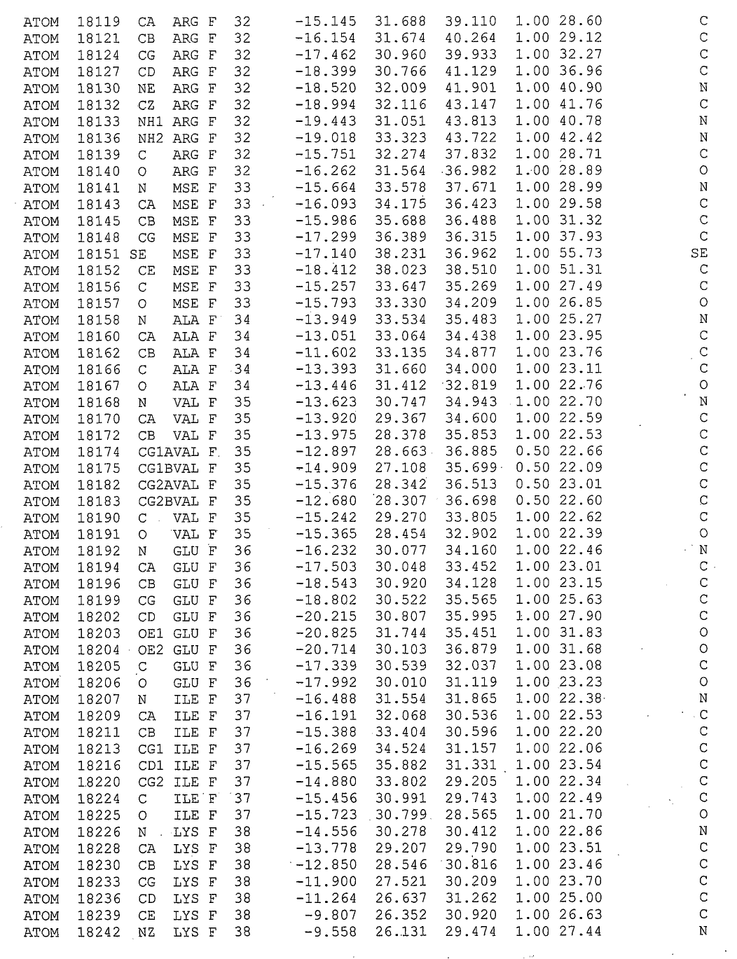

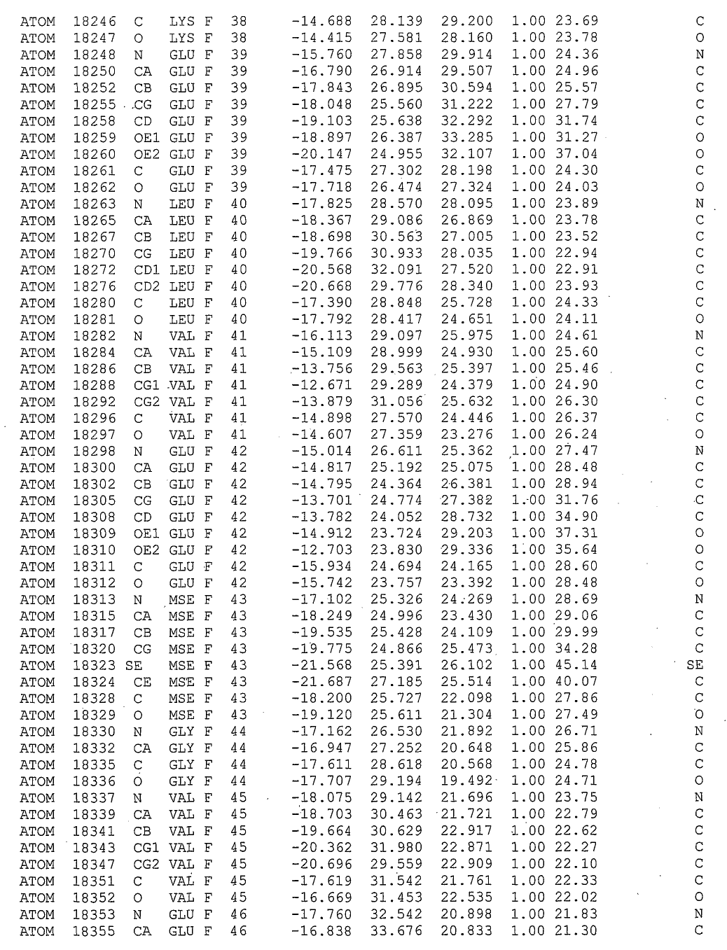

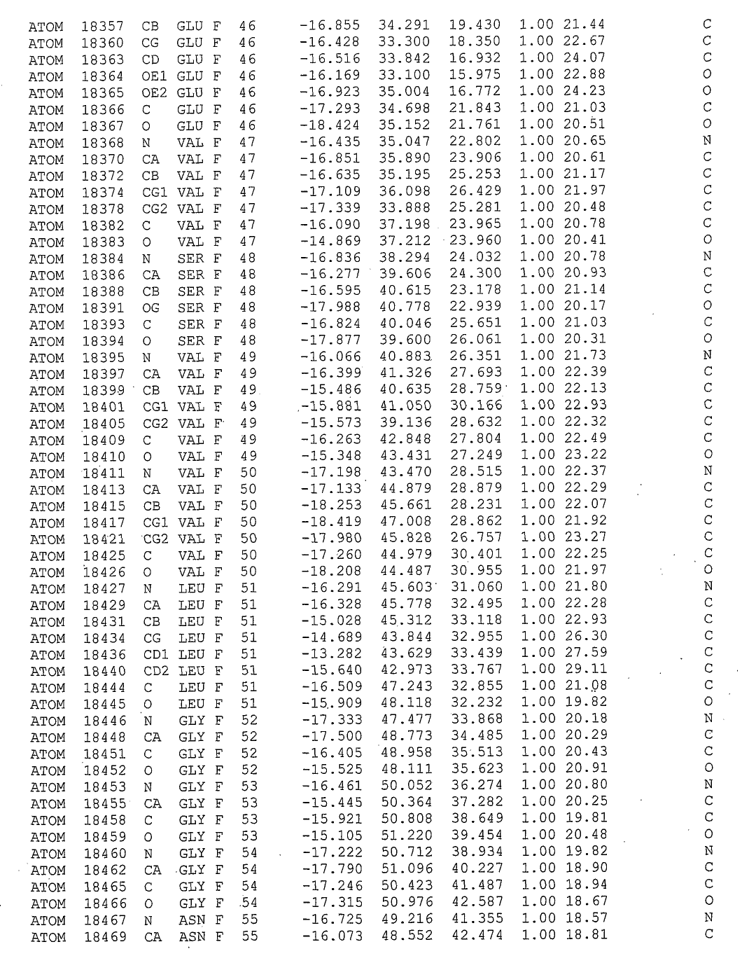

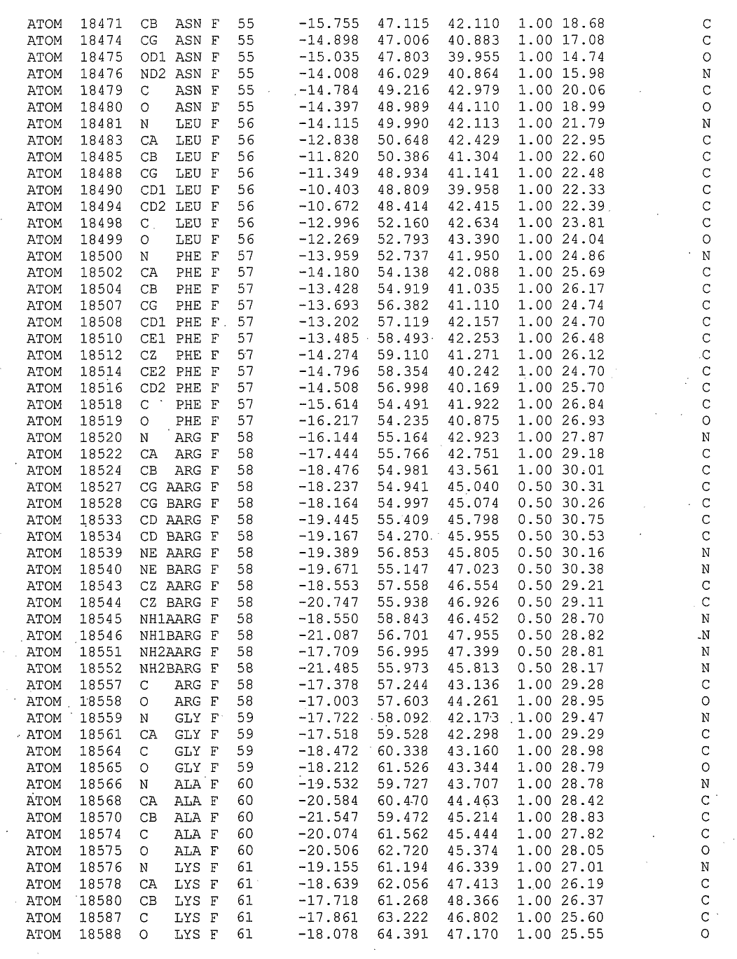

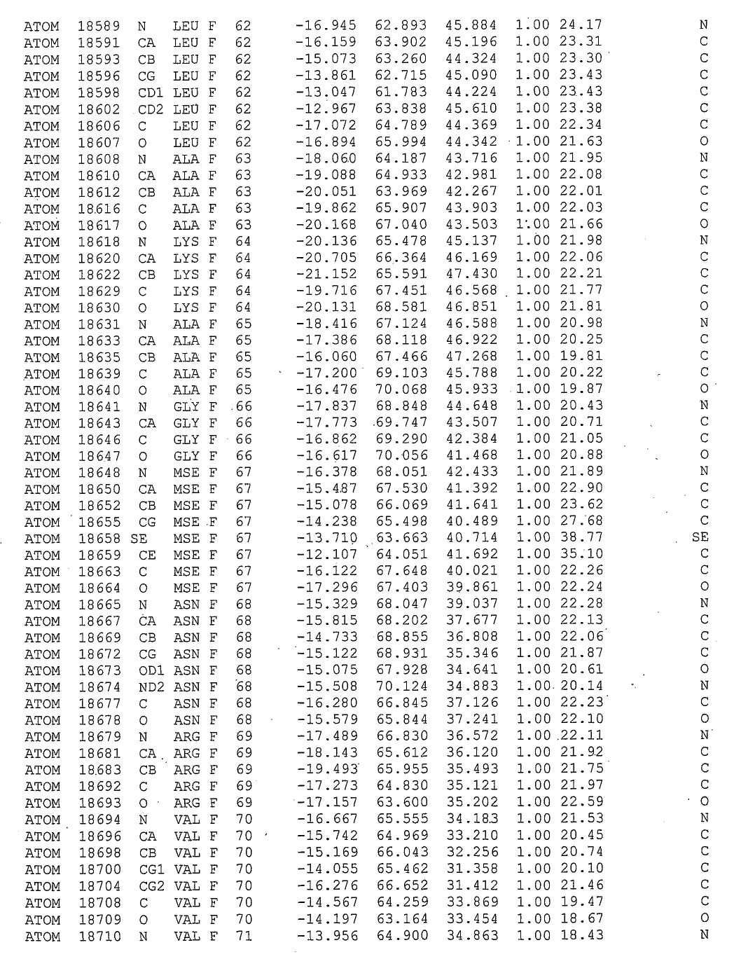

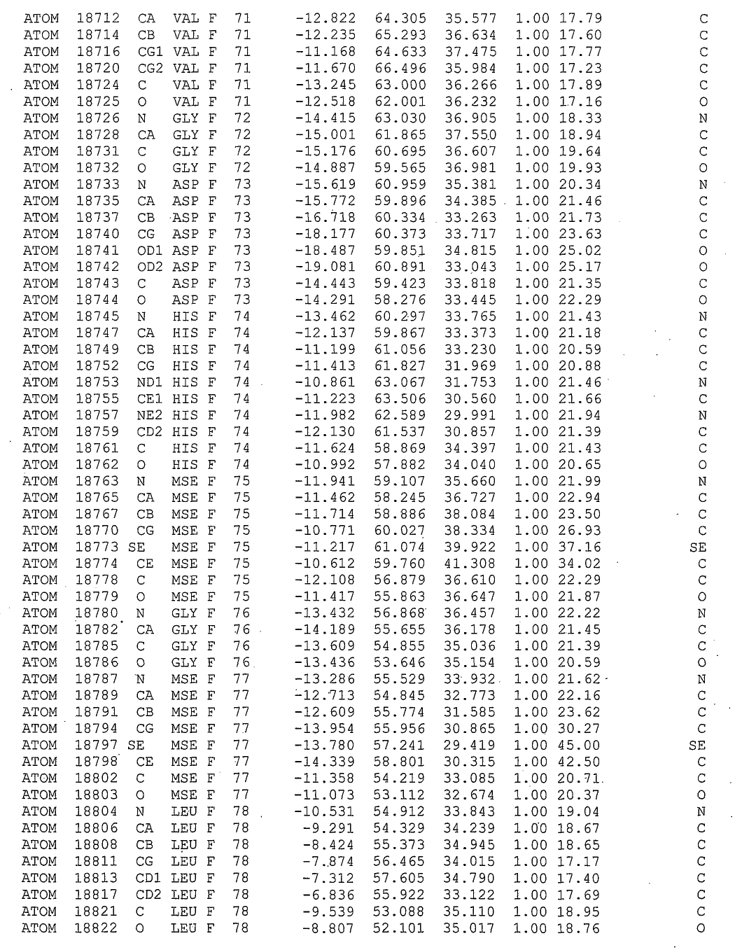

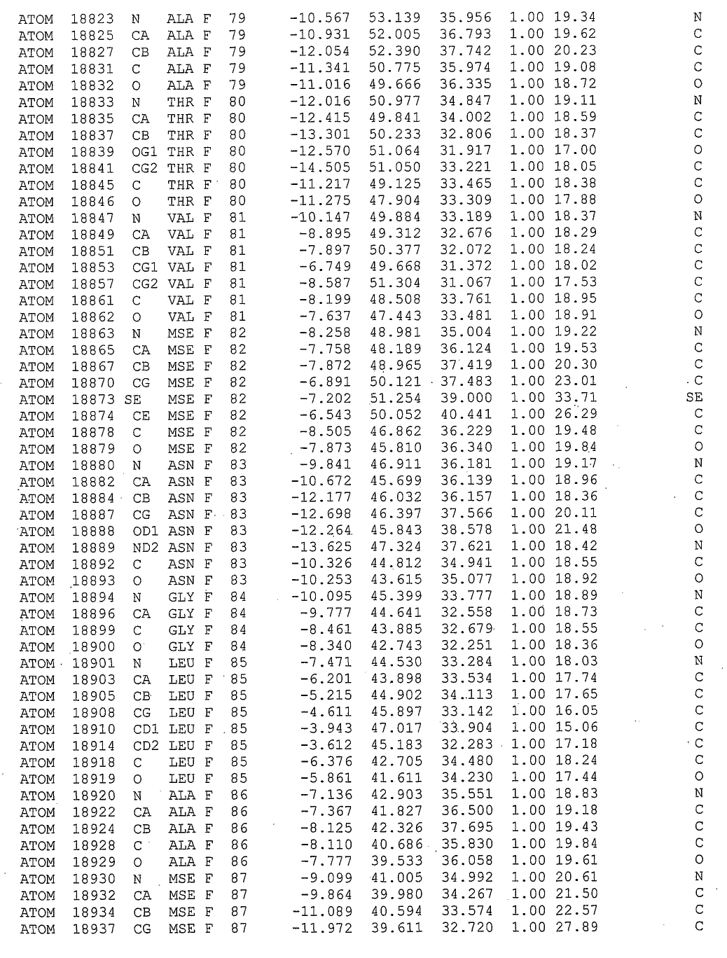

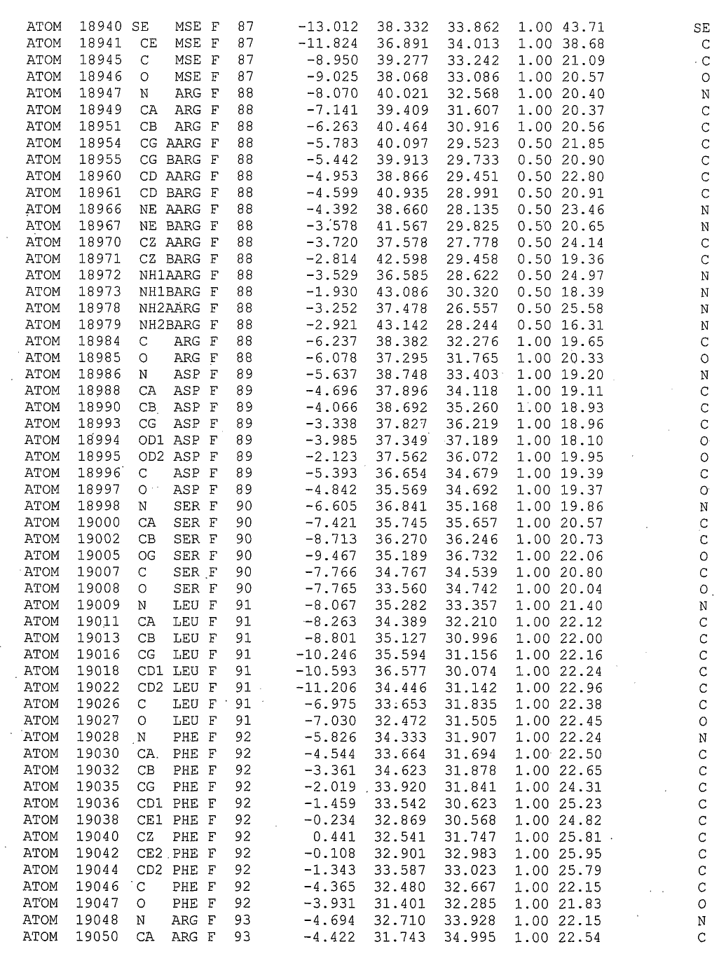

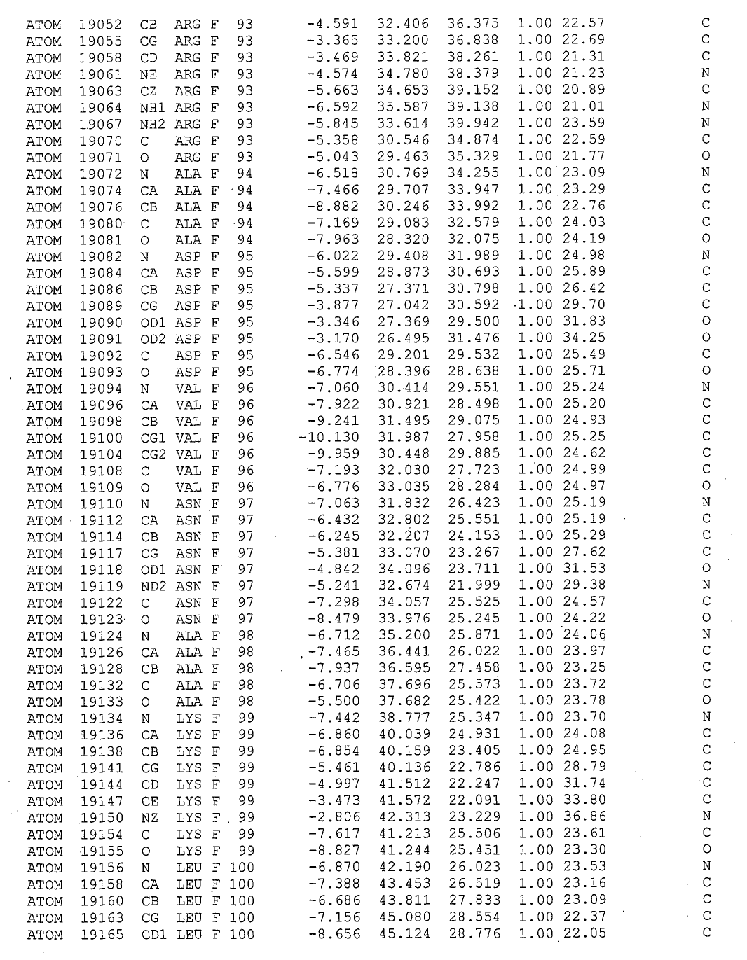

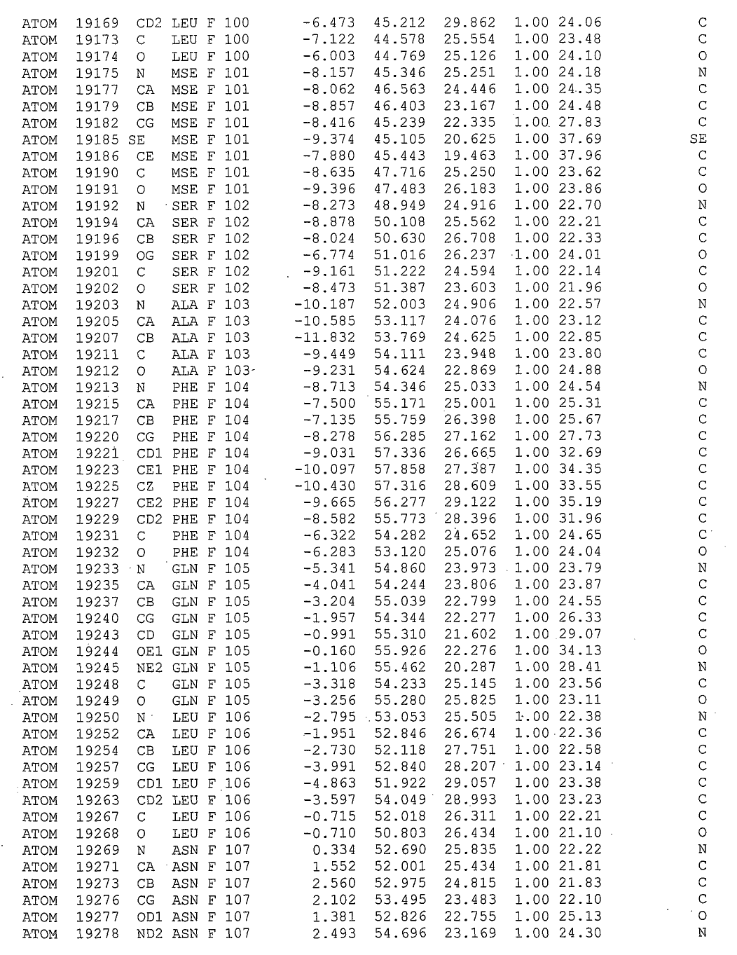

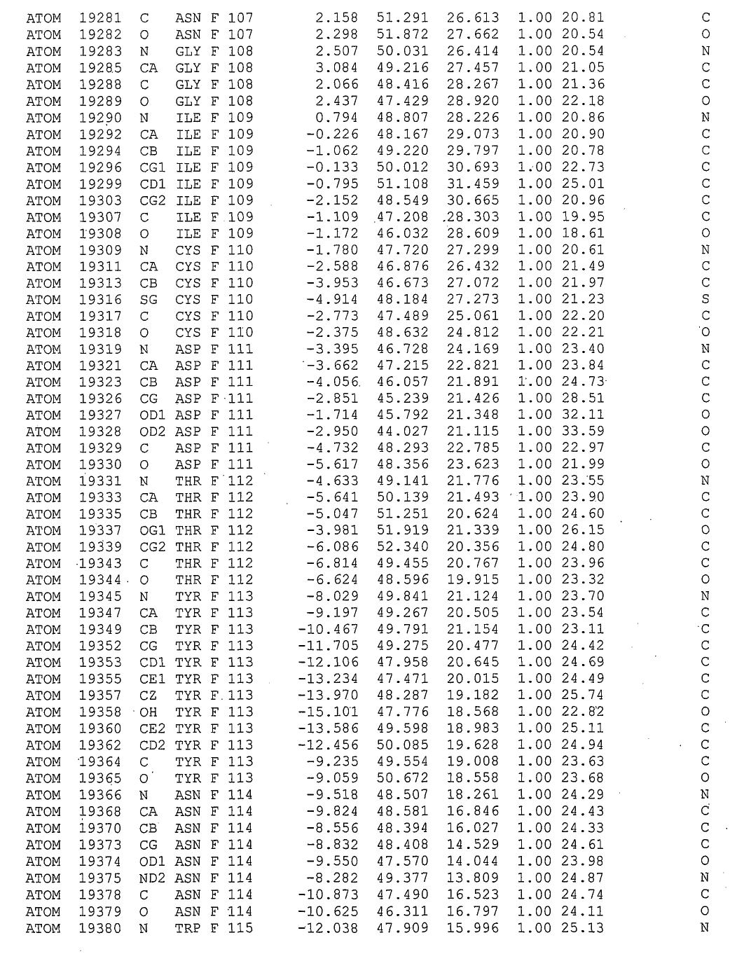

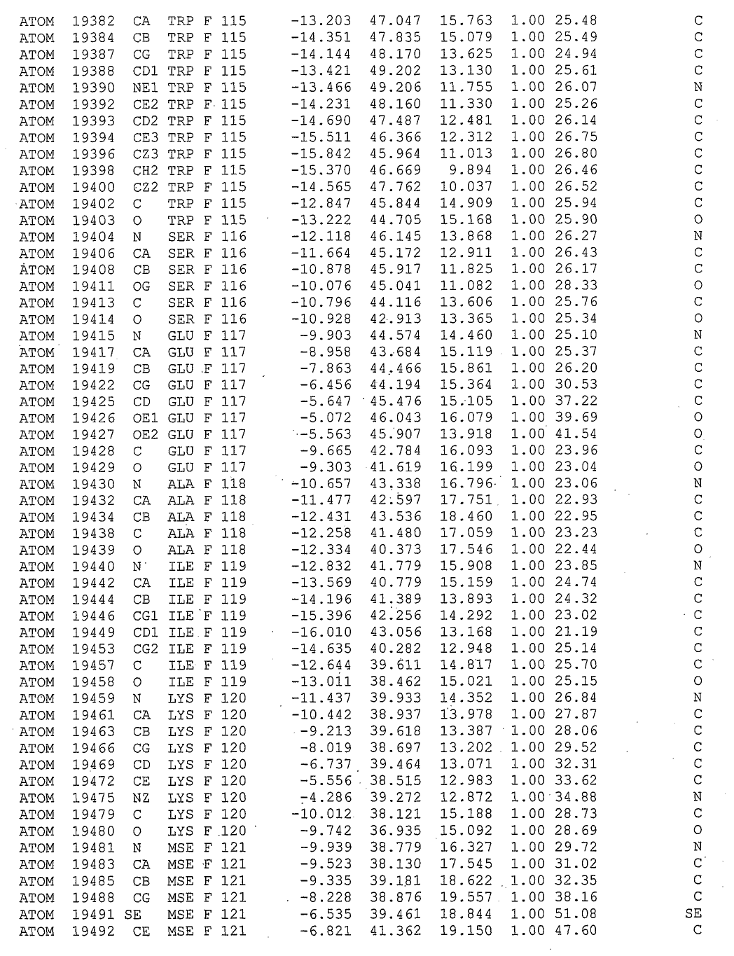

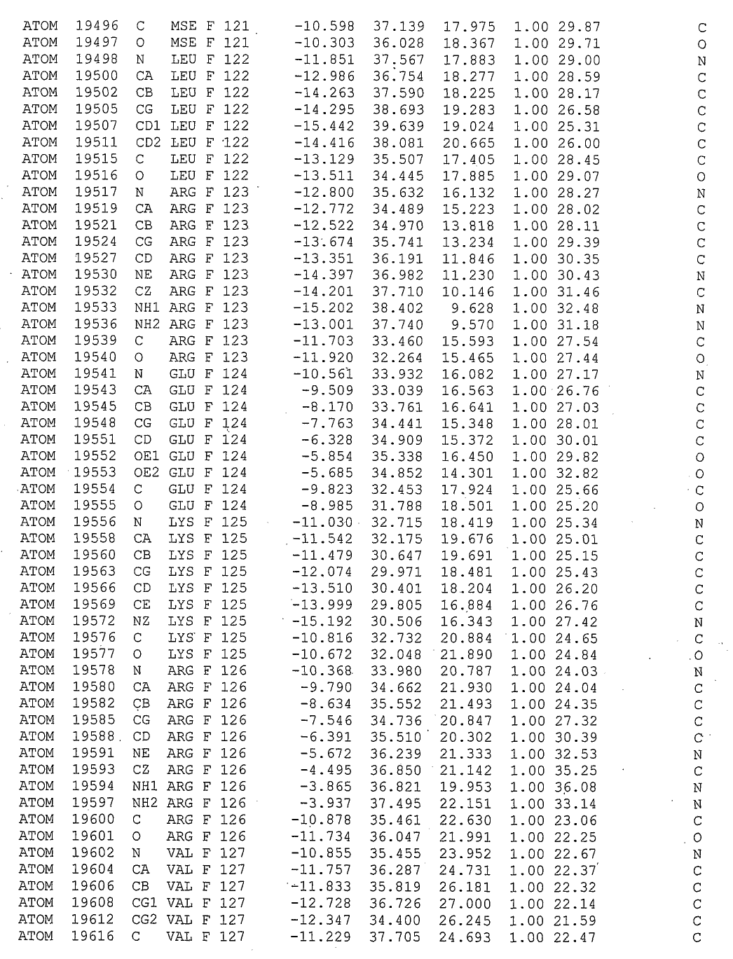

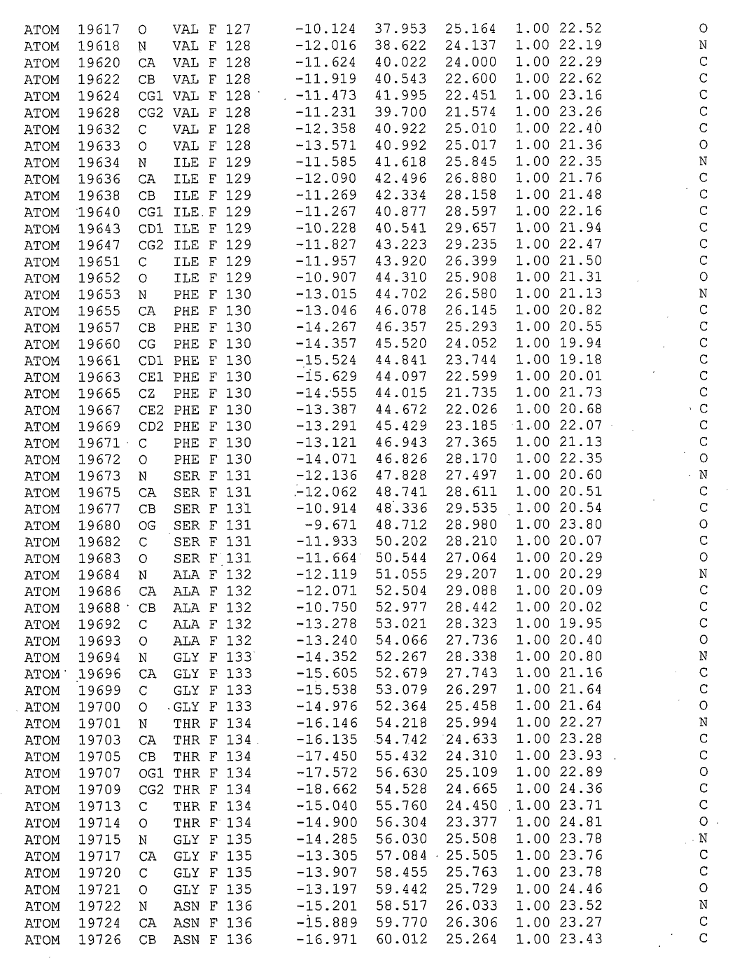

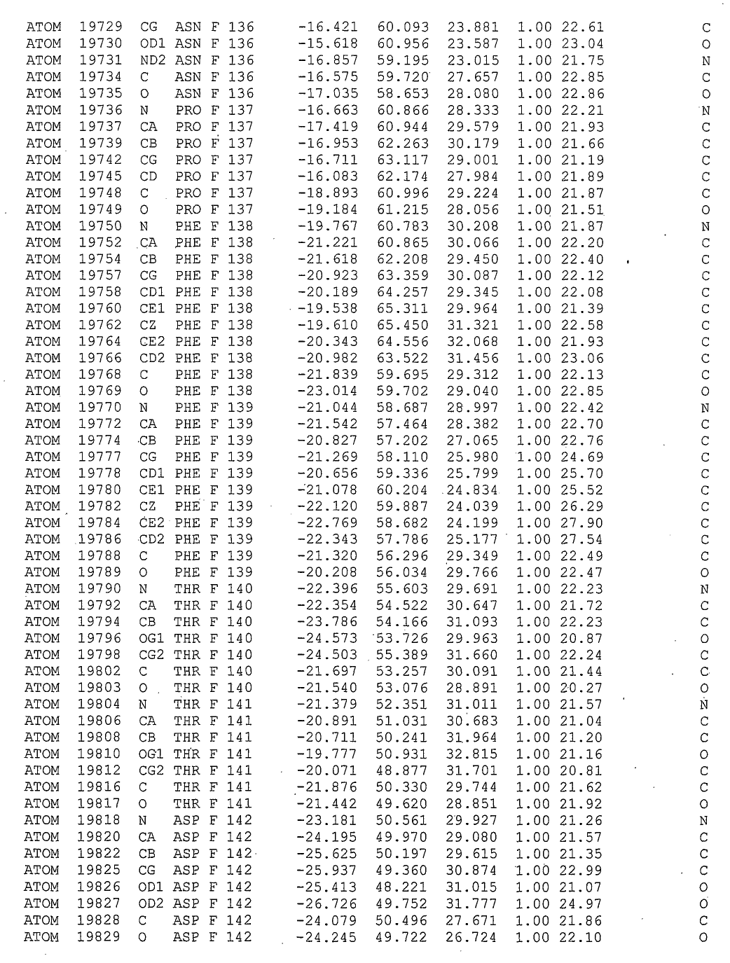

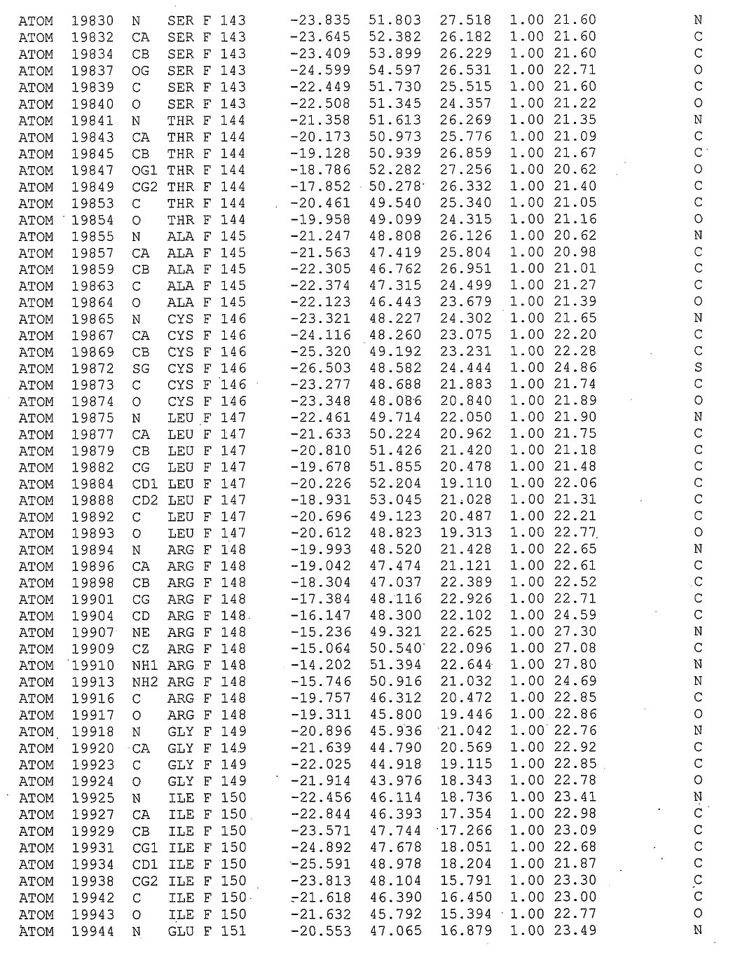

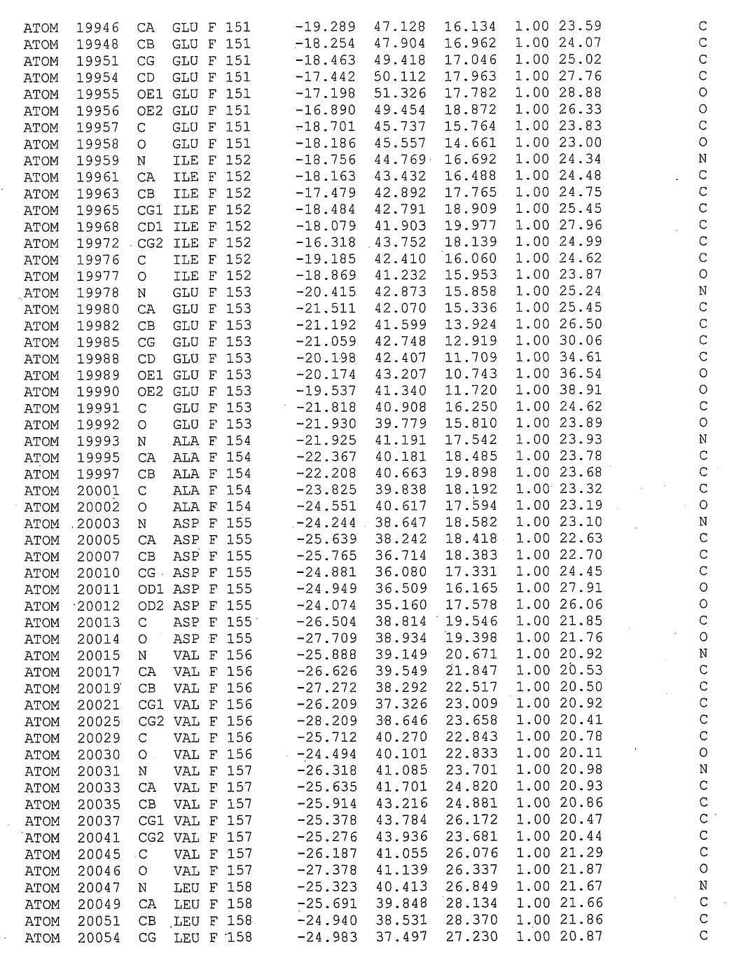

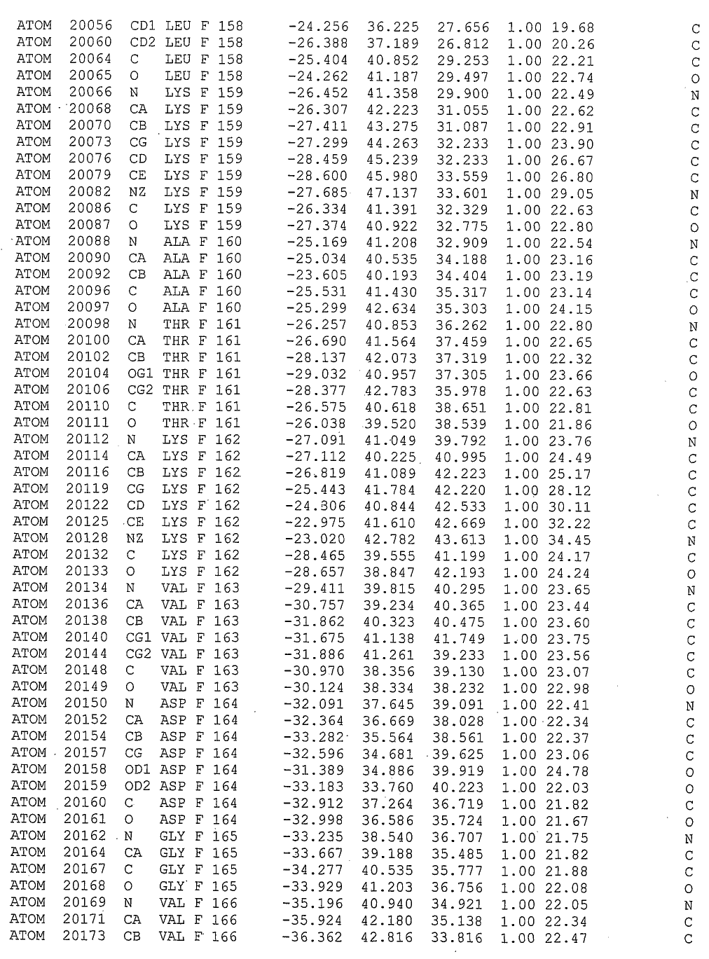

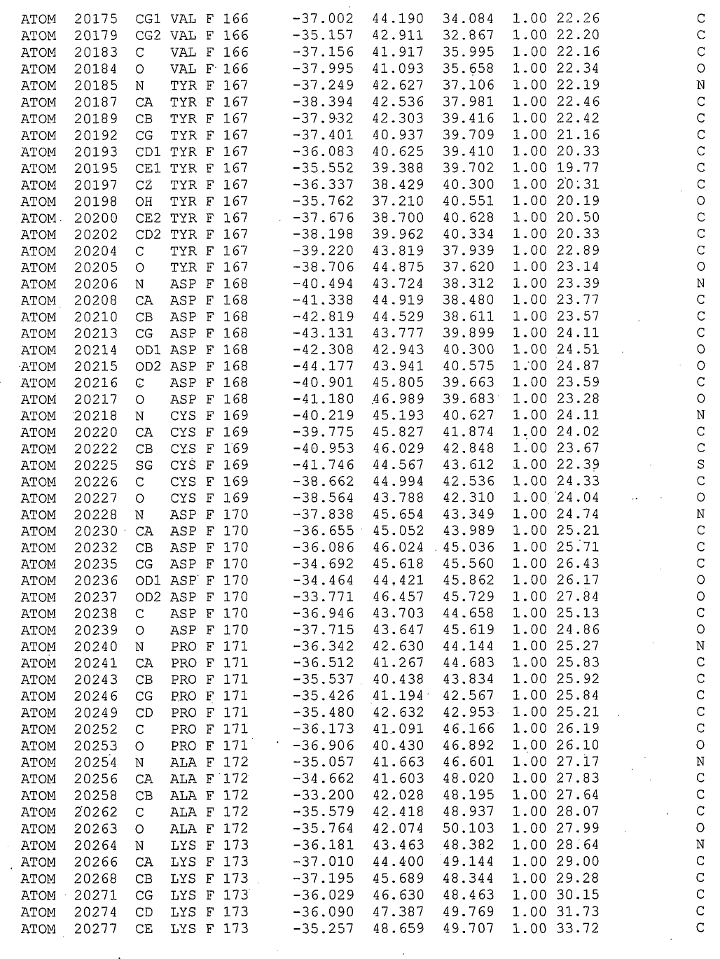

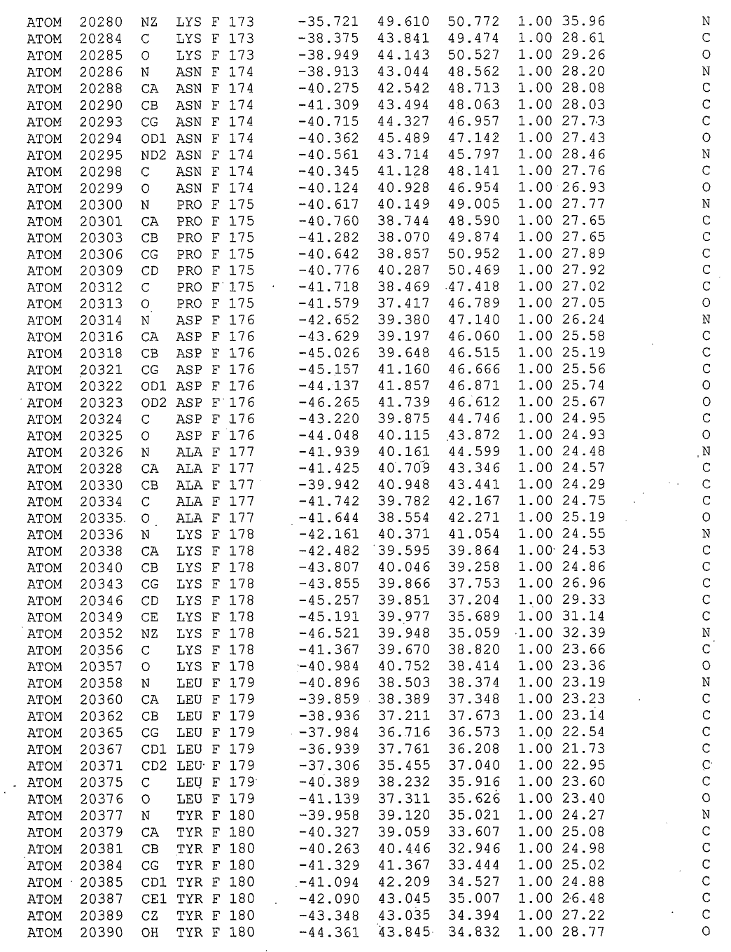

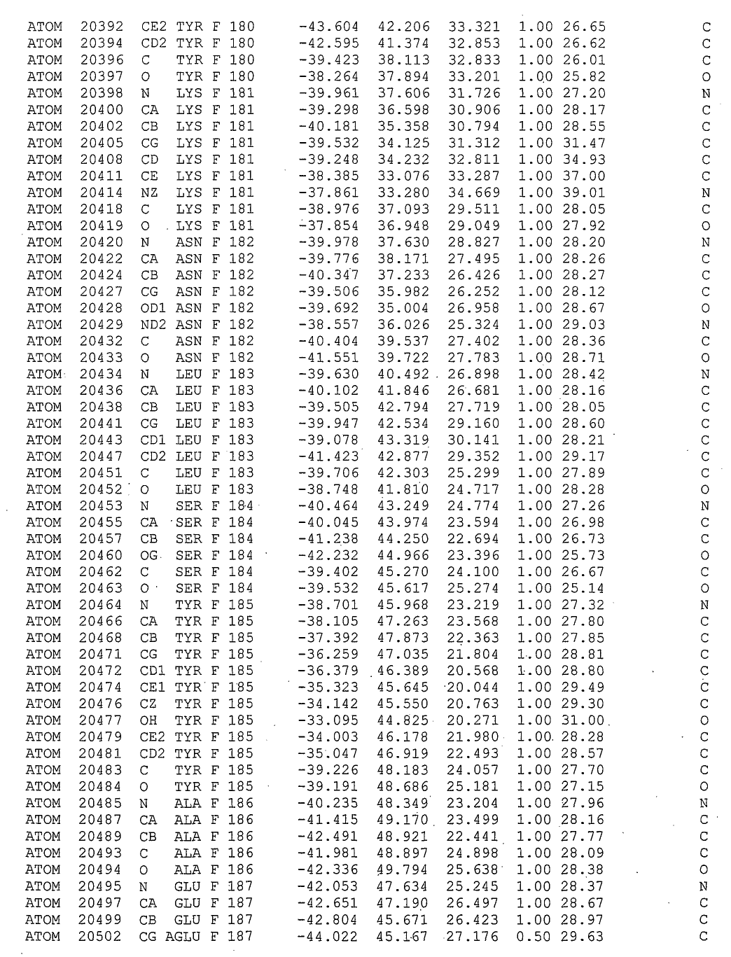

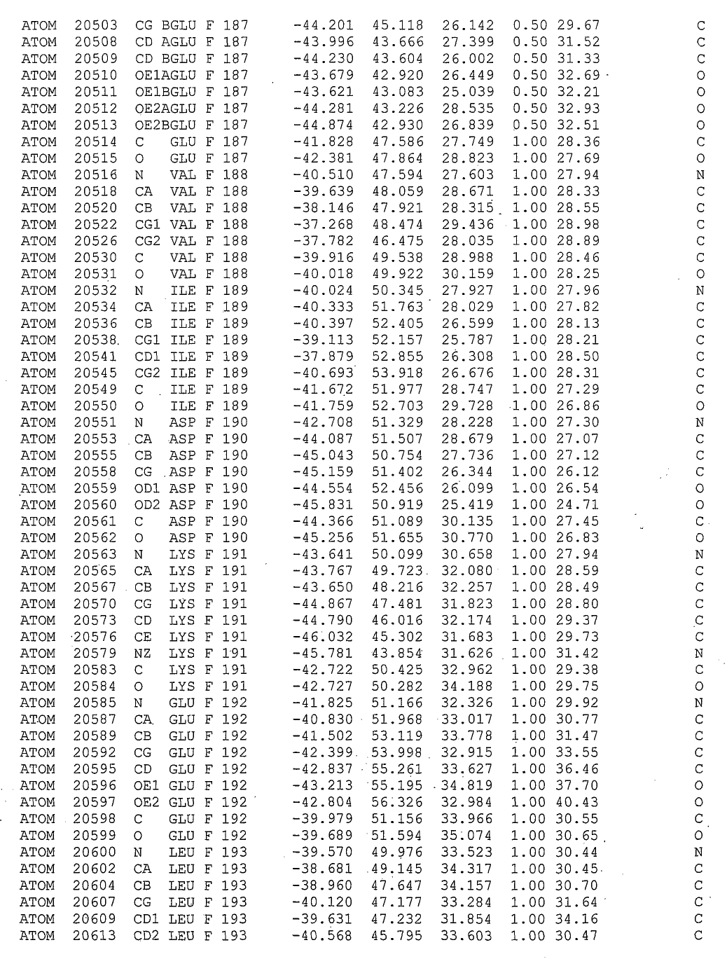

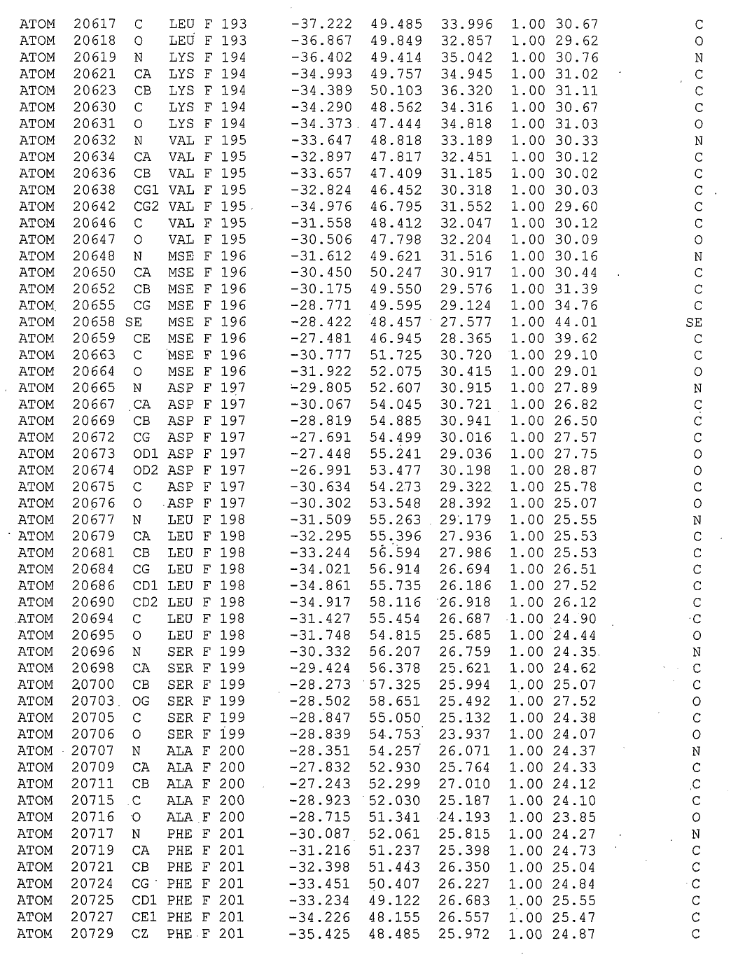

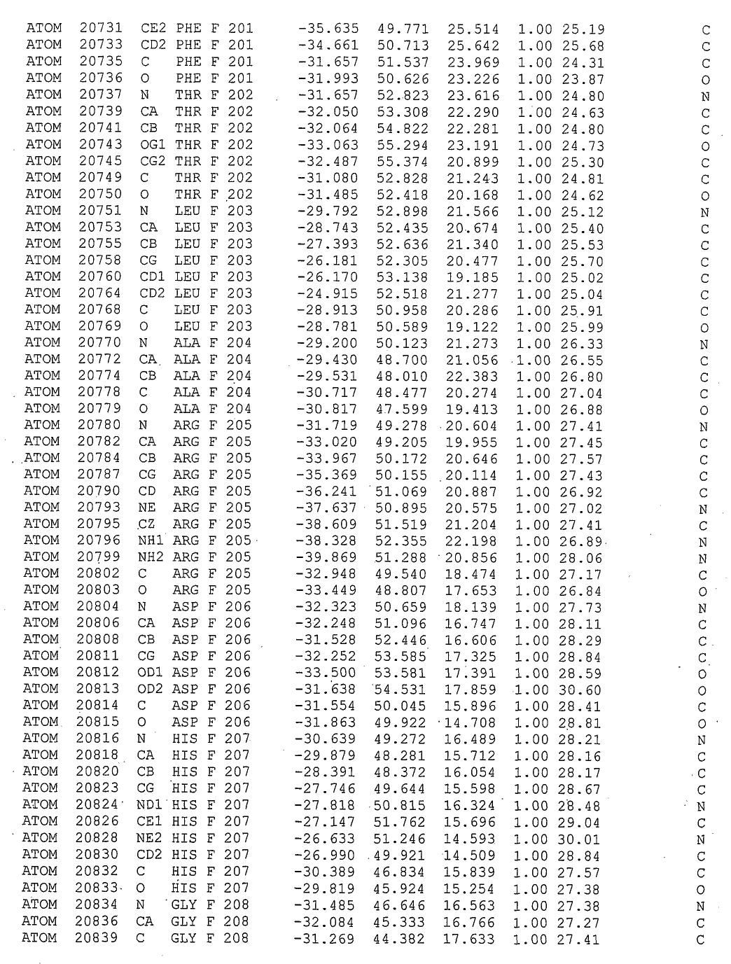

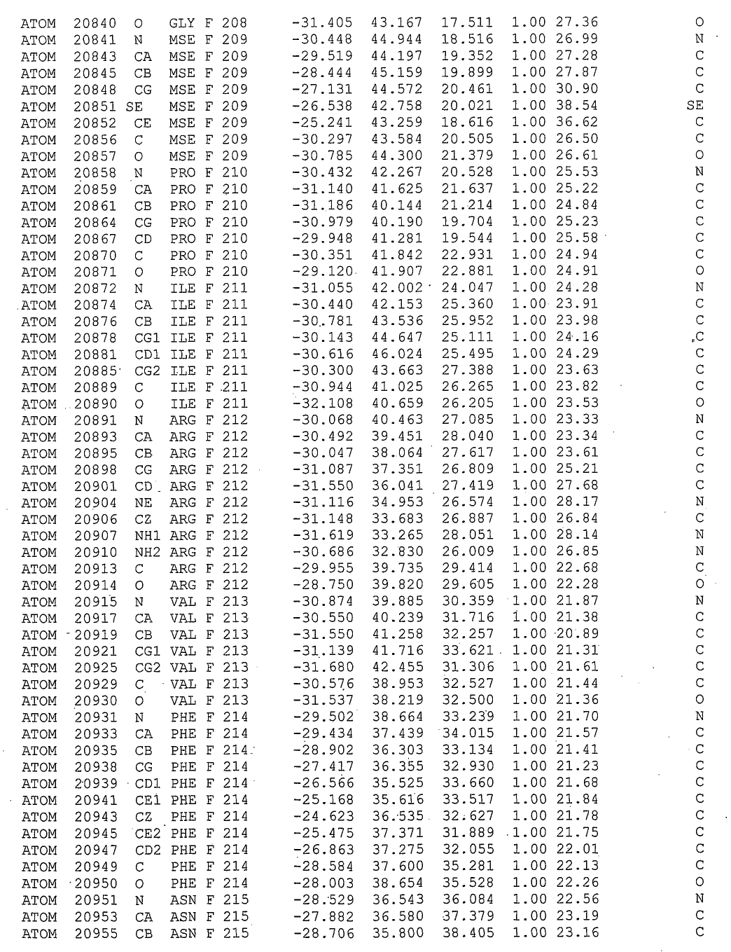

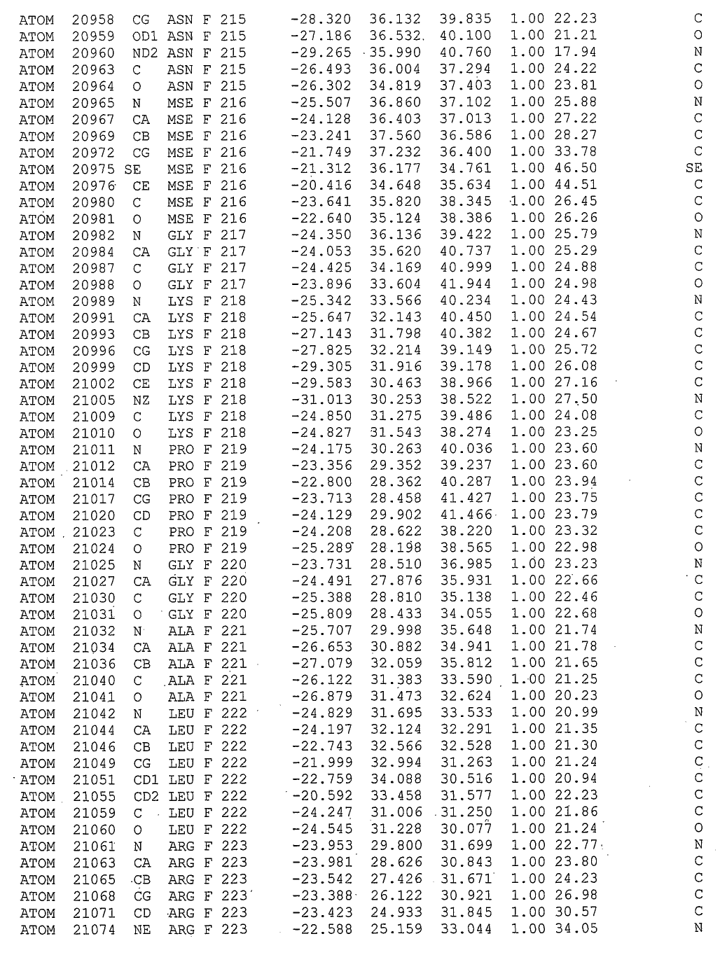

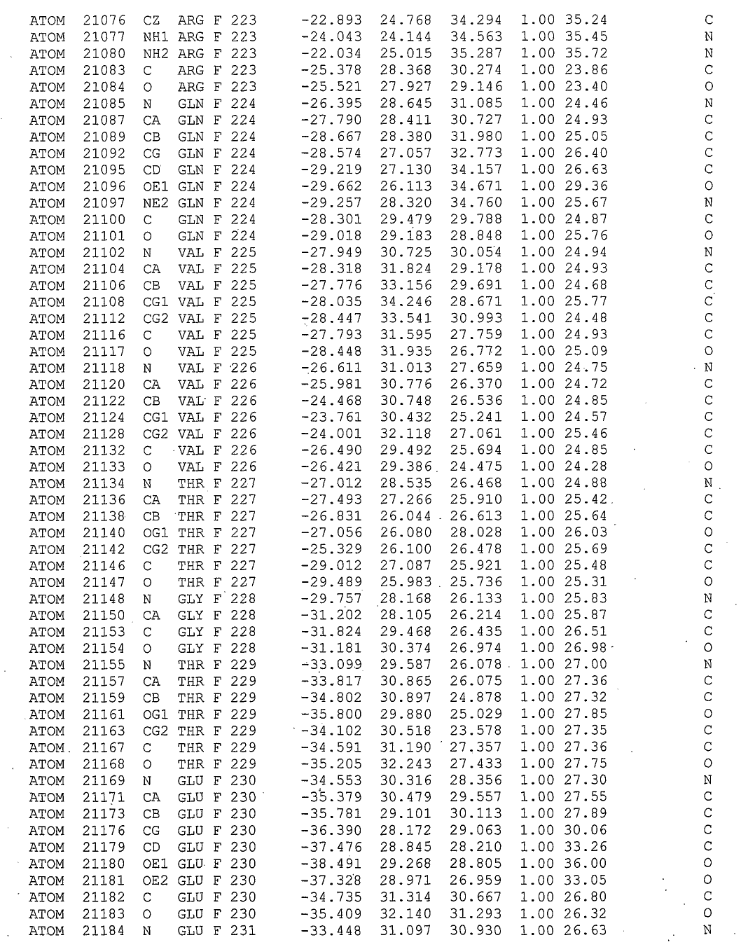

There is a good shape match between the protein and the bound GTP. The purine ring is sandwiched between the sidechains of Phe92 of the first molecule, Val71 of the second molecule, and Trp115 of the third molecule. Binding is not entirely driven by favorable van der Waals interactions. There are a number of polar interactions such as those between the sidechain guanidinium group of Arg88 of the first molecule and the N7 and O6 atoms of the guanine ring, and the backbone amino and sidechain carboxylate groups of Asp89 of the first molecule and the N1 atom and 2-NH₂ group of the guanine ring. The ribose hydroxyl groups are stabilised by a bidentate interaction with the guanidinium group of Arg123 from the third molecule. In addition, there are contacts between Ser116 of the third molecule and the ribose ring. The triphosphate moiety of the bound GTP molecules is poorly ordered, with the beta-phosphate adopting at least three distinct conformations. The alpha-phosphate is stabilised by interactions with the sidechains of Arg126 from the first molecule and of Ser116 from the third molecule. In one of the observed conformations the beta-phosphate is stabilised by interaction with the sidechains of Asn97 and Arg126 of the first molecule. In a second conformation the beta-phosphate is stabilised by interaction with the sidechain of Ser116 from the third molecule, and the backbone carbonyl of Ala98 from the first molecule. In a third conformation the beta-phosphate is stabilised by interactions with the sidechains of Lys99 and Arg126 from the first molecule, and Lys120 from the third molecule. The position of the gamma-phosphate is not well determined and it has been omitted from the final model. Analysis of the relative conformations of the eight molecules in the crystal asymmetric unit (see FIG. 4) suggests that binding of GTP at the allosteric binding site may modulate activity by modulating the position of helix B (residues 60-65), and the subsequent loop (alphaB-alphaC) that links helix B to helix C (residues 66-69) (see FIG. 1). These residues are proposed to form part of the UMP binding site.

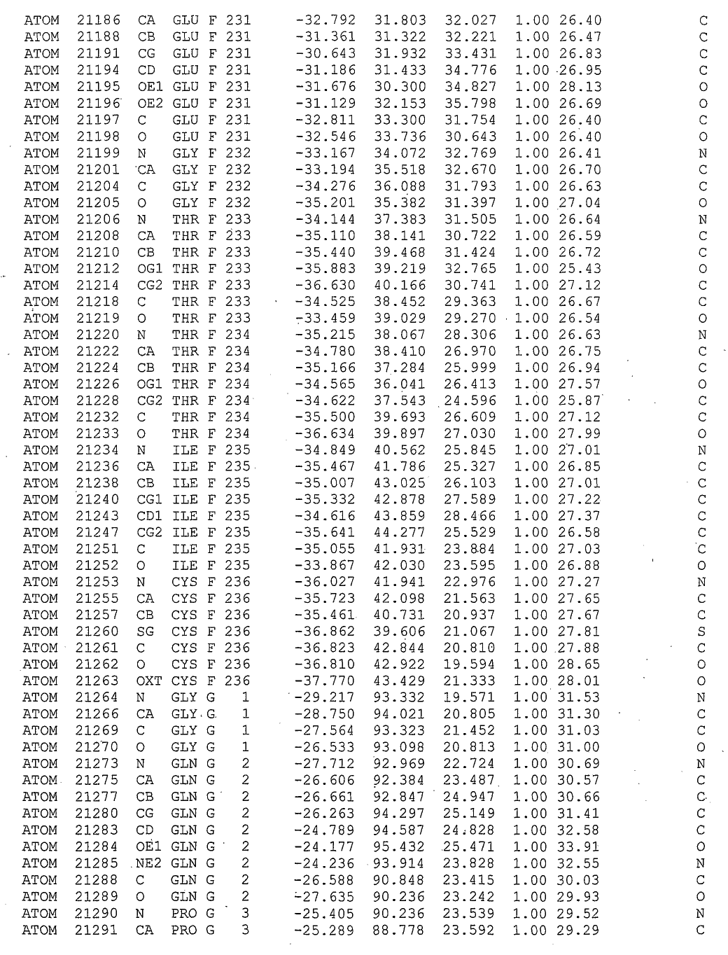

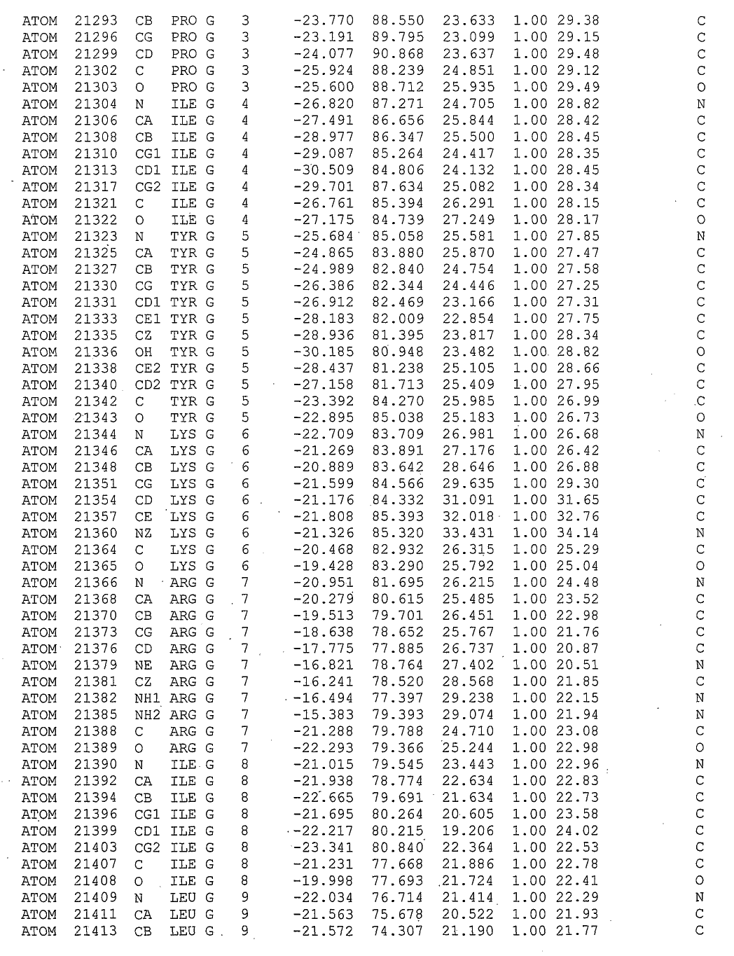

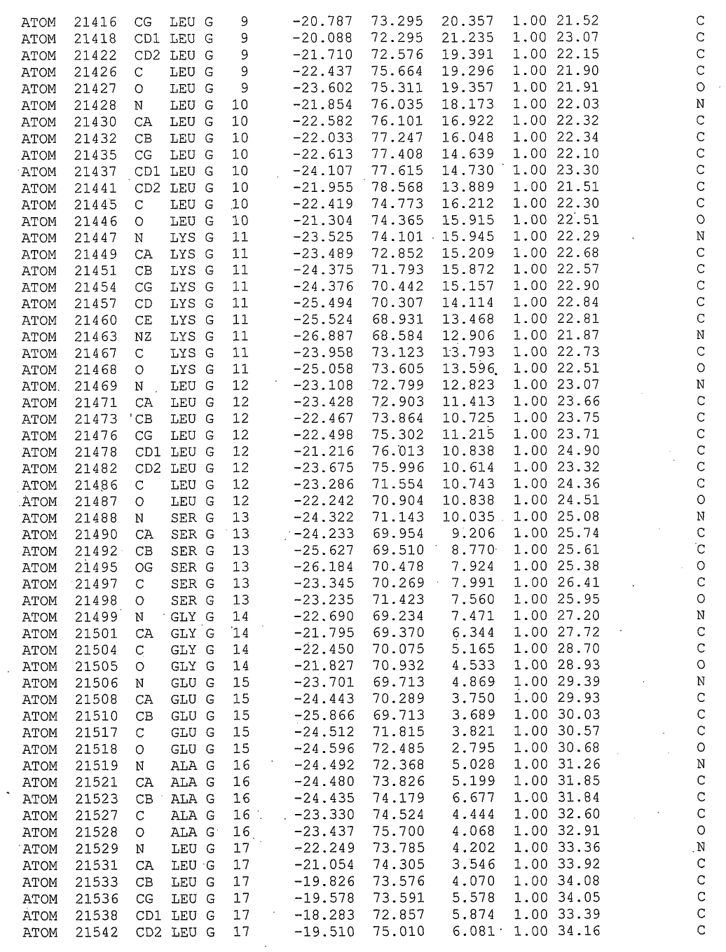

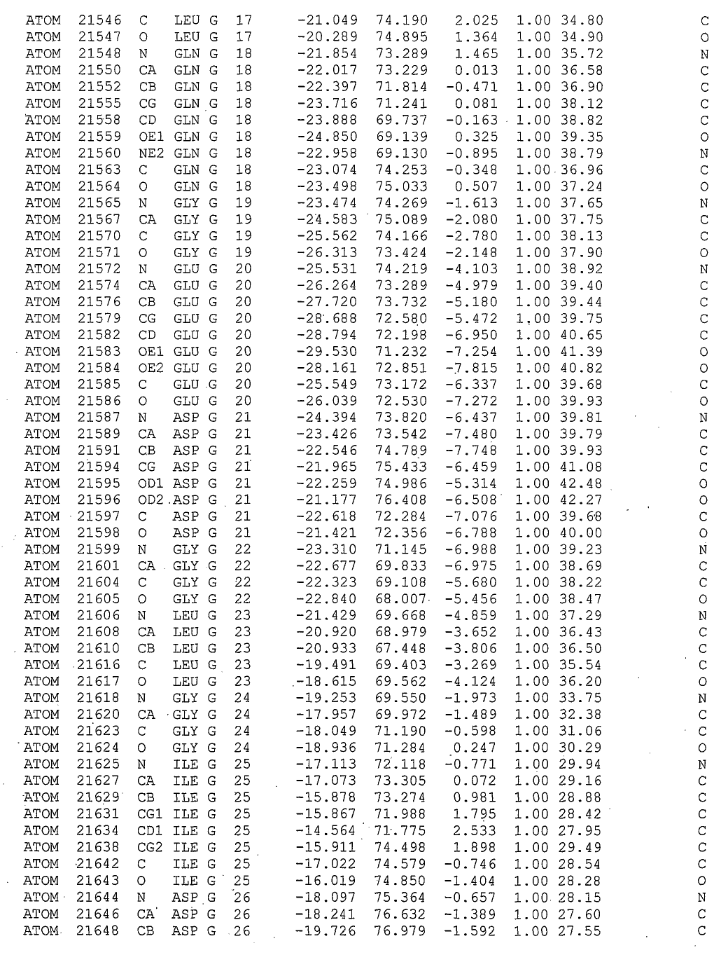

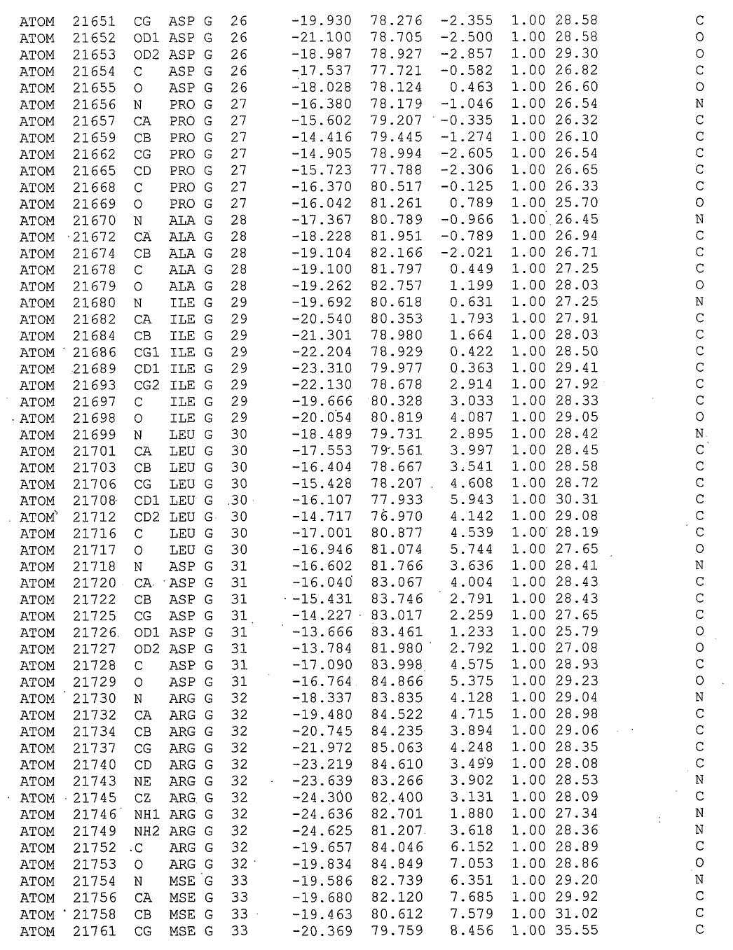

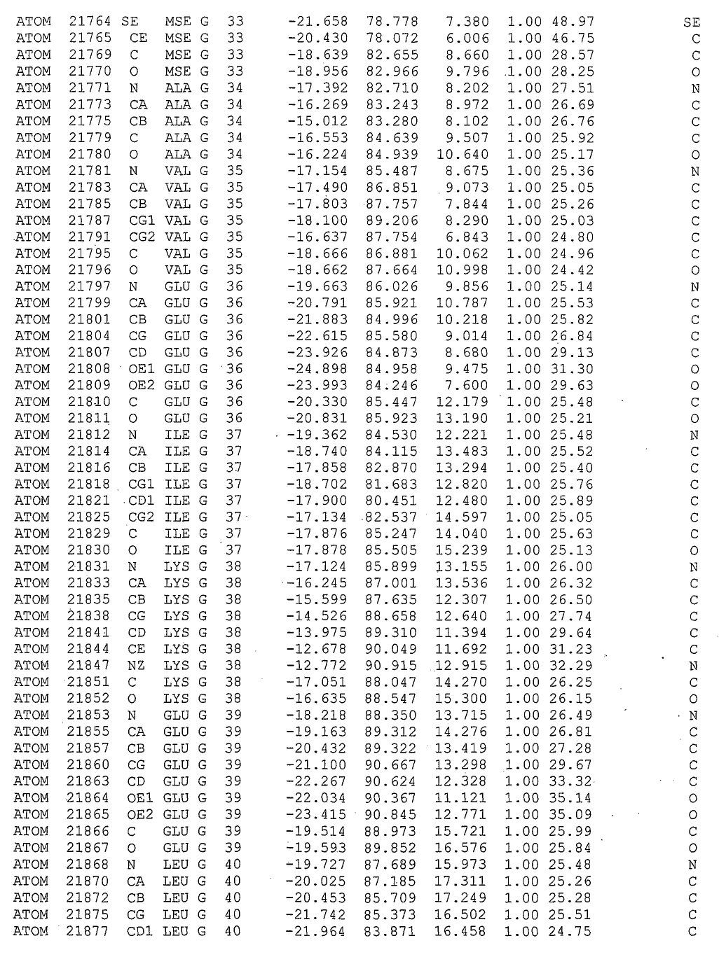

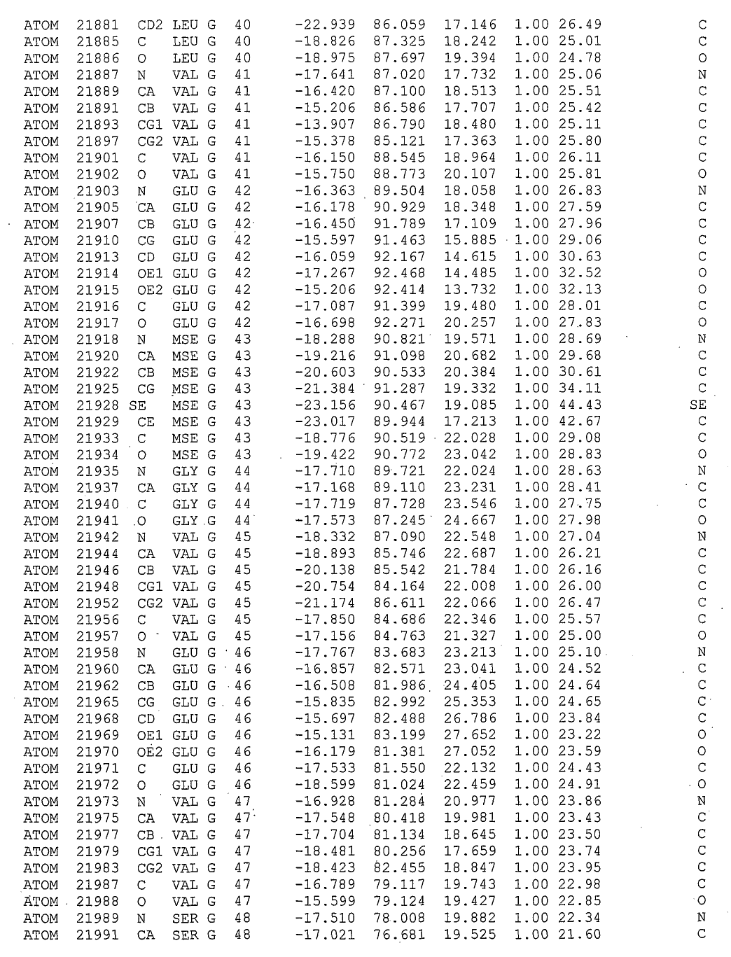

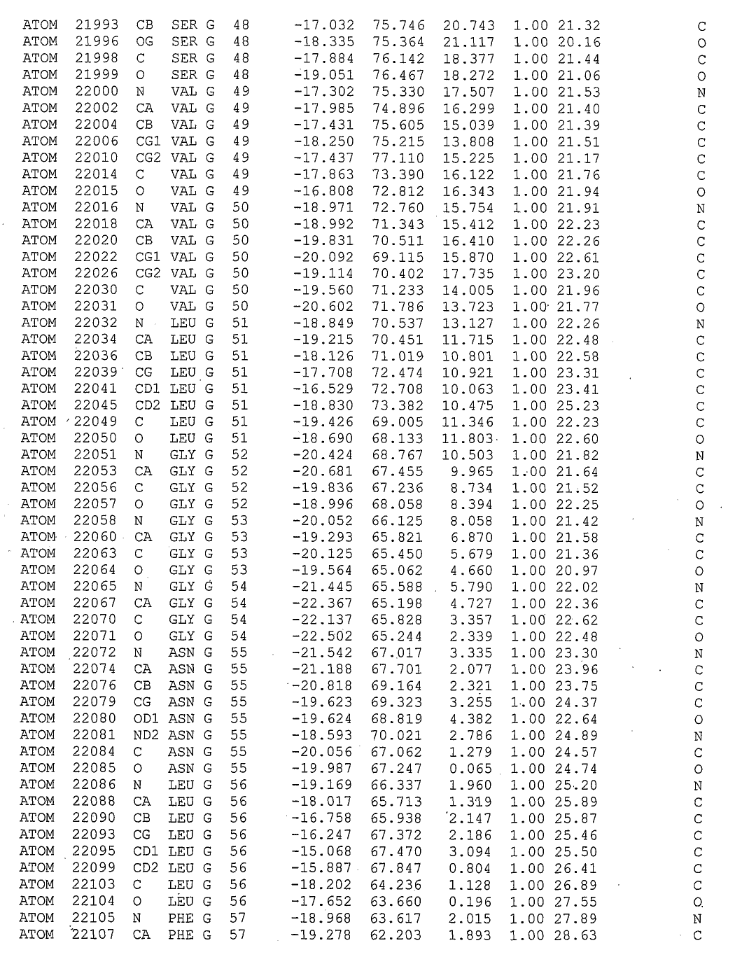

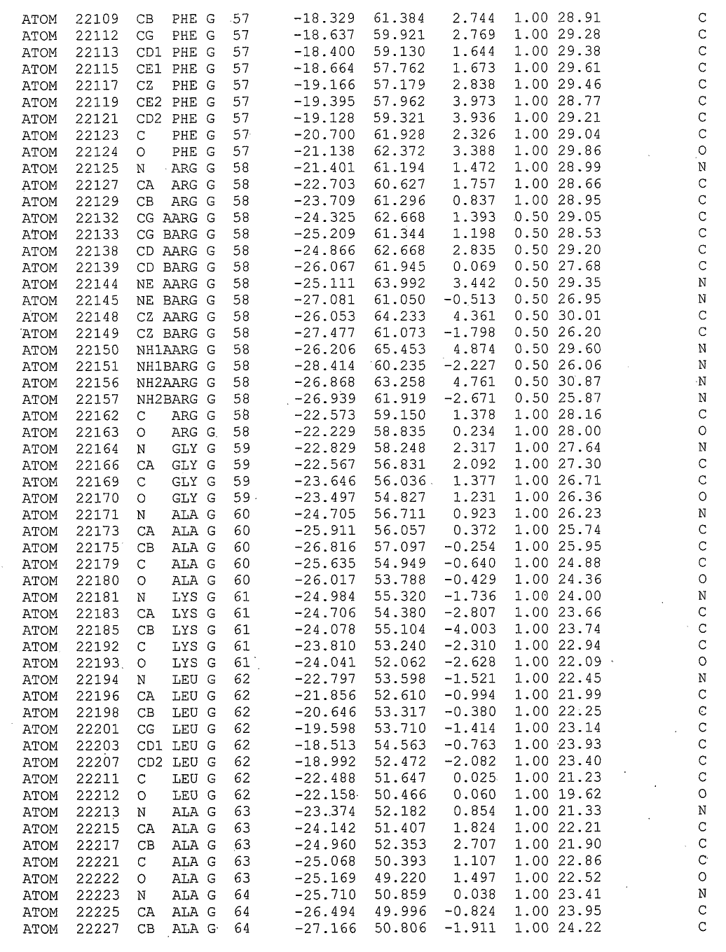

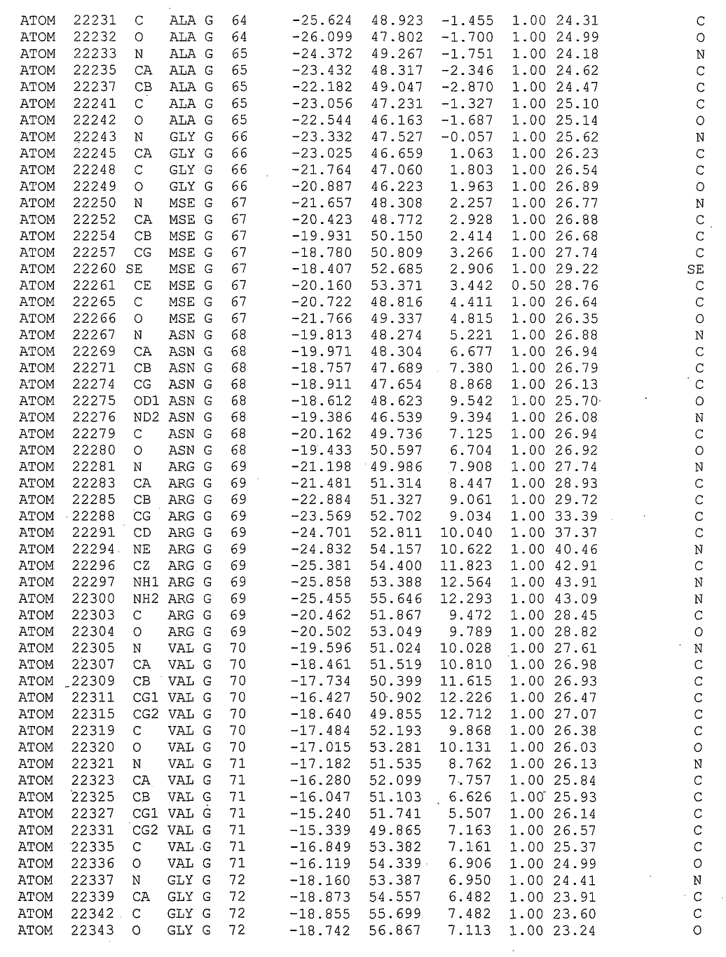

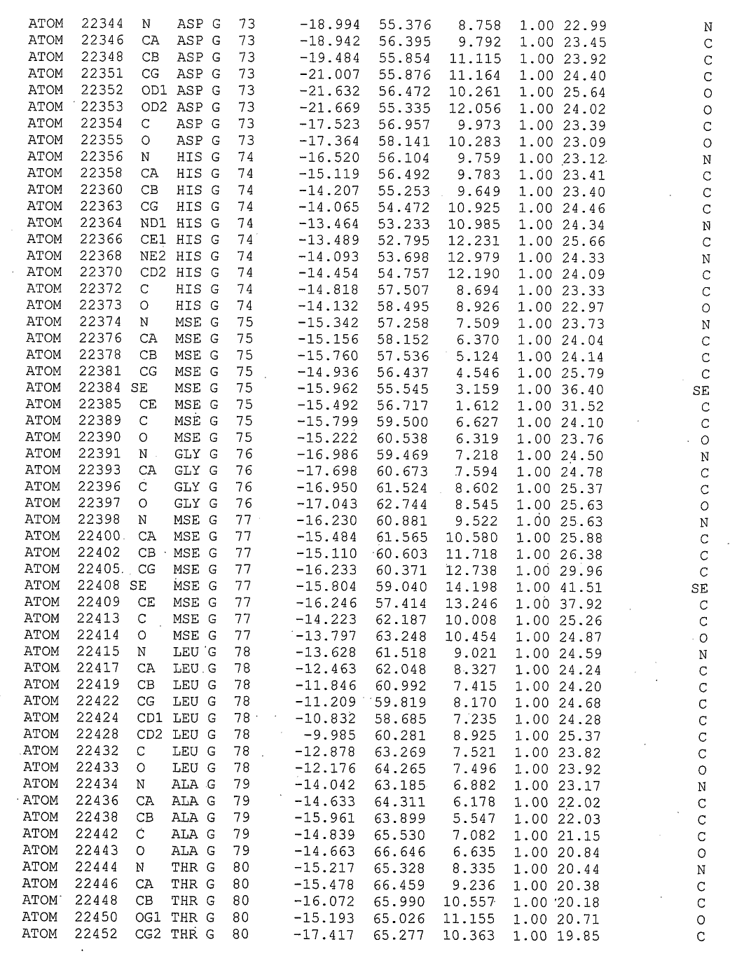

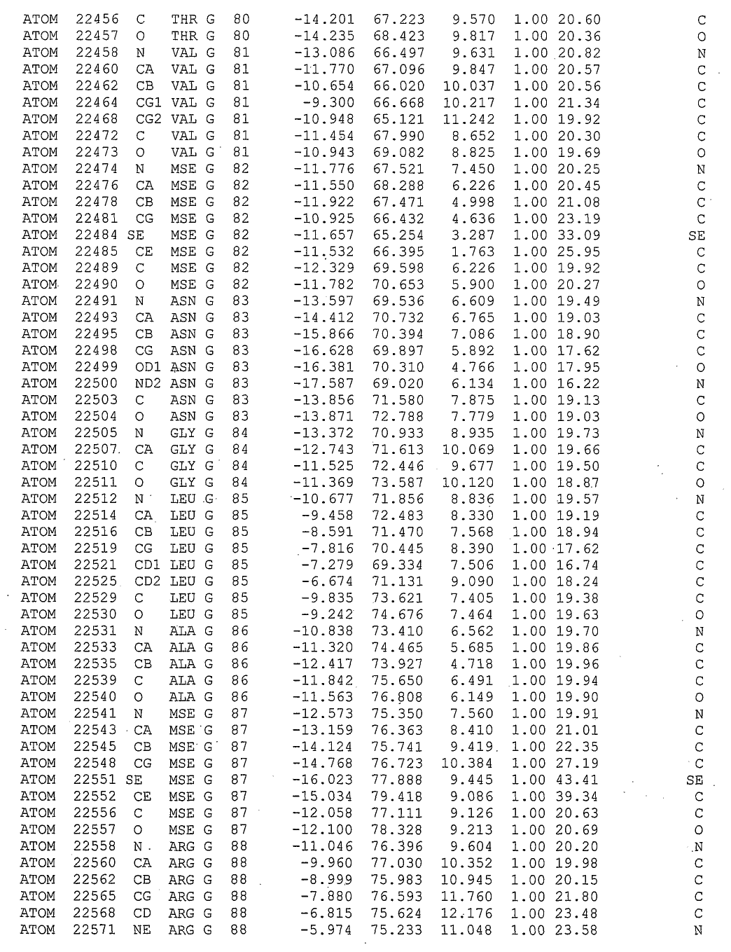

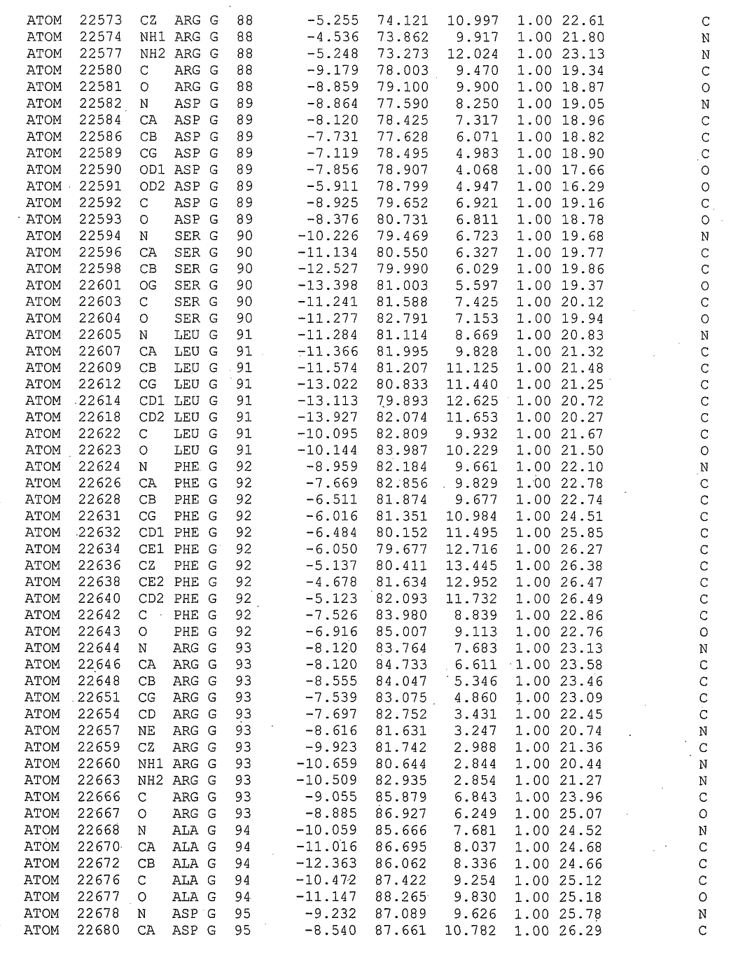

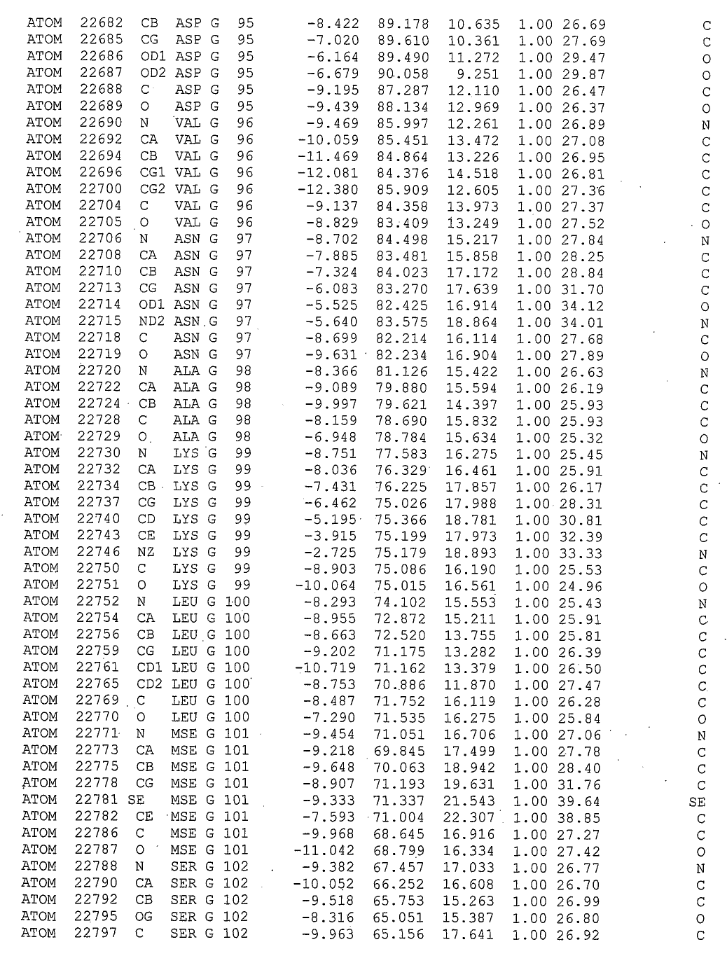

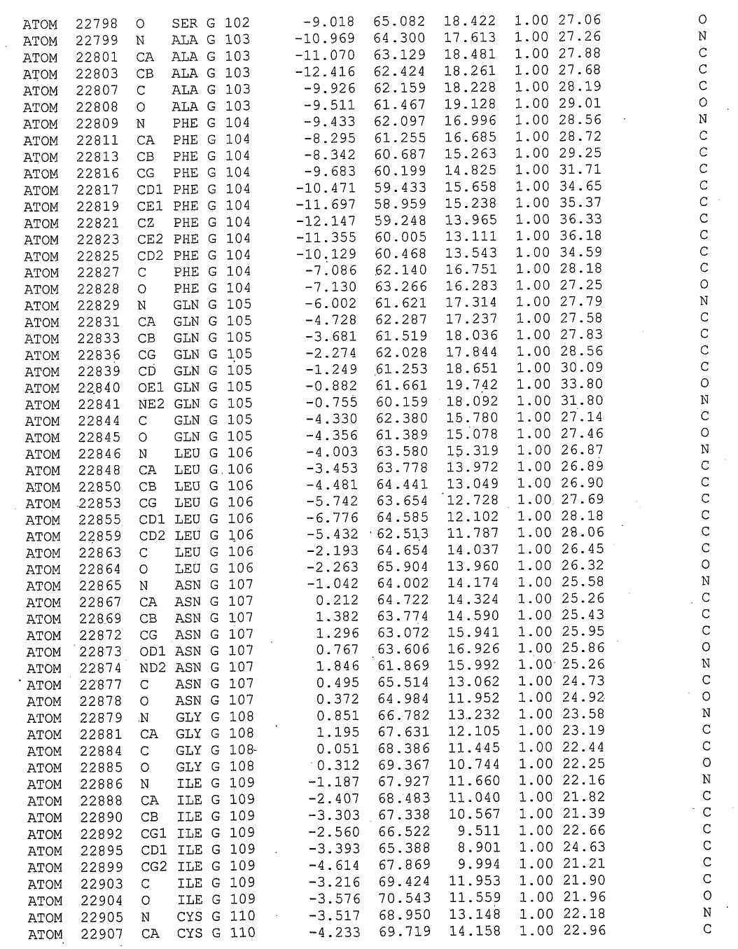

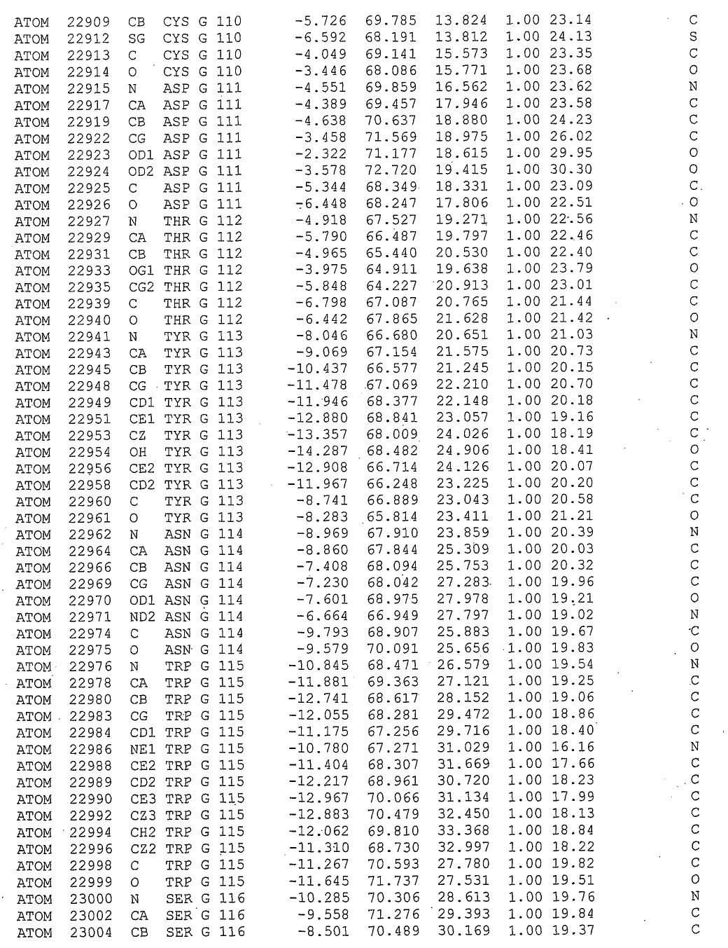

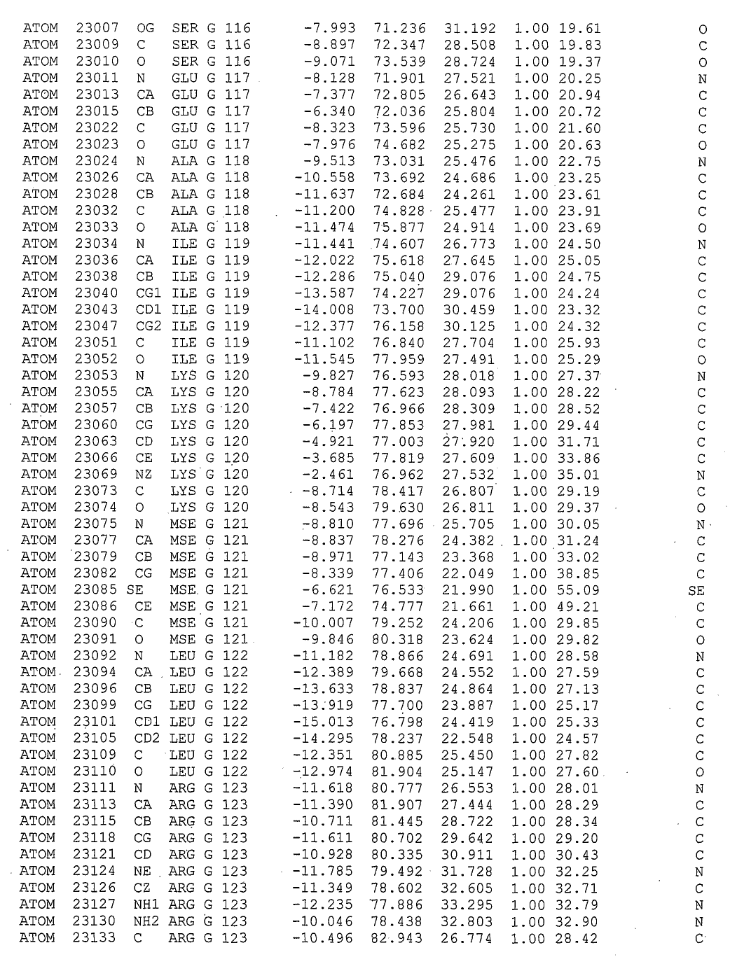

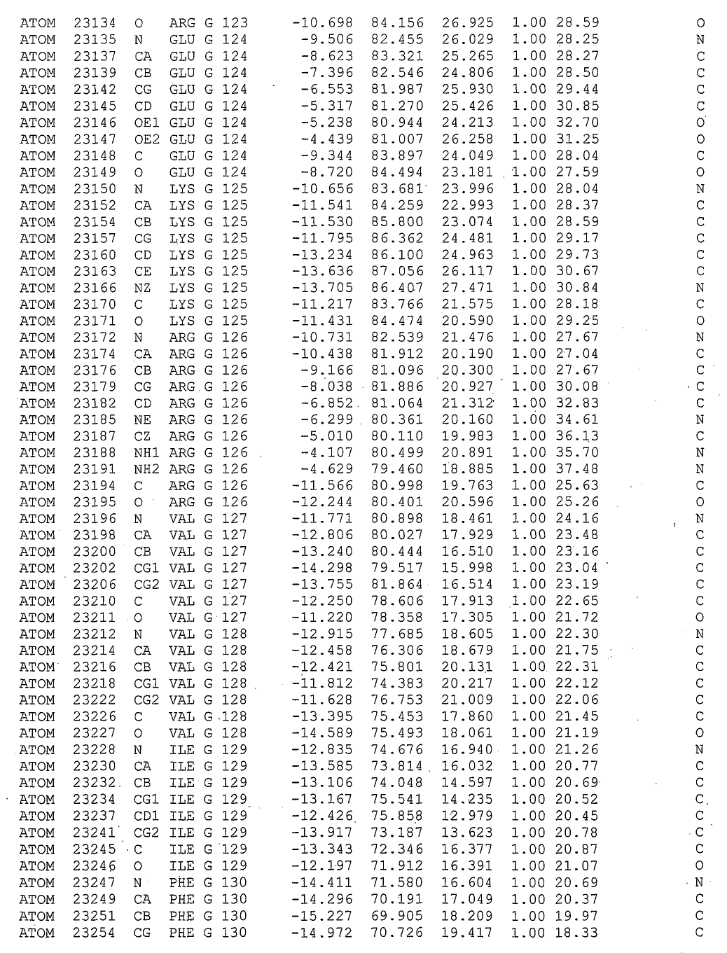

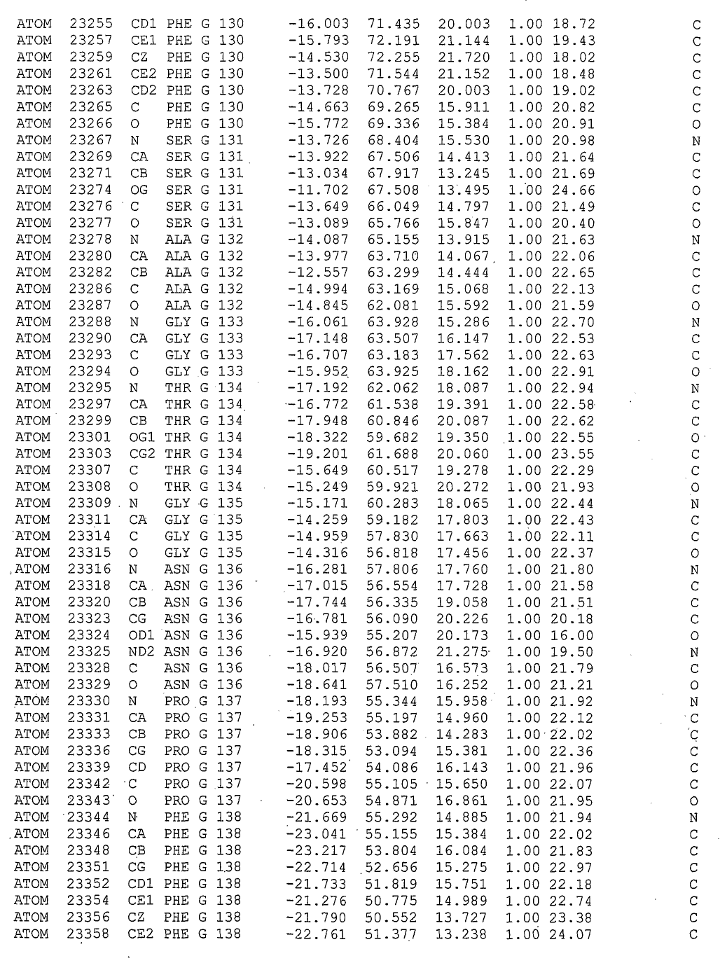

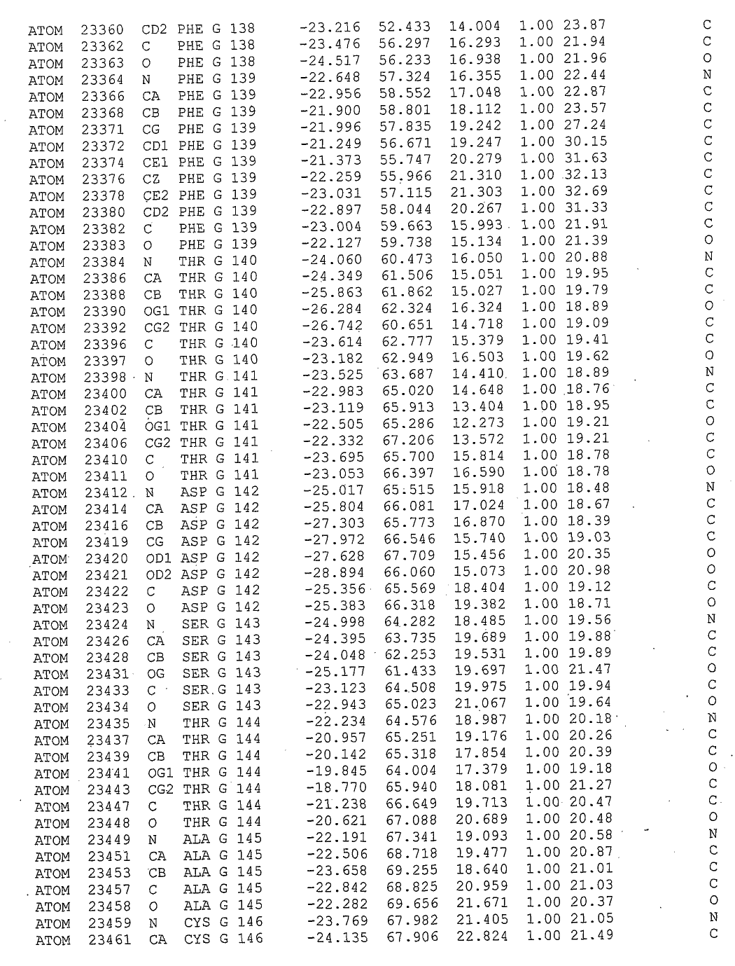

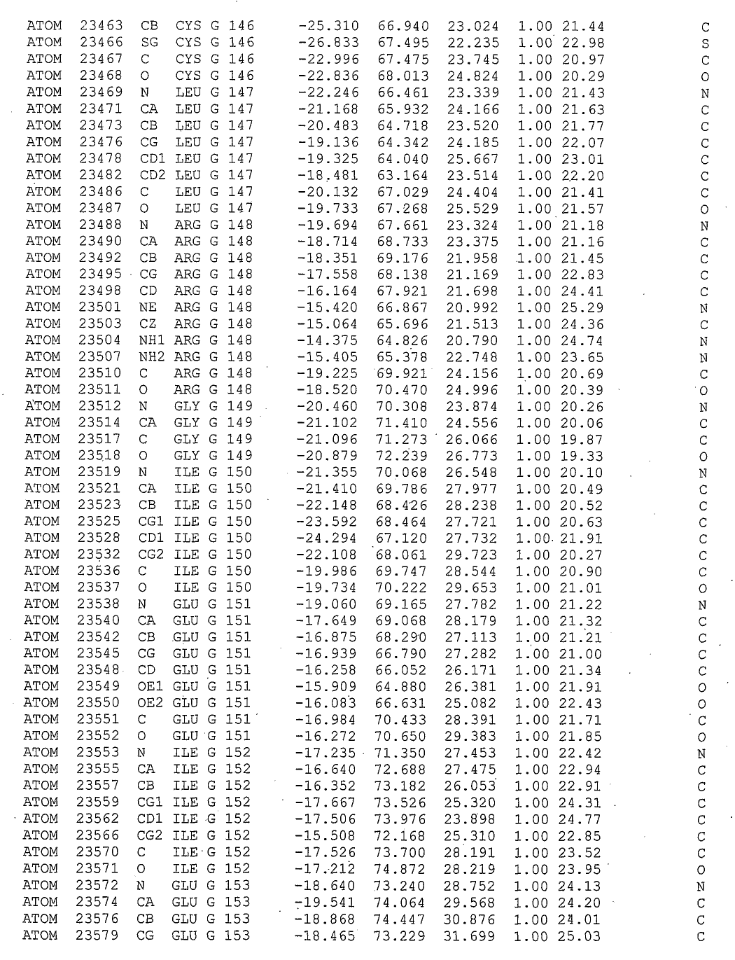

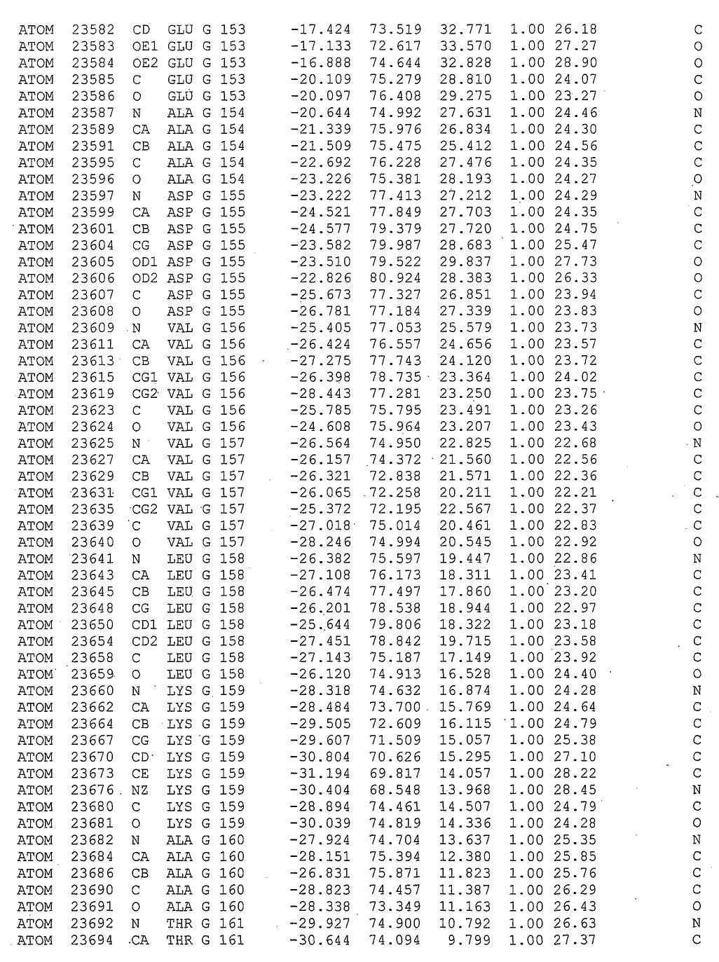

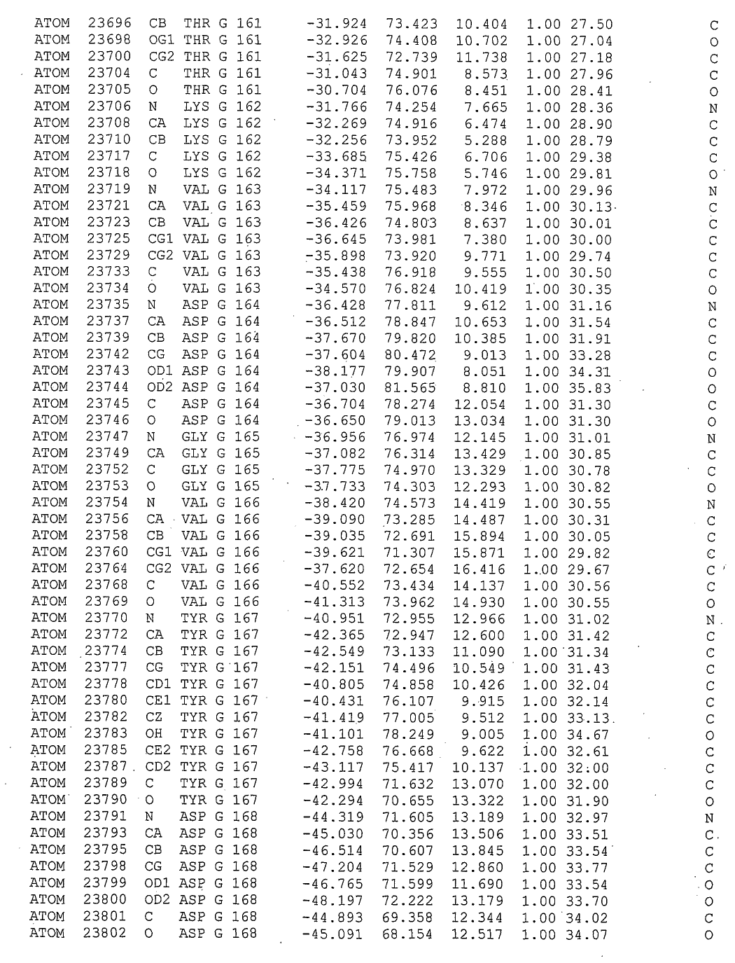

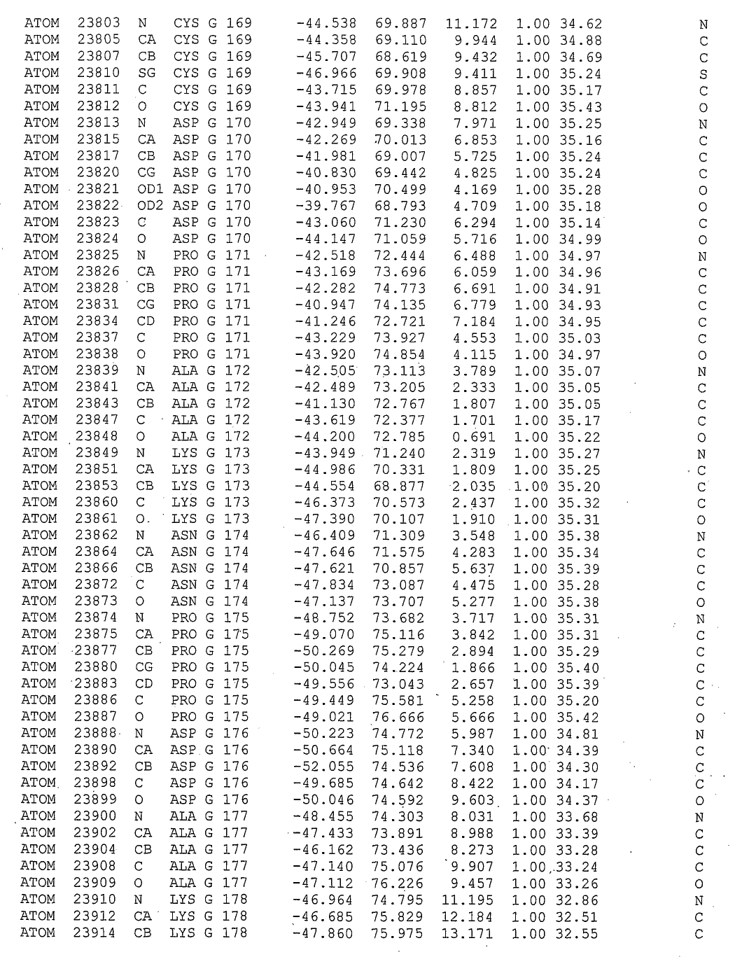

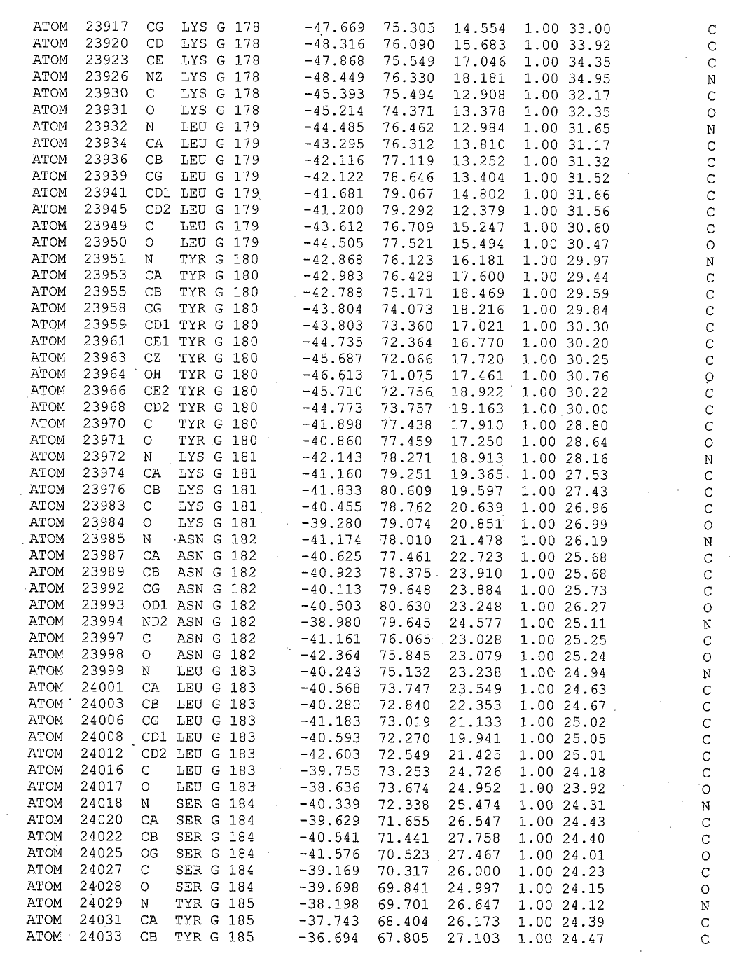

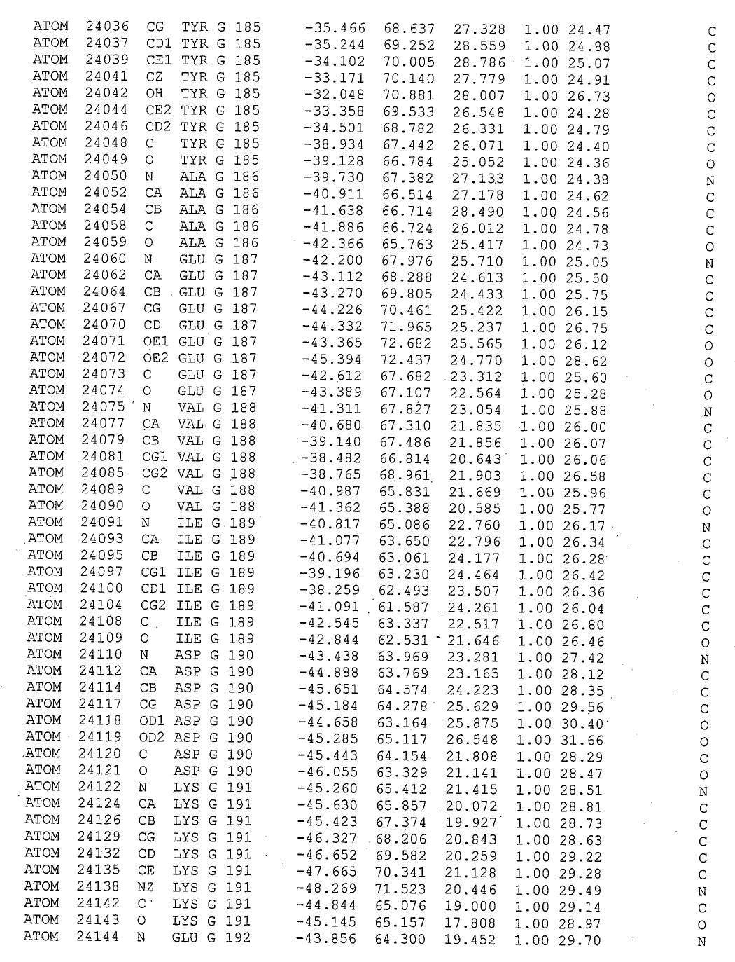

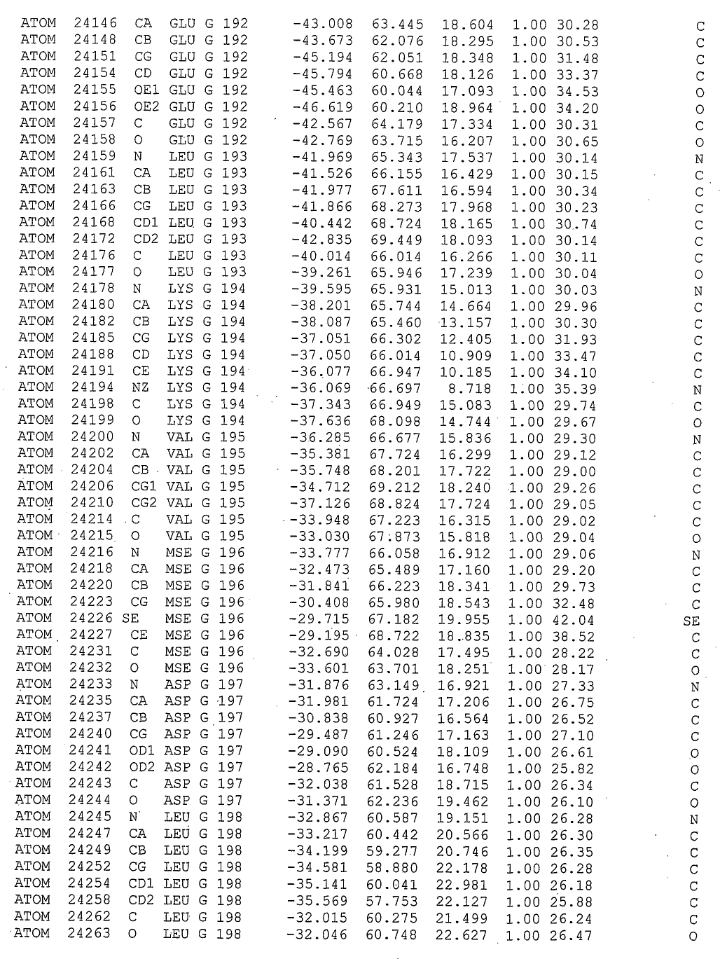

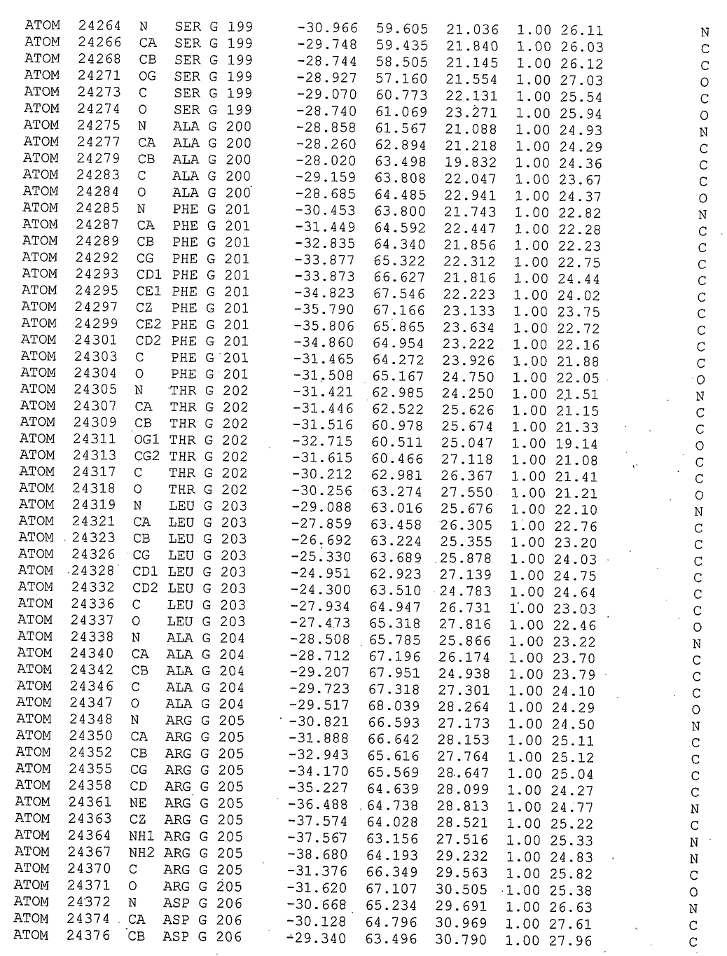

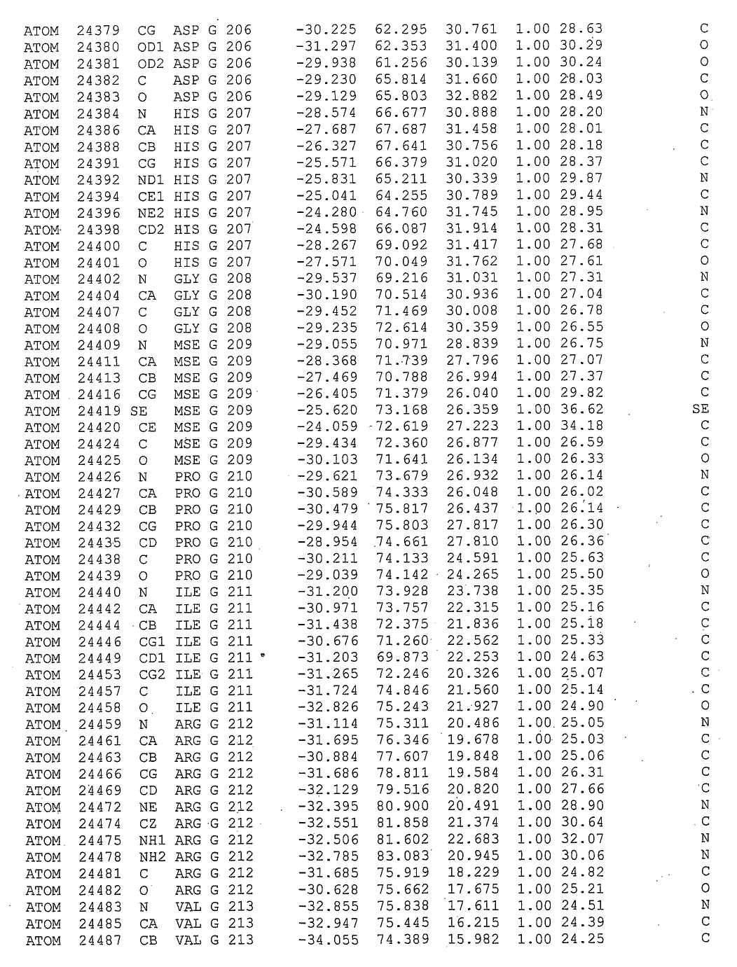

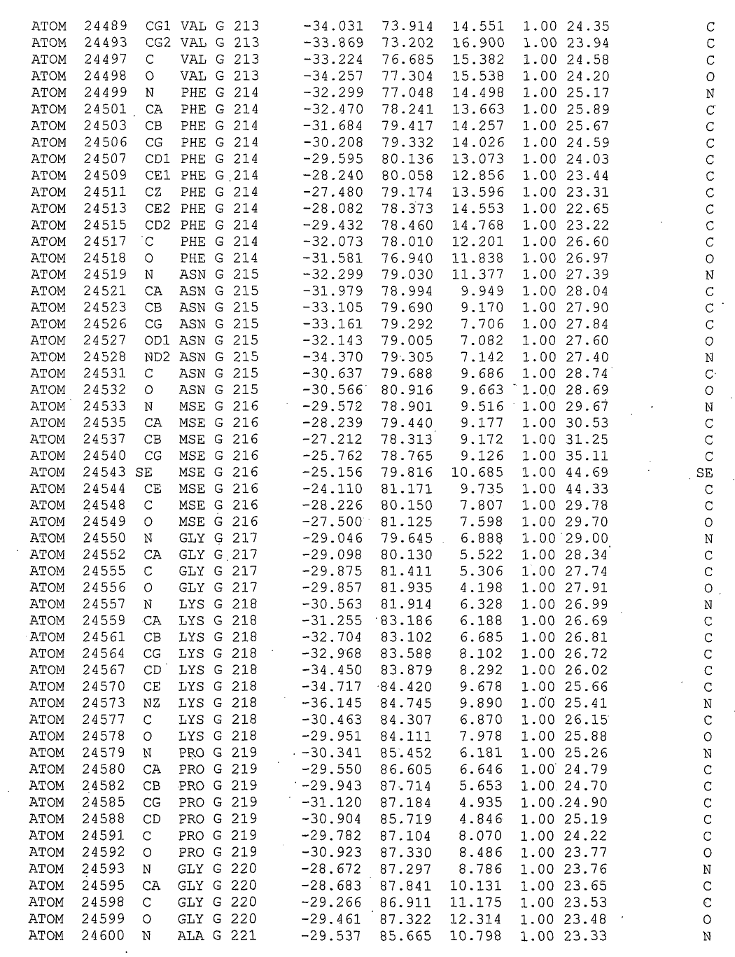

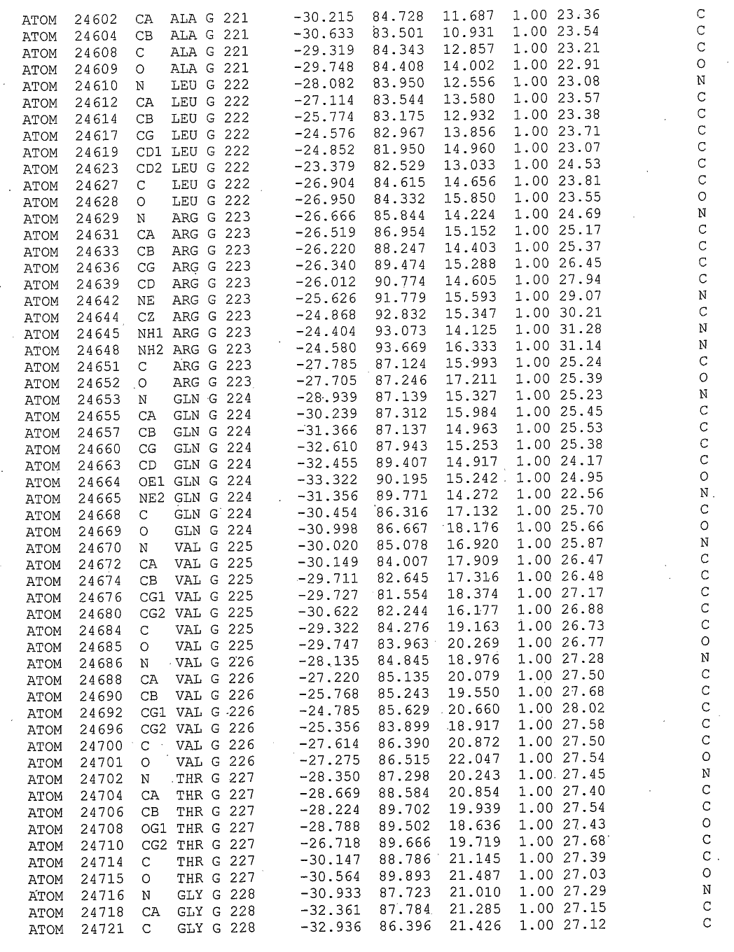

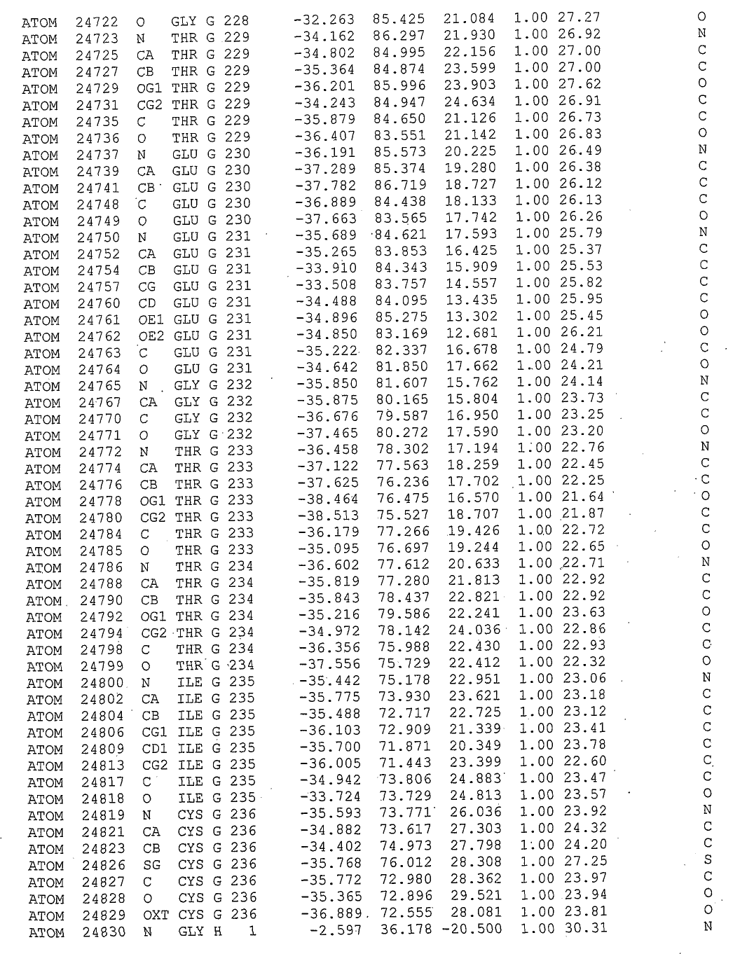

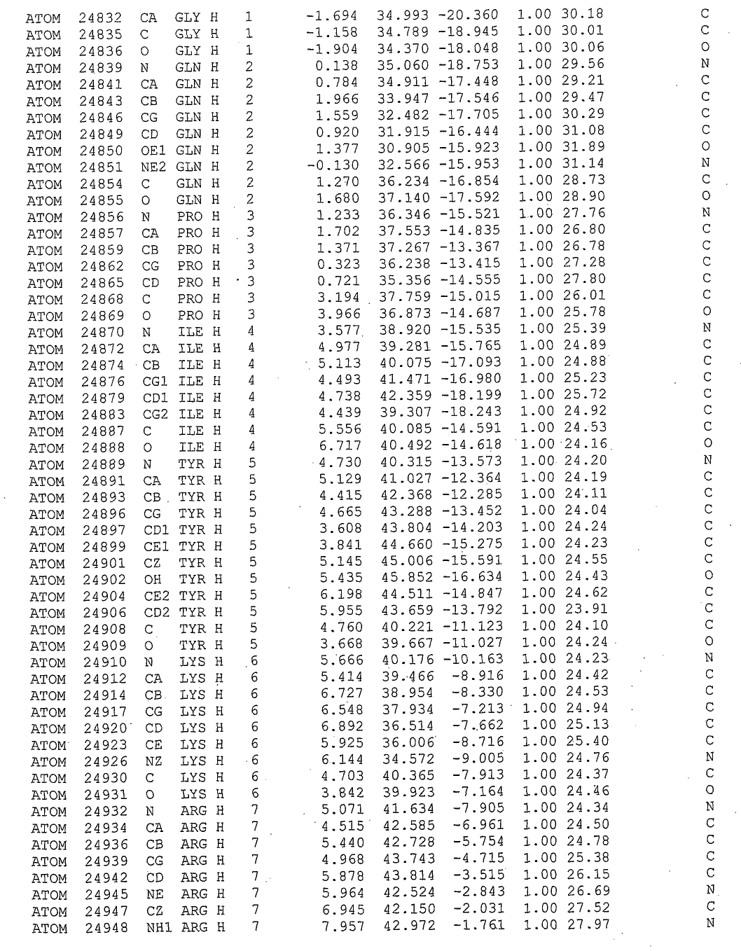

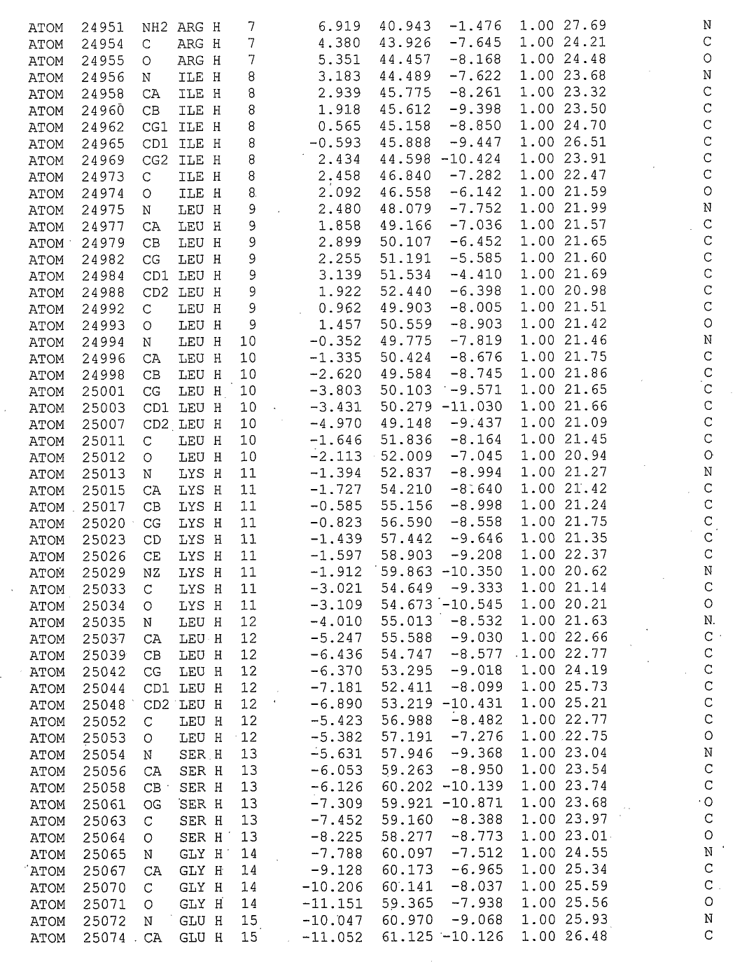

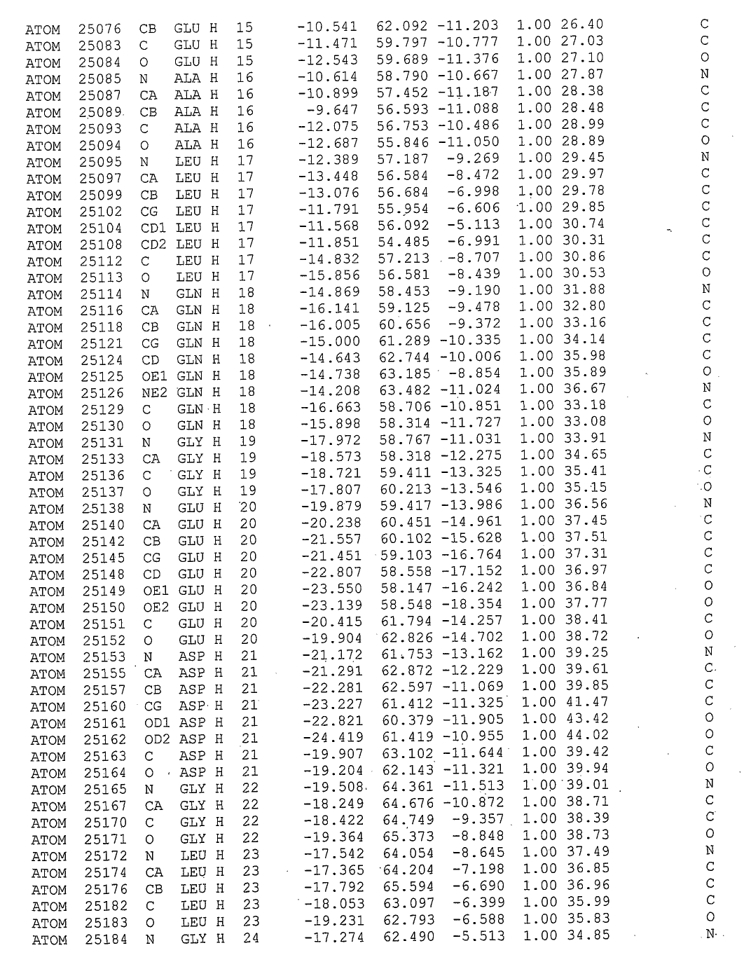

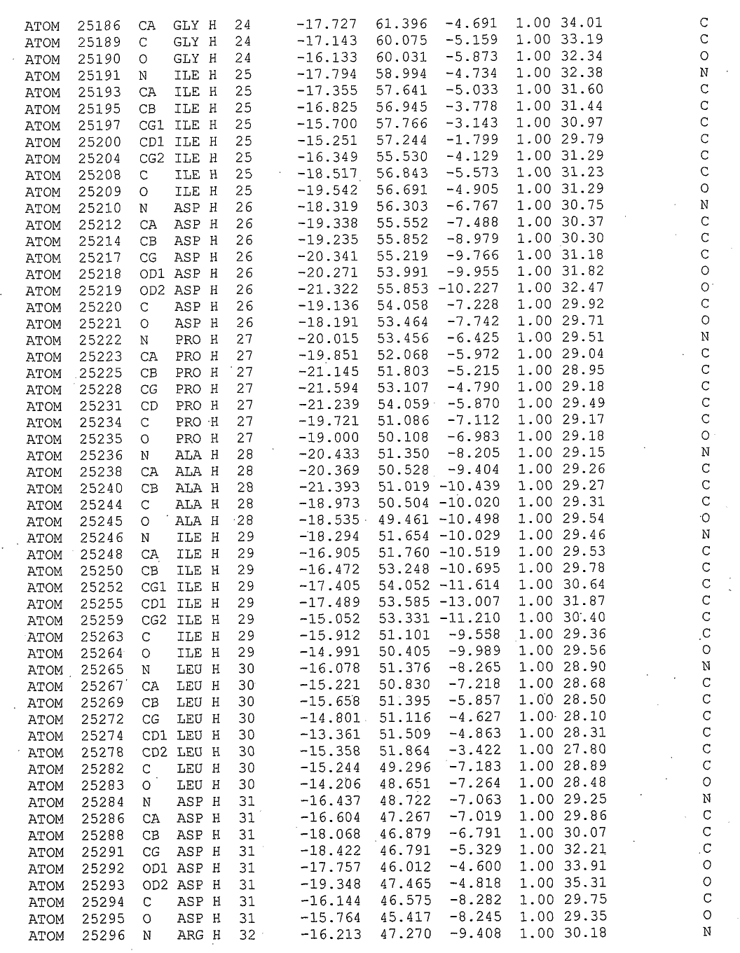

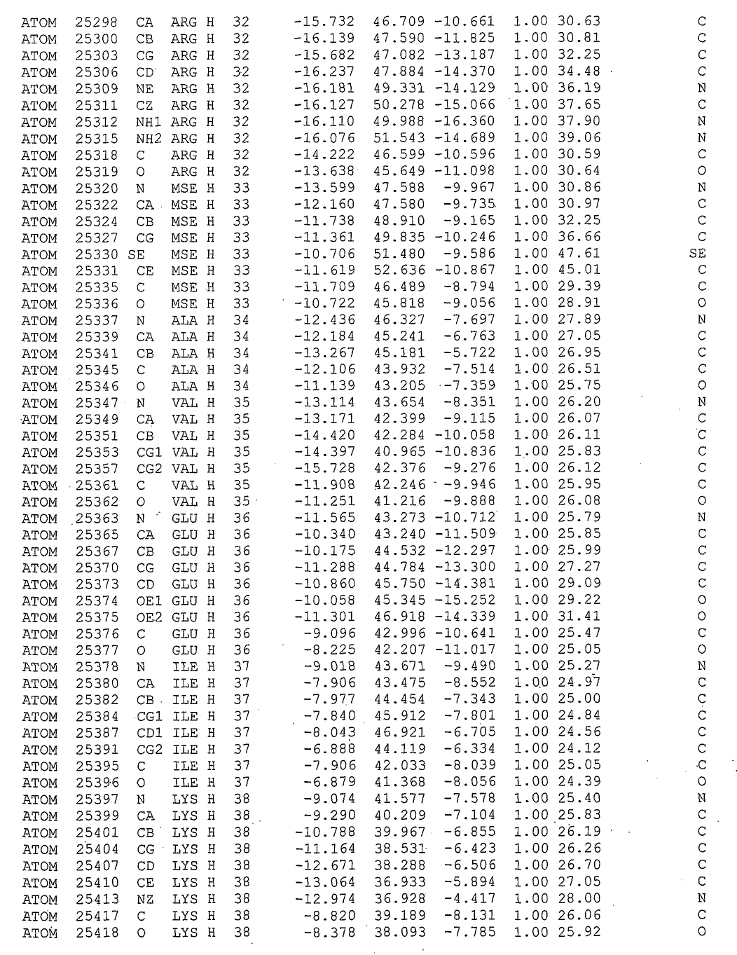

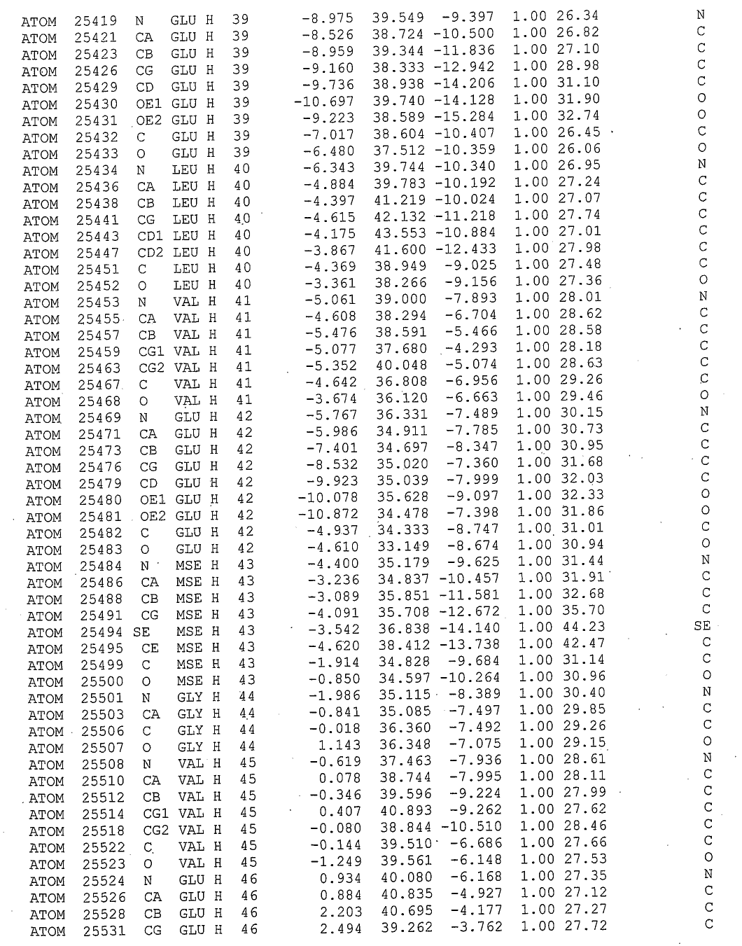

Residues located within a 5 Å radius of the bound GTP molecule include Gly66, Asn68, Val71, Leu85, Arg88, Asp89, Phe92, Arg93, Asn97, Ala98, Lys99, Leu100, Asp111, Asn114, Trp115, Ser116, Glu117, Ile119, Lys120, Met121, Arg123 and Arg126 of SEQ ID NO:14. Of those residues whose side-chains make significant polar or hydrophobic contacts with the bound GTP, Val71, Asp89, Asn97, Lys99, Lys120 and Arg126 are well conserved across 10 bacterial UMP kinases (pyrH) (see FIG. 6). The allosteric effector binding site of pyrH thus minimally comprises residues Asn68, Val71, Arg88, Asp89, Phe92, Asn97, Lys99, Trp115, Ser116, Ile119, Lys120, Arg123 and Arg126 of SEQ ID NO:14, or in amore expanded definition comprises residues Gly66, Asn68, Val71, Leu85, Arg88, Asp89, Phe92, Arg93, Asn97, Ala98, Lys99, Leu100, Asp111, Asn114, Trp115, Ser116, Glu117, Ile119, Lys120, Met121, Arg123 and Arg126 of SEQ ID NO:14, or in a yet further expanded definition, derived using a probe radius of 8 Å, comprises residues Arg7, Gly66, Met67, Asn68, Arg69, Val70, Val71, Gly72, His74, Gly84, Leu85, Ala86, Met87, Arg88, Asp89, Ser90, Leu91, Phe92, Arg93, Asp95, Val96, Asn97, Ala98, Lys99, Leu100, Met101, Ile109, Cys110, Asp111, Asn114, Trp115, Ser116, Glu117, Ala118, Ile119, Lys120, Met121, Arg123, Glu124, Arg126, Val127, Ile129, Glu151, Ile152 and Glu153.

### Phosphate Binding Site

The final model was superposed on the crystal structure of the E. faecalis carbamate kinase (pdb entry 1b7b, Marina et al., Protein Sci., 8:934-940, 1999) using the program TOP (CCP4, Acta Cryst., D50:760-763, 1994; Lu, Protein Data Bank Quarterly Newsletter, 78:10-11, 1996). TOP is an automated program for comparison of pairs of protein structures based upon their tertiary structures as defined by atomic coordinates. When homologous proteins with common secondary structural elements are available, the TOP program can automatically superimpose the three-dimensional models, detect which residues are structurally equivalent among all the structures and provide the residue-to-residue alignment. The program calculates scores of topological and structure diversity, and can produce an overlay of the structure of interest onto a reference structure. This method can be used to identify, align and compare structures that, despite showing limited primary sequence homology, display structural similarity. This showed that the bound phosphate in the H. influenzae pyrH crystal structure overlaid almost exactly on a sulphate molecule that was observed in the E. faecalis carbamate kinase structure. The bound sulphate ion in the E. faecalis carbamate kinase structure is proposed to occupy the site of the gamma-phosphate group that is transferred from ATP to the substrate, carbamate. This hypothesis is supported by the crystal structures of P. furiosus CK-like CPS in complex with MgADP (Ramón-Maiques et al., 2000, supra) and of E. coli NAGK in complex with substrates and transition state analogues (Ramón-Maiques et al., 2002, supra; Gil-Ortiz et al., 2003, supra). We therefore propose that the bound phosphate ion in the H. influenzae pyrH crystal structure occupies the site of the phosphate group transferred from ATP to UMP. Two residues equivalent to those that contact the phosphate ion in H. influenzae pyrH have been mutated in E. coli pyrH (Bucurenci et al., J. Bacteriol., 180(3):473-477, 1998). Mutations Arg62His and Asp146Asn (equivalent to H. influenzae residues Arg58 and Asp 142 respectively) resulted in reductions in the rate of catalysis. The region surrounding the phosphate ion thus likely corresponds to the active centre of the pyrH enzyme. Residues located within a 5A radius of the bound phosphate molecule include Lys11, Ser13, Gly14, Glu15, Gly52, Gly53, Gly54, Thr141 and Asp142 of SEQ ID NO:14. All of these residues are completely conserved among 10 bacterial UMP kinases, with the exception of Glu15 that is an alanine in pyrH from Mycoplasma pneumoniae (see FIG. 6). More specifically, the phosphate ion is stabilised by the formation of hydrogen bonds with the backbone amino groups of Gly14, Gly53 and Gly54, and with the sidechain hydroxyl of Thr141. In a subset of the phosphate binding sites in the crystal, the sidechain carboxyl of Glu15 also contacts a phosphate oxygen. Thus the phosphate binding site of pyrH minimally comprises residues Ser13, Gly14, Gly52, Gly53, Gly54 and Thr141 of SEQ ID NO: 14, or in a more expanded definition comprises residues Lys11, Ser13, Gly14, Glu15, Gly52, Gly53, Gly54, Thr141 and Asp142 of SEQ ID NO:14, or in a yet further expanded definition, derived using an 8 Å probe radius, includes Lys11, Leu12, Ser13, Gly14, Glu15, Ala16, Gln18, Val50, Leu51, Gly52, Gly52, Gly53, Gly54, Asn55, Leu56, Phe57, Asn58, Gly76, Met77, Ala79, Thr80, Asn83, Phe139, Thr140, Thr141, Asp142, Ile159 and Ala160 of SEQ ID NO:14.

### Putative ATP Binding Site

On the basis of superpositions with the P. furiosus CK-like CPS-ADP complex (Ramón-Maiques et al., 2000, supra) and the E. coli NAGK-substrate complexes (Ramón-Maiques et al., 2002, supra; Gil-Ortiz et al., 2003, supra) using the program TOP(CCP4, (1994, supra), an ATP-binding site is proposed. This includes amino acid residues Lys11, Leu12, Ser13, Gly14, Glu15, Ala16, Leu17, Val50, Leu51, Gly52, Gly53, Gly54, Asn55, Arg58, Thr80, Asn83, Thr140, Thr141, Asp142, Ser143, Ala145, Lys159, Ala160, Thr161, Lys162, Val163, Gly165, Val166, Tyr167, Asp168, Cys169, Asp170, Pro171, Lys173, Asp174, Ala177, Lys178, Tyr180, Lys191, Glu192, Leu193, Lys194, Val195, Met196, Asp197, Val213 and Phe214 of SEQ ID NO:14. Of these residues, Lys11, Ser13, Gly14, Gly52, Gly53, Gly54, Asn55, Thr80, Asn83, Thr141, Asp142, Val163, Gly165, Val166, Asp170, Pro171, Ala177, Leu193, Met196, Asp197 and Phe214 are completely conserved across 10 bacterial UMP kinases (pyrH). Leu12, Glu15, Ala16, Leu17, Val50, Leu51 Arg58, Ser143, Ala145, Tyr167, Val195 and Val213 also show a high level of conservation (see FIG. 6). By analogy to the structures of E. faecalis CK (Marina et al., 1999, supra), the P. furiosus CK-like CPS-ADP complex (Ramón-Maiques et al., 2000, supra) and the E. coli NAGK-substrate complexes (Ramón-Maiques et al., 2002, supra; Gil-Ortiz et al., 2003, supra), it is possible that ATP binding is accommodated by changes in the conformation of the flexible loop comprising residues 168 to 176. The formation of two hydrogen bonds between the Tyr167 backbone carbonyl and amino groups and the 6-NH₂ and N1 of adenine respectively, combined with packing of the adenine ring against the Pro171 sidechain and the Cys169-Asp170 peptide linkage, could contribute to specificity for ATP at this site. The side-chain of Asp170 could contact the 2′-hydroxyl of a bound ATP ribose, whilst polar interactions with the backbone amino groups of Gly14, Gly53, Gly54 and Thr161, and side-chain atoms of Lys11, Ser13 and Lys159 may contribute to binding of the triphosphate moiety. The triad of residues Lys11, Asp142 and Lys159 (structurally equivalent to residues Lys215, Asp216 and Lys277 in P. furiosus CK-like CPS, and to Lys8, Asp162 and Lys217 in E. coli NAGK, respectively) are likely to play a significant role in catalysing the phosphotransfer reaction. Lys11 is positioned to stabilise the pentavalent phosphorus of the transition state, and Lys159 is positioned to stabilise the developing negative charge on the beta-phosphate of the reaction product, ADP. Asp142 holds the two lysine sidechains in position, and may be involved in coordinating a magnesium ion that would complex to the triphosphate moiety of the bound substrate, ATP.

In further support of the proposed ATP binding site, mutation of residues in pyrH corresponding to those that contact the bound adenine nucleotide in P. furiosus CK-like CPS and E. coli NAGK has been shown to reduce the rate of catalysis. These mutations were Arg62H is, Asp 146Asn and Asp174Asn in E. coli pyrH (Bucurenci et al., 1998, supra). The corresponding residues in the H. influenzae pyrH are Arg58, Asp 142 and Asp170. A minimal definition of the proposed pyrH ATP-binding site thus comprises residues Lys11, Ser13, Gly14, Glu15, Gly53, Gly54, Asp142, Lys159, Thr161, Val166, Tyr167, Cys169, Asp170 and Pro171, a more expanded definition comprises residues Lys11, Leu12, Ser13, Gly14, Glu15, Ala16, Gly52, Gly53, Gly54, Arg58, Thr141, Asp142, Lys159, Ala160, Thr161, Lys162, Val163, Gly165, Val166, Tyr167, Asp168, Cys169, Asp170, Pro171, Lys191, Glu192, Leu193, Lys194, Val195 and Val213 of SEQ ID NO: 14, and a yet further expanded definition, derived using an 8 Å probe radius, comprises residues Lys11, Leu12, Ser13, Gly14, Glu15, Ala16, Leu17, Val50, Leu51, Gly52, Gly53, Gly54, Asn55, Arg58, Thr80, Asn83, Thr140, Thr141, Asp142, Ser143, Ala145, Lys159, Ala160, Thr161, Lys162, Val163, Gly165, Val166, Tyr167, Asp168, Cys169, Asp170, Pro171, Lys173, Asp174, Ala177, Lys178, Tyr180, Lys191, Glu192, Leu193, Lys194, Val195, Met196, Asp197, Val213 and Phe214 of SEQ ID NO: 14.

### Putative UMP Binding Site

On the basis of conservation of surface residues, and superpositions with the E. coli NAGK-substrate complex, pdbIohb (Ramón-Maiques et al., 2002, supra; Gil-Ortiz et al., 2003, supra), using the program TOP(CCP4, (1994, supra), a UMP-binding site is proposed. This includes amino acid residues Lys11, Leu12, Ser13, Gly14, Glu15, Val50, Leu51, Gly52, Gly53, Gly54, Asn55, Phe57, Arg58, Gly59, Arg69, Val70, Val71, Gly72, Asp73, His74, Met75, Gly76, Met77, Leu78, Ala79, Thr80, Asn83, Ala103, Phe104, Ser131, Ala132, Gly133, Thr134, Gly135, Asn136, Pro137, Phe138, Phe139, Thr140, Thr141, Asp142, Ser143, Thr144, Ala145, Leu147 and Arg148 of SEQ ID NO:14. All of these residues are highly conserved across 10 bacterial UMP kinases (pyrH) (see FIG. 6). Comparison of the conformations of the eight molecules in the crystal asymmetric unit demonstrates flexibility in the positions of certain secondary structural elements surrounding the proposed UMP binding site, namely helix B (residues 60-65), and the subsequent loop (alphaB-alphaC) that links helix B to helix C (residues 66-69). It is possible that UMP binding is accommodated by changes in the conformation of these flexible regions, and that binding of UMP may therefore be influenced by the binding of allosteric modulators at the allosteric binding site. In further support of the proposed UMP binding site, mutation of residues within the proposed binding site has been shown to reduce the rate of catalysis. These mutations were Arg62H is, Asp73Asn and Asp142Asn in E. coli pyrH (Bucurenci et al., 1998, supra). The corresponding residues in the H. influenzae pyrH are Arg58, Asp73 and Asp142. A minimal definition of the proposed pyrH UMP-binding site thus comprises residues Gly52, Gly53, Asp73, Gly76, Met77, Thr134, Asn136, Pro137, Phe139, Thr141 and Thr144, a more expanded definition comprises residues Ser13, Gly14, Gly52, Gly53, Gly54, Gly72, Asp73, His 74, Gly76, Met77, Thr80, Gly133, Thr134, Asn136, Pro137, Phe138, Phe139 and Thr140 of SEQ ID NO: 14, and a yet further expanded definition, derived using an 8 Å probe radius, comprises residues Lys11, Leu12, Ser13, Gly14, Glu15, Val50, Leu51, Gly52, Gly53, Gly54, Asn55, Phe57, Arg58, Gly59, Arg69, Val70, Val71, Gly72, Asp73, His74, Met75, Gly76, Met77, Leu78, Ala79, Thr80, Asn83, Ala103, Phe104, Ser131, Ala132, Gly133, Thr134, Gly135, Asn136, Pro137, Phe138, Phe139, Thr140, Thr141, Asp142, Ser143, Thr144, Ala145, Leu147 and Arg148 of SEQ ID NO: 14.

### Example 7

### Nuclear Magnetic Resonance Spectroscopy Studies of H. influenzae PyrH

Nuclear magnetic resonance (NMR) spectroscopy provides a method to monitor, at the amino acid level, the structure and conformation of a protein in solution. The position of the signals in the spectra is extremely sensitive to the environment of the amino acids, and changes in the position of these signals can be correlated with interactions between the protein and another molecule.

NMR spectroscopy experiments on H. influenzae pyrH were performed at 298K on a Bruker Avance 600 MHz system equipped with a triple resonance (¹H/¹³C/¹⁵N) single-gradient 5 mm cryoprobe. The protein was triply labelled in ²H, ¹⁵N and ¹³C and was provided in Buffer B (50 mM Tris/HCl, pH 8.0, 2 mM EDTA, 2 mM UTP, 150 mM NaCl and 2 mM DTT). Prior to the NMR spectroscopy experiments, protein samples were extensively dialyzed, using Amicon Ultra-15 centrifugal filter devices from Millipore (Billerica, Mass., USA), into the NMR buffer (50 mM HEPES, pH 7.8, 1 mM DTT, and 1 mM UTP or 1 mM GTP). Protein monomer concentration was 0.4 mM. TROSY-HSQC (Pervushin et al., J. Biomol. NMR, 12:345-348, 1998) experiments, were recorded with evolution times of 85 milliseconds in the proton dimension and 32 milliseconds in the nitrogen dimension. The total acquisition time was 15 hours. Data sets were processed with the program nmrPipe (Delaglio et al., J. Biomol. NMR, 6:277-293, 1995) and analyzed with the program SPARKY (Goddard and Kneller, University of California, San Francisco, USA).

The quality of the spectra obtained for H. influenzae pyrH using this protocol is very high, and the expected number of peaks for a protein of this size were evident. This assay was sensitive enough to detect changes in the environment of the protein when UTP was replaced by GTP in the NMR buffer. Thus, NMR can be used to validate hits from high through-put and virtual screening campaigns aimed at the discovery of specific inhibitors of pyrH. Furthermore, NMR can be exploited in structure-based drug design in combination with high-resolution X-ray data. NMR can be used to confirm binding of inhibitors to PyrH.

The foregoing examples are meant to illustrate the invention and are not to be construed to limit the invention in any way. Those skilled in the art will recognize modifications that are within the spirit and scope of the invention.

We claim:

1. A crystal of pyrH.

2. The crystal of claim 1 complexed with an allosteric modulator.

3. The crystal of claim 2, wherein the crystal is further complexed with a phosphate.

4. The crystal of claim 1 pyrH complexed with a substrate.

5. The crystal of claim 4, wherein the substrate is ATP.

6. The crystal of claim 1, wherein the pyrH is from a bacterium.

7. The crystal of claim 6, wherein the pyrH is from Haemophilus influenzae.

8. The crystal of claim 2, wherein said allosteric modulator is bound at a binding site comprising all or any combination of amino acid residues Arg7, Gly66, Met67, Asn68, Arg69, Val70, Val71, Gly72, His74, Gly84, Leu85, Ala86, Met87, Arg88, Asp89, Ser90, Leu91, Phe92, Arg93, Asp95, Val96, Asn97, Ala98, Lys99, Leu100, Met101, Ile109, Cys110, Asp111, Asn114, Trp115, Ser116, Glu117, Ala118, Ile119, Lys120, Met121, Arg123, Glu124, Arg126, Val127, Ile129, Glu151, Ile152 and Glu153 of SEQ ID NO: 14.

9. The crystal of claim 4, wherein said substrate is bound at a binding site comprising all or any combination of amino acid residues Lys11, Leu12, Ser13, Gly14, Glu15, Ala16, Leu17, Val50, Leu51, Gly52, Gly53, Gly54, Asn55, Arg58, Thr80, Asn83, Thr140, Thr141, Asp142, Ser143, Ala145, Lys159, Ala160, Thr161, Lys162, Val163, Gly165, Val166, Tyr167, Asp168, Cys169, Asp170, Pro171, Lys173, Asp174, Ala177, Lys178, Tyr180, Lys191, Glu192, Leu193, Lys194, Val195, Met196, Asp197, Val213 and Phe214 of SEQ ID NO: 14.

10. The crystal of claim 4, wherein said substrate is bound at a binding site comprising all or any combination of amino acid residues Lys11, Leu12, Ser13, Gly14, Glu15, Val50, Leu51, Gly52, Gly53, Gly54, Asn55, Phe57, Arg58, Gly59, Arg69, Val70, Val71, Gly72, Asp73, His74, Met75, Gly76, Met77, Leu78, Ala79, Thr80, Asn83, Ala103, Phe104, Ser131, Ala132, Gly133, Thr134, Gly135, Asn136, Pro137, Phe138, Phe139, Thr140, Thr141, Asp142, Ser143, Thr144, Ala145, Leu147 and Arg148 of SEQ ID NO: 14.

11. The crystal of claim 4, wherein said substrate is bound at a binding site comprising all or any combination of amino acid residues Lys11, Leu12, Ser13, Gly14, Glu15, Ala16, Leu17, Val50, Leu51, Gly52, Gly53, Gly54, Asn55, Phe57, Arg58, Gly59, Arg69, Val70, Val71, Gly72, Asp73, His74, Met75, Gly76, Met77, Leu78, Ala79, Thr80, Asn83, Ala103, Phe104, Ser131, Ala132, Gly133, Thr134, Gly135, Asn136, Pro137, Phe138, Phe139, Thr140, Thr141, Asp142, Ser143, Thr144, Ala145, Leu147, Arg148, Lys159, Ala160, Thr161, Lys162, Val163, Gly165, Val166, Tyr167, Asp168, Cys169, Asp170, Pro171, Lys173, Asp174, Ala177, Lys178, Tyr180, Lys191, Glu192, Leu193, Lys194, Val195, Met196, Asp197, Val213 and Phe214 of SEQ ID NO: 14.

12. The crystal of claim 4, wherein said substrate is bound at a binding site comprising all or any combination of amino acid residues Lys11, Ser13, Gly14, Glu15, Gly52, Gly53, Gly54, Thr141 and Asp142 of SEQ ID NO: 14.

13. The crystal of claim 1, wherein the crystal comprises three dimers, each dimer comprising an intermolecular dimer interface comprising all or any combination of amino acid residues Ile25, Pro27, Leu30, Asp31, Phe57, Lys61, Leu62, Ala65, Gly66, Met67, Asn68, Arg69, Val71, His74, Met75, Gly76, Leu78, Ala79, Val81, Met82, Leu85, Ala86, Arg87, Asp88, Arg89, Phe104, Gln105, Leu106, Asn107, Gly108 and Ile109 of SEQ ID NO: 14.

14. A method of identifying a molecule that binds to pyrH comprising
a) applying a 3-dimensional molecular modeling algorithm to atomic coordinates of pyrH; and
b) electronically screening stored atomic coordinates of a set of candidate compounds against the atomic coordinates of pyrH to identify compounds that bind to pyrH.

15. The method of claim 14, wherein the atomic coordinates are of a molecular interface of pyrH.

16. (canceled)

17. A computer-assisted method of identifying an agent that is an inhibitor of pyrH, comprising:
(a) providing a computer modeling application with a set of atomic coordinates of a crystal of pyrH;
(b) supplying the computer modeling application with a set of atomic coordinates of an agent to be assessed to determine if it binds a molecular interface of pyrH;
(c) comparing the two sets of atomic coordinates; and
(d) determining whether the agent is expected to bind to pyrH; wherein if the agent is expected to bind to pyrH, the agent is an inhibitor of pyrH.

18. The method of claim 17, wherein the set of atomic coordinates is given in FIG. 7.

19. The method of claim 24, wherein the set of atomic coordinates is given in FIG. 7.

20. A computer-assisted method of identifying an agent that is an allosteric modulator of pyrH, comprising:
(a) providing a computer modeling application with a set of atomic coordinates of a crystal of pyrH, or of an allosteric modulator binding site thereof,
(b) supplying the computer modeling application with a set of atomic coordinates of an agent to be assessed to determine if it binds a molecular interface of pyrH;
(c) comparing the two sets of atomic coordinates; and
(d) determining whether the agent is expected to bind to pyrH;
wherein if the agent is expected to bind an allosteric modulator binding site of pyrH, the agent is an allosteric modulator of pyrH.

21. The computer-assisted method of claim 20, wherein the set of atomic coordinates is given in FIG. 7.

22. (canceled)

23. (canceled)

24. A computer-assisted method for designing an inhibitor of pyrH activity, comprising:
(a) supplying to a computer modeling application a set of atomic coordinates of pyrH;
(b) computationally building an agent represented by a set of atomic coordinates; and
(c) determining whether the agent is expected to bind to pyrH, wherein if the agent is expected to bind pyrH, the agent is an inhibitor of pyrH.


SEQUENCE LISTING (14 sequences)

SEQ ID NO: 1 - protein, 237 aa, Streptococcus pneumoniae
MANPKYKRILIKLSGEALAGERGVGIDIQTVQTIAKEIQEVHSLGIEIALVIGGGNLWRGEPAAEAGMDRVQADYTGMLGTVMNALVMADSLQQVGVDTRVQTAIAMQQVAEPYVRGRALRHLEKGRIVIFGAGIGSPYFSTDTTAALRAAEIEADAILMAKNGVDGVYNADPKKDKTAVKFEELTHRDVINKGLRIMDSTASTLSMDNDIDLVVFNMNQSGNIKRVVFGENIGTTV

SEQ ID NO: 2 - protein, 238 aa, Streptococcus pyogenes
EPKYQRILIKLSGEALAGEKGVGIDIPTVQAIAKEIAEVHVSGVQIALVIGGGNLWRGEPAADAGMDRVQADYTGMLGTVMNALVMADSLQHYGVDTRVQTAIPMQNVAEPYIRGRALRHLEKNRIVVFGAGIGSPYFSTDTTAALRAAEIEADAILMAKNGVDGVYNADPKKDANAVKFDELTHGEVIKRGLKIMDATASTLSMDNDIDLVVFNMNEAGNIQRVVFGEHIGTTVSNK

SEQ ID NO: 3 - protein, 233 aa, Staphylococcus aureus
KYKRVVLKLSGEALAGEKGFGINPVIIKSVAEQVAEVAKMDCEIAVIVGGGNIWRGKTGSDLGMDRGTADYMGMLATVMNALALQDSLEQLDCDTRVLTSIEMKQVAEPYIRRRAIRHLEKKRVVIFAAGIGNPYFSTDTTAALRAAEVEADVILMGKNNVDGVYSADPKVNKDAVKYEHLTHIQMLQEGLQVMDSTASSFCMDNNIPLTVFSIMEEGNIKRAVMGEKIGTLI

SEQ ID NO: 4 - protein, 240 aa, Staphylococcus epidermidis
MAQTSKYKRVVLKLSGEALAGDKGFGINPIIIKSVAQQVAEVAKMDCEIAVIVGGGNIWRGKTGSDLGMDRGTADYMGMLATVMNALALQDSLEQLDCDTRVLTSIEMKQVAEPYIRRRAIRHLEKKRVVIFAAGIGNPYFSTDTTAALRAAEVEADVILMGKNNVDGVYSADPKVDANAIKYEHLTHIQMLQEGLQVMDSTASSFCMDNNIPLNVFSIMEEGNIKRAVMGEKIGTLITK

SEQ ID NO: 5 - protein, 240 aa, Bacillus subtilis
MEKPKYKRIVLKLSGEALAGEQGNGINPTVIQSIAKQVKEIAELEVEVAVVVGGGNYGAEKTGSDLGMDRATADYMGMLATVMNSLALQDSLETLGIQSRVQTSIEMRQVAEPYIRRKAIRHLEKKRVVIFAAGTGNPYFSTDTTAALRAAEIEADVILMAKNNVDGVYNADPRKDESAVKYESLSYLDVLKDGLEVMDSTASSLCMDNDIPLIVFSIMEEGNIKRAVIGESIGTIVRGK

SEQ ID NO: 6 - protein, 239 aa, Neisseria meningitides
MTQQIKYKRVLLKLSGESLMGSDPFGINHDTIVQTVGEIAEVVKMGVQVGIVVGGGNIFRGVSAQAGSMDRATADYMGMMATVMNALALKDAFETLGIKARVQSALSMQQIAETYARPKAIQYLEEGKVVIFAAGTGNPFFTTDTAAALRGAEMNCDVMLKATNVDGVYTADPKKDPSATRYETITFDEALLKNLKVMDATAFALCRERKLNIVVFGIAKEGSLKRVITGEDEGTLVHC

SEQ ID NO: 7 - protein, 241 aa, Escherichia coli
MATNAKPVYKRILLKLSGEALQGTEGFGIDASILDRMAQEIKELVELGIQVGVVIGGGNLFRGAGLAKAGMNRVVGDHMGMLATVMNGLAMRDALHRAYVNARLMSAIPLNGVCDSYSWAEAISLLRNNRVVILSAGTGNPFFTTDSAACLRGIEIEADVVLKATKVDGVFTADPAKDPTATMYEQLTYSEVLEKELKVMDLAAFTLARDHKLPIRVFNMNKPGALRRVVMGEKEGTLITE

SEQ ID NO: 8 - protein, 237 aa, Haemophilus influenzae
MSQPIYKRILLKLSGEALQGEDGLGIDPAILDRMAVEIKELVEMGVEVSVVLGGGNLFRGAKLAKAGMNRVVGDHMGMLATVMNGLAMRDSLFRADVNAKLMSAFQLNGICDTYNWSEAIKMLREKRVVIFSAGTGNPFFTTDSTACLRGIEIEADVVLKATKVDGVYDCDPAKNPDAKLYKNLSYAEVIDKELKVMDLSAFTLARDHGMPIRVFNMGKPGALRQVVTGTEEGTTIC

SEQ ID NO: 9 - protein, 248 aa, Chlamydia pneumoniae
MAKQTRRVLFKISGEALSKDSSNRIDEMRLSRLVSELRAVRNNDIEIALVIGGGNILRGLAEQKELQINRVSADQMGMLATLINGMAVADALKAEDIPCLLTSTLSCPQLADLYTPQKSIEALDQGKILICTTGAGSPYLTTDTGAALRACELNVDVLIKATMHVDGVYDKDPRLFPDAVKYDFVSYKDFLSNQLGVMDASAISLCMDSHIPIRVFSFLQHSLEKALFDPTIGTLVSEDVNHVCSPRH

SEQ ID NO: 10 - protein, 235 aa, Mycoplasma pneumoniae
MRLKILIKLSGAGMSADSSEPFSNQFLETVIAQLKQLVPNYQIGIVIGGGNIMRGKSCSDYNITEIAGHHLGIMATVINGAFLKAKFDSHKLNSTLLSAVSCPSLATHIVSQTTIDDAFKNHDIVIFAGGTGNPYFSTDTAVALRATQMQADIILIGKNGVDGVYTADPKKDKHAKFLASLTYAEAIKNDLQIMDITAFTMCKENNLKVIIFNINAEHAIIKALSKQGKYTLIEK

SEQ ID NO: 11 - DNA, 32 bp, Artificial
  Primer1
gaaaaaaccatgggccaaccaatttataaacg

SEQ ID NO: 12 - DNA, 34 bp, Artificial
  Primer2
ttcttgtcgacattcactaacaaatagtggtgcc

SEQ ID NO: 13 - DNA, 714 bp, Haemophilus influenzae
atgggccaaccaatttataaacgtattttattgaaattaagcggtgaagcattacaaggagaagatggtcttggtatcgatcctgcgattctcgatcgtatggctgttgaaattaaagaattagtggagatgggtgtggaagtcagtgtcgttctcggtggtggcaacttattccgtggcgcaaaactagcaaaagcggggatgaatcgcgtggtgggcgatcatatgggaatgcttgctactgtgatgaatggtttggcaatgcgtgattctttattccgtgctgatgtgaacgcaaaattaatgtccgctttccaattaaatggtatttgcgatacttataactggtctgaagctatcaaaatgttacgcgaaaaacgcgtagtcattttctctgcgggaacgggaaatccattctttaccactgattctaccgcttgtttgcgtggtattgaaattgaagctgatgttgtgttgaaagcgactaaagttgatggtgtgtatgattgtgatcctgcgaaaaatcctgatgcaaaactttataaaaatttaagttatgcagaagtgatcgataaagaattaaaagtgatggacttatcggcgtttactttagctcgcgatcatggcatgccgattagagtgttcaatatgggtaaacctggagcattacgtcaagtagtgactggtactgaagaaggcaccactatttgttag

SEQ ID NO: 14 - protein, 237 aa, H. influenzae
MGQPIYKRILLKLSGEALQGEDGLGIDPAILDRMAVEIKELVEMGVEVSVVLGGGNLFRGAKLAKAGMNRVVGDHMGMLATVMNGLAMRDSLFRADVNAKLMSAFQLNGICDTYNWSEAIKMLREKRVVIFSAGTGNPFFTTDSTACLRGIEIEADVVLKATKVDGVYDCDPAKNPDAKLYKNLSYAEVIDKELKVMDLSAFTLARDHGMPIRVFNMGKPGALRQVVTGTEEGTTIC